SEQUENCE LISTING (2 sequences)

SEQ ID NO: 1 - DNA, 20 bp, synthetic construct
  source 1..20: mol_type = other DNA
acaaagaaccctaacaccag

SEQ ID NO: 2 - DNA, 22 bp, synthetic construct
  source 1..22: mol_type = other DNA
  modified_base 11: mod_base = OTHER; note = Biotin modified nucleotide
aacacatctctgccaaacccca

(19) United States
(12) Patent Application Publication
(10) Pub. No.: US 2025/0340516 A1
(43) Pub. Date: Nov. 6, 2025

(54) Isoquinolinones as Modulators of POLRMT

(71) Applicant: Pretzel Therapeutics, Inc., Waltham, MA (US)

(72) Inventor: Andrew Mark Griffin, Quebec (CA)

(73) Assignee: Pretzel Therapeutics, Inc., Waltham, MA (US)

(21) Appl. No.: 19/197,052

(22) Filed: May 2, 2025

Related U.S. Application Data
(60) Provisional application No. 63/643,154, filed on May 6, 2024.

(51) Int. Cl. C07D 217/24; C07D 401/12; C07D 413/12

(52) U.S. Cl. CPC C07D 217/24; C07D 401/12; C07D 413/12

(57) ABSTRACT

The present invention provides novel isoquinolinone compounds that are inhibitors of mitochondrial RNA polymerase for treating various diseases such as cancer and others associated with metabolic disorders and mitochondnal dysfunction.

### SEQUENCE LISTING

The instant application contains a Sequence Listing which has been submitted electronically in XML format and is hereby incorporated by reference in its entirety. Said XML copy, created on Jun. 24, 2025, is named A748-7_3_1B_SL.xml and is 3,337 bytes in size.

### FIELD OF THE INVENTION

The present invention relates to novel POLRMT modulators, their prodrugs, their pharmaceutically acceptable salts, and pharmaceutical compositions thereof. The present invention also relates to methods of using such compounds and compositions, including to inhibit or promote POLRMT, and to treat various neurodegenerative and metabolic disorders, cancer, and also disorders related to aging and mitochondrial diseases.

### BACKGROUND OF THE INVENTION

Human mitochondrial RNA polymerase, POLRMT (also referred to as h-mtRNAP), is a nuclear-encoded single-subunit DNA-dependent RNA polymerase. POLRMT is 1230 amino acids in length and consists of three distinct regions: (1) a C-terminal polymerase domain (CTD) (residues 648-1230); (2) an N-terminal domain (NTD) (residues 369-647); and (3) an N-terminal extension (NTE) (residues 1-368). See, e.g., Arnold, J. J., et al., “Human mitochondrial RNA polymerase: Structure-function, mechanism and inhibition,” Biochim. Biophys. Acta, 1819, 948-960 (2012). It is structurally related to the single-subunit RNA polymerase encoded by bacteriophage T7. The CTD is also known as the catalytic domain due to its function of catalyzing nucleotide incorporation into a growing RNA molecule during transcription. This domain is highly conserved across species, whereas by contrast the NTE demonstrates significant sequence variability, suggesting organism-specific roles for this domain of POLRMT. Regarding the POLRMT NTD, structurally it resembles the N-terminal domain (also called the promoter-binding domain) of T7 RNA polymerase. However, for promoter-specific transcription initiation POLRMT requires assistance from additional transcription factors, whereas T7 RNA polymerase does not.

A primary biological role of POLRMT is to transcribe the mitochondrial genome to produce the RNAs needed for expression of mitochondrial DNA (mtDNA). Initiation, elongation, and termination are the three steps of mitochondrial transcription. Each of a light-strand promoter (LSP) and two heavy-strand promoters (HSP-1 and HSP-2) on the mtDNA contains a transcription initiation site. See, e.g., Basu, U., et al., “Structure, mechanism, and regulation of mitochondrial DNA transcription initiation,” J. Biol. Chem., 295(52), 18406-425 (2020). For promoter-specific transcription initiation, POLRMT requires two transcription factors, TFAM (transcription factor A mitochondrial) and TFB2M (transcription factor B mitochondrial). See id. Various models suggest different mechanisms by which the initiation complex structure with POLRMT, TFAM, and TFB2M comes together to cover the promoter DNA for initiation of transcription. In one current model TFAM recruits POLRMT to the promoter site to form a protein-protein pre-initiation complex, to which TFB2M binds to form the initiation complex, which covers the promoter DNA. See id. During initiation, the RNA is elongated to about 8-10 nucleotides in length. Conformational changes occur at that point, including promoter release and displacement of the initiation factors, converting the initiation complex into an elongation complex at which time transcription occurs. See id.

The mitochondrial genome encodes the various subunits of the electron transport chain. See, e.g., Shokolenko, I. N., et al., “Maintenance and expression of mammalian mitochondrial DNA,” Annu. Rev. Biochem., 85, 133-160 (2016). Specifically, transcription of the mitochondrial genome is necessary for the expression of 13 subunits of the oxidative phosphorylation (OXPHOS) system, as well as two rRNAs and 22 tRNAs. See, e.g., Shokolenko, I. N., et al., “Mitochondrial transcription in mammalian cells,” Frontiers in Bioscience, Landmark, 22, 835-853 (2017). Thus, POLRMT is essential for biogenesis of the OXPHOS system, resulting in ATP production. This, in turn, is vital for energy homeostasis in the cell.

Dysregulation of POLRMT and the OXPHOS system have been implicated in various disease states, in particular cancer. Cancer is now the second leading cause of death in the United States, with projections indicating that almost two million new cases will be diagnosed in 2022 and over 600,000 deaths will be the result of cancer. See Siegel, R. L. et al., “Cancer statistics 2022.” CA Cancer J. Clin. (72) 7-33 (2022). High rates of OXPHOS have been shown to support growth in cancer cell lines, including in a subset of diffuse large B cell lymphoma cells. See, e.g., DeBeradinis, R. J., “A mitochondrial power play in lymphoma,” Cancer Cell, 22, 423-24 (2012). Noteworthy is the observation that metabolic heterogeneity exists not only between different types of cancer, but also among tumors of the same type. Similarly, in a study using melanoma cell lines representative of various stages of tumor progression and that collectively mimic the mixture of cells found in a tumor, it was found that metastatic cells demonstrated a high OXPHOS capacity. Rodrigues, M. F., et al., “Enhanced OXPHOS, glutaminolysis and β-oxidation constitute the metastatic phenotype of melanoma cells,” Biochem. J. 473: 703-715 (2016). These data suggest mitochondria play a role as cells progress toward metastasis, possibly to provide the energy needed for tumor cell migration and invasion.

Relatedly, overexpression of POLRMT has been linked to multiple types of cancers, suggesting that it plays a role in tumor growth. Supporting this hypothesis is, for example, a study involving acute myeloid leukemia (AML) cells, which are known to have high oxidative phosphorylation and mitochondrial mass, as well as low respiratory chain spare reserve capacity. POLRMT knockdown AML cells demonstrated a reduction in POLRMT levels, decreased oxidative phosphorylation, and increased cell death as compared to control AML cells. See Bralha, F. N., et al., “Targeting mitochondrial RNA polymerase in acute myeloid leukemia,” Oncotarget, 6(35), 37216-228 (2015). In other work, injection into nude mice of a human breast cancer cell line that overexpresses POLRMT resulted in increased tumor growth, independent of tumor angiogenesis, suggesting that POLRMT should be considered a tumor promoter or metabolic oncogene. Salem, A. F., et al. “Mitochondrial biogenesis in epithelial cancer cells promotes breast cancer tumor growth and confers autophagy resistance,” Cell Cycle, 11(22), 4174-80 (2012). Recently, the expression of POLRMT in non-small cell lung cancer (NSCLC) has been examined. See Zhou, T. et al., “The requirement of mitochondrial RNA polymerase for non-small cell lung cancer cell growth,” Cell Death and Disease, 12, 751 (2021). While POLRMT mRNA and protein were detected in normal human lung tissue, their levels were significantly higher in cancer tissue. Similar results were obtained when comparing primary lung epithelial cells to NSCLC cells. Using short hairpin RNA (shRNA) to silence POLRMT mRNA and downregulate POLRMT protein resulted in inhibition of NSCLC cell viability, proliferation, migration, and invasion. Moreover, silencing of POLRMT significantly induced apoptosis activation in both primary and established NSCLC cells. Injection of POLRMT shRNA in an adeno-associated virus construct into tumors effectively inhibited NSCLC xenograft growth in mice. Taken together, these data suggest that POLRMT could be an oncogenic gene for NSCLC.

The development of multidrug resistance (MDR) to numerous cancers is associated with poor prognosis and presents significant challenges in the treatment of this disease. Because such resistance encompasses drugs having different structures and mechanisms of action, identifying and targeting a single biochemical pathway that could re-sensitize MDR cancer cells to established chemotherapy would provide a promising treatment strategy. See Yu, H.-J., “Targeting mitochondrial metabolism and RNA polymerase POLRMT to overcome multidrug resistance in cancer,” Front. Chem., 9:775226 (2021). A main reason for the development of MDR is enhanced drug efflux from and decreased drug accumulation in MDR cells due to ATP-dependent protein transporters that pump drugs out of cells. Inhibiting POLRMT and consequently the production of the proteins essential for the OXPHOS system could compromise ATP production and, in turn, the ATP-dependent efflux of chemotherapeutic agents from cancer cells.

Consistent with the findings that the OXPHOS system and POLRMT may be involved in the etiology of and in some cases overexpressed in some cancers, small-molecule inhibitors of POLRMT have been developed. See, e.g., EP 3 598 972 A1; WO 2019/057821 A1; and WO 2020/188049 A1. Some of these inhibitors have been shown to be useful in inhibiting cancer cell proliferation without affecting control cells. See Bonekamp, N. A., et al., “Small-molecule inhibitors of human mitochondrial DNA transcription,” Nature, 588, 712-716 (2020). The cancer cell toxicity was correlated to a considerable increase in the levels of mono- and diphosphate nucleotides with a concomitant decrease in nucleotide triphosphate levels, all the result of a debilitated OXPHOS system. Similarly, treatment with POLRMT inhibitors caused a decrease in citric-acid cycle intermediates and ultimately cellular amino acid levels, the result of which is a state of severe energy and nutrient depletion. See id. Such inhibitors also produced a decrease in tumor volume in mice with no significant toxicity in control animals. Specifically, mtDNA transcript levels in tumor cells were decreased as compared to transcript levels in differentiated tissue. These data highlight the importance of mtDNA expression in rapidly dividing cells as opposed to post-mitotic tissue, a distinction that may be capitalized on using POLRMT inhibitors that are capable of modulating mtDNA transcription and ultimately the OXPHOS system.

While mitochondria are an emerging target for cancer treatment, the resistance mechanisms induced by chronic inhibition of mitochondrial function are poorly understood. In view of the challenges presented by drug resistance in cancer chemotherapy, the development of such resistance to small molecule inhibitors of POLRMT has been investigated. See Mennuni, M. et al., “Metabolic resistance to the inhibition of mitochondrial transcription revealed by CRISPER-Cas9 screen,” EMBO reports, 23: e53054 1-18 (2022). Using a CRISPR-Cas9 whole-genome screen, loss of genes belonging to von Hippel-Lindau (VHL) and mammalian target of rapamycin complex 1 (mTORC1) were the pathways that caused resistance to acute treatment with a POLRMT inhibitor. See id. at pp. 1-2. Moreover, dose-escalated chronic treatment of cells with this molecule resulted in drug-resistant cells that had increased levels of mtDNA, thereby giving rise to increased levels of mitochondrial transcripts and proteins. See id. at p. 5. The drug-resistant cells maintained higher levels of nucleotide levels, tricarboxylic acid cycle intermediates, and amino acids. See id. at p. 7. Notably, the drug-resistant cells did not have mutations in POLRMT that compromise inhibitor binding to the polymerase. See id. The development of resistance to POLRMT inhibitors underscores the importance and need for the development of other POLRMT inhibitors to understand and treat cancers of varying types.

Alterations in the OXPHOS system also have been implicated in the development of insulin resistance and ultimately Type-2 diabetes. In studies involving apoptosis inducing factor (AIF) knockout mice, a primary OXPHOS defect that produced OXPHOS deficiency revealed an increase in insulin sensitivity and resistance to diabetes and obesity. See Pospisilik, J. A., et al., “Targeted deletion of AIF decreases mitochondrial oxidative phosphorylation and protects from obesity and diabetes,” Cell, 131, 476-91 (2007). Correlated with these phenotypic changes were the metabolic alterations of increased glucose uptake and enhanced fuel utilization. Manipulation of the OXPHOS system with POLRMT modulators affords the potential for further understanding the physiological mechanisms involved in diseases such as diabetes and for the development of novel treatments for intervention of such metabolic disorders.

In addition to its critical role in transcription, POLRMT acts as the primase for mtDNA replication, thus playing a part in the regulation of mtDNA levels. Human mtDNA is a circular double-stranded DNA that is packaged in DNA-protein structures called mitochondrial nucleoids, for which TFAM is the most abundant structural component. See, e.g., Filograna, R., et al., “Mitochondrial DNA copy number in human disease: the more the better?” FEBS Letters, 595, 976-1002 (2021). TFAM facilitates mtDNA compaction, which results in regulating the accessibility of the DNA to cellular replication and transcription components. With respect to mtDNA replication, POLRMT is part of the mtDNA replisome along with the hexameric helicase TWINKLE, the heterotrimeric DNA polymerase gamma (POLγ) and the tetrameric mitochondrial single-stranded DNA-binding protein (mtSSB). See id. Its function in this replisome is to synthesize the RNA primers required for the initiation of the synthesis of both strands of mtDNA. While there may be many mechanisms by which mtDNA levels may be regulated, including modulation of POLRMT, what is known to date is that mtDNA copy number can be manipulated through modulation of TFAM expression.

While the correlation is not completely straightforward, changed levels of mtDNA have been implicated in neurogenerative disorders, cancer, and aging. See e.g., Filograna, R., et al., “Mitochondrial DNA copy number in human disease: the more the better?” FEBS Letters, 595, 976-1002 (2021). Particularly challenging is the attempt to understand the relationship between mtDNA copy number and cancer. It appears that such copy number can correlate with both increased and decreased disease burden. As such, tumor type and stage of disease may be important factors in determining the role of mtDNA copy number in the diagnosis and/or prognosis of cancer. With respect to aging, most data show a reduction in mtDNA levels in the older population. That being said, other study data are inconsistent as to the relationship between mtDNA copy number and longevity. By contrast, there appears to be a clearer correlation between neurodegeneration in Alzheimer's disease and reduction in mtDNA levels. Complicating the understanding of the relationship between mtDNA levels and disease is the role that mtDNA mutations have on various disorders. While accumulation of mtDNA mutations appears to occur in almost all types of cancer, it is unclear whether such mutations are causative of the cancer or merely a by-product of rapid replication in fast-dividing cells. Nonetheless, since POLRMT plays a key role in mtDNA replication, POLMRT modulation may provide an effective mechanism by which to understand various disease states and how to slow or alter the progression of disease.

Mutations affecting POLRMT may also cause human disease. See Oláihová, M., et al., “POLRMT mutations impair mitochondrial transcription causing neurological disease.” Nat. Commun., 12, 1135 (2021). POLRMT variants have been identified in a number of unrelated families. Patients present with multiple phenotypes, including global developmental delay, hypotonia, short stature, and speech/intellectual disability in childhood. POLRMT modulation may provide a mechanism to slow or alter the progression of disease.

POLRMT is of fundamental importance for both expression and replication of the human mitochondrial genome. While aspects of POLRMT biochemistry are known, its full physiological role in mitochondrial gene expression and homeostasis, as well as its underlying impact in the etiology of various disease states, remains unclear. Its dysfunction and/or deregulation impacts mitochondrial metabolism, sometimes through the OXPHOS system, which ultimately contributes to many metabolic, degenerative and age-related diseases such as cancer, diabetes, obesity, and Alzheimer's disease. Pharmacological inhibition of POLRMT is one means by which to gain a further understanding of the role of this polymerase in cell physiology and the development of disease. Regulation of metabolic mechanisms, including oxidative phosphorylation, with POLRMT modulators affords an opportunity for intervention in complex disorders. In view of the numerous and varied roles of POLRMT, the need exists for potent and specific modulators of POLRMT.

### SUMMARY OF THE INVENTION

Provided are compounds, pharmaceutically acceptable salts of the compounds, and prodrugs of the compounds; pharmaceutical compositions comprising the compounds or their salts or prodrugs; and methods of using the compounds, salts of the compounds, prodrugs of the compounds, or pharmaceutical compositions of the compounds, their salts, or their prodrugs to treat various neurodegenerative and metabolic disorders, cancer, and also disorders related to aging and mitochondrial diseases. The compounds and their pharmaceutically acceptable salts are particularly useful as modulators of POLRMT.

In one example, the present invention is directed to a compound, a prodrug thereof, or a pharmaceutically acceptable salt thereof, represented by formula (I):

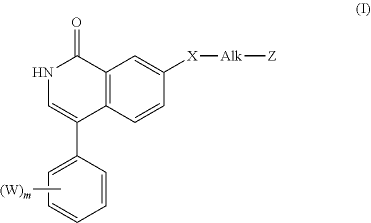

wherein:
W is selected from the group consisting of fluoro, chloro, trifluoromethyl, difluoromethyl, cyano, hydroxy, O-Alk, trifluoromethoxy, C(O)NRR, SO₂R, and Alk, wherein the Alk is optionally substituted with one or more deuterium or fluoro, hydroxy, O—(C₃-C₆) cycloalkyl, trifluoromethyl, NRR, NRC(O)R, or alkoxy that is optionally substituted with one or more deuterium, wherein at least one W is located at the ortho position;
X is O or NR;
Alk is C₁-C₆ alkyl;
Z is:

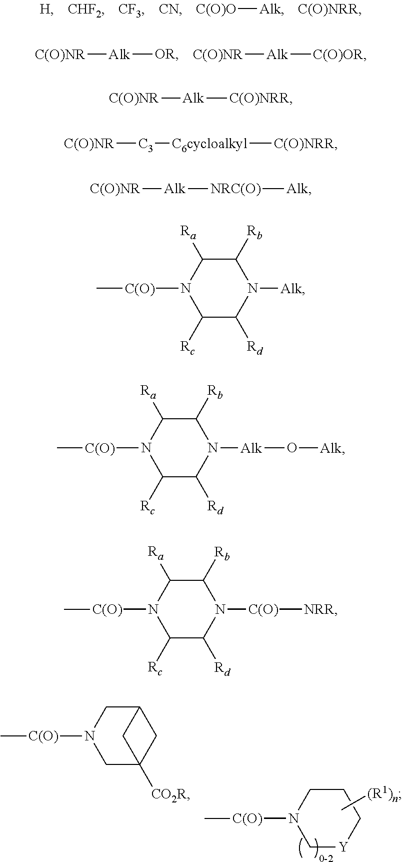

Y is O or CH₂;
R is H or C₁-C₆ alkyl optionally substituted with one or more fluoro groups; Rₐ-R_{d} are independently selected from the group consisting of H, ═O, C(O)OR, and C(O)NRR, wherein either Rₐ or R_{b} optionally forms a 4- to 6-membered cyclic ring with either of R_{c} or R_{d};
R¹ is Alk, C₃-C₆ cycloalkyl, F, NRR, CO₂H, C(O)NRR, NRC(O)R, or NRC(O)NRR, wherein Alk is optionally substituted with one or more deuterium or F, CO₂H, or C(O)NRR, and wherein C₃-C₆ cycloalkyl is substituted with CO₂H or C(O)NRR;
m is 1-5; and
n is 0-4.

In another example, the present invention is directed to a compound, a prodrug thereof, or a pharmaceutically acceptable salt thereof, represented by formula (I):

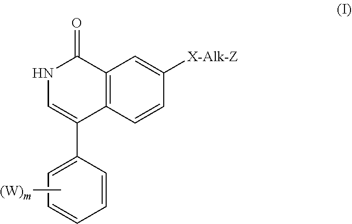

wherein:
W is selected from the group consisting of fluoro, chloro, cyano, trifluoromethoxy, C(O)NRR, and Alk, wherein the Alk is optionally substituted with one or more deuterium or fluoro, hydroxy, NRR, NRC(O)R, or methoxy that is optionally substituted with one or more deuterium, wherein at least one W is located at the ortho position;
X is O or NR;
Alk is C₁-C₃ alkyl;
Z is:

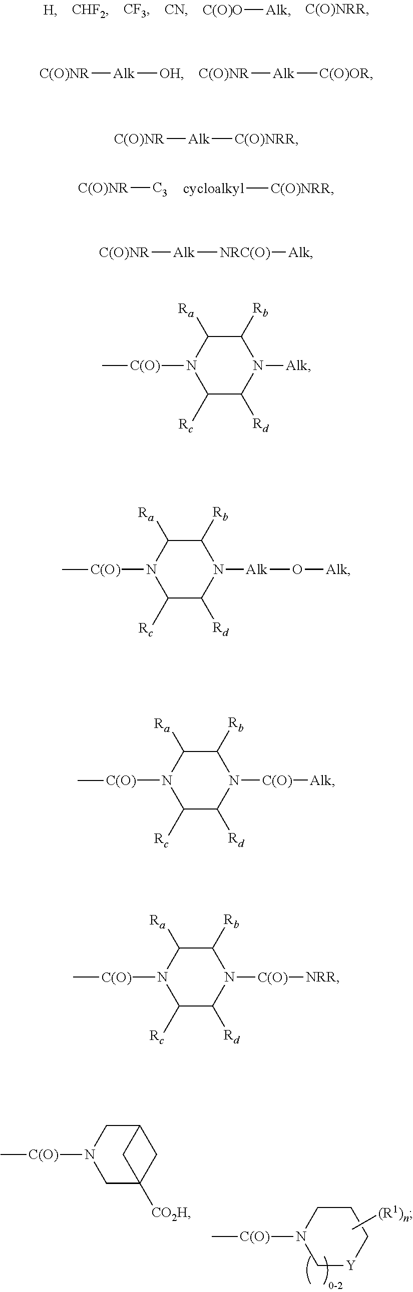

Y is O or CH₂;
R is H or C₁-C₂ alkyl optionally substituted with one or more fluoro groups; Rₐ-R_{d} are independently selected from the group consisting of H and ═O, wherein either Rₐ or R_{b}, optionally forms a 4- or 5-membered cyclic ring with either of R_{c} or R_{d};
R¹ is Alk, C₃-C₄ cycloalkyl, F, NRR, CO₂H, C(O)NRR, NRC(O)R, or NRC(O)NRR, wherein Alk is optionally substituted with one or more deuterium, CO₂H, or C(O)NRR, and wherein C₃-C₄ cycloalkyl is substituted with CO₂H or C(O)NRR;
m is 1-3; and
n is 1-4.

Further examples of the present invention are compounds of the invention (that is, compounds of formula (I)), their pharmaceutically acceptable salts, or prodrugs of the compounds wherein one or more hydrogen is substituted with a deuterium atom.

Additional examples of the invention are pharmaceutical compositions comprising a compound of the invention, a pharmaceutically acceptable salt thereof, or a prodrug thereof and one or more pharmaceutically acceptable excipients.

Further examples of the invention are methods of treating a disease, such methods comprising administering to a subject in need thereof a therapeutically effective amount of a compound of the invention, a prodrug thereof, or a pharmaceutically acceptable salt thereof. In some examples, the disease is selected from the group consisting of adrenal gland cancer, anal cancer, adenocarcinoma, angiosarcoma, bile duct cancer, bladder cancer, blastic plasmacytoid dendritic cell neoplasm, bone cancer, brain cancer, breast cancer, bronchogenic carcinoma, central nervous system (CNS) cancer, cervical cancer, cholangiocarcinoma, chondrosarcoma, colon cancer, choriocarcinoma, colorectal cancer, cancer of connective tissue, esophageal cancer, embryonal carcinoma, fibrosarcoma, gall bladder cancer, gastric cancer, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas), melanoma, Merkel cell carcinoma, mesothelioma, multiple myeloma, muscular cancer, myxosarcoma, neuroblastomas, non-small cell lung cancer, ocular cancer, oral/digestive tract cancer, osteogenic sarcoma, ovarian cancer, papillary carcinoma, pancreatic cancer, polycythemia vera, prostate cancer, rhabdomyosarcoma, renal cancer, retinal cancer, skin cancer, small cell lung carcinoma, stomach cancer, testicular cancer, throat cancer, thyroid cancer, uterine cancer, vaginal cancer, and vulvar cancer. In some examples, the disease is selected from the group consisting of Alzheimer's disease and Parkinson's disease. In some examples, the disease is selected from the group consisting of obesity, diabetes, non-alcoholic steatohepatitis (NASH), and related metabolic syndromes such as non-alcoholic fatty liver disease (NAFLD). In some examples, the disease is related to aging or a mitochondrial disorder.

Additional examples of the invention are methods of treating neurodegenerative disorders and metabolic disorders, such as those identified in Bonekamp, N. A. et al. “Small-molecule inhibitors of human mitochondrial DNA transcription,” Nature, 588, 712-716 (2020), Filograna, R. et al, “Mitochondrial DNA copy number in human disease: the more the better?” FEBS Lett., 595, 976-1002 (2021), Wrendenber, A. et al. “Respiratory chain dysfunction in skeletal muscle does not cause insulin resistance,” Biochem. Biophys. Res. Comm., 350, 202-207 (2006), Pospililik, J. A. et al. “Targeted deletion of ALF decreases mitochondrial oxidative phosphorylation and protects from obesity and diabetes,” Cell, 131, 476-491 (2007), and PCT Published International Publication No. WO 2019/057821 A1 and references therein.

Further examples of the invention are methods of treating disease of aging.

### DETAILED DESCRIPTION OF THE INVENTION

Modulators of POLRMT are useful in compositions and methods suitable for treating many disorders, such as cancer, neurodegenerative disorders, metabolic disorders, as well as diseases related to aging and mitochondrial diseases. Provided herein are compounds of formula (I), pharmaceutically acceptable salts thereof, prodrugs thereof, and pharmaceutical compositions comprising such compounds, their salts, or their prodrugs that are useful in treating a condition or disease, such as cancer, neurodegenerative disorders, and metabolic disorders.

### Definitions

The term “alkyl” or “Alk” as used herein refers to both branched- and straight-chain saturated aliphatic hydrocarbon groups having the specified number of carbon atoms in a specified range. For example, the term Alk could be defined as “C₁-C₆ alkyl,” which means linear or branched chain alkyl groups, including all possible isomers, having 1, 2, 3, 4, 5, or 6 carbon atoms. Furthermore, alkyl groups allow for substituents to be located on any of the carbon atoms. For example, a substituted C₃ alkyl group allows for the substituent to be located on any of the three carbon atoms.

The term “alkoxy” or “alkoxyl” as used herein refers to an —O-alkyl group or an O-Alk group. For example, the term “C₁-C₄ alkoxy” means —O—C₁-C₄ alkyl. Examples of alkoxy include methoxy, ethoxy, propoxy (e.g., n-propoxy and isopropoxy), and the like.

The term “haloalkoxy” or “haloalkoxyl” as used herein refers to an —O-alkyl group in which at least one of the hydrogen atoms of the alkyl group is replaced with a halogen atom.

Examples of haloalkoxyl include trifluoromethoxyl, 2,2,2-trifluoroethoxyl, and the like.

The term “alkanoyl” or “acyl” as used herein refers to an —C(O)-alkyl group. For example, the term “C₁-C₆ alkanoyl” means —C(O)—C₁-C₆ alkyl. Examples of alkanoyl include acetyl, propionyl, butyryl, and the like.

The term “bicyclic” as used herein refers to a saturated or unsaturated 6- to 12-membered ring consisting of two joined cyclic substructures, and includes fused, bridged, and spiro bicyclic rings.

The term “heterobicyclic” as used herein refers to a bicyclic ring that contains 1 or more heteroatom(s) in one or more rings that are optionally substituted or oxidized, and may be selected from nitrogen (including N-oxides), oxygen, sulfur (including oxidized forms such as sulfones and sulfonates), phosphorus (including oxidized forms such as phosphates), boron, etc.

Examples of heterobicyclic rings include, but are not limited to 8-azabicyclo[3.2.1]octan-8-yl, 3-oxa-8-azabicyclo[3.2.1]octan-8-yl, 8-oxa-3-azabicyclo[3.2.1]octan-3-yl, and 5-methyl-2,5-diazabicyclo[2.2.1]heptan-2-yl.

The term “cycloalkyl” as used herein refers to a cyclized alkyl ring having the indicated number of carbon atoms in a specified range. Thus, for example, “C₃-C₆ cycloalkyl” encompasses each of cyclopropyl, cyclobutyl, cyclopentyl, and cyclohexyl.

The term “cycloalkenyl” as used herein refers to partially unsaturated monocyclic, bicyclic, tricyclic, or other polycyclic hydrocarbon groups. A ring carbon (e.g., saturated or unsaturated) is the point of attachment of the cycloalkenyl substituent. In certain examples, a cycloalkenyl is a C₄-C₁₀ cycloalkenyl. In certain examples, a cycloalkenyl is monocyclic, or is bicyclic. Examples of cycloalkenyl, include but are not limited to, cyclopentenyl, cyclohexenyl, cyclohexadienyl, or norbornenyl. In some examples, cycloalkenyl includes groups with more than one double bond such as 1,3- and 1,4-cyclohexadienyl.

The term “aryl” as used herein refers to a monocyclic or fused bicyclic ring system having the characteristics of aromaticity, wherein at least one ring contains a completely conjugated pi-electron system. Typically, aryl groups contain 6 to 14 carbon atoms (“C₆-C₁₄ aryl”) or preferably, 6 to 12 carbon atoms (“C₆-C₁₂ aryl”). Fused aryl groups may include an aryl ring (e.g., a phenyl ring) fused to another aryl ring, or fused to a saturated or partially unsaturated carbocyclic or heterocyclic ring. The point of attachment to the base molecule on such fused aryl ring systems may be a C atom of the aromatic portion or a C or N atom of the non-aromatic portion of the ring system. Examples, without limitation, of aryl groups include phenyl, biphenyl, naphthyl, anthracenyl, indanyl, indenyl, and tetrahydronaphthyl.

The term “cycloaryl” herein refers to a polycyclic group wherein an aryl group is fused to a 5- or 6-membered aliphatic ring. For example, “C₆-C₁₂ cycloaryl” means a C₆-C₁₂ aryl fused to a 5- or 6-membered aliphatic ring.

The term “heteroaryl” as used herein refers to (i) a 5- or 6-membered ring having the characteristics of aromaticity containing at least one heteroatom selected from N, O and S, wherein each N is optionally in the form of an oxide, and (ii) a 9- or 10-membered bicyclic fused ring system, wherein the fused ring system of (ii) contains at least one heteroatom independently selected from N, O and S, wherein each ring in the fused ring system contains zero, one or more than one heteroatoms, at least one ring is aromatic, each N is optionally in the form of an oxide, and each S in a ring which is not aromatic is optionally S(O) or S(O)₂. Typically, heteroaryl groups contain 5 to 14 ring atoms (“5-14 membered heteroaryl”), and preferably 5 to 12 ring atoms (“5- to 12-membered heteroaryl”). Heteroaryl rings are attached to the base molecule via a ring atom of the heteroaromatic ring, such that aromaticity is maintained. Suitable 5- and 6-membered heteroaromatic rings include, for example, pyridyl, 3-fluroropyridyl, 4-fluoropyridyl, 3-methoxypyridyl, 4-methoxypyridyl, pyrrolyl, pyrazinyl, pyrimidinyl, pyridazinyl, triazinyl, thienyl, furanyl, imidazolyl, pyrazolyl, triazolyl (i.e., 1,2,3-triazolyl or 1,2,4-triazolyl), tetrazolyl, oxazolyl, isooxazolyl, oxadiazolyl (i.e., the 1,2,3-, 1,2,4-, 1,2,5-(furazanyl), or 1,3,4-isomer), oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzisoxazolyl, benzoxazolyl, benzothiazolyl, chromenyl, quinolinyl, isoquinolinyl, benzopiperidinyl, benzofuranyl, imidazo[1,2-a]pyridinyl, benzotriazolyl, indazolyl, indolinyl, and isoindolinyl.

The term “heteroaryloxy” or “heteroaryloxyl” as used herein refers to an —O— heteroaryl group.

The term “heterocycle”, “heterocyclyl”, or “heterocyclic” as used herein represents a stable 3- to 10-membered monocyclic, non-aromatic ring that is either saturated or unsaturated, and that consists of carbon atoms and from one to two heteroatoms selected from the group consisting of N, O, and S. Examples include oxiranyl, aziridinyl, oxetanyl, azetidinyl, tetrahydrofuranyl, pyrrolidinyl, tetrahydropyranyl, piperidinyl, 1,4-dioxanyl, morpholinyl, piperazinyl, azepanyl, oxepanyl, and oxazepanyl.

The term “oxo” as used herein refers to a group which consists of oxygen which is double bonded to carbon or any other element.

The term “imine” as used herein refers to a group containing a carbon-nitrogen double bond.

The term “carboxyl” as used herein refers to a combination of two functional groups attached to a single carbon atom, namely, hydroxy (OH) and carbonyl (O).

The term “optionally substituted” or “optional substituents” as used herein means that the groups are either unsubstituted or substituted with one or more of the substituents specified. When the groups are substituted with more than one substituent, the substituents may be the same or different. Furthermore, when using the terms “independently,” “independently are,” and “independently selected from” means that the groups may be the same or different.

The term “deuterium” as used herein refers to an isotope of hydrogen that has one proton and one neutron in its nucleus and that has twice the mass of ordinary hydrogen. Deuterium herein is represented by the symbol “D”.

The term “deuterated” by itself or used to modify a compound or group as used herein refers to the presence of at least one deuterium atom attached to carbon. For example, the term “deuterated compound” refers to a compound which contains one or more carbon-bound deuterium(s). In a deuterated compound of the present invention, when a particular position is designated as having deuterium, it is understood that the abundance of deuterium at that position is substantially greater than the natural abundance of deuterium, which is about 0.015%.

The term “undeuterated” or “non-deuterated” as used herein refers to the ratio of deuterium atoms of which is not more than the natural isotopic deuterium content, which is about 0.015%; in other words, all hydrogen are present at their natural isotopic percentages. Unless otherwise stated, when a position is designated specifically as “H” or “hydrogen”, the position is understood to have hydrogen at its natural abundance isotopic composition.

The term “isotopic enrichment factor” as used herein refers to the ratio between the isotope abundance and the natural abundance of a specified isotope.

The term “isotopologue” as used herein refers to a species in which the chemical structure differs from a specific compound of the invention only in the isotopic composition thereof.

The term “substantially free of other stereoisomers” as used herein means less than 10% of other stereoisomers, preferably less than 5% of other stereoisomers, more preferably less than 2% of other stereoisomers and most preferably less than 1% of other stereoisomers are present.

The term “pharmaceutically acceptable salt” as used herein refers to a salt that is not biologically or otherwise undesirable (e.g., not toxic or otherwise harmful). A salt of a compound of the invention is formed between an acid and a basic group of the compound, or a base and an acidic group of the compound. For example, when the compounds of the invention contain at least one basic group (i.e., groups that can be protonated), the invention includes the compounds in the form of their acid addition salts with organic or inorganic acids such as, for example, but not limited to salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the invention contain one or more acidic groups (e.g., a carboxylic acid), the invention includes the pharmaceutically acceptable salts of the compounds formed with but not limited to alkali metal salts, alkaline earth metal salts or ammonium salts. Examples of such salts include, but are not limited to, sodium salts, potassium salts, calcium salts, magnesium salts or salts with ammonia or organic amines such as, for example, ethylamine, ethanolamine, triethanolamine or amino acids. Additional examples of such salts can be found in Stahl, P. H. et al. Pharmaceutical Salts: Properties, Selection, and Use, 2nd Revised Edition, Wiley, 2011.

The term “prodrug” as used herein refers to derivatives of compounds of the invention which may have reduced pharmacological activity, but can, when administered to a patient, be converted into the inventive compounds. Design and use of prodrugs may be found in “Pro-drugs as Novel Delivery Systems,” Vol. 14, ACS Symposium Series (T Higuchi and W Stella) and “Bioreversible Carriers in Drug Design,” Pergamon Press, 1987 (ed. E B Roche, American Pharmaceutical Association), the disclosures of which are incorporated herein by reference in their entireties. Prodrugs in accordance with the invention can, for example, be produced by replacing appropriate functionalities present in the inventive compounds with certain moieties known to those skilled in the art as ‘pro-moieties’ as described, for example, in “Design of Prodrugs” by H Bundgaard (Elsevier, 1985), the disclosure of which is incorporated herein by reference in its entirety. Some non-limiting examples of prodrugs in accordance with the invention include. (i) where the compound contains a carboxylic acid functionality —(COOH), an ester thereof, for example, replacement of the hydrogen with (C₁-C₆)alkyl; (ii) where the compound contains an alcohol functionality (—OH), an ether thereof, for example, replacement of the hydrogen with (C₁-C₆)alkanoyloxymethyl, or with a phosphate ether group; and (iii) where the compound contains a primary or secondary amino functionality (—NH₂ or —NHR, where R is not H), an amide thereof, for example, replacement of one or both hydrogens with C₁-C₆ alkanoyl. Further examples of replacement groups in accordance with the foregoing examples and examples of other prodrug types may be found in the aforementioned references.

The terms “treatment”, “treating” and “treat” as used herein, include their generally accepted meanings, i.e., the management and care of a patient for the purpose of preventing, reducing the risk in incurring or developing a given condition or disease, prohibiting, restraining, alleviating, ameliorating, slowing, stopping, delaying, or reversing the progression or severity, and holding in check existing characteristics of a disease, disorder, or pathological condition, including the alleviation or relief of symptoms or complications, or the cure or elimination of the disease, disorder, or condition.

The term “therapeutically effective amount” as used herein refers to that amount of compound of the invention that will elicit the biological or medical response of a tissue, system, animal, or human that is being sought by a researcher, veterinarian, medical doctor or other. As will be recognized by a person of ordinary skill in the art, a therapeutically effective amount of the compounds of the invention will vary and will depend on the diseases treated, the severity of the disease, the route of administration, and the gender, age, and general health condition of the subject to whom the compound is being administered. The therapeutically effective amount may be administered as a single dose once a day, or as split doses administered multiple (e.g., two, three or four) times a day. The therapeutically effective amount may also be administered through continuous dosing, such as through infusion or with an implant.

### Compounds

In one example, the present invention is directed to a compound, a prodrug thereof, or a pharmaceutically acceptable salt thereof, represented by formula (I):

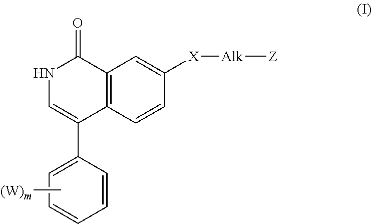

wherein:
W is selected from the group consisting of fluoro, chloro, trifluoromethyl, difluoromethyl, cyano, hydroxy, O-Alk, trifluoromethoxy, C(O)NRR, SO₂R, and Alk, wherein the Alk is optionally substituted with one or more deuterium or fluoro, hydroxy, O—(C₃-C₆) cycloalkyl, trifluoromethyl, NRR, NRC(O)R, or alkoxy that is optionally substituted with one or more deuterium, wherein at least one W is located at the ortho position;
X is O or NR;
Alk is C₁-C₆ alkyl;
Z is:

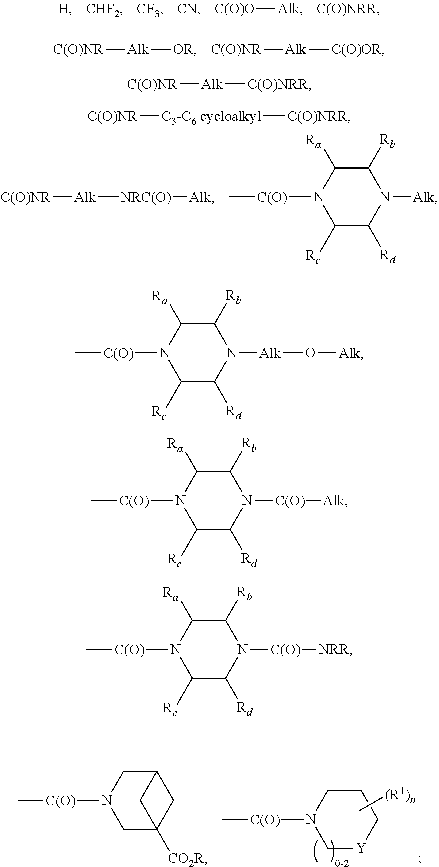

Y is O or CH₂;
R is H or C₁-C₆ alkyl optionally substituted with one or more fluoro groups;
Rₐ-R_{d} are independently selected from the group consisting of H, ═O, C(O)OR, and C(O)NRR, wherein Rₐ or R_{b} optionally forms a 4- to 6-membered cyclic ring with either of R_{c} or R_{d};
R¹ is Alk, C₃-C₆ cycloalkyl, F, NRR, CO₂H, C(O)NRR, NRC(O)R, or NRC(O)NRR, wherein Alk is optionally substituted with one or more deuterium or F, CO₂H, or C(O)NRR, and wherein C₃-C₆ cycloalkyl is substituted with CO₂H or C(O)NRR;
m is 1-5; and
n is 0-4.

In one example, the present invention is directed to a compound, a prodrug thereof, or a pharmaceutically acceptable salt thereof, represented by formula (I):

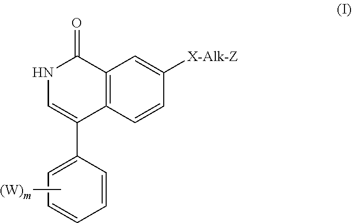

wherein:
W is selected from the group consisting of fluoro, chloro, cyano, trifluoromethoxy, C(O)NRR, and Alk, wherein the Alk is optionally substituted with one or more deuterium or fluoro, hydroxy, NRR, NRC(O)R, or methoxy that is optionally substituted with one or more deuterium, wherein at least one W is located at the ortho position;
X is O or NR;
Alk is C₁-C₃ alkyl;
Z is:

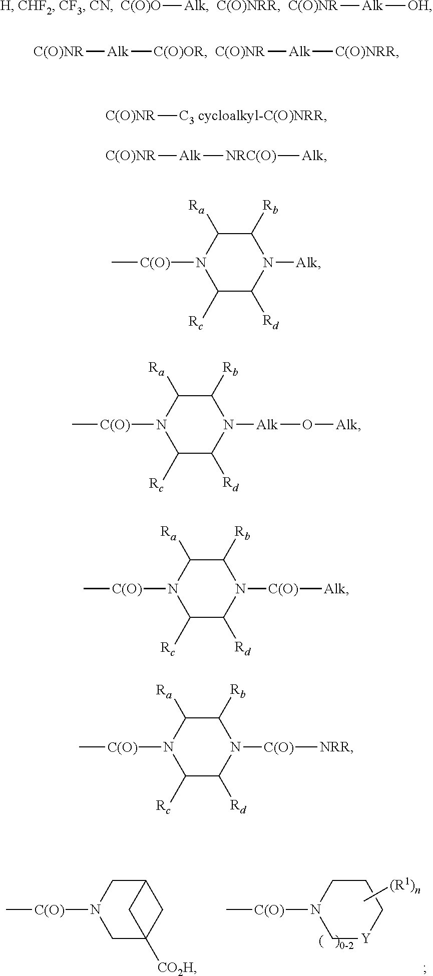

Y is O or CH₂;
R is H or C₁-C₂ alkyl optionally substituted with one or more fluoro groups; Rₐ-R_{d} are independently selected from the group consisting of H, and ═O, wherein either Rₐ or R_{b} optionally forms a 4- or 5-membered cyclic ring with either of R_{c} or R_{d};
R¹ is Alk, C₃-C₄ cycloalkyl, F, NRR, CO₂H, C(O)NRR, NRC(O)R, or NRC(O)NRR, wherein Alk is optionally substituted with one or more deuterium, CO₂H, or C(O)NRR, and wherein C₃-C₄cycloalkyl is substituted with CO₂H or C(O)NRR;
m is 1-3; and
n is 1-4.

In another example, the present invention is directed to a compound, a prodrug thereof, or a pharmaceutically acceptable salt thereof, represented by formula (I):

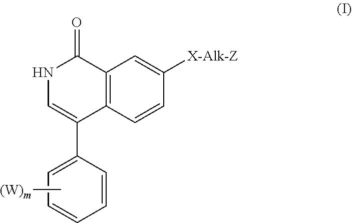

wherein:
W is selected from the group consisting of fluoro, chloro, cyano, and Alk, wherein the Alk is optionally substituted with hydroxy or one or more deuterium, wherein at least one W is located at the ortho position;
X is O or NR;
Alk is C₁-C₃ alkyl;
Z is:

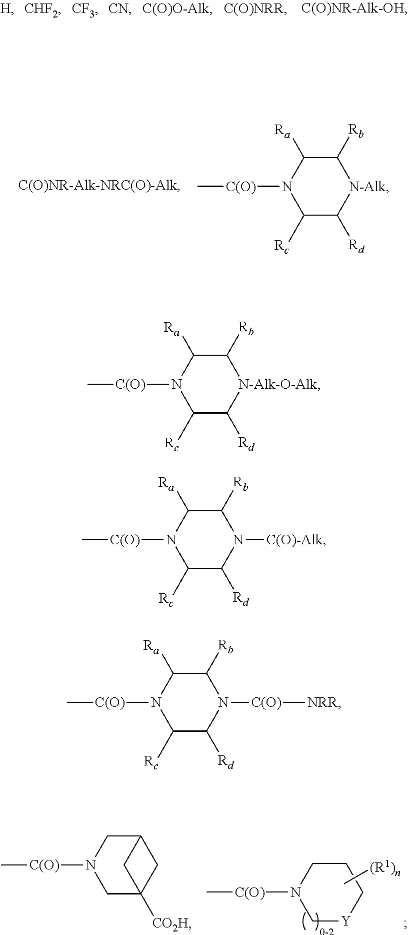

Y is CH₂;
R is H or C₁-C₂ alkyl; Rₐ-R_{d} is H or either Rₐ or R_{b} optionally forms a 4- or 5-membered cyclic ring with either of R_{c} or R_{d};
R¹ is C₁-C₃ alkyl, F, CO₂H, C(O)NRR, or NRC(O)R;
m is 1-3; and
n is 1-4.

In another example, the present invention is directed to a compound, a prodrug thereof, or a pharmaceutically acceptable salt thereof, represented by formula (I):

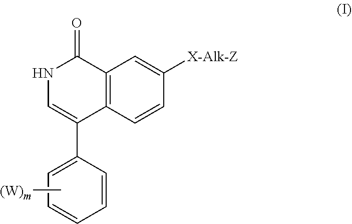

wherein:
W is selected from the group consisting of fluoro, chloro, and C₁ alkyl, wherein the C₁ alkyl is optionally substituted with one or more deuterium, wherein at least one W is located at the ortho position;
X is O or NR;
Alk is C₁-C₃ alkyl;
Z is:

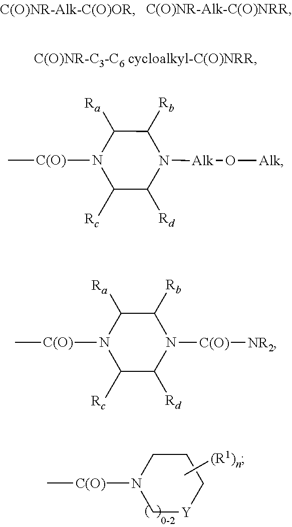

Y is O or CH₂;
R is H, C₁-C₂ alkyl optionally substituted with one or more fluoro groups;
Rₐ-R_{d} are independently selected from the group consisting of H and ═O;
R¹ is Alk, C₃-C₄ cycloalkyl, NRR, or NRC(O)NRR, wherein Alk is optionally substituted with one or more deuterium, CO₂H, or C(O)NRR, and wherein C₃-C₄ cycloalkyl is substituted with CO₂H or C(O)NRR;
m is 1 or 2; and
n is 1 or 2.

In another example, the present invention is directed to a compound, a prodrug thereof, or a pharmaceutically acceptable salt thereof, represented by formula (I):

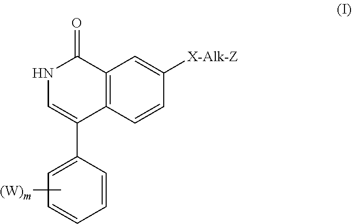

wherein:
W is selected from the group consisting of trifluoromethoxy, C(O)NRR, and C₁-C₂ alkyl, wherein the C₁-C₂ alkyl is optionally substituted with one or more deuterium or fluoro, hydroxy, NRR, NRC(O)R, or methoxy that is optionally substituted with one or more deuterium, wherein at least one W is located at the ortho position;
X is O;
Alk is C₁-C₂ alkyl;
Z is CN;
R is H or C₁ alkyl; and
m is 1-2.

In another example, the present invention is directed to a compound, a prodrug thereof, or a pharmaceutically acceptable salt thereof, represented by formula (I):

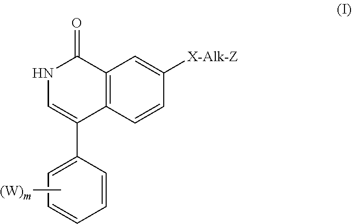

wherein:
W is C₁-C₂ alkyl optionally substituted with hydroxy, wherein at least one W is located at the ortho position;
X is O;
Alk is C₁-C₃ alkyl;
Z is:

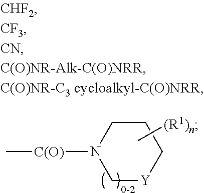

Y is CH₂;
R is H or C₁ alkyl;
R¹ is Alk, C₃ cycloalkyl, or CO₂H, wherein the C₃ cycloalkyl is substituted with CO₂H;
m is 1 or 2; and
n is 1 or 2.

In another example, the present invention is directed to a compound, a prodrug thereof, or a pharmaceutically acceptable salt thereof, represented by formula (I):

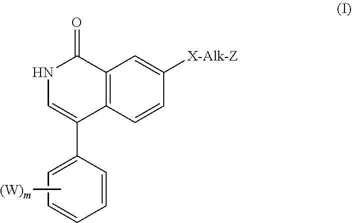

wherein:
W is C₁-C₂ alkyl optionally substituted with hydroxy, wherein at least one W is located at the ortho position;
X is O;
Alk is C₁-C₂ alkyl;
Z is:

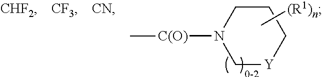

Y is CH₂;
R¹ is C₁-C₃ Alk or CO₂H;
m is 1 or 2; and
n is 2.

In another example, the present invention is directed to a compound, a prodrug thereof, or a pharmaceutically acceptable salt thereof, represented by formula (I):

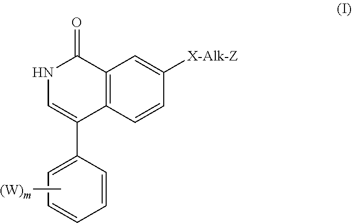

wherein:
W is C₁ alkyl, wherein the C₁ alkyl is located at the ortho position;
X is O;
Alk is C₁-C₂ alkyl;
Z is:

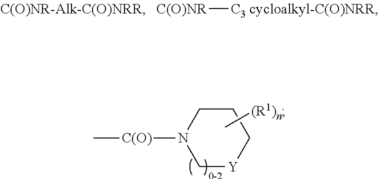

Y is CH₂;
R is H or C₁ alkyl;
R¹ is C₃ cycloalkyl substituted with CO₂H;
mis 1; and
n is 1.

In certain examples, the compound is 1-((S)-1-((R)-2-((4-(2,6-dimethylphenyl)-1-oxo-1,2-dihydroisoquinolin-7-yl)oxy)propanoyl)piperidin-3-yl)cyclopropane-1-carboxylic acid, Example 102, or a pharmaceutically acceptable salt thereof:

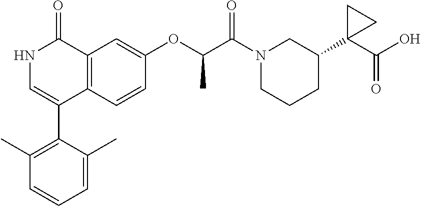 [SMILES: Cc1cccc(C)c1-c1c[nH]c(=O)c2cc(O[C@H](C)C(=O)N3CCC[C@@H](C4(C(=O)O)CC4)C3)ccc12]

In certain examples, the compound is 1-((R)-1-((R)-2-((4-(2,6-dimethylphenyl)-1-oxo-1,2-dihydroisoquinolin-7-yl)oxy)propanoyl)piperidin-3-yl)cyclopropane-1-carboxylic acid, Example 103, or a pharmaceutically acceptable salt thereof:

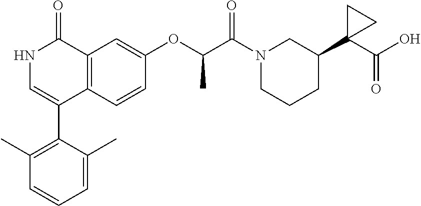 [SMILES: Cc1cccc(C)c1-c1c[nH]c(=O)c2cc(O[C@H](C)C(=O)N3CCC[C@H](C4(C(=O)O)CC4)C3)ccc12]

In certain examples, the compound is 1-((R)-1-((R)-2-((4-(2-chloro-4-fluorophenyl)-1-oxo-1,2-dihydroisoquinolin-7-yl)oxy)propanoyl)piperidin-3-yl)cyclobutane-1-carboxylic acid, Example 104, or a pharmaceutically acceptable salt thereof:

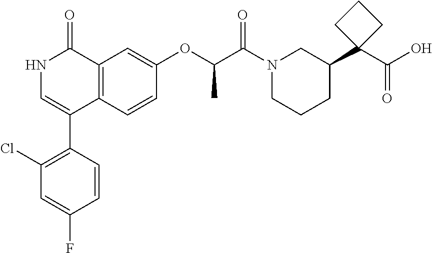 [SMILES: C[C@@H](Oc1ccc2c(-c3ccc(F)cc3Cl)c[nH]c(=O)c2c1)C(=O)N1CCC[C@H](C2(C(=O)O)CCC2)C1]

In certain examples, the compound is 1-((S)-1-((R)-2-((4-(2-chloro-4-fluorophenyl)-1-oxo-1,2-dihydroisoquinolin-7-yl)oxy)propanoyl)piperidin-3-yl)cyclobutane-1-carboxylic acid, Example 105, or a pharmaceutically acceptable salt thereof:

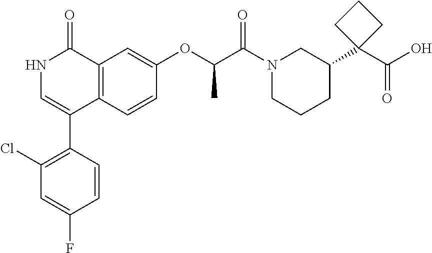 [SMILES: C[C@@H](Oc1ccc2c(-c3ccc(F)cc3Cl)c[nH]c(=O)c2c1)C(=O)N1CCC[C@@H](C2(C(=O)O)CCC2)C1]

In certain examples, the compound is 1-((R)-1-((R)-2-((1-oxo-4-(o-tolyl)-1,2-dihydroisoquinolin-7-yl)oxy)propanoyl)piperidin-3-yl)cyclobutane-1-carboxylic acid, Example 106, or a pharmaceutically acceptable salt thereof:

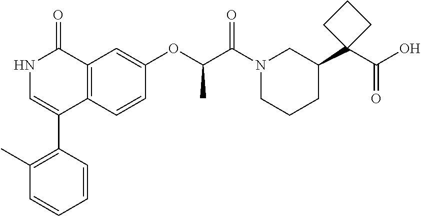 [SMILES: Cc1ccccc1-c1c[nH]c(=O)c2cc(O[C@H](C)C(=O)N3CCC[C@H](C4(C(=O)O)CCC4)C3)ccc12]

In certain examples, the compound is 1-((S)-1-((R)-2-((1-oxo-4-(o-tolyl)-1,2-dihydroisoquinolin-7-yl)oxy)propanoyl)piperidin-3-yl)cyclobutane-1-carboxylic acid, Example 107, or a pharmaceutically acceptable salt thereof:

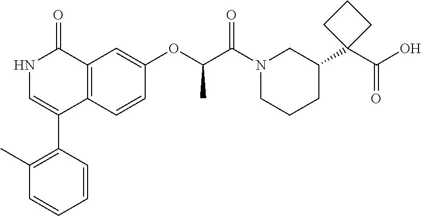 [SMILES: Cc1ccccc1-c1c[nH]c(=O)c2cc(O[C@H](C)C(=O)N3CCC[C@@H](C4(C(=O)O)CCC4)C3)ccc12]

In certain examples, the compound is (S)-1-((R)-2-((4-(2-chloro-4-fluorophenyl)-1-oxo-1,2-dihydroisoquinolin-7-yl)oxy)propanoyl)-3-propylpiperidine-3-carboxylic acid, Example 108, or a pharmaceutically acceptable salt thereof:

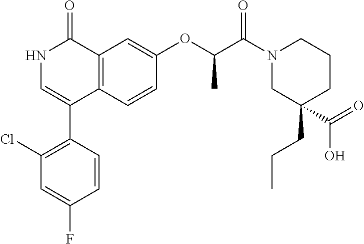 [SMILES: CCC[C@]1(C(=O)O)CCCN(C(=O)[C@@H](C)Oc2ccc3c(-c4ccc(F)cc4Cl)c[nH]c(=O)c3c2)C1]

In certain examples, the compound is (R)-1-((R)-2-((4-(2-chloro-4-fluorophenyl)-1-oxo-1,2-dihydroisoquinolin-7-yl)oxy)propanoyl)-3-propylpiperidine-3-carboxylic acid, Example 109, or a pharmaceutically acceptable salt thereof:

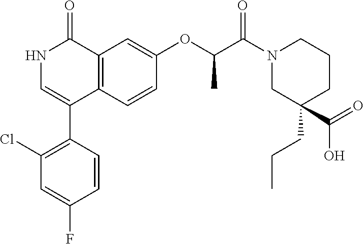 [SMILES: CCC[C@@]1(C(=O)O)CCCN(C(=O)[C@@H](C)Oc2ccc3c(-c4ccc(F)cc4Cl)c[nH]c(=O)c3c2)C1]

In certain examples, the compound is (R)-5,5-difluoro-3-methyl-1-((R)-2-((1-oxo-4-(o-tolyl)-1,2-dihydroisoquinolin-7-yl)oxy)propanoyl)piperidine-3-carboxylic acid, Example 110, or a pharmaceutically acceptable salt thereof:

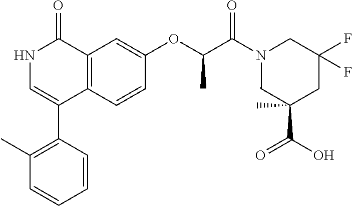 [SMILES: Cc1ccccc1-c1c[nH]c(=O)c2cc(O[C@H](C)C(=O)N3CC(F)(F)C[C@@](C)(C(=O)O)C3)ccc12]

In certain examples, the compound is (S)-5,5-difluoro-3-methyl-1-((R)-2-((1-oxo-4-(o-tolyl)-1,2-dihydroisoquinolin-7-yl)oxy)propanoyl)piperidine-3-carboxylic acid, Example 111, or a pharmaceutically acceptable salt thereof:

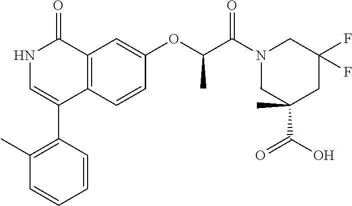 [SMILES: Cc1ccccc1-c1c[nH]c(=O)c2cc(O[C@H](C)C(=O)N3CC(F)(F)C[C@](C)(C(=O)O)C3)ccc12]

In certain examples, the compound is 2-((4-(3-fluoro-2-(hydroxymethyl)-6-methylphenyl)-1-oxo-1,2-dihydroisoquinolin-7-yl)oxy)acetonitrile, Example 112, or a pharmaceutically acceptable salt thereof:

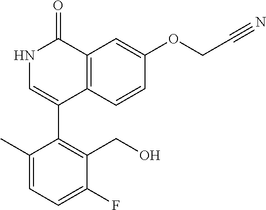 [SMILES: Cc1ccc(F)c(CO)c1-c1c[nH]c(=O)c2cc(OCC#N)ccc12]

In certain examples, the compound is 7-(((R)-1-((R)-6-(dimethylamino)-1,4-oxazepan-4-yl)-1-oxopropan-2-yl)oxy)-4-(o-tolyl)isoquinolin-1(2H)-one, Example 113, or a pharmaceutically acceptable salt thereof:

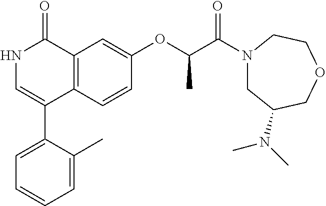 [SMILES: Cc1ccccc1-c1c[nH]c(=O)c2cc(O[C@H](C)C(=O)N3CCOC[C@H](N(C)C)C3)ccc12]

In certain examples, the compound is 7-(((R)-1-((S)-6-(dimethylamino)-1,4-oxazepan-4-yl)-1-oxopropan-2-yl)oxy)-4-(o-tolyl)isoquinolin-1(2H)-one, Example 114, or a pharmaceutically acceptable salt thereof:

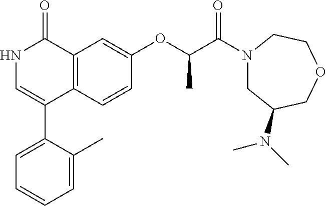 [SMILES: Cc1ccccc1-c1c[nH]c(=O)c2cc(O[C@H](C)C(=O)N3CCOC[C@@H](N(C)C)C3)ccc12]

In certain examples, the compound is 1-((R)-1-((R)-2-((4-(2-(methyl-d₃)phenyl)-1-oxo-1,2-dihydroisoquinolin-7-yl)oxy)propanoyl)piperidin-3-yl)cyclopropane-1-carboxylic acid, Example 115, or a pharmaceutically acceptable salt thereof:

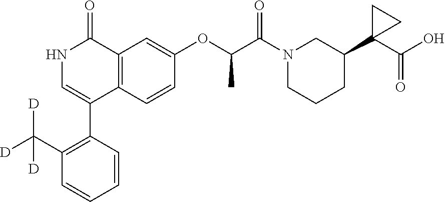 [SMILES: [2H]C([2H])([2H])c1ccccc1-c1c[nH]c(=O)c2cc(O[C@H](C)C(=O)N3CCC[C@H](C4(C(=O)O)CC4)C3)ccc12]

In certain examples, the compound is 1-((S)-1-((R)-2-((4-(2-(methyl-d₃)phenyl)-1-oxo-1,2-dihydroisoquinolin-7-yl)oxy)propanoyl)piperidin-3-yl)cyclopropane-1-carboxylic acid, Example 116, or a pharmaceutically acceptable salt thereof:

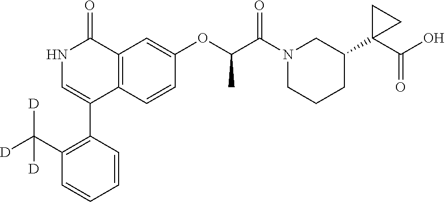 [SMILES: [2H]C([2H])([2H])c1ccccc1-c1c[nH]c(=O)c2cc(O[C@H](C)C(=O)N3CCC[C@@H](C4(C(=O)O)CC4)C3)ccc12]

In certain examples, the compound is 2-(methyl-d₃)-2-((R)-1-((R)-2-((1-oxo-4-(o-tolyl)-1,2-dihydroisoquinolin-7-yl)oxy)propanoyl)piperidin-3-yl)propanoic-3,3,3-da acid, Example 117, or a pharmaceutically acceptable salt thereof:

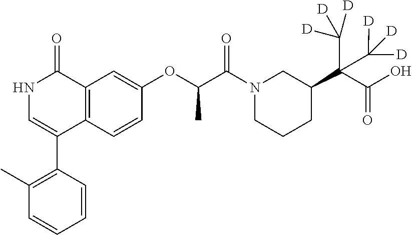 [SMILES: [2H]C([2H])([2H])C(C(=O)O)([C@H]1CCCN(C(=O)[C@@H](C)Oc2ccc3c(-c4ccccc4C)c[nH]c(=O)c3c2)C1)C([2H])([2H])[2H]]

In certain examples, the compound is 2-(methyl-d₃)-2-((S)-1-((R)-2-((1-oxo-4-(o-tolyl)-1,2-dihydroisoquinolin-7-yl)oxy)propanoyl)piperidin-3-yl)propanoic-3,3,3-d₃ acid, Example 118, or a pharmaceutically acceptable salt thereof:

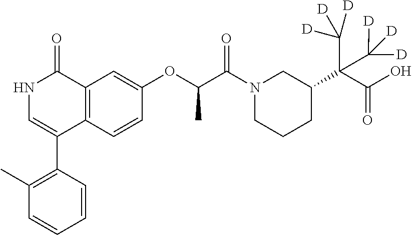 [SMILES: [2H]C([2H])([2H])C(C(=O)O)([C@@H]1CCCN(C(=O)[C@@H](C)Oc2ccc3c(-c4ccccc4C)c[nH]c(=O)c3c2)C1)C([2H])([2H])[2H]]

In certain examples, the compound is 2-((4-(2-(2-(methoxy-d₃)ethyl)phenyl)-1-oxo-1,2-dihydroisoquinolin-7-yl)oxy)acetonitrile, Example 119, or a pharmaceutically acceptable salt thereof:

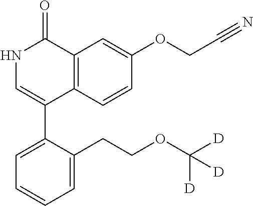 [SMILES: [2H]C([2H])([2H])OCCc1ccccc1-c1c[nH]c(=O)c2cc(OCC#N)ccc12]

In certain examples, the compound is (R)-1-((R)-2-((1-oxo-4-(o-tolyl)-1,2-dihydroisoquinolin-7-yl)oxy)propanoyl)-3-(propyl-d₇)piperidine-3-carboxylic acid, Example 120, or a pharmaceutically acceptable salt thereof:

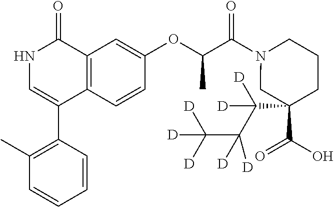 [SMILES: [2H]C([2H])([2H])C([2H])([2H])C([2H])([2H])[C@@]1(C(=O)O)CCCN(C(=O)[C@@H](C)Oc2ccc3c(-c4ccccc4C)c[nH]c(=O)c3c2)C1]

In certain examples, the compound is (S)-1-((R)-2-((1-oxo-4-(o-tolyl)-1,2-dihydroisoquinolin-7-yl)oxy)propanoyl)-3-(propyl-d₇)piperidine-3-carboxylic acid, Example 121, or a pharmaceutically acceptable salt thereof;

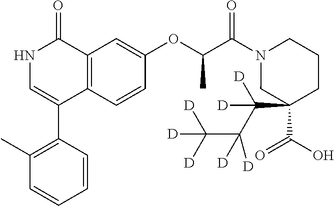 [SMILES: [2H]C([2H])([2H])C([2H])([2H])C([2H])([2H])[C@]1(C(=O)O)CCCN(C(=O)[C@@H](C)Oc2ccc3c(-c4ccccc4C)c[nH]c(=O)c3c2)C1]

In certain examples, the compounds inhibits POLRMT.

In certain examples, the compounds promote POLRMT.

The compounds of the present invention may contain asymmetric carbon atoms (sometimes as the result of a deuterium atom) and thereby can exist as either individual stereoisomers or mixtures of the enantiomers or mixtures of diastereomers. Accordingly, a compound of the present invention may exist as either a racemic mixture, a mixture of diastereomers, or as individual stereoisomers that are substantially free of other stereoisomers. Synthetic, separation, or purification methods to be used to obtain an enantiomer of a given compound are known in the art and are applicable for obtaining the compounds identified herein.

Unless otherwise indicated, when a disclosed compound is named or depicted by a structure without specifying the stereochemistry and has one or more chiral centers, it is understood to represent all possible stereoisomers of the compound. Carbon atoms labelled with * or ** refer to a compound that is chiral but the absolute stereochemistry has not been determined.

The compounds of the present invention may contain double bonds that may exist in more than one geometric isomer. Examples of such double bonds are carbon-carbon double bonds which form alkenes. In the case of carbon-carbon double bonds, the geometric isomers may be E or Z isomers.

Unless otherwise indicated, when a disclosed compound is named or depicted by a structure without specifying the geometric isomerism and has one or more possible geometric isomers, it is understood to represent all possible geometric isomers of the compound.

Certain compounds of the present invention may be able to exist as tautomers. All tautomeric forms of these compounds, whether isolated individually or in mixtures, are within the scope of the present invention. For example, in instances where an —OH substituent is permitted on a heteroaromatic ring and ketoenol tautomerism is possible, it is understood that the substituent might in fact be present, in whole or in part, in the oxo (═O) form.

Compounds of the present invention may exist in amorphous form and/or one or more crystalline forms. As such all amorphous and crystalline forms and mixtures thereof of the compounds of the invention are intended to be included within the scope of the present invention. In addition, some of the compounds of the present invention may form solvates with water (i.e., a hydrate) or common organic solvents. Such solvates and hydrates, particularly the pharmaceutically acceptable solvates and hydrates, of the compounds of this invention are likewise encompassed within the scope of the compounds of the invention and the pharmaceutically acceptable salts thereof, along with un-solvated and anhydrous forms of such compounds.

In one example, deuterium isotope content at the deuterium substituted position is greater than the natural isotopic deuterium content (0.015%), more preferably greater than 50%, more preferably greater than 60%, more preferably greater than 75%, more preferably greater than 90%, more preferably greater than 95%, more preferably greater than 97%, more preferably greater than 99%. It will be understood that some variation of natural isotopic abundance may occur in any compound depending upon the source of the reagents used in the synthesis. Thus, a preparation of undeuterated compounds may inherently contain small amounts of deuterated isotopologues, such amounts being insignificant as compared to the degree of stable isotopic substitution of the deuterated compounds of the invention. See, e.g., Gannes, L Z et al., Comp Biochem Physiol Mol Integr Physiol, 119, 725 (1998). Replacement of hydrogen with deuterium may affect the activity, toxicity, and pharmacokinetics (e.g., absorption, distribution, metabolism, and excretion (“ADME”)) of some drugs. For instance, such replacement may alter the chemical stability and biochemical reactivity of a compound through kinetic isotope effects. Because of the increased mass of deuterium relative to hydrogen, epimerization at stereogenic carbons may be slowed down when hydrogen is replaced with deuterium. See Pirali et al, J. Med Chem. 62, 5276-97 (2019). Additionally, the presence of deuterium may affect how a molecule interacts with enzymes, thereby impacting enzyme kinetics. While in certain cases the increased mass of deuterium as compared to hydrogen can stabilize a compound and thereby improve activity, toxicity, or half-life, such impact is not predictable. In other instances deuteration may have little to no impact on these properties, or may affect them in an undesirable manner. Whether and/or how such replacement will impact drug properties can only be determined if the drug is synthesized, evaluated, and compared to its non-deuterated counterpart. See Fukuto et al., J. Med. Chem. 34, 2871-76 (1991). Because some drugs have multiple sites of metabolism or more than one active sites for binding to a target, it is unpredictable as to which sites may benefit by deuterium replacement or to what extent isotope enrichment is necessary to produce a beneficial effect.

### Preparation of the Compounds

The starting materials and reagents used in each step in the preparation are known and can be readily prepared or purchased from commercial sources.

The compound obtained in each step can also be used for the next reaction as a reaction mixture thereof or after obtaining a crude product thereof. Alternatively, the compound obtained in each step can be isolated and/or purified from the reaction mixture by a separation means such as concentration, crystallization, recrystallization, distillation, solvent extraction, fractionation, chromatography and the like according to a conventional method.

In each reaction step, while the reaction time varies depending on the reagents and solvents to be used, unless otherwise specified, it is generally 1 min to 48 h, preferably 10 min to 8 h.

In the reaction of each step, while the reaction temperature varies depending on the reagents and solvents to be used, unless otherwise specified, it is generally −78° C. to 300° C., preferably −78° C. to 150° C.

In the reaction of each step, unless otherwise specified, a reagent is used in 0.5 equivalent to 20 equivalents, preferably 0.8 equivalent to 5 equivalents, relative to the substrate. When a reagent is used as a catalyst, the reagent is used in 0.001 equivalent to 1 equivalent, preferably 0.01 equivalent to 0.2 equivalent, relative to the substrate. When the reagent is also a reaction solvent, the reagent is used in a solvent amount.

In the reaction of each step, unless otherwise specified, it is performed without solvent or by dissolving or suspending in a suitable solvent. Specific examples of the solvent include the following. Alcohols: methanol, ethanol, tert-butyl alcohol, 2-methoxyethanol and the like; ethers: diethyl ether, diphenyl ether, tetrahydrofuran, 1,2-dimethoxyethane and the like; aromatic hydrocarbons: chlorobenzene, toluene, xylene and the like; saturated hydrocarbons: cyclohexane, hexane and the like, amides: N,N-dimethylformamide, N-methylpyrrolidone and the like; halogenated hydrocarbons: dichloromethane, carbon tetrachloride and the like; nitriles: acetonitrile and the like; sulfoxides: dimethyl sulfoxide and the like; aromatic organic bases: pyridine and the like; acid anhydrides: acetic anhydride and the like; organic acids: formic acid, acetic acid, trifluoroacetic acid and the like, inorganic acids: hydrochloric acid, sulfuric acid and the like; esters: ethyl acetate and the like; ketones: acetone, methyl ethyl ketone and the like; and water.

Two or more kinds of the above-mentioned solvents may be used by mixing at an appropriate ratio.

Unless otherwise specified, the reaction of each step is performed according to a known method, for example, the methods described in “Reactions and Syntheses: In the Organic Chemistry Laboratory 2nd Edition” (Lutz F. Tietze, Theophil Eicher, Ulf Diederichsen, Andreas Speicher, Nina Schützenmeister) Wiley, 2015; “Organic Syntheses Collective Volumes 1-12” (John Wiley & Sons Inc); “Comprehensive Organic Transformations, Third Edition” (Richard C. Larock) Wiley, 2018 and the like.

In each step, protection or deprotection of a functional group is performed by a known method, for example, the methods described in “Protective Groups in Organic Synthesis, 4^{th} Ed.” (Theodora W. Greene, Peter G. M. Wuts) Wiley-Interscience, 2007; “Protecting Groups 3rd Ed.” (P. J. Kocienski) Thieme, 2004 and the like.

Deuterated POLRMT modulators of the present invention can be prepared using chemical reactions known to a person of ordinary skill in the art using deuterated starting materials or reagents. Deuterium-containing reagents are well known in the art and can be prepared using known procedures or purchased from commercial sources. The deuterated compounds obtained can be characterized by analytical techniques known to persons of ordinary skill in the art. For example, nuclear magnetic resonance (“NMR”) can be used to determine a compound's structure while mass spectroscopy (“MS”) can be used to determine the amount of deuterium atom in the compound by comparison to its non-deuterated form.

### Compositions

The present invention further includes pharmaceutical compositions of the compounds, a pharmaceutically acceptable salt of said compounds, or prodrugs of said compounds. In addition to the compound of the invention, a salt thereof, or a prodrug thereof, the pharmaceutical compositions comprise one or more pharmaceutically acceptable excipients, such excipients being compatible with other ingredients in the composition and also being not toxic or otherwise harmful. Examples of excipients include carriers, lubricants, binders, disintegrants, solvents, solubilizing agents, suspending agents, isotonic agents, buffers, soothing agents, preservatives, antioxidants, colorants, taste-modifying agents, absorbents, and/or wetting agents.

The pharmaceutical compositions of the invention include those suitable for oral, rectal, nasal, topical, buccal, sublingual, vaginal or parenteral (including subcutaneous, intramuscular, intravenous and intradermal) administration. Such compositions may be prepared by any methods well known in the art of pharmaceutical formulations and pharmacy. See, e.g., Remington: The Science and Practice of Pharmacy, Elsevier Science, 23rd ed. (2020).

Formulations for parenteral administration include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, hydrogels, gelatin, hydrogenated naphthalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

Alternatively, the compositions can be administered by oral ingestion. Compositions intended for oral use can be prepared in solid or liquid forms, according to any method known to a person of ordinary skill in the art for the manufacture of pharmaceutical compositions. Solid dosage forms for oral administration include capsules (both soft and hard gelatin capsules), tablets, powders, and granules. Generally, these pharmaceutical preparations contain active ingredients admixed with pharmaceutically acceptable excipients. These excipients include, for example, inert diluents, such as calcium carbonate, sodium carbonate, lactose, sucrose, glucose, mannitol, cellulose, starch, calcium phosphate, sodium phosphate, kaolin and the like; binding agents, buffering agents, and/or lubricating agents (e.g., magnesium stearate) may also be used. Tablets and capsules can additionally be prepared with release-controlling coatings such as enteric coatings. The compositions may optionally contain sweetening, flavoring, coloring, perfuming, and preserving agents in order to provide a more palatable preparation.

In another example, a pharmaceutical composition of this invention further comprises a second therapeutic agent. The second therapeutic agent may be selected from any pharmaceutically active compound; preferably the second therapeutic agent is known to treat cancer, neurodegenerative disorders, or metabolic disorders. Alternatively, the compounds of the invention and second therapeutic agent may be administered together (within less than 24 hours of one another, consecutively or simultaneously) but in separate pharmaceutical compositions. In certain examples, the compounds of the invention and second therapeutic agent can be administered separately (e.g., more than 24 hours of one another.) If the second therapeutic agent acts synergistically with the compounds of this invention, the therapeutically effective amount of such compounds and/or the second therapeutic agent may be less that such amount required when either is administered alone.

For the treatment of cancer, the compounds described herein may be administered in combination with a chemotherapeutic agent. Therapeutically effective amounts of the additional chemotherapeutic agent(S) are well known to those skilled in the art. However, it is well within the attending physician to determine the amount of other chemotherapeutic agent(S) to be delivered.

Examples of these chemotherapeutic agents include, but are not limited to, Abitrexate (Methotrexate Injection), Abraxane (Paclitaxel Injection), Actemra (Tocilizumab), Adcetris (Brentuximab Vedotin Injection), Adriamycin (Doxorubicin), Adrucil Injection (5-FU (fluorouracil)), Afinitor (Everolimus), Afinitor Disperz (Everolimus), Aldara (Imiquimod), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Avelumab, Bexxar (Tositumomab), BiCNU (Carmustine), Blenoxane (Bleomycin), Blincyto (Blinatumomab), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Cyramza (Ramucirumab), Dacogen (Decitabine), Darzalex (Daratumumab), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Durvalumab, Eligard (Leuprolide), Ellence (Ellence (epirubicin)), Eloxatin (Eloxatin (oxaliplatin)), Elspar (Asparaginase), Emcyt (Estramustine), Empliciti (Elotuzumab), Enhertu (fam-trastuzumab deruxtecan-nxki), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gazyva (Obinutuzumab), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leflunomide (SU101), Lartruvo (Olaratumab), Leukeran (Chlorambucil), Leukine (Sargramostim), Leustatin (Cladribine), Libtayo (Cemiplimab), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Portrazza (Necitumumab), Prednisone Intensol (Prednisone), Proleukin (Aldesleukin), Purinethol (Mercaptopurine), Reclast (Zoledronic acid), Revlimid (Lenalidomide), Removab (Catumaxomab), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Sarclisa (Isatuximab-irfc), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Tecentriq (Atezolizumab), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Tremelimumab, Trelstar (Triptorelin Injection), Trexall (Methotrexate), Trisenox (Arsenic trioxide), Tykerb (lapatinib), Unituxin (Dinutuximab), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting.

### EXAMPLES

The examples and preparations provided below further illustrate and exemplify the compounds of the present invention and methods of preparing such compounds. It is to be understood that the scope of the present invention is not limited in any way by the scope of the following examples and preparations. The compounds and synthetic procedures described in PCT/US22/42097 are hereby incorporated by reference in their entirety.

The structures of the compounds are confirmed by mass spectrometry and/or NMR, where peaks assigned to the characteristic protons in the title compound are presented where appropriate. ¹H NMR shift (6) are given in parts per million (ppm) down field from an internal reference standard.

Table 1 provides a listing of exemplary compounds of the present invention and their IC₅₀ values for inhibition of POLRMT.

The abbreviations used herein are known to a person of ordinary skill in the art. A partial list of abbreviations that may be used herein include: acetonitrile (MeCN), ammonium carbonate (NH₄)₂CO₃, ammonium chloride (NH4Cl), aqueous (aq.), 1,1′-bis(diphenylphosphino)ferrocene (dppf), 1,3-bis(diphenylphosphino)propane (dppp), bis(pinacolato)diboron (B₂pin₂), N-bromosuccinimide (NBS), bromo-tris-pyrrolidino-phosphonium hexafluorophosphate (PyBroP), boron tribromide (BBrr), butyl lithium (BuLi), calculated (Calcd.), cesium carbonate (Cs₂CO₃), dichloromethane (DCM, CH₂Cl₂), N,N-dicyclohexylcarbodiimide (DCC), dichloroethane (DCE), diethyl azodicarboxylate (DEAD), diisopropyl azodicarboxylate (DIAD), N,N-diisopropylethylamine (DIPEA), 4-dimethylaminopyridine (DMAP), dimethylformamide (DMF), dimethyl sulfoxide (DMSO), di-tert-butyl decarbonate (Boc₂O), 1-ethyl-3-(3-dimethylaminopropyl)carbodiimide (EDC), electrospray ionization (ESI), enantiomeric excess (ee), ethyl acetate (EtOAc), hour (h.), N-[(dimethylamino)-1H-1,2,3-triazolo-[4,5-b]pyridin-1-ylmethylene]-N-methylmethanaminium hexafluorophosphate N-oxide (HATU), high performance liquid chromatography (HPLC), hydroxybenzotriazole (HOBt), isopropyl alcohol (IPA), isopropylamine (IPAmine), lithium hydroxide monohydrate (LiOH·H2O), methanol (MeOH), methyl iodide (Mel), minutes (min), potassium carbonate (K₂CO₃), liquid chromatography-mass spectrometry (LCMS), phenyliodide(III) diacetate (PIDA), propylphosphonic anhydride (T₃P), reverse phase (RP), room/ambient temperature (rt, RT), silver oxide (Ag₂O), sodium hydride (NaH), sodium sulfate (Na₂SO₃), supercritical fluid chromatography (SFC), tetrahydrofuran (THF), triethylamine (Et₃N), thionyl chloride (SOCl₂), triphenylphosphine (PPh₃), dicyclohexyl[2′,4′,6′-tris(propan-2-yl)[1,1′-biphenyl]-2-yl]phosphane (XPhos).

Table 1 provides a listing of exemplary compounds of the present invention and their IC₅₀ values for inhibition of POLRMT.

### Example 1-2: Synthesis of chiral analogs of 5,5-difluoro-1-((R)-2-((1-oxo-4-(o-tolyl)-1,2-dihydroisoquinolin-7-yl)oxy)propanoyl)piperidine-3-carboxylic acid

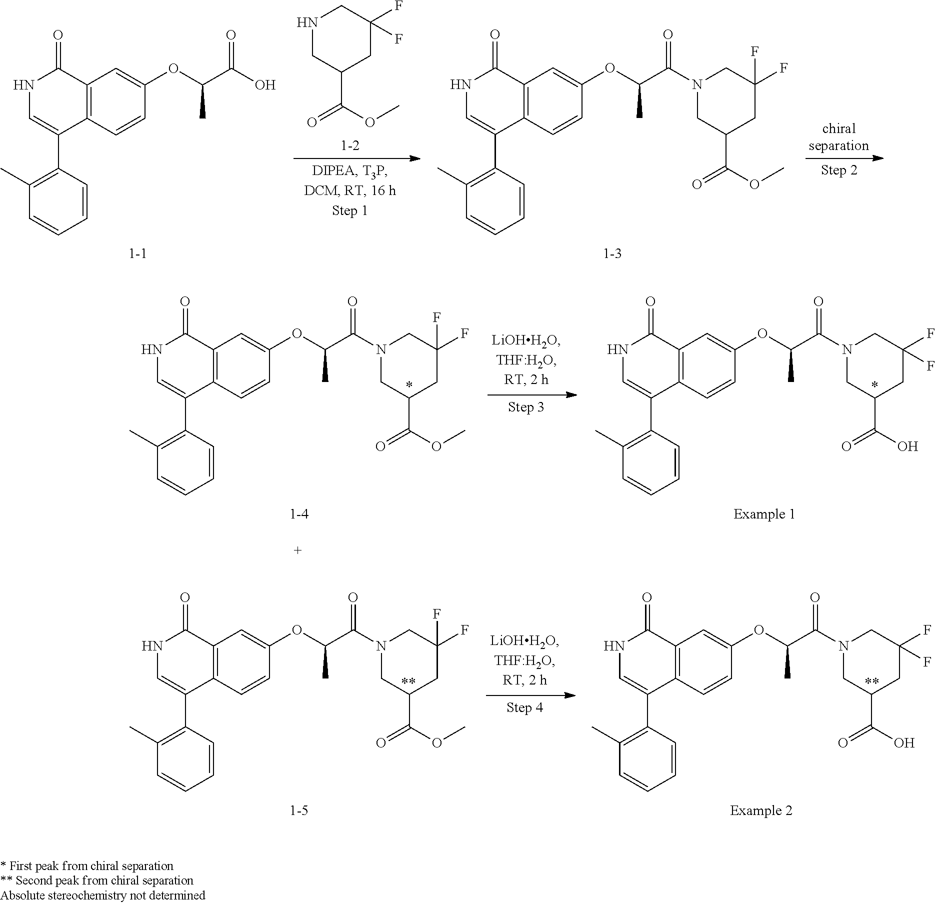

Synthesis of methyl 5,5-difluoro-1-((R)-2-((1-oxo-4-(o-tolyl)-1,2-dihydroisoquinolin-7-yl)oxy)propanoyl)piperidine-3-carboxylate, 3 [Step 1]: To a stirred solution of (R)-2-((1-oxo-4-(o-tolyl)-1,2-dihydroisoquinolin-7-yl)oxy)propanoic acid (1-1, 300 mg, 0.9 mmol) in dichloromethane (6 mL), methyl 5,5-difluoropiperidine-3-carboxylate (2, 165 mg, 0.9 mmol) was added followed by DIPEA (0.6 mL, 3.7 mmol) at 0° C. After 10 min, T₃P (0.85 mL, 1.4 mmol, 50% in ethyl acetate) was added to the reaction mixture at 0° C. and stirred at ambient temperature for 16 h. After completion, the reaction mixture was quenched with ice water and extracted with dichloromethane (twice). The combined organic extracts was washed with brine, dried over anhydrous sodium sulphate and concentrated under reduced pressure to afford methyl 5,5-difluoro-1-((R)-2-((1-oxo-4-(o-tolyl)-1,2-dihydroisoquinolin-7-yl)oxy)propanoyl)piperidine-3-carboxylate (1-3, 230 mg). LCMS (ESI) Calcd. for C₂₆H₂₆F₂N₂O₅: 485, found [M+H]⁺=486.

Synthesis of chiral methyl 5,5-difluoro-1-((R)-2-((1-oxo-4-(o-tolyl)-1,2-dihydroisoquinolin-7-yl)oxy)propanoyl)piperidine-3-carboxylate. 1-4 and 1-5 [Step 2]: The compound methyl 5,5-difluoro-1-((R)-2-((1-oxo-4-(o-tolyl)-1,2-dihydroisoquinolin-7-yl)oxy)propanoyl)piperidine-3-carboxylate (1-3, 230 mg) was submitted to HPLC-SFC for chiral separation. The two fractions were lyophilized to afford the first product as chiral methyl 5,5-difluoro-1-((R)-2-((1-oxo-4-(o-tolyl)-1,2-dihydroisoquinolin-7-yl)oxy)propanoyl)piperidine-3-carboxylate (1-4, 120 mg) as Peak 1 and the second product as chiral methyl 5,5-difluoro-1-((R)-2-((1-oxo-4-(o-tolyl)-1,2-dihydroisoquinolin-7-yl)oxy)propanoyl)piperidine-3-carboxylate (1-5, 130 mg) as Peak 2. The absolute stereochemistries of these compounds were not determined.

1-4: Chiral methyl 5,5-difluoro-1-((R)-2-((1-oxo-4-(o-tolyl)-1,2-dihydroisoquinolin-7-yl)oxy)propanoyl)piperidine-3-carboxylate (Peak 1): LCMS (ESI) Calcd. for C₂₆H₂₆F₂N₂O₅: 485, found [M+H]⁺=486.

1-5: Chiral methyl 5,5-difluoro-1-((R)-2-((1-oxo-4-(o-tolyl)-1,2-dihydroisoquinolin-7-yl)oxy)propanoyl)piperidine-3-carboxylate (Peak 2): LCMS (ESI) Calcd. for C₂₆H₂₆F₂N₂O₅: 485, found [M+H]⁺=485.

Chiral-Prep Method: The chiral separation was performed using a CHLRALPAK IC (250×21 mm), 5μ, which was operated at ambient temperature with a flow rate of 21.0 mL/min. The mobile phase used was a mixture of 70% hexane, 15% dichloromethane, and 15% ethyl alcohol. The instrument was held in isocratic mode for up to 22 min at a wavelength of 228 nm.

Synthesis of 5,5-difluoro-1-((R)-2-((1-oxo-4-(o-tolyl)-1,2-dihydroisoquinolin-7-yl)oxy)propanoyl)piperidine-3-carboxylic acid, Example 1 [Step 3]: To a stirred solution of chiral methyl 5,5-difluoro-1-((R)-2-((1-oxo-4-(o-tolyl)-1,2-dihydroisoquinolin-7-yl)oxy)propanoyl)piperidine-3-carboxylate (1-4, 120 mg. 0.2 mmol) in THF (4 mL) and water (1 mL) mixture, LiOH·H₂O (40 mg, 0.9 mmol) was added portion wise at 0° C. and the reaction mixture was stirred at ambient temperature for 16 h. After completion, the reaction mixture was evaporated under reduced pressure. The reduced mass was diluted with water and washed with ethyl acetate (twice). The aqueous extract was acidified using 1N HCl solution to maintain pH˜3. The mixture was extracted with 10% methanol in dichloromethane (twice). The combined organic extracts was washed with brine, dried over anhydrous sodium sulphate and concentrated under reduced pressure. The product was purified by reverse phase prep-HPLC purification and lyophilized to afford 5,5-difluoro-1-((R)-2-((1-oxo-4-(o-tolyl)-1,2-dihydroisoquinolin-7-yl)oxy)propanoyl)piperidine-3-carboxylic acid (Example 1, 75 mg).

LCMS (ESI) Calcd. for C₂₅H₂₄F₂N₂O₅: 471, found [M+H]⁺=471. ¹H NMR (400 MHz, DMSO-d₆): δ_{H} 11.00 (br s, 1H), 7.71 (s, 1H), 7.34-7.33 (m, 2H), 7.29-7.18 (m, 3H), 6.94-6.91 (m, 1H), 6.84 (s, 1H), 5.44-5.43 (m, 1H), 4.34 (br s, 2H), 3.34 (br s, 2H), 2.53 (br s, 1H)), 2.49 (br s, 1H), 2.40-2.38 (m, 1H), 2.06 (s, 3H), 1.51-1.50 (m, 3H). The absolute stereochemistry was not determined.

Synthesis of 5,5-difluoro-1-((R)-2-((1-oxo-4-(o-tolyl)-1,2-dihydroisoquinolin-7-yl)oxy)propanoyl)piperidine-3-carboxylic acid, Example 2 [Step 4]: To a stirred solution of chiral methyl 5,5-difluoro-1-((R)-2-((1-oxo-4-(o-tolyl)-1,2-dihydroisoquinolin-7-yl)oxy)propanoyl)piperidine-3-carboxylate (1-5, 120 mg, 0.2 mmol) in THF (4 mL) and water (1 mL) mixture, LiOH·H₂O (40 mg, 0.9 mmol) was added portion wise at 0° C. and stirred at ambient temperature for 16 h. After completion, the reaction mixture was evaporated under reduced pressure. The reduced mass was diluted with water and washed with ethyl acetate (twice). The aqueous extract was acidified using 1N HCl solution to maintain pH˜3. The mixture was extracted with 10% methanol in dichloromethane (twice). The combined organic extracts was washed with brine, dried over anhydrous sodium sulphate and concentrated under reduced pressure. The product was purified by reverse phase prep-HPLC purification and lyophilized to afford 5,5-difluoro-1-((R)-2-((1-oxo-4-(o-tolyl)-1,2-dihydroisoquinolin-7-yl)oxy)propanoyl)piperidine-3-carboxylic acid (Example 2, 55 mg). LCMS (ESI) Calcd. for C₂₅H₂₄F₂N₂O₅: 470, found [M+H]⁺=471. ¹H NMR (400 MHz, DMSO-d₆): δ_{H} 10.99 (br s, 1H), 7.71 (s, 1H), 7.34-7.33 (m, 2H), 7.27-7.18 (m, 3H), 6.93-6.90 (m, 1H), 6.83 (s, 1H), 5.40 (br s, 1H), 4.29 (br s, 2H), 3.04 (br s, 2H), 2.58 (br s, 1H), 2.50 (br s, 11H), 2.35-2.32 (m, 1H), 2.06 (s, 3H), 1.53-1.51 (m, 3H). The absolute stereochemistry was not determined.

### Example 3: Synthesis of ethyl N-methyl-N-(1-oxo-4-(o-tolyl)-1,2-dihydroisoquinolin-7-yl)glycinate

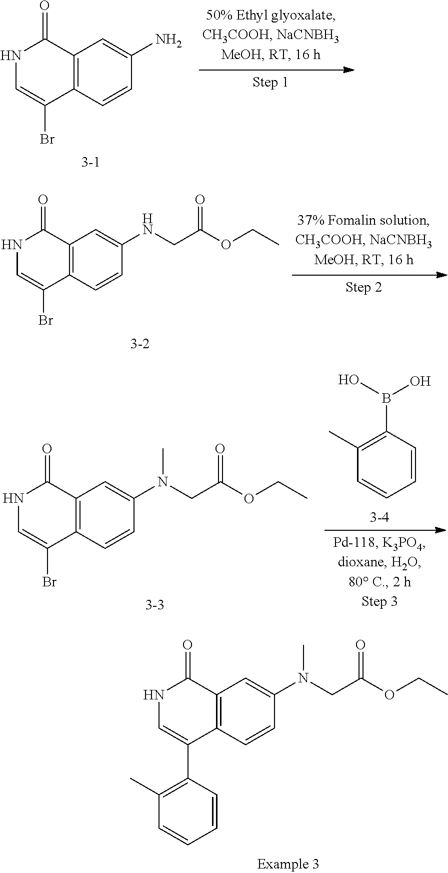

Synthesis of ethyl (4-bromo-1-oxo-1,2-dihydroisoquinolin-7-yl)glycinate, 3-2 [Step 1]: To a stirred solution of 7-amino-4-bromoisoquinolin-1(2H)-one (3-1, 1.0 g, 4.2 mmol) in methanol (10 mL), 50% solution of ethyl 2-oxoacetate in toluene (1.0 mL, 5 mmol) and acetic acid (0.4 mL, 7.0 mmol) were added and the reaction mixture was continued to stir at ambient temperature for 2 h. Then NaBH₃CN (790 mg, 12.5 mmol) was added into the reaction mixture and continued stirring for 16 h. The reaction mixture was quenched with water and extracted with ethyl acetate (twice). The organic phase was washed with brine, dried over anhydrous Na₂SO₄ and concentrated under reduced pressure to afford ethyl (4-bromo-1-oxo-1,2-dihydroisoquinolin-7-yl)glycinate (3-2, 1.0 g). LCMS (ESI) Calcd. for C₁₃H₁₃BrN₂O₃: 325, found [M+H]⁺=327.

Synthesis of ethyl N-(4-bromo-1-oxo-1,2-dihydroisoquinolin-7-yl)-N-methylglycinate, 3-3 [Step 2]: To a stirred solution of ethyl (4-bromo-1-oxo-1,2-dihydroisoquinolin-7-yl)glycinate (3-2, 250 mg, 0.8 mmol) in methanol (4 mL), 37% formalin solution (0.1 mL, 0.9 mmol) was added and continued to stir at ambient temperature for 4 h. Then NaBH₃CN (145 mg, 2.3 mmol) was added into the reaction mixture and continued to stir at ambient temperature for 16 h. The reaction mixture was quenched with water and extracted with ethyl acetate (twice). The combined organic extract was washed with brine, dried over anhydrous Na₂SO₄ and concentrated under reduced pressure. The product was purified by flash chromatography to afford ethyl N-(4-bromo-1-oxo-1,2-dihydroisoquinolin-7-yl)-N-methylglycinate (3-3, 250 mg). LCMS (ESI) Calcd. for C₁₄H₁₅BrN₂O₃: 338, found [M+H]⁺=339.

Synthesis of ethyl N-methyl-N-(1-oxo-4-(o-tolyl)-1,2-dihydroisoquinolin-7-yl)glycinate, Example 3 [Step 3]: To a stirred solution of ethyl N-(4-bromo-1-oxo-1,2-dihydroisoquinolin-7-yl)-N-methylglycinate (3-3, 250 mg, 0.8 mmol) in 1,4-dioxane (6 mL) and water (1.5 mL) mixture, K₃PO₄ (410 mg, 1.9 mmol) and o-tolylboronic acid (3-4, 157 mg, 1.2 mmol) were added into the reaction mixture. The reaction mixture was degassed with nitrogen gas for 10 min and PdCl₂(dtbpf) (50 mg, 0.1 mmol) was added. The reaction mixture was heated at 100° C. for 16 h. After completion, the mixture was filtered through celite bed and the bed was washed with ethyl acetate. The filtrate was washed with water and brine, dried over anhydrous Na₂SO₄ and concentrated under reduced pressure. The product was purified by reverse phase prep HPLC purification method and lyophilized to afford N-methyl-N-(1-oxo-4-(o-tolyl)-1,2-dihydroisoquinolin-7-yl)glycinate (Example 3, 50 mg). LCMS (ESI) Calcd. for C₂₁H₂₂N₂O₃: 350, found [M+H]⁺=351. ¹H NMR (400 MHz, DMSO-d₆): δ_{H} 11.13-11.12 (br s, 1H), 7.39-7.38 (d, 1H), 7.33-7.32 (m, 2H), 7.28-7.24 (m, 1H), 7.18-7.16 (m, 1H), 7.13-7.11 (m, 1H), 6.82-6.80 (d, 1H), 6.73-6.71 (m, 1H), 4.29 (m, 2H), 4.12-4.06 (m, 2H), 3.06-2.98 (s, 3H), 2.04 (s, 3H), 1.18-1.15 (m, 3H).

### Example 4-5: Synthesis of chiral analogs of (S)-1-(N-(4-(2-chloro-4-fluorophenyl)-1-oxo-1,2-dihydroisoquinolin-7-yl)-N-methyl-L-alanyl)piperidine-3-carboxylic acid

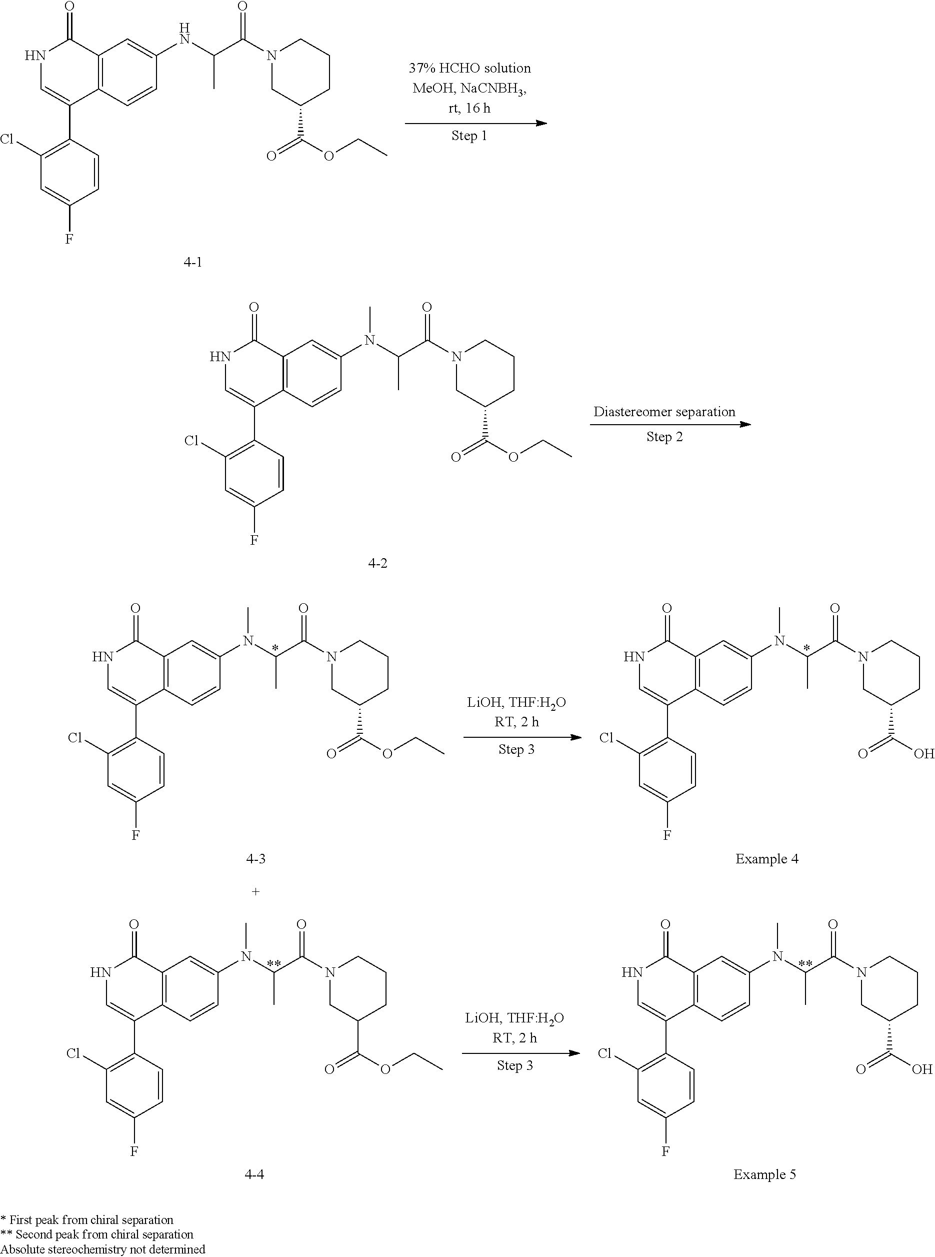 [SMILES: CCOC(=O)[C@H]1CCCN(C(=O)C(C)Nc2ccc3c(-c4ccc(F)cc4Cl)c[nH]c(=O)c3c2)C1 ; CCOC(=O)[C@H]1CCCN(C(=O)C(C)N(C)c2ccc3c(-c4ccc(F)cc4Cl)c[nH]c(=O)c3c2)C1 ; CCOC(=O)[C@H]1CCCN(C(=O)C(C)N(C)c2ccc3c(-c4ccc(F)cc4Cl)c[nH]c(=O)c3c2)C1 ; CC(C(=O)N1CCC[C@H](C(=O)O)C1)N(C)c1ccc2c(-c3ccc(F)cc3Cl)c[nH]c(=O)c2c1 ; CCOC(=O)C1CCCN(C(=O)C(C)N(C)c2ccc3c(-c4ccc(F)cc4Cl)c[nH]c(=O)c3c2)C1 ; CC(C(=O)N1CCC[C@H](C(=O)O)C1)N(C)c1ccc2c(-c3ccc(F)cc3Cl)c[nH]c(=O)c2c1]

Synthesis of ethyl (3S)-1-(N-(4-(2-chloro-4-fluorophenyl)-1-oxo-1,2-dihydroisoquinolin-7-yl)-N-methylalanyl)piperidine-3-carboxylate, 4-2 [Step 1]: To a stirred solution of ethyl (3S)-1-((4-(2-chloro-4-fluorophenyl)-1-oxo-1,2-dihydroisoquinolin-7-yl)alanyl)piperidine-3-carboxylate (4-1, 100 mg, 0.2 mmol) in methanol (1 mL), HCHO solution (0.5 mL, 0.6 mmol, 37%) and acetic acid (0.006 mL, 0.1 mmol) were added and the reaction mixture was allowed to stir at ambient temperature for 4 h. To this reaction mixture, NaBH₃CN (40 mg, 0.6 mmol) was added and the reaction mixture was allowed to stir at ambient temperature for 16 h. The volatiles were removed under reduced pressure. The reduced mass was dissolved in ethyl acetate and washed with water, brine, dried over anhydrous Na₂SO₄ and concentrated under reduced pressure to afford ethyl (3S)-1-(N-(4-(2-chloro-4-fluorophenyl)-1-oxo-1,2-dihydroisoquinolin-7-yl)-N-methylalanyl)piperidine-3-carboxylate (4-2, 80 mg). LCMS (ESI) Calcd. for C₂₇H₂₉ClFN₃O₄: 513, found [M+H]⁺=514.

Synthesis of chiral ethyl (3S)-1-(N-(4-(2-chloro-4-fluorophenyl)-1-oxo-1,2-dihydroisoquinolin-7-yl)-N-methylalanyl)piperidine-3-carboxylate, 4-3 and 4-4 [Step 2]: Diastereomeric mixture of ethyl (3S)-1-(N-(4-(2-chloro-4-fluorophenyl)-1-oxo-1,2-dihydroisoquinolin-7-yl)-N-methylalanyl)piperidine-3-carboxylate (4-2, 260 mg, 0.5 mmol) was separated by normal phase chiral prep HPLC purification method and lyophilized to afford the first compound as chiral ethyl (3S)-1-(N-(4-(2-chloro-4-fluorophenyl)-1-oxo-1,2-dihydroisoquinolin-7-yl)-N-methylalanyl)piperidine-3-carboxylate (4-3, 40 mg) as Peak 1 and the second product as chiral ethyl (3S)-1-(N-(4-(2-chloro-4-fluorophenyl)-1-oxo-1,2-dihydroisoquinolin-7-yl)-N-methylalanyl)piperidine-3-carboxylate (4-4, 60 mg) as Peak 2. The absolute stereochemistries of these compounds were not determined.

Chiral Prep HPLC Method: Chiral separation was performed on an Agilent 1200 series instrument using CHIRALPAK IG (250×21 mm), 5μ, which was operated at ambient temperature with a flow rate of 21.0 mL/min. The mobile phase was a mixture of 50% hexane, 25% DCM, 25% ethyl alcohol and 0.1% IPAmine. The instrument was held in isocratic mode for up to 25 min with a wavelength of 314 nm.

4-3: Chiral ethyl (3S)-1-(N-(4-(2-chloro-4-fluorophenyl)-1-oxo-1,2-dihydroisoquinolin-7-yl)-N-methylalanyl)piperidine-3-carboxylate (Peak 1): LCMS (ESI) Calcd. for C₂₇H₂₉ClFN₃O₄: 513, found [M+H]⁺=514.

4-4: Chiral ethyl (3S)-1-(N-(4-(2-chloro-4-fluorophenyl)-1-oxo-1,2-dihydroisoquinolin-7-yl)-N-methylalanyl)piperidine-3-carboxylate (Peak 2): LCMS (ESI) Calcd. for C₂₇H₂₉ClFN₃O₄: 513, found [M+H]⁺=514.

Synthesis of (S)-1-(N-(4-(2-chloro-4-fluorophenyl)-1-oxo-1,2-dihydroisoquinolin-7-yl)-N-methyl-alanyl)piperidine-3-carboxylic acid, Example 4 [Step 3]: To a stirred solution of chiral ethyl (3S)-1-(N-(4-(2-chloro-4-fluorophenyl)-1-oxo-1,2-dihydroisoquinolin-7-yl)-N-methylalanyl)piperidine-3-carboxylate (Peak 1) (4-3, 60 mg, 0.1 mmol) in THF (2 mL) an aqueous solution (0.5 mL) of LiOH·H₂O (15 mg, 0.4 mmol) was added at ambient temperature and the reaction mixture was allowed to stir for 2 h. The reaction mixture was acidified with citric acid to pH=3 and extracted with ethyl acetate (twice). The organic extract was washed with water, brine, dried over anhydrous Na₂SO₄ and concentrated under reduced pressure. The product was purified by reverse phase prep HPLC purification method and lyophilized to afford (S)-1-(N-(4-(2-chloro-4-fluorophenyl)-1-oxo-1,2-dihydroisoquinolin-7-yl)-N-methyl-alanyl)piperidine-3-carboxylic acid (Example 4, 24 mg). LCMS (ESI) Calcd. for C₂₅H₂₅ClFN₃O₄: 485, found [M+H]⁺=486. ¹H NMR (400 MHz, DMSO-d₆): δ_{H} 11.25 (br s, 1H), 7.59-7.58 (m, 1H), 7.49-7.45 (m, 2H), 7.35-7.30 (m, 2H), 6.89-6.84 (m, 2H), 4.95-4.93 (m, 11H), 4.52-4.49 (m, 1H), 3.80-3.75 (m, 1H), 3.59-3.55 (m, 1H), 2.96-2.90 (m, 1H), 2.82 (s, 3H), 2.71-2.65 (m, 1H), 2.07 (br s, 1H), 1.91 (br s, 1H), 1.47-1.44 (m, 2H), 1.23-1.19 (m, 3H). The absolute stereochemistry was not determined.

Synthesis of (S)-1-(N-(4-(2-chloro-4-fluorophenyl)-1-oxo-1,2-dihydroisoquinolin-7-yl)-N-methyl-alanyl)piperidine-3-carboxylic acid, Example 5 [Step 4]: To a stirred solution of chiral ethyl (3S)-1-(N-(4-(2-chloro-4-fluorophenyl)-1-oxo-1,2-dihydroisoquinolin-7-yl)-N-methylalanyl)piperidine-3-carboxylate (Peak 2) (4-4, 40 mg, 0.1 mmol) in THF (2 mL), an aqueous solution (0.5 mL) of LiOH·H₂O (10 mg, 0.2 mmol) was added. The reaction mixture was allowed to stir for 2 h at ambient temperature. The reaction mixture was acidified with citric acid to pH=3 and extracted with ethyl acetate (thrice). The combined organic extract was washed with water, brine, dried over anhydrous Na₂SO₄ and concentrated under reduced pressure. The product was purified by reverse phase prep HPLC purification method and lyophilized to afford (S)-1-(N-(4-(2-chloro-4-fluorophenyl)-1-oxo-1,2-dihydroisoquinolin-7-yl)-N-methyl-alanyl)piperidine-3-carboxylic acid (Example 5, 20 mg). LCMS (ESI) Calcd. for C₂₅H₂₅ClFN₃O₄: 485, found [M+H]⁺=486. ¹H NMR (400 MHz, DMSO-d₆): δ_{H} 11.23 (br s, 1H), 7.60-7.57 (m, 1H), 7.48-7.45 (m, 2H), 7.32-7.27 (m, 2H), 6.89-6.84 (m, 2H), 5.07-4.92 (m, 1H), 4.09 (br s, 1H), 3.82-3.76 (m, 1H), 3.58-3.55 (m, 1H), 3.11-2.99 (m, 2H), 2.78-2.75 (m, 3H), 2.24 (br s, 1H), 1.55 (m, 2H), 1.33 (m, 1H), 1.23-1.16 (m, 3H). The absolute stereochemistry was not determined.

### Example 6: Synthesis of 7-(((2R)-1-(5-acetyl-2,5-diazabicyclo[2.2.1]heptan-2-yl)-1-oxopropan-2-yl)oxy)-4-(2-chloro-4-fluorophenyl)isoquinolin-1(2H)-one

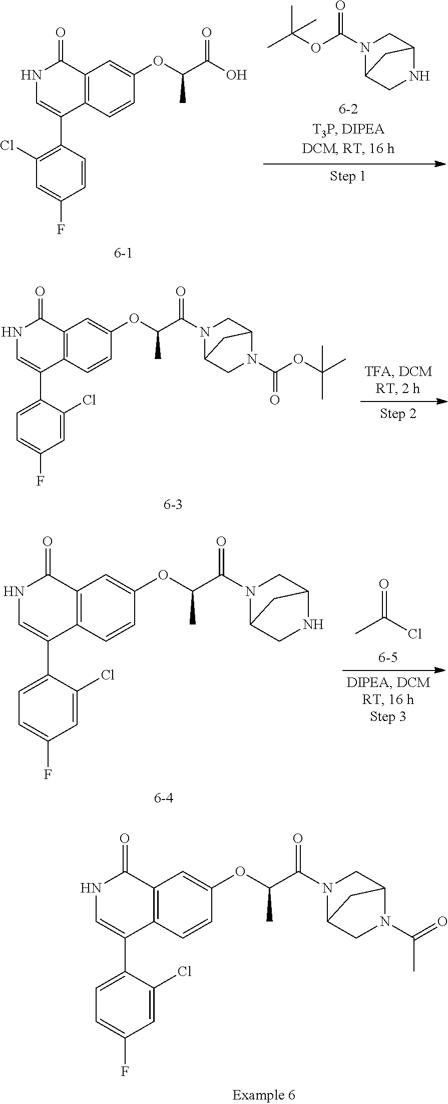

Synthesis of tert-butyl 5-((R)-2-((4-(2-chloro-4-fluorophenyl)-1-oxo-1,2-dihydroisoquinolin-7-yl)oxy)propanoyl)-2,5-diazabicyclo[2.2.1]heptane-2-carboxylate, 6-3 [Step 1]: To a stirred solution of (R)-2-((4-(2-chloro-4-fluorophenyl)-1-oxo-1,2-dihydroisoquinolin-7-yl)oxy)propanoic acid (6-1, 100 mg, 0.3 mmol) in dichloromethane (4 mL), DIPEA (0.2 mL, 0.9 mmol) and tert-butyl 2,5-diazabicyclo[2.2.1]heptane-2-carboxylate (6-2, 80 mg, 0.4 mmol) were added. The reaction mixture was cooled to 0° C., and into this cold mixture T₃P (0.5 mL, 0.8 mmol, 50% in ethyl acetate) was added. The reaction mixture was allowed to warm up to ambient temperature and stirred at ambient temperature for 16 h. The reaction mixture was quenched with ice cold water and extracted with ethyl acetate (twice). The combined organic extract was washed with brine, dried over anhydrous Na₂SO₄ and concentrated under reduced pressure. The product was purified by flash chromatography to afford tert-butyl 5-((R)-2-((4-(2-chloro-4-fluorophenyl)-1-oxo-1,2-dihydroisoquinolin-7-yl)oxy)propanoyl)-2,5-diazabicyclo[2.2.1]heptane-2-carboxylate (6-3, 140 mg). LCMS (ESI) Calcd. for C₂₈H₂₉ClFN₃O₅: 541, found [M+H]⁺=542.

Synthesis of 7-(((2R)-1-(2,5-diazabicyclo[2.2.1]heptan-2-yl)-1-oxopropan-2-yl)oxy)-4-(2-chloro-4-fluorophenyl)isoquinolin-1(2H)-one, 6-4 [Step 2]: A stirred solution of tert-butyl 5-((R)-2-((4-(2-chloro-4-fluorophenyl)-1-oxo-1,2-dihydroisoquinolin-7-yl)oxy)propanoyl)-2,5-diazabicyclo[2.2.1]heptane-2-carboxylate (6-3, 140 mg, 0.26 mmol) in dichloromethane (4 mL) was cooled to 0° C. and into this reaction mixture trifluoroacetic acid (2.0 mL, 26.0 mmol) was added drop wise. The reaction mixture was allowed to warm up to ambient temperature and stirred for 2 h. The volatiles were removed under reduced pressure and azeotroped with dichloroethane, and finally concentrated under reduced pressure to afford 7-(((2R)-1-(2,5-diazabicyclo[2.2.1]heptan-2-yl)-1-oxopropan-2-yl)oxy)-4-(2-chloro-4-fluorophenyl)isoquinolin-1(2H)-one (6-4, 110 mg). LCMS (ESI) Calcd. for C₂₃H₂₁ClFN₃O₃: 441, found [M+H]⁺=442.

Synthesis of 7-(((2R)-1-(5-acetyl-2,5-diazabicyclo[2.2.1]heptan-2-yl)-1-oxopropan-2-yl)oxy)-4-(2-chloro-4-fluorophenyl)isoquinolin-1(2H)-one, Example 6 [Step 3]: A solution of 7-(((2R)-1-(2,5-diazabicyclo[2.2.1]heptan-2-yl)-1-oxopropan-2-yl)oxy)-4-(2-chloro-4-fluorophenyl)isoquinolin-1(2H)-one (6-4, 120 mg, 0.3 mmol) in dichloromethane (2 mL) was cooled at 0° C. and then into the reaction mixture DIPEA (0.5 mL, 3 mmol) and acetyl chloride (6-5, 0.05 mL, 0.5 mmol) were added. The reaction mixture was allowed to warm up to ambient temperature and continued to stir at ambient temperature for 16 h. The volatiles were removed under reduced pressure, diluted with water, and extracted with ethyl acetate. The combined organic extract was washed with water and brine, dried over anhydrous Na₂SO₄ and concentrated under reduced pressure. The product was purified by reverse phase prep HPLC purification method and lyophilized to afford 7-(((2R)-1-(5-acetyl-2,5-diazabicyclo[2.2.1]heptan-2-yl)-1-oxopropan-2-yl)oxy)-4-(2-chloro-4-fluorophenyl)isoquinolin-1(2H)-one (Example 6, 60 mg). LCMS (ESI) Calcd. for C₂₁H₂₂N₂O₃: 350, found [M+H]⁺=351. ¹H NMR (400 MHz, DMSO-d₆): δ_{H} 11.43 (br s, 1H), 7.62-7.57 (m, 1H), 7.33-7.32 (d, 1H), 7.54-7.40 (m, 2H), 7.37-7.29 (m, 1H), 7.00-6.91 (m, 2H), 5.36-4.61 (m, 3H), 3.70-3.52 (m, 2H), 3.41-3.19 (m, 1H), 2.01-1.99 (s, 3H), 1.90-1.74 (m, 2H), 1.49-1.43 (m, 3H). The final product was isolated as a mixture of diastereomers.

### Example 7-8: Synthesis of chiral analogs of (S)-1-(N-methyl-N-(1-oxo-4-(0-tolyl)-1,2-dihydroisoquinolin-7-yl)-alanyl)piperidine-3-carboxylic acid

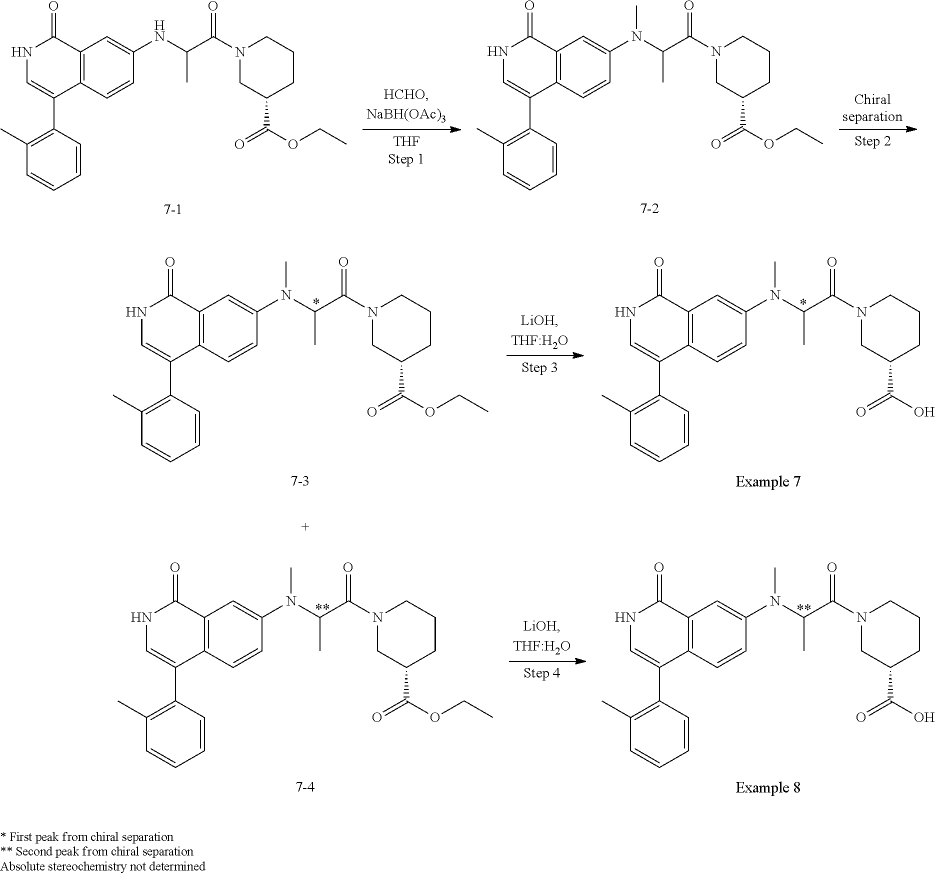 [SMILES: CCOC(=O)[C@H]1CCCN(C(=O)C(C)Nc2ccc3c(-c4ccccc4C)c[nH]c(=O)c3c2)C1 ; CCOC(=O)[C@H]1CCCN(C(=O)C(C)N(C)c2ccc3c(-c4ccccc4C)c[nH]c(=O)c3c2)C1 ; CCOC(=O)[C@H]1CCCN(C(=O)C(C)N(C)c2ccc3c(-c4ccccc4C)c[nH]c(=O)c3c2)C1 ; Cc1ccccc1-c1c[nH]c(=O)c2cc(N(C)C(C)C(=O)N3CCC[C@H](C(=O)O)C3)ccc12 ; CCOC(=O)[C@H]1CCCN(C(=O)C(C)N(C)c2ccc3c(-c4ccccc4C)c[nH]c(=O)c3c2)C1 ; Cc1ccccc1-c1c[nH]c(=O)c2cc(N(C)C(C)C(=O)N3CCC[C@H](C(=O)O)C3)ccc12]

Synthesis of ethyl (3S)-1-(N-methyl-N-(1-oxo-4-(o-tolyl)-1,2-dihydroisoquinolin-7-yl)alanyl)piperidine-3-carboxylate, 7-2 [Step 1]: To a stirred solution of ethyl (3S)-1-((1-oxo-4-(o-tolyl)-1,2-dihydroisoquinolin-7-yl)alanyl)piperidine-3-carboxylate (7-1, 460 mg, 1.0 mmol) in methanol (6 mL), HCHO solution (0.7 mL, 3.0 mmol, 37 wt % in H₂O) was added followed by acetic acid (0.03 mL, 0.5 mmol) and stirred at ambient temperature for 4 h. Into the mixture, NaBH₃CN (190 mg, 3.0 mmol) was added and stirred at ambient temperature for 16 h. After completion, the volatiles were removed under reduced pressure and the reduced mass was partitioned between water and ethyl acetate. The organic extract was separated. The aqueous layer was further extracted with ethyl acetate (twice). The combined organic extract was washed with water and brine, dried over anhydrous sodium sulphate and concentrated under reduced pressure to afford ethyl (3S)-1-(N-methyl-N-(1-oxo-4-(o-tolyl)-1,2-dihydroisoquinolin-7-yl)alanyl)piperidine-3-carboxylate (7-2, 400 mg). LCMS (ESI) Calcd. for C₂₈H₃₃N₃O₄: 476, found [M+H]⁺=477.

Synthesis of chiral ethyl (3S)-1-(N-methyl-N-(1-oxo-4-(o-tolyl)-1,2-dihydroisoquinolin-7-yl)alanyl)piperidine-3-carboxylate, 7-3 and 7-4 [Step 2]: The racemic compound ethyl (3S)-1-(N-methyl-N-(1-oxo-4-(o-tolyl)-1,2-dihydroisoquinolin-7-yl)alanyl)piperidine-3-carboxylate (7-2, 400 mg) was purified by normal phase chiral-HPLC and lyophilized to afford the first product chiral ethyl (3S)-1-(N-methyl-N-(1-oxo-4-(o-tolyl)-1,2-dihydroisoquinolin-7-yl)alanyl)piperidine-3-carboxylate (7-3, 90 mg) as Peak 2 and the second product as chiral ethyl (3S)-1-(N-methyl-N-(1-oxo-4-(o-tolyl)-1,2-dihydroisoquinolin-7-yl)alanyl)piperidine-3-carboxylate (7-4, 110 mg) as Peak 3. The absolute stereochemistry of both diastereomers were not determined.

Chiral Prep HPLC Method: Chiral separation was performed on an Agilent 1200 series instrument using CHIRALPAK IG (250×21 mm), 5μ, which was operated at ambient temperature with a flow rate of 21.0 mL/min. The mobile phase was a mixture of 50% hexane, 25% DCM and 25% EtOH. The instrument was held in isocratic mode for up to 20 min with a wavelength of 314 nm.

7-3: Chiral ethyl (3S)-1-(N-methyl-N-(1-oxo-4-(o-tolyl)-1,2-dihydroisoquinolin-7-yl)alanyl)piperidine-3-carboxylate (Peak 2): LCMS (ESI) Calcd. for C₂₈H₃₃N₃O₄: 476, found [M+H]⁺=477.

7-4: Chiral ethyl (3S)-1-(N-methyl-N-(1-oxo-4-(o-tolyl)-1,2-dihydroisoquinolin-7-yl)alanyl)piperidine-3-carboxylate (Peak 3): LCMS (ESI) Calcd. for C₂₈H₃₃N₃O₄: 476, found [M+H]⁺=477.

Synthesis of (S)-1-(N-methyl-N-(1-oxo-4-(o-tolyl)-1,2-dihydroisoquinolin-7-yl)-alanyl)piperidine-3-carboxylic acid, Example 7 [Step 3]: To a stirred solution of chiral ethyl (3S)-1-(N-methyl-N-(1-oxo-4-(o-tolyl)-1,2-dihydroisoquinolin-7-yl)alanyl)piperidine-3-carboxylate, peak 2 (7-3, 100 mg, 0.2 mmol) in THF (5 mL), LiOH·H₂O (35 mg, 0.8 mmol) in water (1.5 mL) was added dropwise at 0° C. and stirring was continued for 2 h at ambient temperature. After completion of the reaction, THF was removed under reduced pressure and the pH was adjusted to 5-6 by using 4N HCl and lyophilized. The product was purified by reverse phase PREP-HPLC and lyophilized to afford (S)-1-(N-methyl-N-(1-oxo-4-(o-tolyl)-1,2-dihydroisoquinolin-7-yl)-alanyl) piperidine-3-carboxylic acid (Example 7, 48 mg). LCMS (ESI) Calcd. for C₂₆H₂₉N₃O₄: 448, found [M−H]⁻=446. ¹H NMR (400 MHz, DMSO-d₆, 0.100° C.): δ_{H} 7.56 (br s, 1H), 7.33-7.17 (m, 5H), 6.88 (d, 1H), 6.71 (br s, 1H), 4.92-4.87 (m, 1H), 3.79 (br s, 1H), 3.10-2.90 (m, 4H), 2.87 (s, 3H), 2.33 (br s, 1H), 2.08 (br s, 3H), 1.96-1.93 (m, 1H), 1.60-1.52 (m, 2H), 1.38-1.32 (m, 1H), 1.27-1.26 (m, 3H). The absolute stereochemistry was not determined.

Synthesis of (S)-1-(N-methyl-N-(1-oxo-4-(o-tolyl)-1,2-dihydroisoquinolin-7-yl)-alanyl)piperidine-3-carboxylic acid, Example 8 [Step 4]: To a stirred solution of chiral ethyl (3S)-1-(N-methyl-N-(1-oxo-4-(o-tolyl)-1,2-dihydroisoquinolin-7-yl)alanyl)piperidine-3-carboxylate, peak 3 (7-4, 110 mg, 0.2 mmol) in THF (5 mL), LiOH·H₂O (40 mg, 0.9 mmol) in water (1.5 mL) was added dropwise at 0° C. and stirring was continued for 2 h at ambient temperature. After completion of the reaction, THF was removed under reduced pressure and the pH was adjusted to 5-6 by using 4N HCl and lyophilized. The product was purified by reverse phase PREP-HPLC and lyophilized to afford (S)-1-(N-methyl-N-(1-oxo-4-(o-tolyl)-1,2-dihydroisoquinolin-7-yl)-alanyl) piperidine-3-carboxylic acid (Example 8, 55 mg). LCMS (ESI) Calcd. for C₂₆H₂₉N₃O₄: 448, found [M−H]⁻=446. ¹H NMR (400 MHz, DMSO-d₆, 0.100° C.): δ_{H} 7.56 (br s, 1H), 7.33-7.17 (m, 5H), 6.87 (d, 1H), 6.70 (br s, 1H), 4.94 (br s, 1H), 3.79 (br s, 1H), 3.22 (br s, 2H), 3.10-3.04 (m, 2H), 2.82 (s, 3H), 2.33 (br s, 1H), 2.07 (br s, 3H), 1.87 (br s, 1H), 1.68-1.59 (m, 2H), 1.40-1.38 (m, 1H), 1.27-1.23 (m, 3H). The absolute stereochemistry was not determined.

### Example 9-10: Synthesis of chiral analogs of methyl 1-((R)-2-((4-(2-chloro-4-fluorophenyl)-1-oxo-1,2-dihydroisoquinolin-7-yl)oxy)propanoyl)-5,5-difluoropiperidine-3-carboxylate

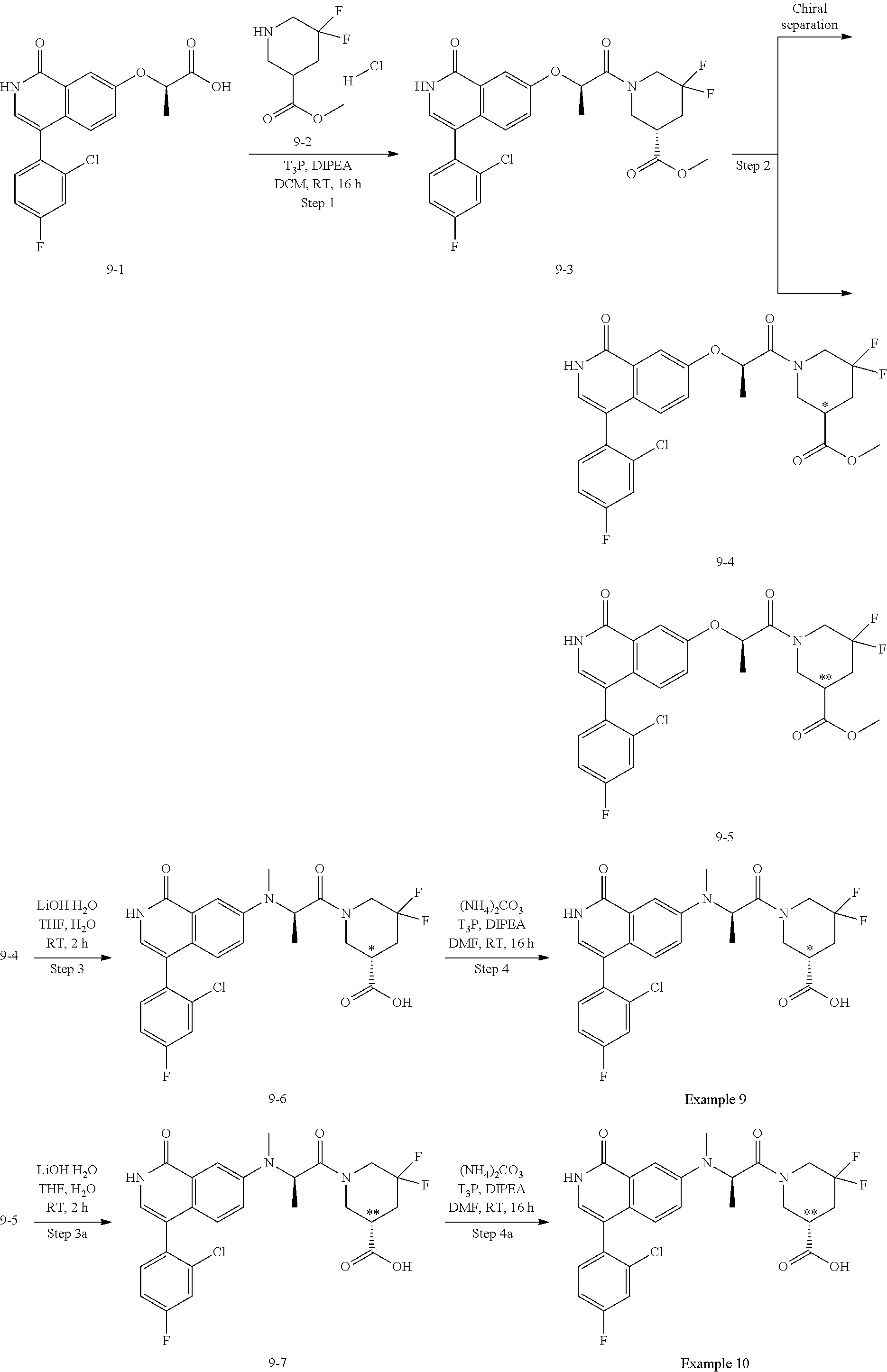

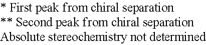

Synthesis of methyl 1-((R)-2-((4-(2-chloro-4-fluorophenyl)-1-oxo-1,2-dihydroisoquinolin-7-yl)oxy)propanoyl)-5,5-difluoropiperidine-3-carboxylate, 9-3 [Step 1]: To a stirred solution of (R)-2-((4-(2-chloro-4-fluorophenyl)-1-oxo-1,2-dihydroisoquinolin-7-yl)oxy)propanoic acid (9-1, 290 mg, 0.8 mmol) in dichloromethane (6 mL), methyl 5,5-difluoropiperidine-3-carboxylate hydrochloride (2, 175 mg, 0.8 mmol) was added followed by DIPEA (0.6 mL, 3.2 mmol) at 0° C. After 10 min, T₃P (0.7 mL, 1.2 mmol, 50% in ethyl acetate) was added to the reaction mixture at 0° C. and stirred at ambient temperature for 16 h. After completion, the reaction mixture was quenched with ice water and extracted with dichloromethane (twice). The combined organic extracts was washed with brine, dried over anhydrous sodium sulphate and concentrated under reduced pressure. The product was purified by column chromatography to afford methyl 1-((R)-2-((4-(2-chloro-4-fluorophenyl)-1-oxo-1,2-dihydroisoquinolin-7-yl)oxy)propanoyl)-5,5-difluoropiperidine-3-carboxylate (9-3, 220 mg). LCMS (ESI) Calcd. for C₂₅H₂₂ClF₃N₂O₅: 523, found [M+H]⁺=523.

Synthesis of chiral methyl 1-((R)-2-((4-(2-chloro-4-fluorophenyl)-1-oxo-1,2-dihydroisoquinolin-7-yl)oxy)propanoyl)-5,5-difluoropiperidine-3-carboxylate, 9-4 and 9-5 [Step 2]: Racemic methyl 1-((R)-2-((4-(2-chloro-4-fluorophenyl)-1-oxo-1,2-dihydroisoquinolin-7-yl)oxy)propanoyl)-5,5-difluoropiperidine-3-carboxylate (9-3, 220 mg) was submitted to normal phase for chiral separation. After chiral separation two fractions were lyophilized to afford the first product as chiral methyl 1-((R)-2-((4-(2-chloro-4-fluorophenyl)-1-oxo-1,2-dihydroisoquinolin-7-yl)oxy)propanoyl)-5,5-difluoropiperidine-3-carboxylate (9-4, 80 mg) as Peak 1 and the second product as chiral methyl 1-((R)-2-((4-(2-chloro-4-fluorophenyl)-1-oxo-1,2-dihydroisoquinolin-7-yl)oxy)propanoyl)-5,5-difluoropiperidine-3-carboxylate (9-5, 85 mg) as Peak 2. The absolute stereochemistries of these compounds were not determined.

Chiral separation method: Chiral separation was performed using an Agilent 1200 series instrument with CHIRALART CELLULOSE SC (250×20 mm), 5μ, which was operated at ambient temperature with a flow rate of 18.0 mL/min. Mobile phase used was a mixture of 60% hexane, 20% DCM, and 20% EtOH. The instrument was held in isocratic mode for up to 20 min at a wavelength of 228 nm.

Chiral methyl 1-((R)-2-((4-(2-chloro-4-fluorophenyl)-1-oxo-1,2-dihydroisoquinolin-7-yl)oxy)propanoyl)-5,5-difluoropiperidine-3-carboxylate, 9-4: LCMS (ESI) Calcd. for C₂₅H₂₂ClF₃N₂O₅: 523, found [M+H]⁺=523.

Chiral methyl 1-((R)-2-((4-(2-chloro-4-fluorophenyl)-1-oxo-1,2-dihydroisoquinolin-7-yl)oxy)propanoyl)-5,5-difluoropiperidine-3-carboxylate, 9-5: LCMS (ESI) Calcd. for C₂₅H₂₂ClF₃N₂O₅: 523, found [M+H]⁺=523.

Synthesis of 1-((R)-2-((4-(2-chloro-4-fluorophenyl)-1-oxo-1,2-dihydroisoquinolin-7-yl)oxy)propanoyl)-5,5-difluoropiperidine-3-carboxylic acid, 9-6 [Step 3]: To a stirred solution of chiral methyl 1-((R)-2-((4-(2-chloro-4-fluorophenyl)-1-oxo-1,2-dihydroisoquinolin-7-yl)oxy)propanoyl)-5,5-difluoropiperidine-3-carboxylate (9-4, 100 mg, 0.2 mmol) in tetrahydrofuran (4 mL) and water (1 mL) mixture, LiOH·H₂O (110 mg, 2.6 mmol) was added portion wise at 0° C. and stirred at ambient temperature for 2 h. After completion, the reaction mixture was evaporated under reduced pressure. The reduced material was diluted with water and washed with ethyl acetate (twice). The aqueous extract was acidified with 1N HCl solution to maintain pH˜3. The mixture was extracted with 10% methanol in dichloromethane (twice). The combined organic extracts was washed with brine, dried over anhydrous sodium sulphate and concentrated under reduced pressure to 1-((R)-2-((4-(2-chloro-4-fluorophenyl)-1-oxo-1,2-dihydroisoquinolin-7-yl)oxy)propanoyl)-5,5-difluoropiperidine-3-carboxylic acid (9-6, 90 mg). LCMS (ESI) Calcd. for C₂₄H₂₀ClF₃N₂O₅: 509, found [M+H]⁺=509. The absolute stereochemistry was not determined.

Synthesis of 1-((R)-2-((4-(2-chloro-4-fluorophenyl)-1-oxo-1,2-dihydroisoquinolin-7-yl)oxy)propanoyl)-5,5-difluoropiperidine-3-carboxylic acid, 9-7 [Step 3A]: To a stirred solution of chiral methyl 1-((R)-2-((4-(2-chloro-4-fluorophenyl)-1-oxo-1,2-dihydroisoquinolin-7-yl)oxy)propanoyl)-5,5-difluoropiperidine-3-carboxylate (9-5, 100 mg, 0.2 mmol) in tetrahydrofuran (4 mL) and water (1 mL) mixture, LiOH·H₂O (30 mg, 0.7 mmol) was added portion wise at 0° C. and stirred at ambient temperature for 2 h. After completion, the reaction mixture was evaporated under reduced pressure. The reduced material was diluted with water and washed with ethyl acetate (twice). The aqueous extract was acidified with 1N HCl solution to maintain pH˜3. The mixture was extracted with 10% methanol in dichloromethane (twice). The combined organic extracts was washed with brine, dried over anhydrous sodium sulphate and concentrated under reduced pressure to afford 1-((R)-2-((4-(2-chloro-4-fluorophenyl)-1-oxo-1,2-dihydroisoquinolin-7-yl)oxy)propanoyl)-5,5-difluoropiperidine-3-carboxylic acid (9-7, 95 mg). LCMS (ESI) Calcd. for C₂₄H₂₀ClF₃N₂O₅: 509, found [M+H]⁺=509. The absolute stereochemistry was not determined.

Synthesis of 1-((R)-2-((4-(2-chloro-4-fluorophenyl)-1-oxo-1,2-dihydroisoquinolin-7-yl)oxy)propanoyl)-5,5-difluoropiperidine-3-carboxamide, Example 9 [Step 4]: To a solution of 1-((R)-2-((4-(2-chloro-4-fluorophenyl)-1-oxo-1,2-dihydroisoquinolin-7-yl)oxy)propanoyl)-5,5-difluoropiperidine-3-carboxylic acid (9-6, 100 mg, 0.2 mmol) in DMF (3 mL), DIPEA (0.2 mL, 1.4 mmol) was added followed by (NH₄)₂CO₃ (285 mg, 2.9 mmol). The reaction mixture was cooled to 0° C. and T₃P (0.23 mL, 0.4 mmol, 50% in ethyl acetate) was added and the reaction mixture was stirred at ambient temperature for 16 h. After completion of the reaction, it was quenched with ice cold water and extracted with ethyl acetate (twice). The combined organic extracts was washed with brine solution, dried over anhydrous Na₂SO₄, and concentrated under reduced pressure. The product was purified by reverse phase prep HPLC purification and lyophilized to afford 1-((R)-2-((4-(2-chloro-4-fluorophenyl)-1-oxo-1,2-dihydroisoquinolin-7-yl)oxy)propanoyl)-5,5-difluoropiperidine-3-carboxamide (Example 9, 35 mg). LCMS (ESI) Calcd. for C₂₄H₂₁ClF₃N₃O₄: 508, found [M+H]⁺=508. ¹H NMR (400 MHz, DMSO-d₆): δ_{H} 11.10 (br s, 1H), 7.71 (br s, 1H), 7.52-7.44 (m, 2H), 7.31-7.24 (m, 2H), 7.31-7.29 (m, 1H), 7.26-7.24 (m, 1H), 6.97-6.94 (m, 2H), 5.49 (br s, 1H), 4.73 (br s, 1H), 4.33 (br s, 1H), 3.25 (br s, 2H), 2.81 (br s, 1H), 2.32 (br s, 1H), 2.23-2.11 (m, 1H), 51 (d, 3H). The absolute stereochemistry was not determined.

Synthesis of 1-((R)-2-((4-(2-chloro-4-fluorophenyl)-1-oxo-1,2-dihydroisoquinolin-7-yl)oxy)propanoyl)-5,5-difluoropiperidine-3-carboxamide, Example 10 [Step 4A]: To a stirred solution of 1-((R)-2-((4-(2-chloro-4-fluorophenyl)-1-oxo-1,2-dihydroisoquinolin-7-yl)oxy)propanoyl)-5,5-difluoropiperidine-3-carboxylic acid (9-7, 120 mg, 0.24 mmol) in DMF (3 mL), DIPEA (0.3 mL, 1.7 mmol) was added followed by (NH₄)₂CO₃ (340 mg, 3.5 mmol). The reaction mixture was cooled to 0° C. and T₃P (0.3 mL, 0.47 mmol) was added and the reaction mixture was stirred at ambient temperature for 16 h. After completion, the reaction mixture was quenched with ice cold water, extracted with ethyl acetate (twice). The combined organic extracts was washed with brine solution, dried over anhydrous Na₂SO₄, and concentrated under reduced pressure. The product was purified by reverse phase prep HPLC purification and lyophilized to afford 1-((R)-2-((4-(2-chloro-4-fluorophenyl)-1-oxo-1,2-dihydroisoquinolin-7-yl)oxy)propanoyl)-5,5-difluoropiperidine-3-carboxamide (Example 10, 45 mg). LCMS (ESI) Calcd. for C₂₄H₂₁ClF₃N₃O₄: 508, found [M+H]⁺=508. ¹H NMR (400 MHz, DMSO-d₆): δ_{H} 11.08 (br s, 1H), 7.72 (br s, 1H), 7.52-7.50 (m, 1H), 7.47-7.44 (m, 1H), 7.31-7.29 (m, 1H), 7.26-7.24 (m, 1H), 6.97-6.94 (m, 2H), 5.44 (br s, 1H), 4.43 (br s, 1H), 4.32 (br s, 1H), 3.31 (br s, 2H), 2.61 (br s, 1H), 2.32 (br s, 1H), 2.23-2.11 (m, 1H), 1.52 (d, 3H). The absolute stereochemistry was not determined.

### Example 11: Synthesis of 4-(2-(1-hydroxyethyl)phenyl)-7-methoxyisoquinolin-1(2H)-one

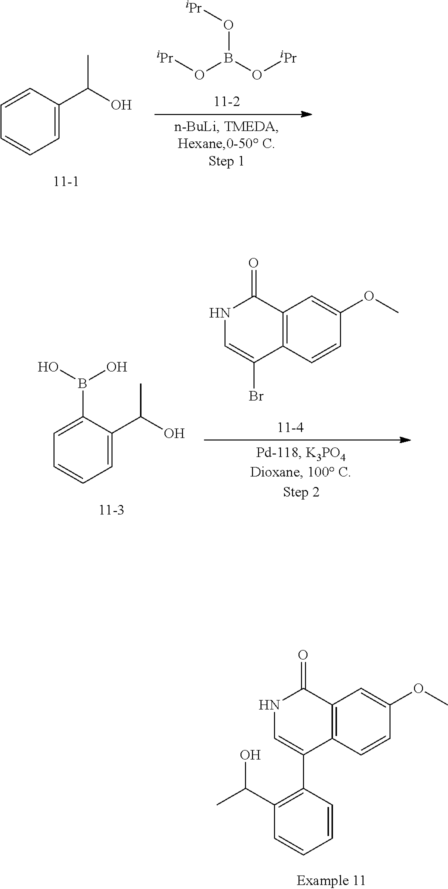

Synthesis of (2-(1-hydroxyethyl)phenyl)boronic acid, 11-3 [Step 1]: A stirred solution of 1-phenylethan-1-ol (11-1, 1.2 g, 9.8 mmol), tetramethylethylenediamine (1.8 mL, 11.8 mmol) in hexane (20 mL) was cooled to 0° C. n-BuLi (7.8 mL, 19.6 mmol, 2.5M in hexane) was added dropwise into the mixture. The reaction mixture was heated at 50° C. for 1 h and then allowed to cool to ambient temperature. Triisopropyl borate (11-2, 4.5 mL, 19.6 mmol) was added dropwise into the mixture at ambient temperature and stirred at 40° C. for 2 h. The reaction mixture was cooled to 0° C. and 2N HCl was added dropwise and stirred for 15 min. The organic layer was separated and the aqueous layer was again extracted with hexane. The combined organic extract was washed with brine and concentrated under reduced pressure to afford (2-(1-hydroxyethyl)phenyl)boronic acid (11-3, 500 mg). The product was used in the next reaction without further purification.

Synthesis of 4-(2-(1-hydroxyethyl)phenyl)-7-methoxyisoquinolin-1(2H)-one, Example 11 [Step 2]: In a sealed vial 4-bromo-7-methoxyisoquinolin-1(2H)-one (114, 50 mg, 0.2 mmol) and (2-(1-hydroxyethyl)phenyl)boronic acid (11-3, 50 mg, 0.3 mmol) and K₃PO₄ (85 mg, 0.4 mmol) were taken. 1,4-Dioxane (2 mL) and water (0.5 mL) were added and nitrogen gas was purged for 10 min. [1,1′-Bis(di-tert-butylphosphino)ferrocene]dichloropalladium(II) (15 mg, 0.02 mmol) was added into the mixture and heated at 100° C. for 18 h. After completion, the reaction mixture was diluted with ethyl acetate and washed with brine. The organic extract was dried over anhydrous Na₂SO₄ and concentrated under reduced pressure. The product was purified by reverse phase HPLC prep chromatography to afford 4-(2-(1-hydroxyethyl)phenyl)-7-methoxyisoquinolin-1(2H)-one (Example 11, 25 mg). LCMS (ESI) Calcd. for C₁₈H₁₇NO₃: 295, found [M+H]⁺=296. ¹H NMR (400 MHz, DMSO-d₆): δ_{H} 11.06 (s, 1H), 7.74 (s, 1H), 7.69 (t, 1H), 7.45 (t, 1H), 734-7.24 (m, 2H), 7.13 (t, 1H), 7.06-6.81 (m, 2H), 4.67 (br s, 1H), 4.49 (br s, 1H), 3.89 (s, 3H), 1.27-1.11 (m, 3H). The product was a mixture of atropisomers.

### Example 12-13: Synthesis of chiral analogs of methyl 2-(1-((R)-2-((1-oxo-4-(o-tolyl)-1,2-dihydroisoquinolin-7-yl)oxy)propanoyl)piperidin-3-yl)acetate

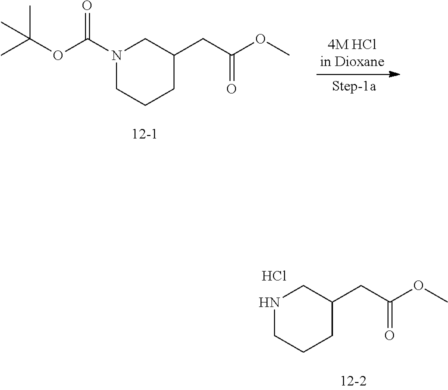 [SMILES: COC(=O)CC1CCCN(C(=O)OC(C)(C)C)C1 ; Cl ; COC(=O)CC1CCCNC1]

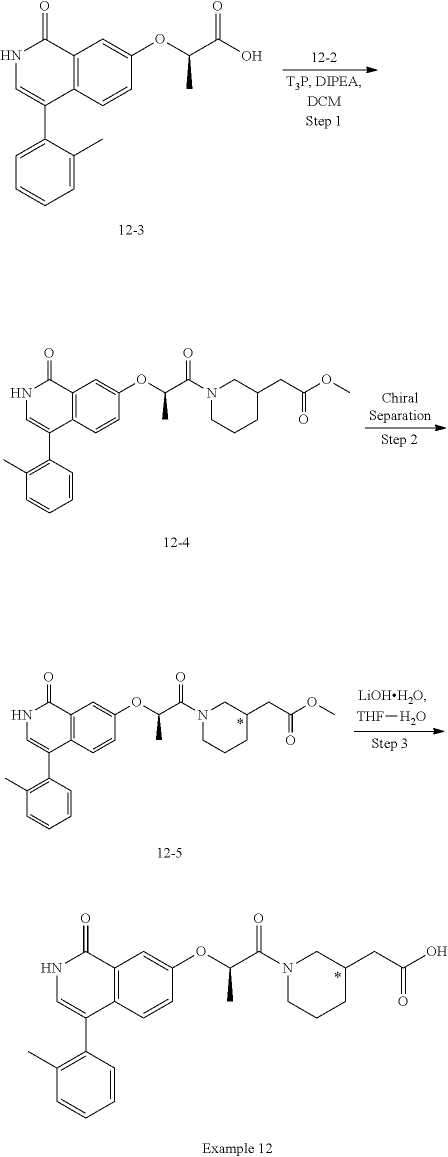

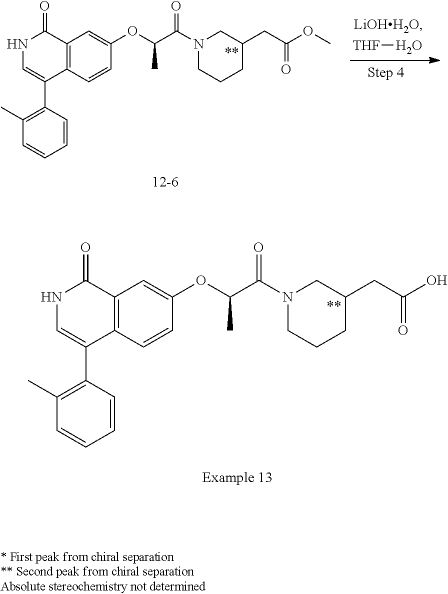

Synthesis of methyl 2-(piperidin-3-yl)acetate hydrochloride, 12-2 [Step 1a]: To a stirred solution of tert-butyl 3-(2-methoxy-2-oxoethyl)piperidine-1-carboxylate (12-1, 1.0 g, 3.9 mmol) in dichloromethane (2 mL) (4N) HCl in 1,4-dioxane (7.0 mL, 11.7 mmol) was added dropwise at 0° C. and the reaction mixture was stirred at ambient temperature for 3 h under inert atmosphere. After completion, the reaction mixture was concentrated under reduced pressure and azeotroped with n-pentane and diethyl ether (2:1) to afford methyl 2-(piperidin-3-yl)acetate hydrochloride (12-2, 700 mg). ¹H NMR (400 MHz, DMSO-d₆): δ_{H} 9.17 (d, 1H), 3.60 (s, 3H), 3.17 (br s, 2H), 2.69 (br s, 1H), 2.69 (br s, 1H), 2.39-2.15 (m, 3H), 1.75-1.47 (m, 3H), 1.23-1.14 (m, 2H).

Synthesis of methyl 2-(1-((R)-2-((1-oxo-4-(o-tolyl)-1,2-dihydroisoquinolin-7-yl)oxy)propanoyl)piperidin-3-yl)acetate, 12-4 [Step 1]: To a stirred solution of (R)-2-((1-oxo-4-(o-tolyl)-1,2-dihydroisoquinolin-7-yl)oxy)propanoic acid (12-3, 250 mg, 0.7 mmol) in dichloromethane (6 mL), methyl 2-(piperidin-3-yl)acetate hydrochloride (225 mg, 1.2 mmol) was added followed by DIPEA (0.7 mL, 3.9 mmol) at 0° C. After 10 min, TP (0.7 mL, 1.2 mmol, 50% in ethyl acetate) was added to the reaction mixture and stirred at ambient temperature for 16 h. After completion, the reaction mixture was diluted with dichloromethane and washed with water (twice) and brine (twice). The organic layer was dried over anhydrous Na₂SO₄, filtered and concentrated under reduced pressure. The product was purified by column chromatography to afford methyl 2-(1-((R)-2-((1-oxo-4-(o-tolyl)-1,2-dihydroisoquinolin-7-yl)oxy)propanoyl)piperidin-3-yl)acetate (12-4, 320 mg). LCMS (ESI) Calcd. for C₂₇H₃₀N₂O₅: 462, found [M+H]⁺=463.

Synthesis of chiral methyl 2-(1-((R)-2-((1-oxo-4-(o-tolyl)-1.2-dihydroisoquinolin-7-yl)oxy)propanoyl)piperidin-3-yl)acetate, 12-5 and 12-6 [Step 2]: The racemic methyl 24(1-((R)-2-((1-oxo-4-(o-tolyl)-1,2-dihydroisoquinolin-7-yl)oxy)propanoyl)piperidin-3-yl)acetate (12-4, 300 mg) was separated using chiral separation (normal phase) to afford the first product as chiral methyl 2-(1-((R)-2-((1-oxo-4-(o-tolyl)-1,2-dihydroisoquinolin-7-yl)oxy)propanoyl)piperidin-3-yl)acetate (12-5, 100 mg) as Peak 1 and the second product as chiral methyl 2-(1-((R)-2-((1-oxo-4-(o-tolyl)-1,2-dihydroisoquinolin-7-yl)oxy)propanoyl)piperidin-3-yl)acetate (12-6, 100 mg) as Peak 2. The absolute stereochemistries were not determined and was arbitrarily assigned.

12-5: Chiral methyl 2-(1-((R)-2-((1-oxo-4-(o-tolyl)-1,2-dihydroisoquinolin-7-yl)oxy)propanoyl)piperidin-3-yl)acetate (Peak 1): LCMS (ESI) Calcd. for C₂₇H₃₀N₂O₅: 462, found [M+H]⁺=463.

12-6: Chiral methyl 2-(1-((R)-2-((1-oxo-4-(o-tolyl)-1,2-dihydroisoquinolin-7-yl)oxy)propanoyl)piperidin-3-yl)acetate (Peak 2): LCMS (ESI) Calcd. for C₂₇H₃₀N₂O₅: 462, found [M+H]⁺=463.

Chiral prep method: Chiral separation was performed on an Agilent 1200 series instrument using CHIRALPAK IA (250×20 mm), 5μ, which was operated at ambient temperature with a flow rate of 18.0 mL/min. Mobile phase was a mixture of 85% hexane, and 15% ethanol. The instrument was held in isocratic mode for up to 30 min with a wavelength of nm.

Synthesis of 2-(1-((R)-2-((1-oxo-4-(o-tolyl)-1,2-dihydroisoquinolin-7-yl)oxy)propanoyl)piperidin-3-yl)acetic acid, Example 12 [Step 3]: To a stirred solution of chiral methyl 2-(1-((R)-2-((1-oxo-4-(o-tolyl)-1,2-dihydroisoquinolin-7-yl)oxy)propanoyl)piperidin-3-yl)acetate, Peak 1 (12-5, 100 mg, 0.2 mmol) in THF (4 mL), LiOH·H₂O (35 mg, 0.9 mmol) in water (1 mL) was added at 0° C. and the mixture was stirred at ambient temperature for 12 h. The volatiles were removed under reduced pressure. The product was purified by Prep HPLC (reverse phase) and lyophilized to afford 2-(1-((R)-2-((1-oxo-4-(o-tolyl)-1,2-dihydroisoquinolin-7-yl)oxy)propanoyl)piperidin-3-yl)acetic acid (Example 12, 25 mg). LCMS (ESI) Calcd. for C₂₆H₂₈N₂O₅: 448, found [M+H]⁺=449. ¹HNMR (400 MHz, DMSO-d₆, 100° C.): δ_{H} 10.90 (br s, 1H), 7.69 (s, 1H), 7.34-7.20 (m, 5H), 6.92 (d, 1H), 6.84 (s, 1H), 5.29 (br s, 1H), 4.09 (d, 2H), 2.78 (br s, 1H), 2.54 (s, 1H), 2.16 (br s, 2H), 2.07 (s, 3H), 1.84 (d, 1H), 1.68 (br s, 1H), 1.50 (d, 3H), 1.43-1.27 (m, 2H). The absolute stereochemistry was not determined.

Synthesis of 2-(1-((R)-2-((1-oxo-4-(o-tolyl)-1,2-dihydroisoquinolin-7-yl)oxy)propanoyl)piperidin-3-yl)acetic acid, Example 13 [Step 4]: To a stirred solution of chiral methyl 2-(1-((R)-2-((1-oxo-4-(o-tolyl)-1,2-dihydroisoquinolin-7-yl)oxy)propanoyl)piperidin-3-yl)acetate, Peak 2 (12-6, 100 mg, 0.2 mmol) in THF (4 mL), LiOH·H₂O (36 mg, 0.9 mmol) in water (1 mL) was added at 0° C. and the mixture was stirred at ambient temperature for 12 h. The volatiles were removed under reduced pressure. The product was purified by Prep HPLC (reverse phase) and lyophilized to afford 2-(1-((R)-2-((1-oxo-4-(o-tolyl)-1,2-dihydroisoquinolin-7-yl)oxy)propanoyl)piperidin-3-yl)acetic acid (Example 13, 35 mg). LCMS (ESI) Calcd. for C₂₆H₂₈N₂O₅: 448, found [M+H]⁺=449. ¹HNMR (400 MHz, DMSO-d₆, 100° C.): δ_{H} 10.90 (br s, 1H), 7.69 (s, 1H), 7.34-7.20 (m, 5H), 6.92 (d, 1H), 6.84 (s, 1H), 5.29 (br s, 1H), 4.09 (d, 2H), 2.78 (br s, 1H), 2.54 (s, 1H), 2.16 (br s, 2H), 2.07 (s, 3H), 1.84 (d, 1H), 1.68 (br s, 1H), 1.50 (d, 3H), 1.43-1.27 (m, 2H). The absolute stereochemistry was not determined.

### Example 14: Synthesis of(R)-1-(2-((4-(2-chloro-4-fluorophenyl)-1-oxo-1,2-dihydroisoquinolin-7-yl)oxy)propanoyl)-4-methylpiperidine-4-carboxylic acid

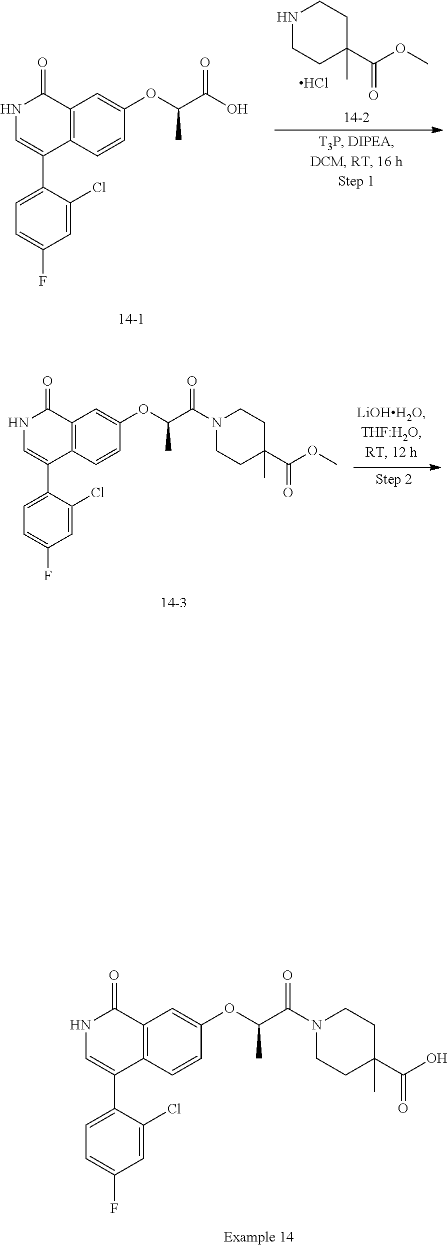 [SMILES: C[C@@H](Oc1ccc2c(-c3ccc(F)cc3Cl)c[nH]c(=O)c2c1)C(=O)O ; COC(=O)C1(C)CCNCC1 ; Cl ; COC(=O)C1(C)CCN(C(=O)[C@@H](C)Oc2ccc3c(-c4ccc(F)cc4Cl)c[nH]c(=O)c3c2)CC1 ; C[C@@H](Oc1ccc2c(-c3ccc(F)cc3Cl)c[nH]c(=O)c2c1)C(=O)N1CCC(C)(C(=O)O)CC1]

Synthesis of methyl (R)-1-(2-((4-(2-chloro-4-fluorophenyl)-1-oxo-1,2-dihydroisoquinolin-7-yl)oxy)propanoyl)-4-methylpiperidine-4-carboxylate, 14-3 [Step 1]: To a stirred solution of (R)-2-((4-(2-chloro-4-fluorophenyl)-1-oxo-1,2-dihydroisoquinolin-7-yl)oxy)propanoic acid (14-1, 150 mg, 0.4 mmol) in dichloromethane (6 mL), methyl 4-methylpiperidine-4-carboxylate hydrochloride (14-2, 80 mg, 0.4 mmol) was added followed by DIPEA (0.2 mL, 1.2 mmol) at 0° C. After 10 min, T₃P (0.4 mL, 0.6 mmol, 50% in ethyl acetate) was added. The reaction mixture was stirred at ambient temperature for 16 h and quenched with ice-cold water and extracted with dichloromethane (twice). The combined organic extracts were washed with brine, dried over anhydrous sodium sulphate, filtered and concentrated under reduced pressure to afford methyl (R)-1-(2-((4-(2-chloro-4-fluorophenyl)-1-oxo-1,2-dihydroisoquinolin-7-yl)oxy)propanoyl)-4-methylpiperidine-4-carboxylate (14-3, 140 mg). LCMS (ESI) Calcd. for C₂₆H₂₆ClFN₂O₅: 500, found [M+H]⁺=501.

Synthesis of (R)-1-(2-((4-(2-chloro-4-fluorophenyl)-1-oxo-1,2-dihydroisoquinolin-7-yl)oxy)propanoyl)-4-methylpiperidine-4-carboxylic acid, Example 14 [Step 2]: To a stirred solution of methyl (R)-1-(2-((4-(2-chloro-4-fluorophenyl)-1-oxo-1,2-dihydroisoquinolin-7-yl)oxy)propanoyl)-4-methylpiperidine-4-carboxylate (14-3, 140 mg, 0.3 mmol) in tetrahydrofuran (6 mL) and water (1.5 mL) mixture, lithium hydroxide monohydrate (17 mg, 0.5 mmol) was added portion wise at 0° C. The reaction mixture was stirred at ambient temperature for 16 h and volatiles were evaporated under reduced pressure. The residue was diluted with water and washed with ethyl acetate. The aqueous phase was acidified with 1N HCl solution to pH˜3 and extracted with a mixture of 10% methanol in dichloromethane (twice). The combined organic extracts were washed with brine, dried over anhydrous sodium sulphate and concentrated under reduced pressure. The product was purified by reverse phase prep-HPLC to afford (R)-1-(2-((4-(2-chloro-4-fluorophenyl)-1-oxo-1,2-dihydroisoquinolin-7-yl)oxy)propanoyl)-4-methylpiperidine-4-carboxylic acid (Example 14, 21 mg). LCMS (ESI) Calcd. for C₂₅H₂₄ClFN₂O₅: 486, found [M+H]⁺=487. ¹H NMR (400 MHz, DMSO-d₆ at 100° C.): δ_{H} 10.98 (br s, 1H), 7.66 (s, 1H), 7.51 (d, 1H), 7.47-7.44 (m, 1H), 7.32-7.28 (m, 1H), 7.25-7.23 (m, 1H), 6.97-6.93 (m, 2H), 5.42-5.39 (m, 1H), 3.89-3.86 (m, 2H), 3.26-3.16 (m, 2H), 2.00-1.97 (m, 2H), 1.49 (d, 3H), 1.37-1.32 (m, 2H), 1.19 (m, 3H).

### Example 15. Synthesis of (R)-4-methyl-1-(2-((1-oxo-4-(o-tolyl)-1,2-dihydroisoquinolin-7-yl)oxy)propanoyl)piperidine-4-carboxylic acid

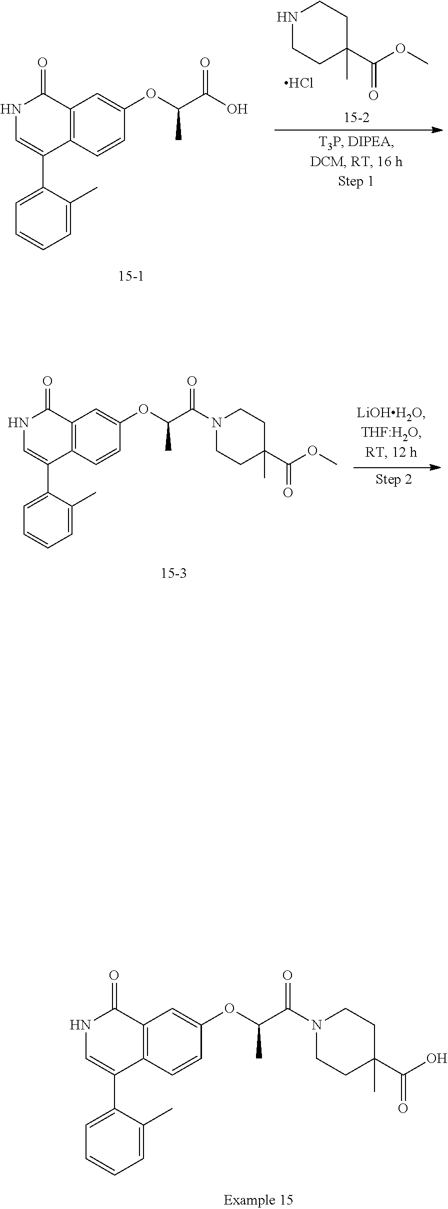 [SMILES: Cc1ccccc1-c1c[nH]c(=O)c2cc(O[C@H](C)C(=O)O)ccc12 ; Cl ; COC(=O)C1(C)CCNCC1 ; COC(=O)C1(C)CCN(C(=O)[C@@H](C)Oc2ccc3c(-c4ccccc4C)c[nH]c(=O)c3c2)CC1 ; Cc1ccccc1-c1c[nH]c(=O)c2cc(O[C@H](C)C(=O)N3CCC(C)(C(=O)O)CC3)ccc12]

Synthesis of methyl (R)-4-methyl-1-(2-((1-oxo-4-(o-tolyl)-1,2-dihydroisoquinolin-7-yl)oxy)propanoyl)piperidine-4-carboxylate, 15-3 [Step 1]: To a stirred solution of (R)-2-((1-oxo-4-(o-tolyl)-1,2-dihydroisoquinolin-7-yl)oxy)propanoic acid (15-1, 150 mg, 0.4 mmol) in dichloromethane (6 mL), methyl 4-methylpiperidine-4-carboxylate hydrochloride (15-2, 80 mg, 0.42 mmol) was added followed by DIPEA (0.2 mL, 1.2 mmol) at 0° C. After 10 min, T₃P (0.4 mL, 0.6 mmol, 50% in ethyl acetate) was added. The reaction mixture was stirred at ambient temperature for 16 h and quenched with ice-cold water and extracted with dichloromethane (twice). The combined organic extracts were washed with brine, dried over anhydrous sodium sulphate, filtered and concentrated under reduced pressure to give methyl (R)-4-methyl-1-(2-((1-oxo-4-(o-tolyl)-1,2-dihydroisoquinolin-7-yl)oxy)propanoyl)piperidine-4-carboxylate (15-3, 140 mg). LCMS (ESI) Calcd. for C₂₆H₂₆ClFN₂O₅: 500, found [M+H]⁻=501.

Synthesis of (R)-4-methyl-1-(2-((1-oxo-4-(o-tolyl)-1,2-dihydroisoquinolin-7-yl)oxy)propanoyl)piperidine-4-carboxylic acid, Example 15 [Step 2]: To a stirred solution of methyl (R)-4-methyl-1-(2-((1-oxo-4-(o-tolyl)-1,2-dihydroisoquinolin-7-yl)oxy)propanoyl)piperidine-4-carboxylate (15-3, 140 mg, 0.3 mmol) in tetrahydrofuran (6 mL) and water (1.5 mL) mixture, lithium hydroxide monohydrate (20 mg, 0.5 mmol) was added portion wise at 0° C. The reaction mixture was stirred at ambient temperature for 16 h and volatiles were evaporated under reduced pressure. The residue was diluted with water and washed with ethyl acetate. The aqueous phase was acidified with 1N HCl solution to pH˜3 and extracted with a mixture of 10% methanol in dichloromethane (twice). The combined organic extracts were washed with brine, dried over anhydrous sodium sulphate and concentrated under reduced pressure. The product was purified by reverse phase prep-HPLC to afford (R)-4-methyl-1-(2-((1-oxo-4-(o-tolyl)-1,2-dihydroisoquinolin-7-yl)oxy)propanoyl)piperidine-4-carboxylic acid (Example 15, 45 mg). LCMS (ESI) Calcd. for C₂₆H₂₈N₂O₅: 448, found [M+H]⁺=449. ¹H NMR (400 MHz, DMSO-d₆ at 100° C.): δ_{H} 10.85 (br s, 1H), 7.66 (s, 1H), 7.34-7.33 (m, 2H), 7.28-7.27 (m, 1H), 7.23-7.18 (m, 2H), 6.93 (d, 1H), 6.84 (s, 1H), 5.34-5.30 (m, 1H), 3.89-3.86 (m, 2H), 3.20 (br s, 2H), 2.07 (s, 3H), 2.00-1.98 (m, 2H), 1.49 (d, 3H), 1.37-1.32 (m, 2H), 1.20 (s, 3H).

### Example 16: Synthesis of (R)-4-(2-((1-oxo-4-(o-tolyl)-1,2-dihydroisoquinolin-7-yl)oxy)propanoyl)piperazine-1-carboxamide

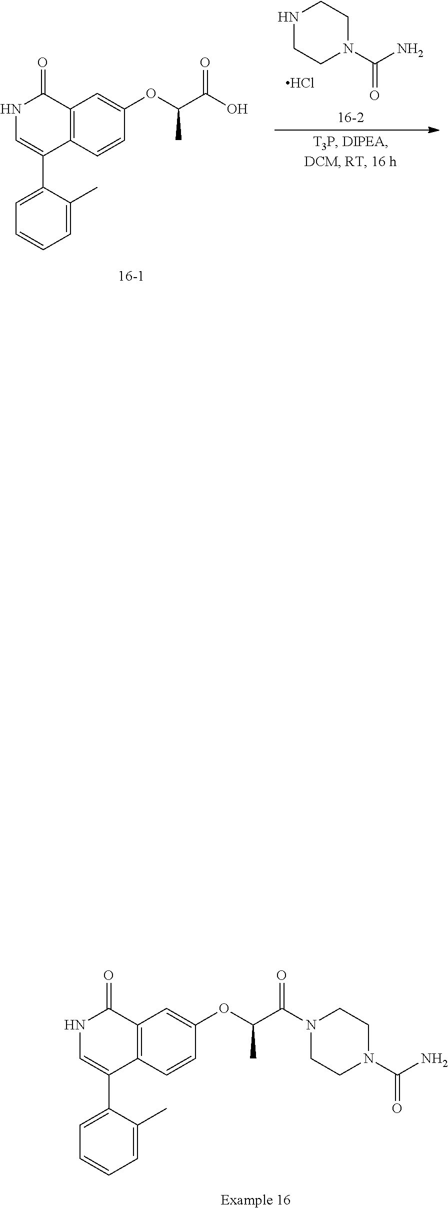 [SMILES: Cc1ccccc1-c1c[nH]c(=O)c2cc(O[C@H](C)C(=O)O)ccc12 ; Cl ; NC(=O)N1CCNCC1 ; Cc1ccccc1-c1c[nH]c(=O)c2cc(O[C@H](C)C(=O)N3CCN(C(N)=O)CC3)ccc12]

To a stirred solution of (R)-2-((1-oxo-4-(o-tolyl)-1,2-dihydroisoquinolin-7-yl)oxy)propanoic acid (16-1, 100 mg, 0.3 mmol) in dichloromethane (5 mL), piperazine-1-carboxamide hydrochloride (16-2, 60 mg, 0.4 mmol) was added followed by DIPEA (0.1 mL, 0.8 mmol) at 0° C. After 10 min, propanephosphonic acid anhydride (0.4 mL, 0.6 mmol, 50% in ethyl acetate) was added. The reaction mixture was stirred at ambient temperature for 16 h, quenched with ice-cold water and extracted with dichloromethane. The combined organic extracts were washed with brine, dried over anhydrous sodium sulphate, filtered and concentrated under reduced pressure. The product was purified by reverse phase prep-HPLC to afford (R)-4-(2-((1-oxo-4-(o-tolyl)-1,2-dihydroisoquinolin-7-yl)oxy)propanoyl)piperazine-1-carboxamide (Example 16, 46 mg). LCMS (ESI) Calcd. for C₂₄H₂₆N₄O₄: 434, found [M+H]⁺=435. ¹H NMR (400 MHz, DMSO-d₆ at 100° C.): δ_{H} 10.99 (s, 1H), 7.70 (br s, 1H), 7.35-7.34 (m, 2H) 7.30-7.18 (m, 3H), 6.94 (d, 1H), 6.84 (s, 1H), 5.74 (br s, 2H), 5.39-5.34 (m, 1H), 3.55-3.35 (m, 8H), 2.07 (s, 3H), 1.51 (d, 3H).

### Example 17. Synthesis of (R)-N-(2-(N-methylacetamido)ethyl)-2-((1-oxo-4-(o-tolyl)-1,2-dihydroisoquinolin-7-yl)oxy)propanamide

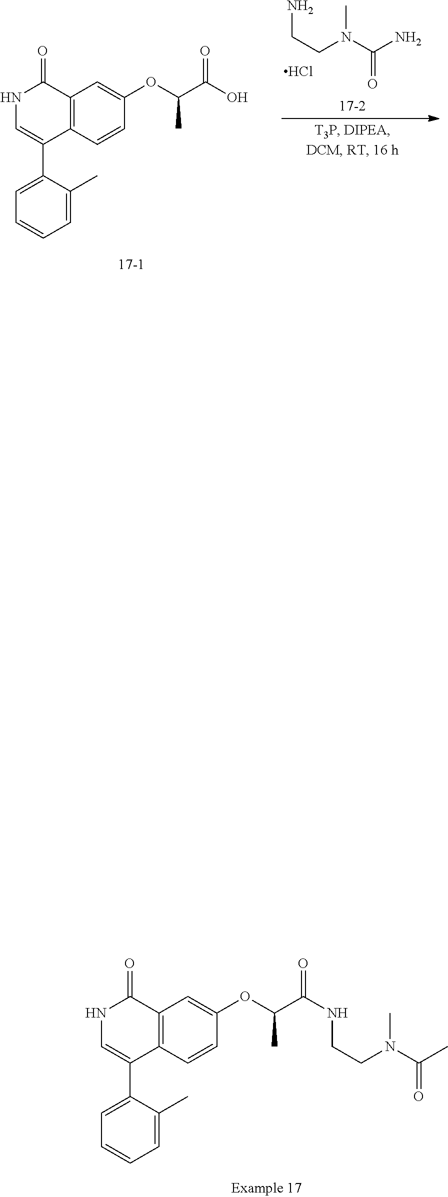

To a stirred solution of (R)-2-((1-oxo-4-(o-tolyl)-1,2-dihydroisoquinolin-7-yl)oxy)propanoic acid (17-1, 100 mg, 0.3 mmol) in dichloromethane (5 mL), N-(2-aminoethyl)-N-methyl-acetamide hydrochloride (17-2, 55 mg, 0.4 mmol) was added followed by DIPEA (0.2 mL, 1.0 mmol) at 0° C. After 10 min, T₃P (0.3 mL, 0.5 mmol, 50% in ethyl acetate) was added. The reaction mixture was stirred at ambient temperature for 16 h, quenched with ice-cold water, and extracted with dichloromethane. The combined organic extracts were washed with brine, dried over anhydrous sodium sulfate, filtered, and concentrated under reduced pressure. The product was purified by reverse phase prep-HPLC to afford (R)-N-(2-(N-methylacetamido)ethyl)-2-((1-oxo-4-(o-tolyl)-1,2-dihydroisoquinolin-7-yl)oxy)propanamide (Example 17, 54 mg). LCMS (ESI) Calcd. for C₂₄H₂₇N₃O₄: 421, found [M+H]⁺=422. ¹H NMR (400 MHz, DMSO-d₆, at 100° C.): δ_{H} 11.01 (br s, 1H), 8.06-7.90 (m, 1H), 7.75 (s, 1H), 7.35-7.27 (m, 4H), 7.20-7.18 (m, 1H), 6.96-6.93 (m, 1H), 6.85 (s, 1H), 4.79 (s, 1H), 3.34-3.28 (m, 4H), 2.89-2.78 (m, 3H), 2.07 (s, 3H), 1.92 (s, 3H), 1.47 (d, 3H).

### Example 18: Synthesis of (R)-7-((1-(4-ethylpiperazin-1-yl)-1-oxopropan-2-yl)oxy)-4-(0-tolyl)isoquinolin-1(2H)-one

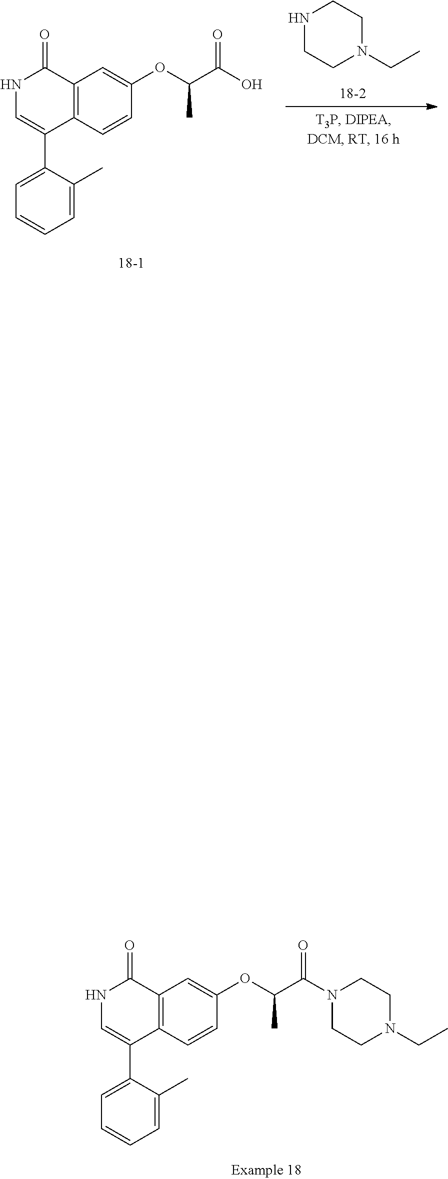

To a stirred solution of (R)-2-((1-oxo-4-(o-tolyl)-1,2-dihydroisoquinolin-7-yl)oxy)propanoic acid (18-1, 100 mg, 0.3 mmol) in dichloromethane (5 mL), 1-ethyl piperazine (18-2, 40 mg, 0.4 mmol) was added followed by DIPEA (0.2 mL, 0.9 mmol) at 0° C. After 10 min, T₃P (0.3 mL, 0.5 mmol, 50% in ethyl acetate) was added. The reaction mixture was stirred at ambient temperature for 16 h, quenched with ice-cold water, and extracted with dichloromethane. The combined organic extracts were washed with brine, dried over anhydrous sodium sulfate, filtered, and concentrated under reduced pressure. The product was purified by reverse phase prep-HPLC to afford (R)-7-((1-(4-ethylpiperazin-1-yl)-1-oxopropan-2-yl)oxy)-4-(o-tolyl)isoquinolin-1(2H)-one (Example 18, 60 mg). LCMS (ESI) Calcd. for C₂₅H₂₉N₃O₃: 419, found [M+H]⁺=420. ¹H NMR (400 MHz, DMSO-d₆): δ_{H} 11.32 (d, 1H), 7.61-7.59 (m, 1H), 7.35-7.18 (m, 5H), 6.91-6.87 (m, 2H), 5.44-5.39 (m, 1H) 3.76-3.73 (m, 1H), 3.64-3.62 (m, J H), 3.55-3.50 (m, 1H), 3.31-3.27 (m, 1H), 2.39-2.34 (m, 5H), 2.26 (br s, 1H), 2.04 (d, 3H), 1.45 (d, 3H), 1.00 (t, 3H).

### Example 19: Synthesis of (R)-N-(2-acetamidoethyl)-N-methyl-2-((1-oxo-4-(o-tolyl)-1,2-dihydroisoquinolin-7-yl)oxy)propanamide

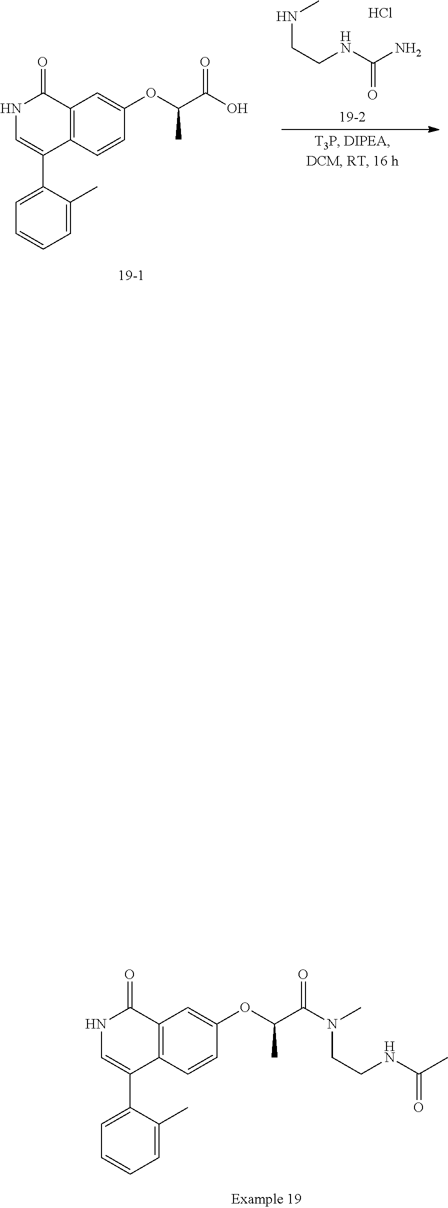

To a stirred solution of (R)-2-((1-oxo-4-(o-tolyl)-1,2-dihydroisoquinolin-7-yl)oxy)propanoic acid (19-1, 100 mg, 0.31 mmol) in dichloromethane (5 mL), N-(2-(methylamino)ethyl)acetamide hydrochloride (19-2, 55 mg, 0.4 mmol) was added followed by DIPEA (0.2 mL, 0.9 mmol) at 0° C. After 10 min, T₃P (0.3 mL, 0.5 mmol, 50% in ethyl acetate) was added. The reaction mixture was stirred at ambient temperature for 16 h, quenched with ice-cold water, and extracted with dichloromethane. The combined organic extracts were washed with brine, dried over anhydrous sodium sulfate, filtered, and concentrated under reduced pressure. The product was purified by reverse phase prep-HPLC to afford (R)-N-(2-acetamidoethyl)-N-methyl-2-((1-oxo-4-(o-tolyl)-1,2-dihydroisoquinolin-7-yl)oxy)propenamide (Example 19, 34 mg). LCMS (ESI) Calcd. for C₂₄H₂₇N₃O₄: 421, found [M+H]⁺=422. ¹H NMR (400 MHz, DMSO-d₆ at 100° C.): δ_{H} 11.05 (br s, 1H), 7.65 (br s, 2H), 7.34-7.18 (m, 5H), 6.94 (d, 1H), 6.85 (s, 1H), 5.28-5.24 (m, 1H), 3.47-3.06 (m, 7H), 2.07 (s, 3H), 1.77 (s, 3H), 1.50 (d, 3H).

### Example 20: Synthesis of (R)-1-(2-((4-(2,6-dimethylphenyl)-1-oxo-1,2-dihydroisoquinolin-7-yl)oxy)propanoyl)-4-methylpiperidine-4-carboxylic acid

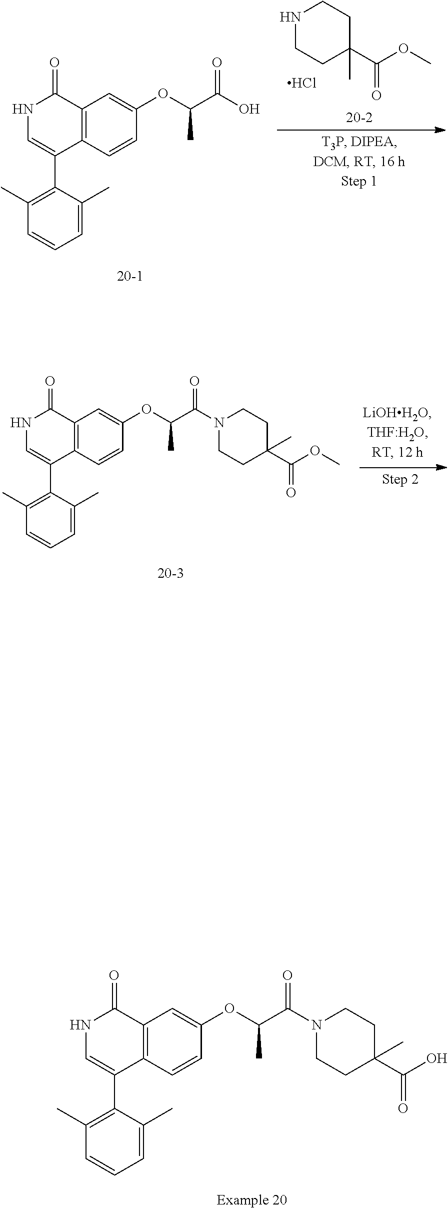

Synthesis of methyl (R)-1-(2-((4-(2,6-dimethylphenyl)-1-oxo-1,2-dihydroisoquinolin-7-yl)oxy)propanoyl)-4-methylpiperidine-4-carboxylate, 20-3 [Step 1]: To a stirred solution of (R)-2-((4-(2,6-dimethylphenyl)-1-oxo-1,2-dihydroisoquinolin-7-yl)oxy)propanoic acid (20-1, 200 mg, 0.6 mmol) in dichloromethane (8 mL), N,N-Diisopropylethylamine (0.1 mL, 0.6 mmol), methyl 4-methylpiperidine-4-carboxylate hydrochloride (20-2, 115 mg, 0.6 mmol) and T₃P (0.55 mL, 0.9 mmol, 50% in ethyl acetate) were added. The resulting reaction mixture was stirred at ambient temperature for 16 h. After completion, the reaction mixture was diluted with dichloromethane and washed with water and brine. The organic extract was dried over anhydrous sodium sulphate, filtered and concentrated under reduced pressure to afford methyl (R)-1-(2-((4-(2,6-dimethylphenyl)-1-oxo-1,2-dihydroisoquinolin-7-yl)oxy)propanoyl)-4-methylpiperidine-4-carboxylate (20-3, 150 mg). LCMS (ESI) Calcd. for C₂₈H₃₂N₂O₅: 476, found [M+H]⁺=477.

Synthesis of (R)-1-(2-((4-(2,6-dimethylphenyl)-1-oxo-1,2-dihydroisoquinolin-7-yl)oxy)propanoyl)-4-methylpiperidine-4-carboxylic acid, Example 20 [Step 2]: To a stirred solution of methyl (R)-1-(2-((4-(2,6-dimethylphenyl)-1-oxo-1,2-dihydroisoquinolin-7-yl)oxy)propanoyl)-4-methylpiperidine-4-carboxylate (20-3, 140 mg, 0.3 mmol) in tetrahydrofuran (6 mL) and water (1.5 mL) mixture, lithium hydroxide monohydrate (20 mg, 0.5 mmol) was added. The resulting reaction mixture was stirred at ambient temperature for 16 h. After completion, the volatiles were evaporated under reduced pressure. The residue was diluted with water and washed with ethyl acetate. The aqueous phase was acidified with 1N HCl solution to pH˜3 and extracted with a mixture of 10% methanol in dichloromethane (twice). The combined organic extracts was washed with brine, dried over anhydrous sodium sulphate and concentrated under reduced pressure. The product was purified by reverse phase prep-HPLC to afford (R)-1-(2-((4-(2,6-dimethylphenyl)-1-oxo-1,2-dihydroisoquinolin-7-yl)oxy)propanoyl)-4-methylpiperidine-4-carboxylic acid (Example 20, 52 mg). LCMS (ESI) Calcd. for C₂₇H₃₀N₂O₅: 462, found [M+H]⁺=463. ¹H NMR (400 MHz, DMSO-d₆): δ_{H} 10.99 (br s, 1H), 7.67 (s, 1H), 7.25-7.16 (m, 4H), 6.80-6.78 (m, 2H), 5.32-5.30 (m, 1H), 3.95-3.86 (m, 3H), 3.21 (br s, 2H), 2.00 (br s, 8H), 1.49 (d, 3H), 1.35 (br s, 2H), 1.99 (s, 3H).

### Example 21: Synthesis of (R)-N-((R)-1-amino-1-oxopropan-2-yl)-2-((1-oxo-4-(o-tolyl)-1,2-dihydroisoquinolin-7-yl)oxy)propanamide

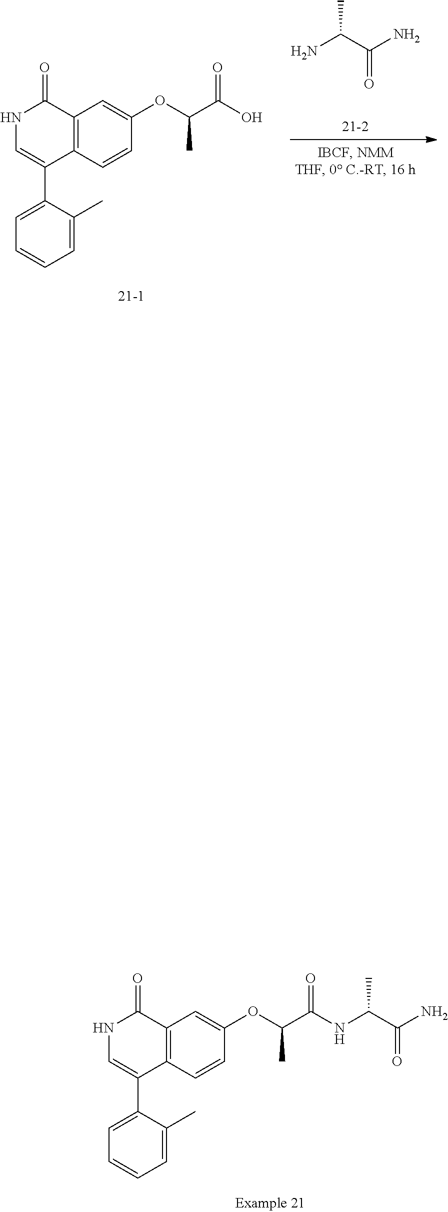

To a stirred solution of (R)-2-((1-oxo-4-(o-tolyl)-1,2-dihydroisoquinolin-7-yl)oxy)propanoic acid (21-1, 100 mg, 0.3 mmol) in THF (3 mL) was added IBCF (0.04 mL, 0.3 mmol) followed by NMM (0.05 mL, 0.5 mmol) at 0° C. and stirred for 30 min. Into the reaction mixture was added (R)-2-aminopropanamide (21-2, 27 mg, 0.3 mmol) and NMM (0.03 mL, 0.3 mmol) at 0° C. and allowed stir for 2 h. After completion, the reaction mixture was diluted with ethyl acetate and washed with water, brine, dried over anhydrous Na₂SO₄ and concentrated under reduced pressure. The product was purified by reverse phase prep HPLC purification method and lyophilized to afford (R)-N-((R)-1-amino-1-oxopropan-2-yl)-2-((1-oxo-4-(o-tolyl)-1,2-dihydroisoquinolin-7-yl)oxy)propanamide (Example 21, 50 mg). LCMS (ESI) Calcd. for C₂₂H₂₃N₃O₄: 339, found [M+H]⁺=394. ¹H NMR (400 MHz, DMSO-d₆): δ_{H} 11.35 (br s, 1H), 8.33-8.28 (m, 1H), 7.68 (s, 1H), 7.35-7.26 (m, 5H), 7.19-7.18 (m, 1H), 7.03 (m, 1H), 6.91-6.88 (m, 2H), 4.96-4.95 (m, 1H), 4.27-4.24 (m, 1H), 2.04-2.03 (m, 3H), 1.45-1.44 (m, 3H), 1.25 (d, 3H).

### Example 22: Synthesis of (R)-N-methyl-N-(2-(N-methylacetamido)ethyl)-2-((1-oxo-4-(o-tolyl)-1,2-dihydroisoquinolin-7-yl)oxy)propanamide

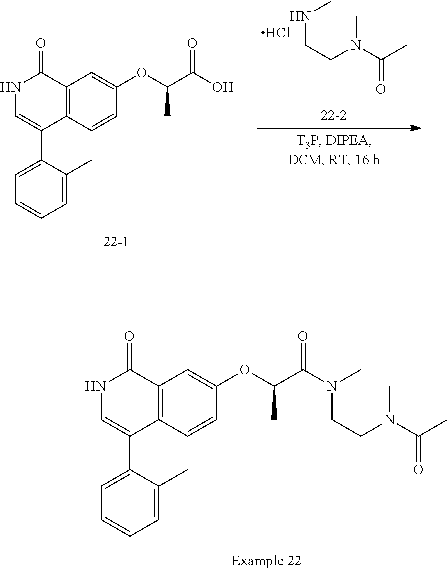

To a stirred solution of (R)-2-((1-oxo-4-(o-tolyl)-1,2-dihydroisoquinolin-7-yl)oxy)propanoic acid (22-1, 100 mg, 0.3 mmol) in dichloromethane (5 mL) at 0° C., N-methyl-N-(2-(methylamino)ethyl)acetamide hydrochloride (22-2, 50 mg, 0.4 mmol) and diisopropylethylamine (0.2 mL, 0.9 mmol) were added. After 10 min, T₃P (0.4 mL, 0.5 mmol, 50% in ethyl acetate) was added and the reaction mixture was stirred at ambient temperature for additional 16 h. After completion, the reaction mixture was quenched with ice-cold water, and extracted with dichloromethane (twice). The combined organic extracts was washed with brine, dried over anhydrous sodium sulphate, filtered, and concentrated under reduced pressure. The product was purified by reverse phase prep-HPLC to afford (R)-N-methyl-N-(2-(N-methylacetamido)ethyl)-2-((1-oxo-4-(o-tolyl)-1,2-dihydroisoquinolin-7-yl)oxy)propanamide (Example 22, 33 mg). LCMS (ESI) Calcd. for C₂₅H₂₉N₃O₄: 435, found [M+H]⁺=436. ¹H NMR (400 MHz, DMSO-d₆ at 100° C.): δ_{H} 10.99 (s, 1H), 7.68 (s, 1H), 7.33 (s, 2H), 7.27-7.18 (m, 3H), 6.93 (d, 1H), 6.84 (s, 1H), 5.29 (s, 1H), 3.51 (m, 5H), 3.10 (br s, 2H), 2.92-2.83 (m, 3H), 2.07 (s, 3H), 1.93 (br s, 3H), 1.48 (d, 3H).

### Example 23: Synthesis of (R)-7-((1-(4-(2-methoxyethyl)-3-oxopiperazin-1-yl)-1-oxopropan-2-yl)oxy)-4-(o-tolyl)isoquinolin-1(21)-one

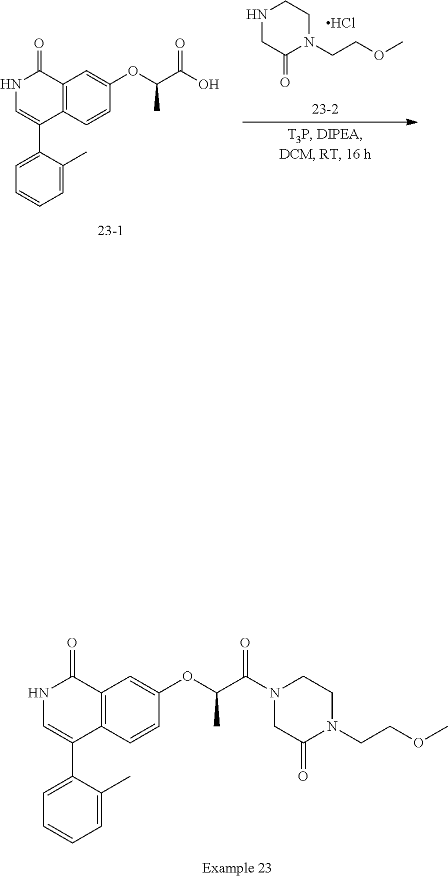 [SMILES: Cc1ccccc1-c1c[nH]c(=O)c2cc(O[C@H](C)C(=O)O)ccc12 ; COCCN1CCNCC1=O ; Cl ; COCCN1CCN(C(=O)[C@@H](C)Oc2ccc3c(-c4ccccc4C)c[nH]c(=O)c3c2)CC1=O]

To a stirred solution of (R)-2-((1-oxo-4-(o-tolyl)-1,2-dihydroisoquinolin-7-yl)oxy)propanoic acid (23-1, 100 mg, 0.3 mmol) in dichloromethane (5 mL) at 0° C., 1-(2-methoxyethyl)piperazin-2-one hydrochloride (23-2, 50 mg, 0.31 mmol) and diisopropylethylamine (0.1 mL, 0.8 mmol) were added. After 10 min, T₃P (0.4 mL, 0.5 mmol, 50% in ethyl acetate) was added and the reaction mixture was stirred at ambient temperature for additional 16 h. After completion, the reaction mixture was quenched with ice-cold water, and extracted with dichloromethane (twice). The combined organic extracts was washed with brine, dried over anhydrous sodium sulphate, filtered, and concentrated under reduced pressure. The product was purified by reverse phase prep-HPLC to afford (R)-7-((1-(4-(2-methoxyethyl)-3-oxopiperazin-1-yl)-1-oxopropan-2-yl)oxy)-4-(o-tolyl)isoquinolin-1(2H)-one (Example 23, 48 mg). LCMS (ESI) Calcd. for C₂₆H₂₉N₃O₅: 463, found [M+H]⁺=464. ¹H NMR (400 MHz, DMSO-d₆ at 100° C.): δ_{H} 11.03 (br s, 1H), 7.71 (s, 1H), 7.35-7.34 (m, 2H), 7.29-7.23 (m, 2H), 7.19-7.18 (m, 1H), 6.94 (d, 1H), 6.85 (s, 1H), 5.38-5.36 (m, 1H), 4.13 (br s, 2H), 3.78 (br s, 2H), 3.47-3.41 (m, 6H), 3.24 (s, 3H), 2.07 (s, 3H), 1.50 (d, 3H).

### Example 24: Synthesis of(S)-N-((R)-1-amino-1-oxopropan-2-yl)-2-((1-oxo-4-(o-tolyl)-1,2-dihydroisoquinolin-7-yl)oxy)propanamide

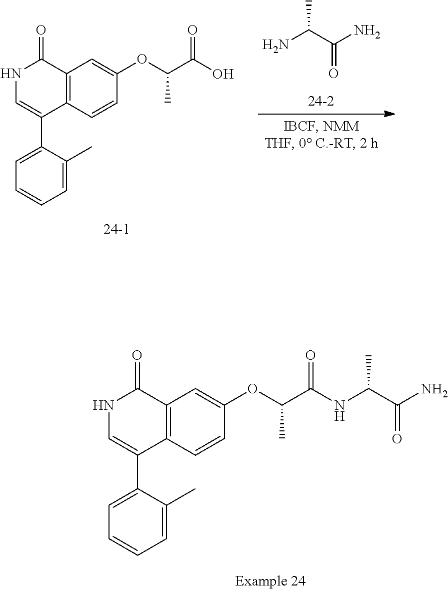

To a stirred solution of (S)-2-((1-oxo-4-(o-tolyl)-1,2-dihydroisoquinolin-7-yl)oxy)propanoic acid (24-1, 100 mg, 0.3 mmol) in THF (3 mL), IBCF (0.036 mL, 0.3 mmol) was added followed by NMM (0.05 mL, 0.5 mmol) at 0° C. and stirred for 30 min. (R)-2-aminopropanamide (24-2, 25 mg, 0.3 mmol) was added followed by NMM (0.03 mL, 0.3 mmol) to the reaction mixture at 0° C. and the reaction mixture was allowed to warm up to ambient temperature and stirred for 2 h. The reaction mixture was diluted with ethyl acetate and washed with water, brine, dried over anhydrous Na₂SO₄ and concentrated under reduced pressure. The product was purified by reverse phase prep HPLC purification method and lyophilized to afford (S)-N-((R)-1-amino-1-oxopropan-2-yl)-2-((1-oxo-4-(o-tolyl)-1,2-dihydroisoquinolin-7-yl)oxy)propanamide (Example 24, 40 mg). LCMS (ESI) Calcd. for C₂₂H₂₃N₃O₄: 393, found [M+H]⁺=394. ¹H NMR (400 MHz, DMSO-d₆): δ_{H} 11.34-11.33 (m, 1H), 8.14-8.10 (m, 1H), 7.69-7.68 (m, 1H), 7.35-7.26 (m, 5H), 7.20-7.18 (m, 11H), 7.06 (s, 11H), 6.92-6.88 (m, 2H), 4.92-4.90 (m, 1H), 4.23-4.19 (m, 1H), 2.03 (s, 3H), 1.46 (d, 3H), 1.18-1.15 (m, 3H).

### Example 25-26: Synthesis of methyl (S)-N-methyl-N-(2-((1-oxo-4-(o-tolyl)-1,2-dihydroisoquinolin-7-yl)oxy)propanoyl)glycinate and (S)-N-methyl-N-(2-((1-oxo-4-(o-tolyl)-1,2-dihydroisoquinolin-7-yl)oxy)propanoyl)glycine

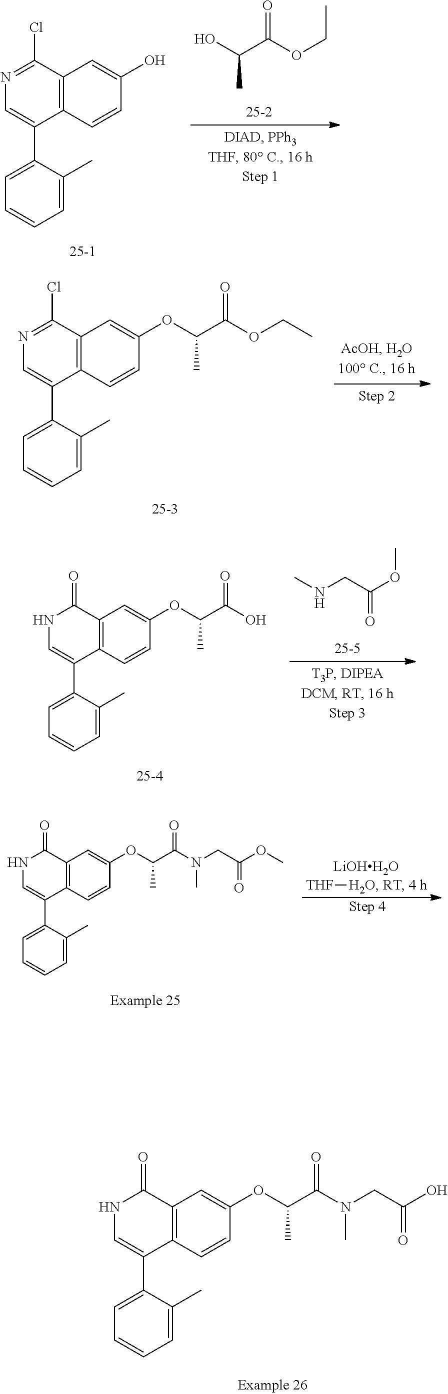

Synthesis of ethyl (S)-2-((1-chloro-4-(o-tolyl)isoquinolin-7-yl)oxy)propanoate, 25-3 [Step 1]: To a stirred solution of 1-chloro-4-(o-tolyl)isoquinolin-7-ol (25-1, 1.2 g, 4.5 mmol) and ethyl (R)-2-hydroxypropanoate (25-2, 1.2 g, 10.2 mmol) in dry THF (30 mL) was added PPh₃ (3.50 g, 13.3 mmol). The reaction mixture was cooled to 0° C. and degassed with N₂ for 5 minutes. Diisopropyl azodicarboxylate (2.6 mL, 13.3 mmol) was added dropwise in ice-cold condition and the reaction mixture was slowly warmed up to 80° C. The reaction mixture was stirred at 80° C. for 16 h. After completion, the reaction mixture was diluted with ethyl acetate, washed with water and brine. The organic extract was dried over anhydrous Na₂SO₄ and concentrated under reduced pressure. The product was purified by column chromatography to afford ethyl (S)-2-((1-chloro-4-(o-tolyl)isoquinolin-7-yl)oxy)propanoate (25-3, 1.3 g). LCMS (ESI) Calcd. for C₂₁H₂₀ClNO₃: 369, found [M+H]⁺=370. ¹H NMR (400 MHz, DMSO-d₆): δ_{H} 8.07 (s, 1H), 7.56-7.50 (m, 2H), 7.45-7.42 (m, 2H), 7.37-7.34 (m, 2H), 7.25 (t, 11H), 5.25 (q, 1H), 4.19 (q, 2H), 1.97 (d, 3H), 1.60 (d, 3H), 1.21 (t, 3H).

Synthesis of (S)-2-((1-oxo-4-(o-tolyl)-1,2-dihydroisoquinolin-7-yl)oxy)propanoic acid, 25-4 [Step 2]: To a stirred solution of ethyl (S)-2-((1-chloro-4-(o-tolyl)isoquinolin-7-yl)oxy)propanoate (25-3, 2 g, 5.4 mmol) in acetic acid (9.3 mL, 162 mmol) was added water (1.9 mL, 108 mmol). Then the reaction mixture was heated at 120° C. for 12 h. The volatiles were removed under reduced pressure. The residue was dissolved in ethyl acetate and washed with water and brine. The organic extract was dried over anhydrous Na₂SO₄ and concentrated under reduced pressure to afford (S)-2-((1-oxo-4-(o-tolyl)-1,2-dihydroisoquinolin-7-yl)oxy)propanoic acid (25-4, 1.6 g). LCMS (ESI) Calcd. for C₁₉H₁₇NO₄: 323, found [M+H]⁺=324.

Synthesis of methyl (S)-N-methyl-N-(2-((1-oxo-4-(o-tolyl)-1,2-dihydroisoquinolin-7-yl)oxy)propanoyl)glycinate, Example 25 [Step 3]: To a stirred solution of (S)-2-((1-oxo-4-(o-tolyl)-1,2-dihydroisoquinolin-7-yl)oxy)propanoic acid (25-4, 250 mg, 0.8 mmol) in dichloromethane (5 mL) was added methyl 2-(methylamino)acetate hydrochloride (25-5, 160 mg, 1.2 mmol) and DIPEA (0.4 mL, 2.3 mmol). The reaction mixture cooled to 0° C. Into this cold reaction mixture, T₃P (0.7 mL, 1.2 mmol, 50% in ethyl acetate) was added and the reaction mixture was allowed to warm up to ambient temperature and stirred for 16 h. After completion, the reaction mixture was quenched with water and extracted with dichloromethane (twice). The combined organic extract was washed with water and brine, dried over anhydrous Na₂SO₄ and concentrated under reduced pressure. The product was purified by reverse phase prep HPLC purification to afford methyl (S)-N-methyl-N-(2-((1-oxo-4-(o-tolyl)-1,2-dihydroisoquinolin-7-yl)oxy)propanoyl)glycinate (Example 25, 30 mg). LCMS (ESI) Calcd. for C₂₃H₂₄N₂O₅: 408, found [M+H]⁺=409. ¹H NMR (400 MHz, DMSO-d₆): δ 11.33 (br s, 1H), 7.64-7.61 (m, 1H), 7.35-7.30 (m, 2H), 7.28-7.24 (m, 1H), 7.22-7.18 (m, 2H), 6.91-6.88 (m, 2H), 5.46-5.36 (m, 1H), 4.41-4.31 (m, 1H), 4.16-4.02 (m, 2H), 3.61 (s, 3H), 3.19 (d, 2H), 2.04 (s, 3H), 1.49-1.40 (m, 3H).

Synthesis of (S)-N-methyl-N-(2-((1-oxo-4-(o-tolyl)-1,2-dihydroisoquinolin-7-yl)oxy)propanoyl)glycine, Example 26 [Step 4]: To a stirred solution of methyl (S)-N-methyl-N-(2-((1-oxo-4-(o-tolyl)-1,2-dihydroisoquinolin-7-yl)oxy)propanoyl)glycinate (Example 25, 100 mg, 0.2 mmol) in tetrahydrofuran (4 mL) was added an aqueous solution (1 mL) of LiOH·H₂O (30 mg, 0.7 mmol) at 0° C. The reaction mixture was stirred at ambient temperature for 4 h. After completion, the reaction mixture was acidified with 1N HCl to pH=4 and extracted with ethyl acetate (twice). The combined organic extract was washed with brine, dried over anhydrous Na₂SO₄ and concentrated under reduced pressure. The product was purified by reverse phase prep HPLC purification to afford (S)-N-methyl-N-(2-((1-oxo-4-(o-tolyl)-1,2-dihydroisoquinolin-7-yl)oxy)propanoyl)glycine (Example 26, 70 mg). LCMS (ESI) Calcd. for C₂₂H₂₂N₂O₅: 394, found [M+H]⁺=395. ¹H NMR (400 MHz, DMSO-d₆): δ_{H} 11.35 (br s, 1H), 7.60 (br s, 1H), 7.34-7.33 (m, 2H), 7.29-7.22 (m, 2H), 7.18-7.17 (m, 1H), 6.89-6.82 (m, 2H), 5.39-5.14 (m, 1H), 3.99-3.73 (m, 2H), 3.13-2.77 (m, 3H), 2.03 (s, 3H), 1.47-1.41 (m, 3H).

### Example 27: Synthesis of 7-(((2R)-1-(8-(2-methoxyethyl)-3,8-diazabicyclo[3.2.1]octan-3-yl)-1-oxopropan-2-yl)oxy)-4-(o-tolyl)isoquinolin-1(2H)-one

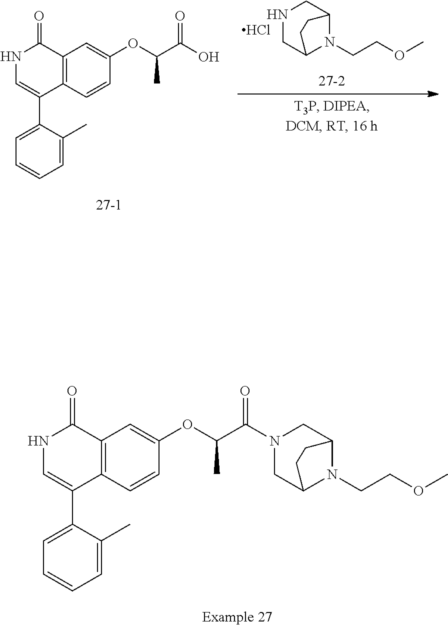 [SMILES: Cc1ccccc1-c1c[nH]c(=O)c2cc(O[C@H](C)C(=O)O)ccc12 ; Cl ; COCCN1C2CCC1CNC2 ; COCCN1C2CCC1CN(C(=O)[C@@H](C)Oc1ccc3c(-c4ccccc4C)c[nH]c(=O)c3c1)C2]

To a stirred solution of (R)-2-((1-oxo-4-(o-tolyl)-1,2-dihydroisoquinolin-7-yl)oxy)propanoic acid (27-1, 100 mg, 0.3 mmol) in dichloromethane (5 mL) at 0° C., 8-(2-methoxyethyl)-3,8-diazabicyclo[3.2.1]octane hydrochloride (27-2, 50 mg, 0.3 mmol) and diisopropylethylamine (0.2 mL, 0.93 mmol) were added. After 10 min, T₃P (0.4 mL, 0.46 mmol, 50% in ethyl acetate) was added and the reaction mixture was stirred at ambient temperature for additional 16 h. After completion, the reaction mixture was quenched with ice-cold water, and extracted with dichloromethane (twice). The combined organic extract was washed with brine, dried over anhydrous sodium sulphate, filtered, and concentrated under reduced pressure. The product was purified by reverse prep-HPLC to afford 7-(((2R)-1-(8-(2-methoxyethyl)-3,8-diazabicyclo[3.2.1]octan-3-yl)-1-oxopropan-2-yl)oxy)-4-(o-tolyl)isoquinolin-1(2H)-one (Example 27, 57 mg). LCMS (ESI) Calcd. for C₂₈H₃₃N₃O₄: 476, found [M+H]⁺=477. ¹H NMR (400 MHz, DMSO-d₆ at 100° C.): δ_{H} 11.01 (br s, 1H), 7.66 (s, 1H), 7.34 (s, 2H), 7.28 (s, 1H), 7.23-7.18 (m, 2H), 6.93 (d, 1H), 6.85 (s, 1H), 5.28 (br s, 1H), 3.87-3.75 (m, 2H), 3.45 (br s, 3H), 3.26 (br s, 6H), 2.80 (br s, 1H), 2.07 (s, 3H), 1.82 (br s, 3H), 1.46 (br s, 5H).

### Example 28: Synthesis of N-((S)-1-((R)-2-((1-oxo-4-(o-tolyl)-1,2-dihydroisoquinolin-7-yl)oxy)propanoyl)piperidin-3-yl)acetamide

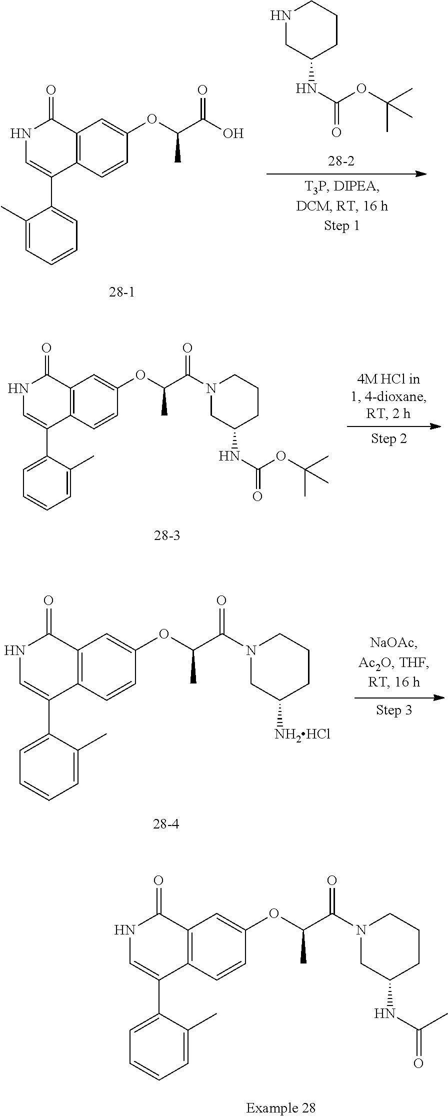

Synthesis of tert-butyl ((S)-1-((R)-2-((1-oxo-4-(o-tolyl)-1,2-dihydroisoquinolin-7-yl)oxy)propanoyl)piperidin-3-yl)carbamate, 28-3 [Step 1]: To a stirred solution of (S)-2-((1-oxo-4-(o-tolyl)-1,2-dihydroisoquinolin-7-yl)oxy)propanoic acid (28-1, 150 mg, 0.5 mmol) in dichloromethane (8 mL) at 0° C., DIPEA (0.1 mL, 0.5 mmol) and tert-butyl (R)-piperidin-3-ylcarbamate (28-2, 95 mg, 0.5 mmol) were added. After 10 min, T₃P (0.4 mL, 0.7 mmol, 50% in ethyl acetate) was added. The reaction mixture was stirred at ambient temperature for an additional 16 h, quenched with ice-cold water, and extracted with dichloromethane (twice). The combined organic extracts were washed with brine, dried over anhydrous sodium sulphate, filtered, and concentrated under reduced pressure to afford tert-butyl ((S)-1-((R)-2-((1-oxo-4-(o-tolyl)-1,2-dihydroisoquinolin-7-yl)oxy)propanoyl)piperidin-3-yl)carbamate (28-3, 160 mg). LCMS (ESI) Calcd. for C₂₉H₃₅N₃O₅: 505, found [M+H]⁻=506.

Synthesis of 7-(((R)-1-((S)-3-aminopiperidin-1-yl)-1-oxopropan-2-yl)oxy)-4-(o-tolyl)isoquinolin-1(2H)-one hydrochloride, 28-4 [Step 2]: To a stirred solution of tert-butyl ((S)-1-((R)-2-((1-oxo-4-(o-tolyl)-1,2-dihydroisoquinolin-7-yl)oxy)propanoyl)piperidin-3-yl)carbamate (28-3, 160 mg, 0.3 mmol) in 1,4-dioxane (4 mL) at 0° C., a solution of hydrochloric acid (4M) in 1,4-dioxane (2.0 mL) was added dropwise. The resulting reaction mixture was stirred at ambient temperature for 16 h and volatiles were evaporated. The product was triturated with diethyl ether to afford 7-(((R)-1-((S)-3-aminopiperidin-1-yl)-1-oxopropan-2-yl)oxy)-4-(o-tolyl)isoquinolin-1(2H)-one hydrochloride (28-4, 120 mg). LCMS (ESI) Calcd. for C₂₄H₂₇N₃O₃: 405, found [M+H]⁺=406.

Synthesis of N-((S)-1-((R)-2-((1-oxo-4-(o-tolyl)-1,2-dihydroisoquinolin-7-yl)oxy)propanoyl)piperidin-3-yl)acetamide, Example 28 [Step 3]: To a stirred solution of 7-(((R)-1-((S)-3-aminopiperidin-1-yl)-1-oxopropan-2-yl)oxy)-4-(o-tolyl)isoquinolin-1(2H)-one hydrochloride (284, 100 mg, 0.3 mmol) in THF (5 mL), sodium acetate (65 mg, 0.6 mmol) and acetic anhydride (85 mg, 0.6 mmol) were added. The reaction mixture was stirred at ambient temperature under nitrogen atmosphere for 16 h, diluted with ethyl acetate and washed with a saturated solution of aqueous sodium bicarbonate followed by brine. The combined organic extracts was dried over anhydrous sodium sulphate, filtered and concentrated under reduced pressure. The product was purified by reverse phase preparative HPLC to afford N-((S)-1-((R)-2-((1-oxo-4-(o-tolyl)-1,2-dihydroisoquinolin-7-yl)oxy)propanoyl)piperidin-3-yl)acetamide (Example 28, 41 mg). LCMS (ESI) Calcd. for C₂₆H₂₉N₃O₄: 447, found [M+H]⁺=448. ¹H NMR (400 MHz, DMSO-d₆ at 100° C.): δ_{H} 11.12 (br s, 1H), 7.91-7.55 (m, 2H), 7.35 (s, 2H), 7.28-7.20 (m, 3H), 6.94-6.92 (m, 1H), 6.86 (s, 1H) 5.35-5.18 (m, 1H), 4.23-4.12 (m, 1H), 3.92-3.69 (m, 2H), 3.29 (br s, 1H), 2.06 (s, 3H), 1.88-1.78 (m, 6H), 1.50 (br s, 5H).

### Example 29: Synthesis of N-((R)-1-((R)-2-((1-oxo-4-(o-tolyl)-1,2-dihydroisoquinolin-7-yl)oxy)propanoyl)piperidin-3-yl)acetamide

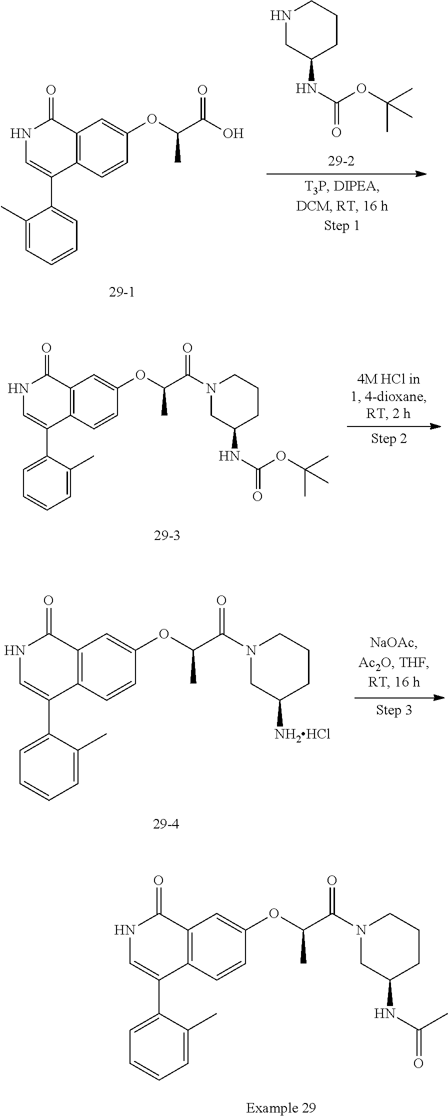

Synthesis of tert-butyl ((R)-1-((R)-2-((1-oxo-4-(o-tolyl)-1,2-dihydroisoquinolin-7-yl)oxy)propanoyl)piperidin-3-yl)carbamate, 29-3 [Step 1]: To a stirred solution of (R)-2-((1-oxo-4-(o-tolyl)-1,2-dihydroisoquinolin-7-yl)oxy)propanoic acid (29-1, 100 mg, 0.3 mmol) in dichloromethane (5 mL) at 0° C., DIPEA (0.2 mL, 0.9 mmol) and tert-butyl (R)-piperidin-3-ylcarbamate (29-2, 75 mg, 0.4 mmol) were added. After 10 min, T₃P (0.3 mL, 0.5 mmol, 50% in ethyl acetate) was added. The reaction mixture was stirred at ambient temperature for an additional 16 h, quenched with ice-cold water, and extracted with dichloromethane (twice). The combined organic extracts were washed with brine, dried over anhydrous sodium sulphate, filtered, and concentrated under reduced pressure to afford tert-butyl ((R)-1-((R)-2-((1-oxo-4-(o-tolyl)-1,2-dihydroisoquinolin-7-yl)oxy)propanoyl)piperidin-3-yl)carbamate (29-3, 180 mg). LCMS (ESI) Calcd. for C₂₉H₃₅N₃O₅: 505, found [M+H]⁻=506.

Synthesis of 7-(((R)-1-((R)-3-aminopiperidin-1-yl)-1-oxopropan-2-yl)oxy)-4-(o-tolyl)isoquinolin-1(2H)-one hydrochloride, 29-4 [Step 2]: To a stirred solution of tert-butyl ((R)-1-((R)-2-((1-oxo-4-(o-tolyl)-1,2-dihydroisoquinolin-7-yl)oxy)propanoyl)piperidin-3-yl)carbamate (29-3, 180 mg, 0.36 mmol) in 1,4-dioxane (3 mL) at 0° C., a solution of hydrochloric acid (4M) in 1,4-dioxane (0.9 mL) was added dropwise. The resulting reaction mixture was stirred at ambient temperature for 16 h and volatiles were evaporated. The product was triturated with diethyl ether to afford 7-(((R)-1-((R)-3-aminopiperidin-1-yl)-1-oxopropan-2-yl)oxy)-4-(o-tolyl)isoquinolin-1(2H)-one hydrochloride (29-4, 160 mg). LCMS (ESI) Calcd. for C₂₄H₂₇N₃O₃: 405, found [M+H]⁺=406.

Synthesis of N-((R)-1-((R)-2-((1-oxo-4-(o-tolyl)-1,2-dihydroisoquinolin-7-yl)oxy)propanoyl)piperidin-3-yl)acetamide, Example 29 [Step 3]: To a stirred solution of 7-(((R)-1-((R)-3-aminopiperidin-1-yl)-1-oxopropan-2-yl)oxy)-4-(o-tolyl)isoquinolin-1(2H)-one hydrochloride (294, 130 mg, 0.3 mmol) in tetrahydrofuran (5 mL), sodium acetate (110 mg, 0.8 mmol) and acetic anhydride (0.1 mL, 0.8 mmol) were added. The reaction mixture was stirred at ambient temperature under nitrogen atmosphere for 16 h, diluted with ethyl acetate and washed with a saturated solution of aqueous sodium bicarbonate followed by brine. The combined organic extracts was dried over anhydrous sodium sulfate, filtered and concentrated under reduced pressure. The product was purified by reverse phase prep HPLC to afford N-((R)-1-((R)-2-((1-oxo-4-(o-tolyl)-1,2-dihydroisoquinolin-7-yl)oxy)propanoyl)piperidin-3-yl)acetamide (Example 29, 21 mg). LCMS (ESI) Calcd. for C₂₆H₂₉N₃O₄: 447, found [M+H]⁺=448. ¹H NMR (400 MHz, DMSO-d₆ at 100° C.): δ_{H} 11.01 (br s, 1H), 7.69 (s, 1H), 7.49 (br s, 1H), 7.35-7.34 (m, 2H), 7.30-7.26 (m, 1H), 7.24-7.18 (m, 2H), 6.94 (d, 1H), 6.85 (s, 1H), 5.27 (br s, 1H), 3.82-3.74 (m, 3H), 3.09 (br s, 1H), 2.06 (s, 3H), 1.88 (br s, 2H), 1.79-1.75 (m, 3H), 1.51 (d, 6H).

### Example 30: Synthesis of (R)-1-(1-(2-((1-oxo-4-(o-tolyl)-1,2-dihydroisoquinolin-7-yl)oxy)propanoyl)piperidin-4-yl)urea

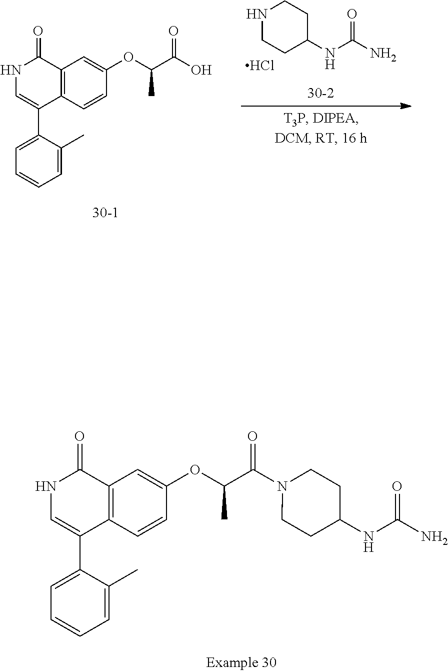 [SMILES: Cc1ccccc1-c1c[nH]c(=O)c2cc(O[C@H](C)C(=O)O)ccc12 ; Cl ; NC(=O)NC1CCNCC1 ; Cc1ccccc1-c1c[nH]c(=O)c2cc(O[C@H](C)C(=O)N3CCC(NC(N)=O)CC3)ccc12]

To a stirred solution of (R)-2-((1-oxo-4-(o-tolyl)-1,2-dihydroisoquinolin-7-yl)oxy)propanoic acid (30-1, 150 mg, 0.5 mmol) in dichloromethane (5 mL) at 0° C., 1-(piperidin-4-yl)urea hydrochloride (30-2, 100 mg, 0.6 mmol) and DIPEA (0.25 mL, 1.4 mmol) were added. After 10 min, T₃P (0.4 mL, 0.7 mmol, 50% in ethyl acetate) was added. The reaction mixture was stirred at ambient temperature for an additional 16 h, quenched with ice-cold water, and extracted with dichloromethane (twice). The combined organic extracts were washed with brine, dried over anhydrous sodium sulfate, filtered, and concentrated under reduced pressure. The product was purified by reverse phase prep HPLC to afford (R)-1-(1-(2-((1-oxo-4-(o-tolyl)-1,2-dihydroisoquinolin-7-yl)oxy)propanoyl)piperidin-4-yl)urea (Example 30, 55 mg). LCMS (ESI) Calcd. for C₂₅H₂₈N₄O₄: 448, found [M+H]⁺=449. ¹H NMR (400 MHz, DMSO-d₆): δ_{H} 11.34-11.27 (m, 1H), 7.60-7.58 (m, 1H), 7.35-7.18 (m, 5H), 6.91-6.88 (m, 2H), 6.03-5.98 (m, 1H), 5.43-5.38 (m, 3H), 4.21-3.90 (m, 2H), 3.62 (br s, 1H), 3.21-2.71 (m, 2H), 2.04 (s, 3H), 1.91-1.76 (m, 2H), 1.46-1.43 (m, 3H), 1.23-1.16 (m, 2H).

### Example 31: Synthesis of (R)-7-((1-(4-(dimethylamino)piperidin-1-yl)-1-oxopropan-2-yl)oxy)-4-(o-tolyl)isoquinolin-1(2H)-one

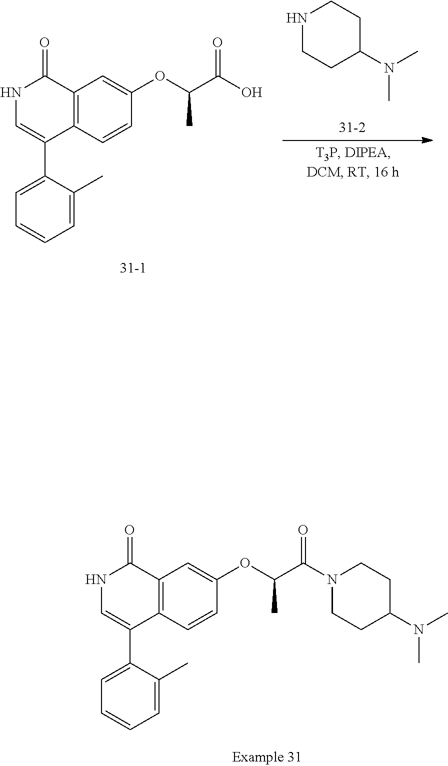

To a stirred solution of (R)-2-((1-oxo-4-(o-tolyl)-1,2-dihydroisoquinolin-7-yl)oxy)propanoic acid (31-1, 100 mg, 0.3 mmol) in dichloromethane (5 mL) at 0° C., N,N-dimethylpiperidin-4-amine (31-2, 45 mg, 0.4 mmol) and DIPEA (0.2 mL, 0.9 mmol) were added. After 10 min, T₃P (0.3 mL, 0.5 mmol, 50% in ethyl acetate) was added. The reaction mixture was stirred at ambient temperature for an additional 16 h, quenched with ice-cold water, and extracted with dichloromethane (twice). The combined organic extracts were washed with brine, dried over anhydrous sodium sulfate, filtered, and concentrated under reduced pressure. The product was purified by reverse phase prep HPLC to afford (R)-7-((1-(4-(dimethylamino)piperidin-1-yl)-1-oxopropan-2-yl)oxy)-4-(o-tolyl)isoquinolin-1(2H)-one (Example 31, 53 mg). LCMS (ESI) Calcd. for C₂₆H₃₁N₃O₃: 433, found [M+H]⁺=434. ¹H NMR (400 MHz, DMSO-d₆): δ_{H} 11.39-11.33 (m, 1H), 7.58-7.56 (m, 1H), 7.35-7.34 (m, 2H), 7.30-7.23 (m, 2H), 7.19-7.18 (m, 1H), 6.91-6.89 (m, 2H), 5.49-5.38 (m, 1H), 4.41-4.10 (m, 2H), 3.19-3.01 (m, 1H), 2.73-2.54 (m, 2H), 2.34 (s, 3H), 2.19 (s, 2H), 2.04-2.03 (m, 3H), 1.82-1.70 (m, 3H), 1.48-1.45 (m, 3H), 1.26-1.10 (m, 2H).

### Example 32: Synthesis of 7-(((R)-1-((S)-3-(dimethylamino)piperidin-1-yl)-1-oxopropan-2-yl)oxy)-4-(o-tolyl)isoquinolin-1(2H)-one

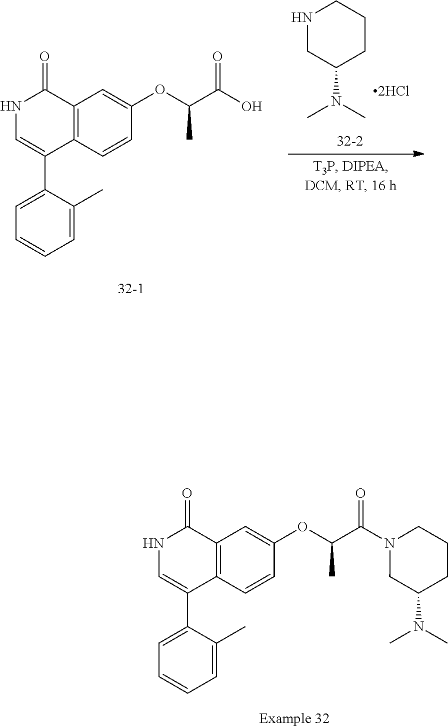

To a stirred solution of (R)-2-((1-oxo-4-(o-tolyl)-1,2-dihydroisoquinolin-7-yl)oxy)propanoic acid (32-1, 100 mg, 0.3 mmol) in dichloromethane (5 mL) at 0° C. (S)-N,N-dimethylpiperidin-3-amine dihydrochloride (32-2, 75 mg, 0.4 mmol) and diisopropylethylamine (0.2 mL, 0.9 mmol) were added. After 10 min, T₃P (0.14 mL, 0.5 mmol, 50% in ethyl acetate) was added and the reaction mixture was stirred at ambient temperature for an additional 16 h. After completion, the reaction was quenched with ice-cold water, and extracted with dichloromethane (twice). The combined organic extracts were washed with brine, dried over anhydrous sodium sulfate, filtered, and concentrated under reduced pressure. The product was purified by reverse prep-HPLC to afford 7-(((R)-1-((S)-3-(dimethylamino)piperidin-1-yl)-1-oxopropan-2-yl)oxy)-4-(o-tolyl)isoquinolin-1(2H)-one (Example 32, 52 mg). LCMS (ESI) Calcd. for C₂₆H₃₁N₃O₃: 433, found [M+H]⁺=434. ¹H NMR (400 MHz, DMSO-d₆ at 20° C.): δ_{H} 11.34-11.31 (m, 11H), 7.61-7.55 (m, 1H), 7.35-7.34 (m, 2H), 7.30-7.18 (m, 3H), 6.91-6.87 (m, 2H), 5.42-5.37 (m, 1H), 4.37-4.34 (m, 1H), 4.25-4.13 (m, 1H), 3.99-3.96 (m, 1H), 3.05-2.93 (m, 1H), 2.57-2.54 (m, 1H), 2.19-2.17 (m, 7H), 2.05-2.03 (m, 3H), 1.96-1.91 (m, 1H), 1.84-1.66 (m, 2H), 1.49-1.43 (m, 3H).

### Example 33: Synthesis of chiral analogs of 7-(((R)-1-(3-(dimethylamino)piperidin-1-yl)-1-oxopropan-2-yl)oxy)-4-(o-tolyl)isoquinolin-1(2H)-one

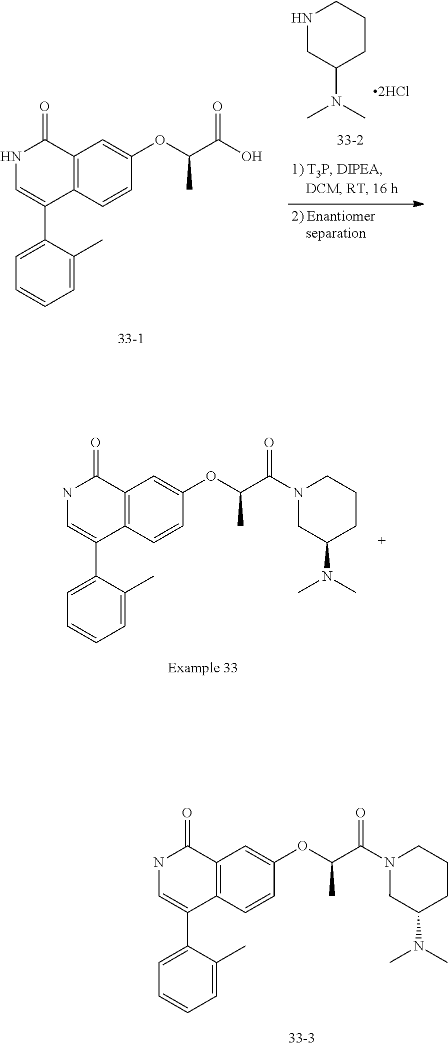

To a stirred solution of (R)-2-((1-oxo-4-(o-tolyl)-1,2-dihydroisoquinolin-7-yl)oxy)propanoic acid (33-1, 200 mg, 0.6 mmol) in dichloromethane (5 mL) at 0° C., N,N-dimethylpiperidin-3-amine dihydrochloride (33-2, 50 mg, 0.3 mmol) and diisopropylethylamine (0.3 mL, 1.9 mmol) were added. After 10 min, T₃P (0.4 mL, 0.5 mmol, 50% in ethyl acetate) was added and the reaction mixture was stirred at ambient temperature for an additional 16 h. After completion, the reaction mixture was quenched with ice-cold water, and extracted with dichloromethane (twice). The combined organic extracts were washed with brine, dried over anhydrous sodium sulfate, filtered, and concentrated under reduced pressure. The product was purified by reverse prep HPLC and enantiomers were separated by normal phase chiral preparative SFC HPLC to afford 7-(((R)-1-((S)-3-(dimethylamino)piperidin-1-yl)-1-oxopropan-2-yl)oxy)-4-(o-tolyl)isoquinolin-1(2H)-one as Peak 1(33-3) and 7-(((R)-1-((R)-3-(dimethylamino)piperidin-1-yl)-1-oxopropan-2-yl)oxy)-4-(o-tolyl)isoquinolin-1(2H)-one as Peak 2 (Example 33, 24.59 mg). The absolute stereochemistry of Example 33 was confirmed by comparison with Example 32.

Example 33: 7-(((R)-1-((R)-3-(dimethylamino)piperidin-1-yl)-1-oxopropan-2-yl)oxy)-4-(o-tolyl)isoquinolin-1(2H)-one (Peak 2): LCMS (ESI) Calcd. for C₂₆H₃₁N₃O₃: 433, found [M+H]⁺=434. ¹H NMR (400 MHz, DMSO-d₆ at 100° C.): δ_{H} 11.04 (s, 1H), 7.67 (s, 1H), 7.35-7.34 (m, 2H), 7.28-18 (m, 3H), 6.93 (d, 1H), 6.85 (m, 1H), 5.33-5.32 (m, 1H), 4.33 (br s, 1H), 4.01 (br s, 1H), 2.85 (br s, 1H), 2.33 (s, 6H), 2.06 (m, 3H), 1.95 (br s, 1H), 1.76 (br s, 1H), 1.50-1.49 (m, 4H), 1.27 (s, 2H).

Chiral HPLC prep-Method (SFC): SFC prep purification was performed on a Waters THAR SFC-80 instrument using Chiralpak ID (21 mm×250 mm), 5μ, which was operated at 35° C. temperature with a flow rate of 70 mL/min. The mobile phase was a mixture of 55% CO₂ in super critical state and 45% of (0.3% isopropyl amine in MeOH). The instrument was held in isocratic mode for up to 15 min and at an isobaric condition of 110 bar with a wavelength of 220 nm.

### Example 34-35: Synthesis of chiral analogs of 1-(1-((R)-2-((1-oxo-4-(o-tolyl)-1,2-dihydroisoquinolin-7-yl)oxy)propanoyl)piperidin-3-yl)cyclopropane-1-carboxylic acid

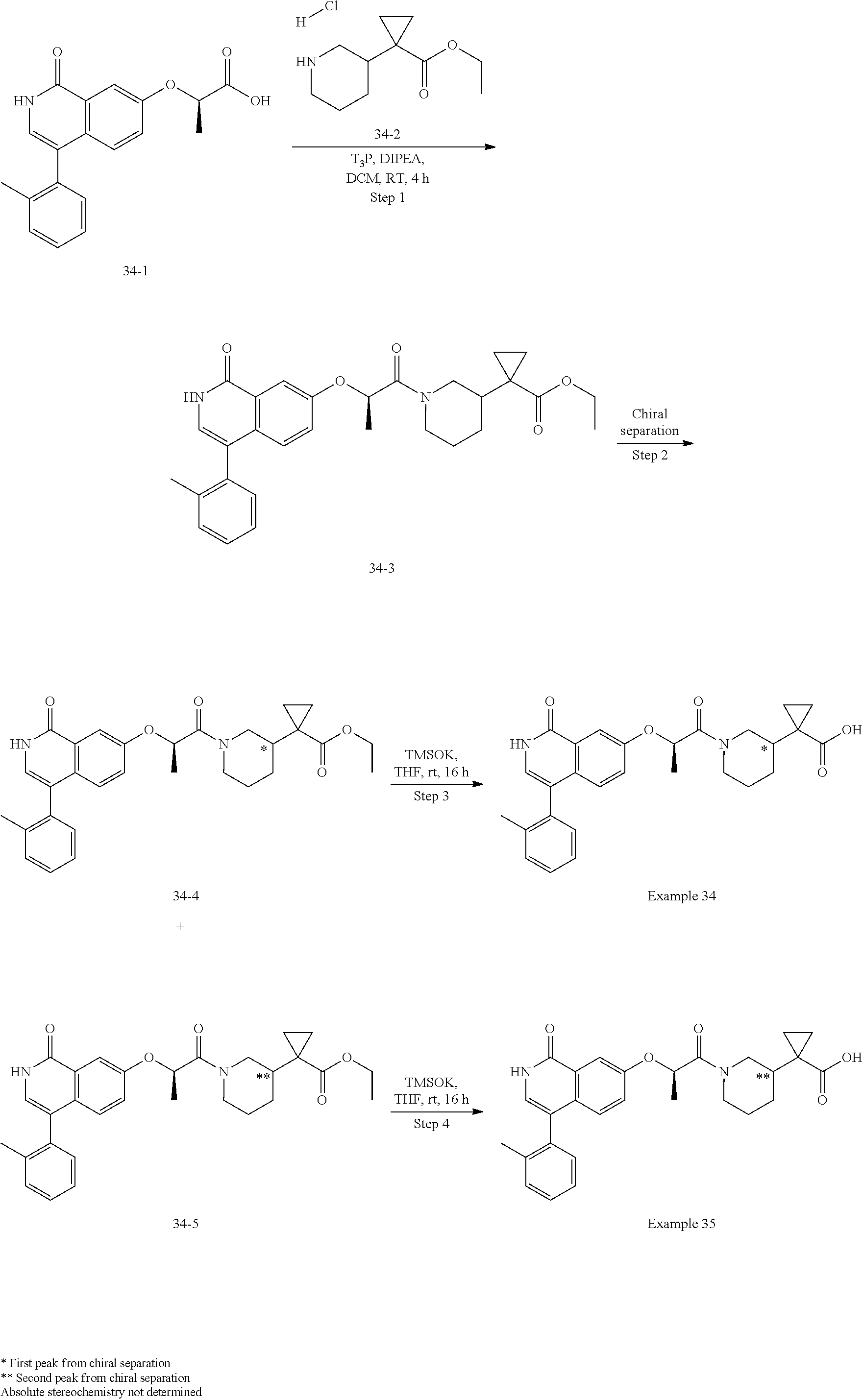

Synthesis of ethyl 1-(1-((R)-2-((1-oxo-4-(o-tolyl)-1,2-dihydroisoquinolin-7-yl)oxy)propanoyl)piperidin-3-yl)cyclopropane-1-carboxylate, 34-3 [Step 1]: To a stirred solution of (R)-2-((1-oxo-4-(o-tolyl)-1,2-dihydroisoquinolin-7-yl)oxy)propanoic acid (34-1, 450 mg, 1.4 mmol) and ethyl 1-(piperidin-3-yl)cyclopropane-1-carboxylate hydrochloride (34-2, 650 mg, 2.8 mmol) in dichloromethane (8 mL), DIPEA (0.9 mL, 5.5 mmol) followed by T₃P (1.7 mL, 2.8 mmol, 50% in ethyl acetate) were added dropwise at 0° C. and stirred at ambient temperature for 4 h. The reaction mixture was diluted with dichloromethane and washed with water (twice). The organic extract was collected, washed with brine, dried with anhydrous Na₂SO₄ and concentrated under reduced pressure. The product was purified by column chromatography to afford ethyl 1-(1-((R)-2-((1-oxo-4-(o-tolyl)-1,2-dihydroisoquinolin-7-yl)oxy)propanoyl)piperidin-3-yl)cyclopropane-1-carboxylate (34-3, 350 mg). LCMS (ESI) Calcd. for C₃₀H₃₄N₂O₅: 503, found [M+H]⁺=504.

Synthesis of chiral ethyl 1-(1-((R)-2-((1-oxo-4-(o-tolyl)-1,2-dihydroisoquinolin-7-yl)oxy)propanoyl)piperidin-3-yl)cyclopropane-1-carboxylate, 34-4 and 34-5 [Step 2]: Racemic ethyl 1-(1-((R)-2-((1-oxo-4-(o-tolyl)-1,2-dihydroisoquinolin-7-yl)oxy)propanoyl)piperidin-3-yl)cyclopropane-1-carboxylate (34-3, 330 mg) was purified by Prep-SFC purification and lyophilized to afford the first product as chiral ethyl 1-(1-((R)-2-((1-oxo-4-(o-tolyl)-1,2-dihydroisoquinolin-7-yl)oxy)propanoyl)piperidin-3-yl)cyclopropane-1-carboxylate (34-4, 80 mg) as Peak 1 and the second product as chiral ethyl 1-(1-((R)-2-((1-oxo-4-(o-tolyl)-1,2-dihydroisoquinolin-7-yl)oxy)propanoyl)piperidin-3-yl)cyclopropane-1-carboxylate (34-5, 100 mg) as Peak 2. The absolute stereochemistry were not determined.

Prep-SFC method: SFC prep purification was performed on a Pic Solution 175 instrument equipped with Knauer UV Detector 40D by using Chiralcel OJ-H (21 mm×250 mm), 5μ, which was operated at 35° C. temperature with a flow rate of 60 gm/min. The mobile phase used was a mixture of 90% CO₂ in super critical state and 10% of (100% MeOH). The instrument was held in isocratic mode for up to 10 min and at an isobaric condition of 120 bar with a wavelength of 224 nm.

34-4: Chiral ethyl 1-(1-((R)-2-((1-oxo-4-(o-tolyl)-1,2-dihydroisoquinolin-7-yl)oxy)propanoyl)piperidin-3-yl)cyclopropane-1-carboxylate (Peak 1): LCMS (ESI) Calcd. for C₃₀H₃₄N₂O₅: 503, [M+H]⁺=504.

34-5: Chiral ethyl 1-(1-((R)-2-((1-oxo-4-(o-tolyl)-1,2-dihydroisoquinolin-7-yl)oxy)propanoyl)piperidin-3-yl)cyclopropane-1-carboxylate (Peak 2): LCMS (ESI) Calcd. for C₃₀H₃₄N₂O₅: 503, [M+H]⁺=504.

Synthesis of 1-(1-((R)-2-((1-oxo-4-(o-tolyl)-1,2-dihydroisoquinolin-7-yl)oxy)propanoyl)piperidin-3-yl)cyclopropane-1-carboxylic acid, Example 34 [Step 3]: To a stirred solution of chiral ethyl 1-(1-((R)-2-((1-oxo-4-(o-tolyl)-1,2-dihydroisoquinolin-7-yl)oxy)propanoyl)piperidin-3-yl)cyclopropane-1-carboxylate (34-4.90 mg, 0.2 mmol) in THF (8 mL), potassium trimethylsilanolate (70 mg, 0.5 mmol) was added and stirred at ambient temperature for 16 h. The reaction mixture was quenched with 1N HCl solution to adjust to pH 3 and extracted with ethyl acetate (twice). The combined organic extract was collected, dried over anhydrous Na₂SO₄ and concentrated under reduced pressure. The product was purified by reverse phase prep HPLC and lyophilized to afford 1-(1-((R)-2-((1-oxo-4-(o-tolyl)-1,2-dihydroisoquinolin-7-yl)oxy)propanoyl)piperidin-3-yl)cyclopropane-1-carboxylic acid (Example 34, 32 mg). LCMS (ESI) Calcd. for C₂₈H₃₀N₂O₅: 475, found [M+H]⁺=475. ¹H NMR (400 MHz, DMSO-d₆ at 100° C.): δ_{H} 10.95 (br s, 2H), 7.68 (d, 1H), 7.34 (d, 2H), 7.31-7.25 (m, 1H), 7.23-7.17 (m, 2H), 6.92 (d, 1H), 6.83 (s, 1H), 5.31 (q, 1H), 4.21 (br s, 3H), 2.08 (s, 3H), 1.71-1.68 (m, 4H), 1.50 (d, 3H), 1.37-1.34 (m, 2H), 1.06-1.03 (m, 2H), 0.85 (br s, 2H). The absolute stereochemistry was not determined.

Synthesis of 1-(1-((R)-2-((1-oxo-4-(o-tolyl)-1,2-dihydroisoquinolin-7-yl)oxy)propanoyl)piperidin-3-yl)cyclopropane-1-carboxylic acid, Example 35 [Step 4]: To a stirred solution of chiral ethyl 1-(1-((R)-2-((1-oxo-4-(o-tolyl)-1,2-dihydroisoquinolin-7-yl)oxy)propanoyl)piperidin-3-yl)cyclopropane-1-carboxylate (34-5, 110 mg, 0.2 mmol) in THF (5 mL), potassium trimethylsilanolate (85 mg, 0.7 mmol) was added and stirred at ambient temperature for 16 h. The reaction mixture was quenched with 1N HCl solution to adjust to pH 3 and extracted with ethyl acetate (twice). The combined organic extract was collected, dried over anhydrous Na₂SO₄ and concentrated under reduced pressure. The product was purified by reverse phase Prep HPLC and lyophilized to afford 1-(1-((R)-2-((1-oxo-4-(o-tolyl)-1,2-dihydroisoquinolin-7-yl)oxy)propanoyl)piperidin-3-yl)cyclopropane-1-carboxylic acid (Example 35, 15 mg). LCMS (ESI) Calcd. for C₂₈H₃₀N₂O₅: 475, found [M+H]⁺=475. ¹H NMR (400 MHz, DMSO-d₆ at 100° C.): δ_{H} 10.97 (br s, 2H), 7.68 (d, 1H), 7.34 (d, 2H), 7.29-7.17 (m, 3H), 6.92 (d, 1H), 6.83 (s, 1H), 5.29 (q, 1H), 4.26 (br s, 3H), 2.08 (s, 3H), 1.71-1.69 (m, 3H), 1.70-1.35 (m, 6H), 1.06-1.03 (m, 2H), 0.85 (br s, 2H). The absolute stereochemistry was not determined.

### Example 36-37: Synthesis of chiral analogs of 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylic acid

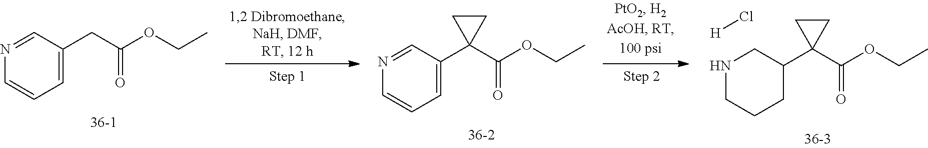

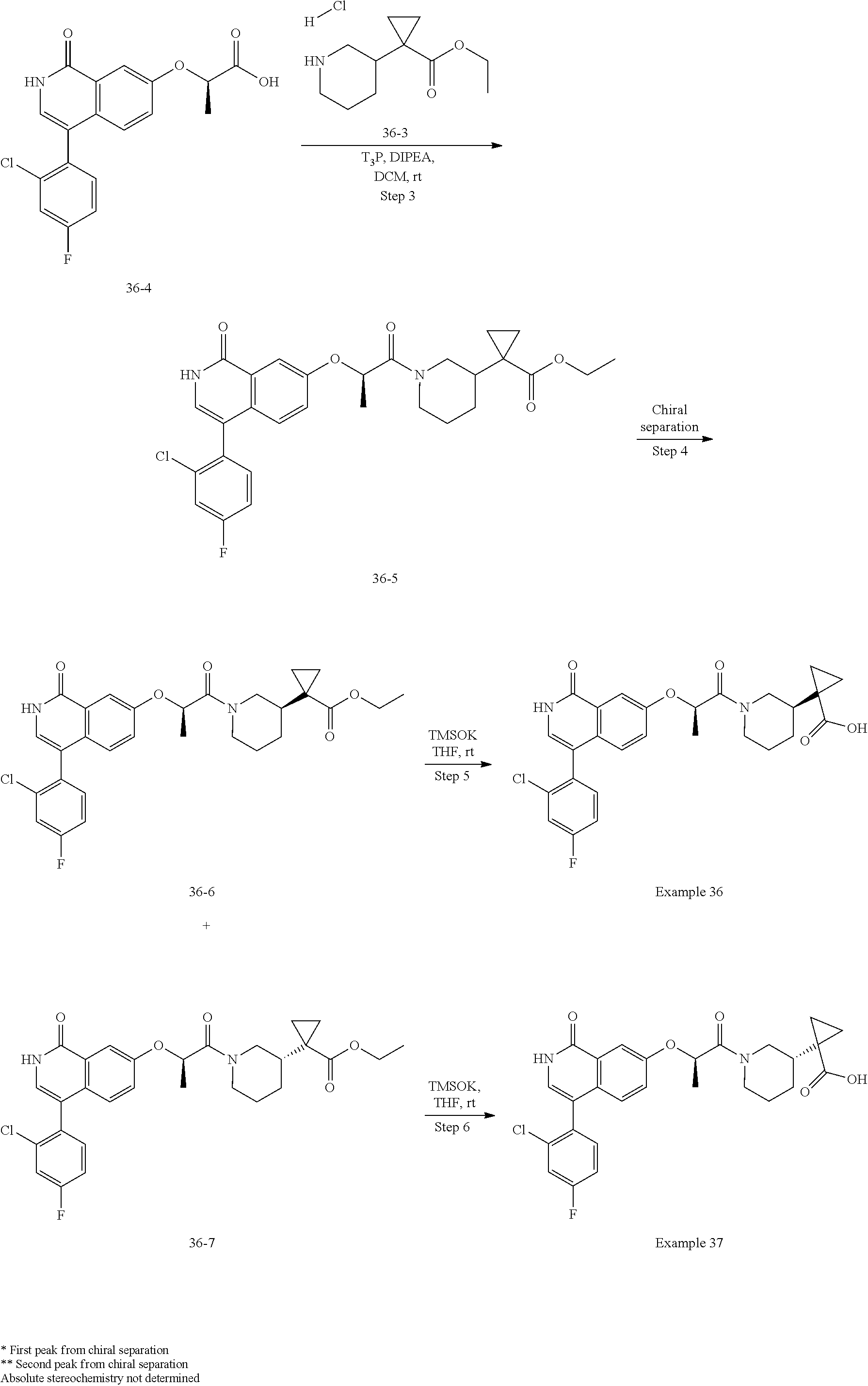

Synthesis of ethyl 1-(pyridin-3-yl)cyclopropane-1-carboxylate, 36-2 [Step 1]: To a stirred solution of ethyl 2-(pyridin-3-yl)acetate (36-1, 4.0 g, 24.2 mmol) in DMF (20 mL), NaH (1.7 g, 72.6 mmol, 60% in oil) was added portion wise at 0° C. After 15 min, 1,2 dibromoethane (4.2 mL, 48.4 mmol) was added to the reaction mixture and stirred at 0° C. for another 3 h. After completion, the reaction mixture was quenched with water and diluted with ethyl acetate. The organic phase was separated and washed with ice cold brine solution (thrice). It was dried over anhydrous Na₂SO₄, filtered and concentrated under reduced pressure. The product was purified by column chromatography to afford ethyl 1-(pyridin-3-yl)cyclopropane-1-carboxylate (36-2, 1.5 g). LCMS (ESI) Calcd. for C₁₁H₁₁NO₂: 191, found [M+H]⁺=192.

Synthesis of ethyl 1-(piperidin-3-yl)cyclopropane-1-carboxylate hydrochloride, 36-3 [Step 2]: To a stirred solution of ethyl 1-(pyridin-3-yl)cyclopropane-1-carboxylate (36-2, 210 mg, 1.1 mol) in dichloromethane (2 mL), 4M HCl in dioxane (2 mL) was added and stirred at ambient temperature for 30 min. The reaction mixture was concentrated under reduced pressure. The reduced mass was dissolved in methanol (35 mL) and acetic acid (4 mL). The mixture was degassed for 10 min with argon gas and PtO₂ (50 mg, 0.2 mmol) was added. The reaction mixture was stirred at ambient temperature in an autoclave reactor under hydrogen atmosphere at psi for 24 h. After completion, the reaction mixture was filtered through celite bed and the bed was washed with ethyl acetate (twice). The combined filtrate was concentrated under reduced pressure to afford ethyl 1-(piperidin-3-yl)cyclopropane-1-carboxylate hydrochloride (36-3, 220 mg). ¹H NMR (400 MHz, DMSO-d₆) δ_{H} 4.05-4.00 (m, 2H), 3.74-3.66 (m, 1H), 3.49-(m, 1H), 3.12 (d, 2H), 2.63 (t, 1H), 1.89 (s, 4H), 1.73-1.62 (m, 3H), 1.56 (t, 1H), 1.41 (d, 1H), 1.17 (t, 2H), 0.86 (t, 2H).

Synthesis of ethyl 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylate, 36-5 [Step 3]: To a stirred solution of (R)-2-((4-(2-chloro-4-fluorophenyl)-1-oxo-1,2-dihydroisoquinolin-7-yl)oxy)propanoic acid (36-4, 250 mg, 0.7 mmol) in dichloromethane (4 mL), ethyl 1-(piperidin-3-yl)cyclopropane-1-carboxylate hydrochloride (36-3, 70 mg, 0.4 mmol) and DIPEA (0.6 mL, 3.4 mmol) were added followed by T₃P (0.7 g, 1.1 mmol, 50% in ethyl acetate) dropwise at 0° C. and stirred at ambient temperature for 4 h. The reaction mixture was quenched with water and extracted with dichloromethane (twice). The combined organic layer was collected, dried over anhydrous Na₂SO₄ and concentrated under reduced pressure to afford ethyl 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylate (36-5, 280 mg). LCMS (ESI) Calcd. for C₂₉H₀ClFN₂O: 541, found [M+H]¹: 541.

Synthesis of chiral ethyl 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylate, 36-6 and 36-7 [Step 4]: The racemic ethyl 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylate (36-5, 280 mg, 0.5 mmol) was purified by normal phase prep-HPLC chiral and lyophilized to afford the first product as chiral ethyl 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylate (36-6, 60 mg) as Peak 1 and the second product as chiral ethyl 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylate (36-7, 60 mg) as Peak 2. The absolute stereochemistry were not determined.

Prep-HPLC chiral method: Chiral separation was performed on an Agilent 1200 series instrument using a CHLRALPAK IG (250×21 mm), 5μ, which was operated at ambient temperature with a flow rate of 21.0 mL/min. The mobile phase used was a mixture of 80% hexane, 10% dichloromethane, 10% ethyl alcohol and 0.1% IPAmine. The instrument was held in isocratic mode for up to 60 min with a wavelength of 292 nm.

Chiral ethyl 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylate, 36-6 [Peak 1]: LCMS (ESI) Calcd. for C₂₉H₃₀ClFN₂O₅: 541, found [M+H]⁺: =541. ¹H NMR (400 MHz, DMSO-d₆): δ_{H} 7.62 (t, 1H), 7.47 (d, 1H), 7.36 (d, 1H), 7.26 (d, 1H), 7.00 (d, 1H), 6.94 (d, 1H), 5.39 (d, 1H), 4.37 (d, 1H), 4.05-3.99 (m, 3H), 3.23 (s, 1H), 1.73-1.65 (m, 3H), 1.48-1.44 (t, 3H), 1.38 (s, 1H), 1.16-1.01 (m, 9H), 0.98 (t, 2H).

Chiral ethyl 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylate, 36-7 [Peak 2]: LCMS (ESI) Calcd. for C₂₉H₃₀ClFN₂O₅: 541, found [M+H]⁺: 541. ¹H NMR (400 MHz, DMSO-d₆): δ_{H} 7.62 (t, 1H), 7.47 (d, 1H), 7.36 (d, 1H), 7.26 (d, 1H), 7.00 (d, 1H), 6.94 (d, 1H), 5.39 (d, 1H), 4.37 (d, 1H), 4.05-3.99 (m, 3H), 3.23 (s, 1H), 1.73-1.65 (m, 3H), 1.48-1.44 (t, 3H), 1.38 (s, 1H), 1.16-1.01 (m, 9H), 0.98 (t, 2H).

Synthesis of 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylic acid, Example 36 [Step 5]: To a stirred solution of chiral ethyl 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylate (36-6, 60 mg, 0.1 mmol) in THF (8 mL), potassium trimethylsilanolate (45 mg, 0.3 mmol) was added and stirred at ambient temperature for 16 h. The reaction mixture was quenched with 1N HCl solution to adjust to pH 3 and extracted with ethyl acetate (twice). The organic extract was collected, dried over anhydrous Na₂SO₄ and concentrated under reduced pressure. The product was purified by reverse phase prep-HPLC and lyophilized to afford 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylic acid (Example 36, 25 mg). LCMS (ESI) Calcd. for C₂₇H₂₆ClFN₂O₅: 513, found [M+H]⁺=513. ¹H NMR (400 MHz, DMSO-d₆ at 100° C.): δ_{H} 10.56 (br s, 1H), 7.66 (s, 1H), 7.52-7.43 (m, 2H), 7.31-7.23 (m, 2H), 6.97-6.93 (m, 2H), 5.33 (br s, 1H), 4.25 (br s, 3H), 2.53 (s, 1H), 1.70 (br s, 2H), 1.58-1.55 (m, 1H), 1.50 (d, 3H), 1.36-1.27 (m, 1H), 1.18 (br s, 1H), 1.01-0.96 (m, 2H), 0.70 (br s, 2H). The absolute stereochemistry was not determined.

Synthesis of 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylic acid, Example 37 [Step 6]: To a stirred solution of chiral ethyl 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylate (36-7, 60 mg, 0.1 mmol) in THF (8 mL), potassium trimethylsilanolate (45 mg, 0.3 mmol) was added and stirred at ambient temperature for 16 h. The reaction mixture was quenched with 1N HCl solution to adjust to pH 3 and extracted with ethyl acetate (twice). The organic extract was collected, dried over anhydrous Na₂SO₄ and concentrated under reduced pressure. The product was purified by reverse phase prep-HPLC and lyophilized to afford 1-(1-((R)-2-((4-(2-chloro-4-fluorophenyl)-1-oxo-1,2-dihydroisoquinolin-7-yl)oxy)propanoyl)piperidin-3-yl)cyclopropane-1-carboxylic acid (Example 37, 15 mg). LCMS (ESI) Calcd. for C₂₇H₂₆ClFN₂O₅: 513, found [M+H]⁺=513. ¹H NMR (400 MHz, DMSO-d₆ at 100° C.): δ_{H} 10.99 (br s, 1H), 7.66 (s, 1H), 7.52-7.44 (m, 2H), 7.32-7.23 (m, 2H), 6.96-6.93 (m, 2H), 5.32 (br s, 1H), 4.28 (br s, 3H), 2.53 (s, 1H), 1.69 (br s, 2H), 1.60-1.49 (m, 6H), 1.06-0.99 (m, 2H), 0.73 (br s, 2H). The absolute stereochemistry was not determined.

### Example 38. Synthesis of 2-((R)-1-((R)-2-((1-oxo-4-(o-tolyl)-1,2-dihydroisoquinolin-7-yl)oxy)propanoyl)piperidin-3-yl)acetamide

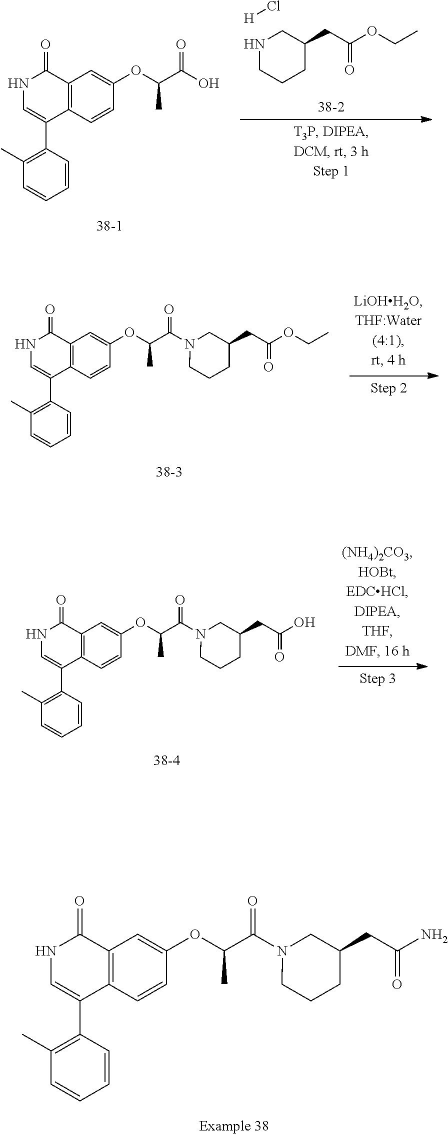

Synthesis of ethyl 2-((R)-1-((R)-2-((1-oxo-4-(o-tolyl)-1,2-dihydroisoquinolin-7-yl)oxy)propanoyl)piperidin-3-yl)acetate, 38-3 [Step 1]: To a stirred solution of (R)-2-((1-oxo-4-(o-tolyl)-1,2-dihydroisoquinolin-7-yl)oxy)propanoic acid (38-1, 170 mg, 0.5 mmol) and ethyl (R)-2-(piperidin-3-yl)acetate hydrochloride (38-2, 120 mg, 0.6 mmol) in dichloromethane (5 mL), DIPEA (0.6 mL, 3.4 mmol) was added followed by T₃P (1.4 mL, 2.3 mmol, 50% in ethyl acetate) dropwise at 0° C. and stirred the reaction mixture at ambient temperature for 3 h. After completion, the reaction mixture was diluted with dichloromethane and washed with water (twice). The organic extract was collected, washed with brine, dried with anhydrous Na₂SO₄ and concentrated under reduced pressure. The product was purified by column chromatography to afford ethyl 2-((R)-1-((R)-2-((1-oxo-4-(0-tolyl)-1,2-dihydroisoquinolin-7-yl)oxy)propanoyl)piperidin-3-yl)acetate (38-3, 240 mg). LCMS (ESI) Calcd. for C₂H₃₂N₂O₅: 477, found [M+H]⁺=477.

Synthesis of 2-((R)-1-((R)-2-((1-oxo-4-(o-tolyl)-1,2-dihydroisoquinolin-7-yl)oxy)propanoyl)piperidin-3-yl)acetic acid, 38-4 [Step 2]: To a stirred solution of ethyl 2-((R)-1-((R)-2-((1-oxo-4-(o-tolyl)-1,2-dihydroisoquinolin-7-yl)oxy)propanoyl)piperidin-3-yl)acetate (38-3, 250 mg, 0.5 mmol) in THF (8 mL) and water (2 mL), LiOH·H₂O (65 mg, 1.6 mmol) was added and stirred at ambient temperature for 4 h. After completion, the reaction mixture was quenched with 1N HCl solution to adjust the pH 3 and extracted with ethyl acetate (twice). The organic extract was collected, dried over anhydrous Na₂SO₄ and concentrated under reduced pressure to afford 2-((R)-1-((R)-2-((1-oxo-4-(o-tolyl)-1,2-dihydroisoquinolin-7-yl)oxy)propanoyl)piperidin-3-yl)acetic acid (38-4, 240 mg). LCMS (ESI) Calcd. for C₂₆H₂₈N₂O₅: 449; found [M+H]⁺=449.

Synthesis of 2-((R)-1-((R)-2-((1-oxo-4-(o-tolyl)-1,2-dihydroisoquinolin-7-yl)oxy)propanoyl)piperidin-3-yl)acetamide, Example 38 [Step 3]: To a stirred solution of 2-((R)-1-((R)-2-((1-oxo-4-(o-tolyl)-1,2-dihydroisoquinolin-7-yl)oxy)propanoyl)piperidin-3-yl)acetic acid (38-4, 200 mg, 0.4 mmol) in THF (6 mL) and DMF (3 mL) mixture, HOBt (90 mg, 0.7 mmol), EDC·HCl (130 mg, 0.7 mmol) and DIPEA (0.3 mL, 1.8 mmol) were added followed by (NH₄)₂CO₃ (170 mg, 1.8 mmol) and stirred at ambient temperature for 16 h. After completion, the reaction mixture was diluted with water and extracted with ethyl acetate (twice). The combined organic extract was collected, washed with brine, dried over anhydrous Na₂SO₄ and, concentrated under reduced pressure. The product was purified by reverse phase prep-HPLC and lyophilized to afford 2-((R)-1-((R)-2-((1-oxo-4-(v-tolyl)-1,2-dihydroisoquinolin-7-yl)oxy)propanoyl)piperidin-3-yl)acetamide (Example 38, 45 mg). LCMS (ESI) Calcd. for C₂₆H₂₉N₃O₄: 448, found [M+H]⁺=448. ¹H NMR (400 MHz, DMSO-d₆ at 100° C.): δ_{H} 11.08 (br s, 1H), 7.67 (s, 1H), 7.34 (d, 2H), 7.29-7.18 (m, 3H), 6.92 (d, 1H), 6.84 (s, 1H), 6.60 (br s, 2H), 5.30 (d, 1H), 4.07-3.89 (m, 3H), 2.06 (s, 5H), 1.88-1.79 (m, 2H), 1.63 (br s, 2H), 1.49 (d, 3H), 1.27 (br s, 2H).

### Example 39: Synthesis of 2-((S)-1-((R)-2-((1-oxo-4-(o-tolyl)-1,2-dihydroisoquinolin-7-yl)oxy)propanoyl)piperidin-3-yl)acetamide

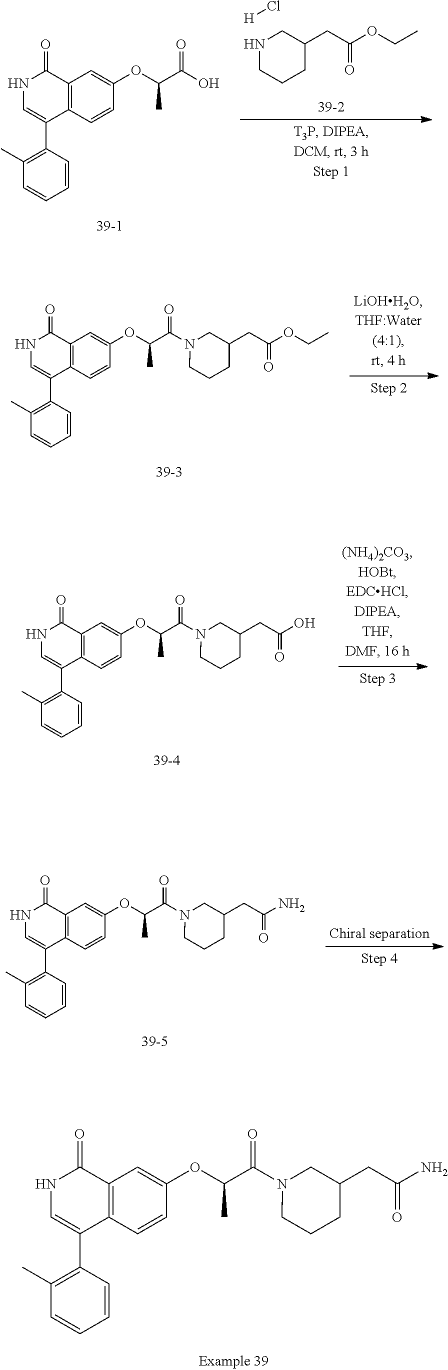

Synthesis of ethyl 2-(1-((R)-2-((1-oxo-4-(o-tolyl)-1,2-dihydroisoquinolin-7-yl)oxy)propanoyl)piperidin-3-yl)acetate, 39-3 [Step 1]: To a stirred solution of (R)-2-((1-oxo-4-(o-tolyl)-1,2-dihydroisoquinolin-7-yl)oxy)propanoic acid (39-1, 250 mg, 0.8 mmol) and ethyl 2-(piperidin-3-yl)acetate (39-2, 160 mg, 0.9 mmol) in dichloromethane (2 mL), DIPEA (0.4 mL, 2.3 mmol) was added followed by T₃P (0.7 mL, 1.2 mmol, 50% in ethyl acetate) dropwise at 0° C. and stirred at ambient temperature for 3 h. The reaction mixture was diluted with dichloromethane and washed with water (twice). The organic extract was collected, dried with anhydrous Na₂SO₄ and concentrated under reduced pressure to afford ethyl 2-(1-((R)-2-((1-oxo-4-(o-tolyl)-1,2-dihydroisoquinolin-7-yl)oxy)propanoyl)piperidin-3-yl)acetate (39-3, 330 mg). LCMS (ESI) Calcd. for C₂₈H₃₂N₂O₅: 477, found [M+H]⁺=477.

Synthesis of 2-(1-((R)-2-((1-oxo-4-(o-tolyl)-1,2-dihydroisoquinolin-7-yl)oxy)propanoyl)piperidin-3-yl)acetic acid, 39-4 [Step 2]: To a stirred solution of ethyl 2-(1-((R)-2-((1-oxo-4-(o-tolyl)-1,2-dihydroisoquinolin-7-yl)oxy)propanoyl)piperidin-3-yl)acetate (39-3,350 mg, 0.7 mmol) in THF (8 mL) and water (2 mL) mixture, LiOH·H₂O (95 mg, 2.2 mmol) was added and stirred at ambient temperature for 4 h. The reaction mixture was quenched with 1N HCl solution to adjust to pH 3 and extracted with ethyl acetate. The organic extract was collected, dried over anhydrous Na₂SO₄ and concentrated under reduced pressure to afford 2-(1-((R)-2-((1-oxo-4-(o-tolyl)-1,2-dihydroisoquinolin-7-yl)oxy)propanoyl)piperidin-3-yl)acetic acid (394, 300 mg). LCMS (ESI) Calcd. for C₂₆H₂₈N₂O₅: 449, found [M+H]⁺=449.

Synthesis of 2-(1-((R)-2-((1-oxo-4-(o-tolyl)-1,2-dihydroisoquinolin-7-yl)oxy)propanoyl)piperidin-3-yl)acetamide, 39-5 [Step 3]: To a stirred solution of 2-(1-((R)-2-((1-oxo-4-(o-tolyl)-1,2-dihydroisoquinolin-7-yl)oxy)propanoyl)piperidin-3-yl)acetic acid (394, 300 mg, 0.7 mmol) in THF (6 mL) and DMF (3 mL) mixture HOBt (135 mg, 1.0 mmol), EDC·HCl (190 mg, 1.0 mmol) and DIPEA (0.5 mL, 2.7 mmol) were added followed by (NH₄)₂CO₃(255 mg, 2.7 mmol) and stirred at ambient temperature for 16 h. The reaction mixture was quenched with water and extracted with ethyl acetate (twice). The combined organic extract was washed with brine, dried over anhydrous Na₂SO₄ and concentrated under reduced pressure to afford 2-(1-((R)-2-((1-oxo-4-(o-tolyl)-1,2-dihydroisoquinolin-7-yl)oxy)propanoyl)piperidin-3-yl)acetamide (39-5, 280 mg). LCMS (ESI) Calcd. for C₂₆H₂₉N₃O₄: 448; found [M+H]⁺=448.

Synthesis of 2-((S)-1-((R)-2-((1-oxo-4-(o-tolyl)-1,2-dihydroisoquinolin-7-yl)oxy)propanoyl)piperidin-3-yl)acetamide. Example 39 [Step 4]: Racemic 2-(1-((R)-2-((1-oxo-4-(o-tolyl)-1,2-dihydroisoquinolin-7-yl)oxy)propanoyl)piperidin-3-yl)acetamide (39-5, 260 mg) was purified by prep-SFC purification and lyophilized to afford the first product as 2-((S)-1-((R)-2-((1-oxo-4-(o-tolyl)-1,2-dihydroisoquinolin-7-yl)oxy)propanoyl)piperidin-3-yl)acetamide (Example 39, 25 mg) as Peak 1. The stereochemistry was confirmed by comparing with the HPLC retention time for absolute stereoisomer 2-((R)-1-((R)-2-((1-oxo-4-(o-tolyl)-1,2-dihydroisoquinolin-7-yl)oxy)propanoyl)piperidin-3-yl)acetamide (Example 38).

Prep-SFC method: SFC prep purification was performed on a Pic Solution 175 instrument equipped with Knauer UV Detector 40D using (RR) Whelk-O-1 (21.1 mm×250 mm), 5μ, which was operated at 35° C. temperature with a flow rate of 70 mL/min. The mobile phase used was a mixture of 75% CO₂ in super critical state and 25% of [0.1% IPAmine in MeOH+ACN (1:1)]. The instrument was held in isocratic mode for up to 20 min and at an isobaric condition of 100 bar and a wavelength of 240 nm.

Example 39: 2-((S)-1-((R)-2-((1-oxo-4-(o-tolyl)-1,2-dihydroisoquinolin-7-yl)oxy)propanoyl)piperidin-3-yl)acetamide (Peak 1): LCMS (ESI) Calcd. for C₂₆H₂₉N₃O₄: 448, found [M−H]⁻=446. HPLC chiral purity: 100% de. ¹H NMR (400 MHz, DMSO-d₆ at 100° C.): δ_{H} 10.98 (br s, 1H), 7.68 (s, 1H), 7.34 (d, 2H), 7.29-7.18 (m, 3H), 6.93 (d, 1H), 6.84 (s, 1H), 6.63 (br s, 2H), 5.29 (d, 1H), 4.03-3.99 (m, 1H), 2.98-2.80 (m, 2H), 2.06 (s, 5H), 1.88-1.79 (m, 2H), 1.70 (br s, 2H), 1.49 (d, 3H), 1.27 (br s, 2H).

### Example 40-41: Synthesis of chiral analogs of 1-(1-((R)-2-((1-oxo-4-(o-tolyl)-1,2-dihydroisoquinolin-7-yl)oxy)propanoyl)piperidin-3-yl)cyclopropane-1-carboxamide

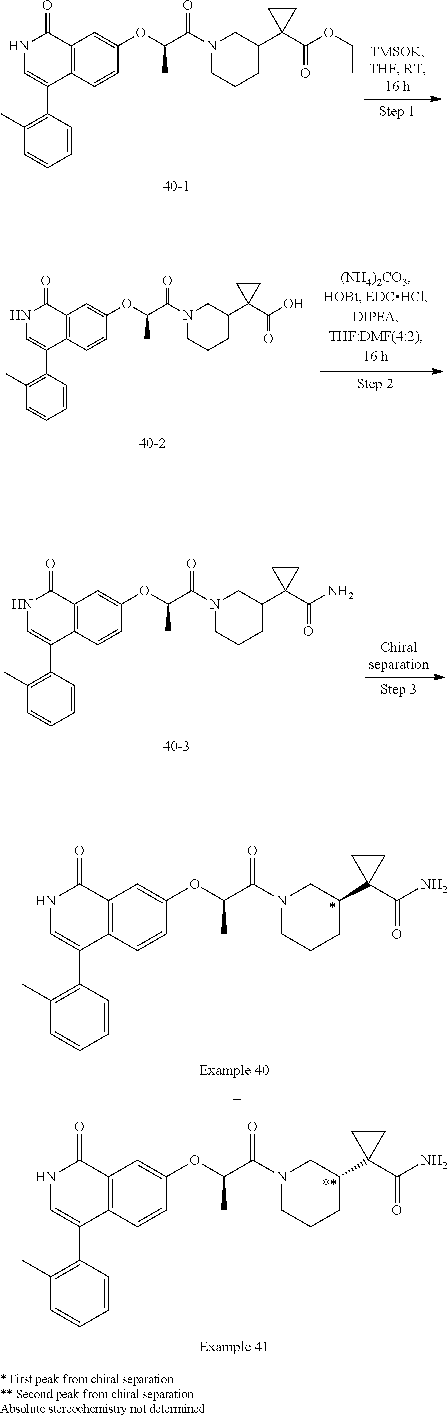

Synthesis of 1-(1-((R)-2-((1-oxo-4-(o-tolyl)-1,2-dihydroisoquinolin-7-yl)oxy)propanoyl)piperidin-3-yl)cyclopropane-1-carboxylic acid, 40-2 [Step 1]: To a stirred solution of ethyl 1-(1-((R)-2-((1-oxo-4-(o-tolyl)-1,2-dihydroisoquinolin-7-yl)oxy)propanoyl)piperidin-3-yl)cyclopropane-1-carboxylate (40-1, 300 mg, 0.6 mmol) in THF (5 mL), potassium trimethylsilanolate (230 mg, 1.8 mmol) was added and stirred at ambient temperature for 16 h. The reaction mixture was quenched with 1N HCl solutions to adjust to pH and extracted with ethyl acetate (twice). The organic extract was collected, dried over anhydrous Na₂SO₄ and concentrated under reduced pressure to afford 1-(1-((R)-2-((1-oxo-4-(o-tolyl)-1,2-dihydroisoquinolin-7-yl)oxy)propanoyl)piperidin-3-yl)cyclopropane-1-carboxylic acid (40-2, 280 mg). LCMS (ESI) Calcd. for C₂₈H₃₀N₂O₅: 475, found [M+H]⁺=475.

Synthesis of 1-(1-((R)-2-((1-oxo-4-(o-tolyl)-1,2-dihydroisoquinolin-7-yl)oxy)propanoyl)piperidin-3-yl)cyclopropane-1-carboxamide, 40-3 [Step 2]: To a stirred solution of 1-(1-((R)-2-((1-oxo-4-(o-tolyl)-1,2-dihydroisoquinolin-7-yl)oxy)propanoyl)piperidin-3-yl)cyclopropane-1-carboxylic acid (40-2, 300 mg, 0.6 mmol) in a mixture THF (6 mL) and DMF (3 mL), HOBt (130 mg, 0.9 mmol), EDC·HCl (180 mg, 0.9 mmol) and DIPEA (0.4 mL, 2.5 mmol) were added followed by (NH₄)₂CO₃ (245 mg, 2.5 mmol) and stirred at ambient temperature for 16 h. The reaction mixture was diluted with ethyl acetate and washed with water. The organic extract was collected, washed with brine, dried over anhydrous Na₂SO₄ and, concentrated under reduced pressure. The product was purified by reverse phase prep-HPLC to afford 1-(1-((R)-2-((1-oxo-4-(o-tolyl)-1,2-dihydroisoquinolin-7-yl)oxy)propanoyl)piperidin-3-yl)cyclopropane-1-carboxamide (40-3, 100 mg). LCMS (ESI) Calcd. for C₂₈H₃₁N₃O₄. 474, found [M+H]⁺=474.

Synthesis of chiral 1-(1-((R)-2-((1-oxo-4-(o-tolyl)-1,2-dihydroisoquinolin-7-yl)oxy)propanoyl)piperidin-3-yl)cyclopropane-1-carboxamide, Example 40 and Example 41 [Step 3]: Racemic 1-(1-((R)-2-((1-oxo-4-(o-tolyl)-1,2-dihydroisoquinolin-7-yl)oxy)propanoyl)piperidin-3-yl)cyclopropane-1-carboxamide (40-3, 100 mg) was purified by prep-SFC purification and lyophilized to afford the first product as chiral 1-(1-((R)-2-((1-oxo-4-(o-tolyl)-1,2-dihydroisoquinolin-7-yl)oxy)propanoyl)piperidin-3-yl)cyclopropane-1-carboxamide (Example 40, 25 mg) as Peak 1 and the second product as chiral 1-(1-((R)-2-((1-oxo-4-(o-tolyl)-1,2-dihydroisoquinolin-7-yl)oxy)propanoyl)piperidin-3-yl)cyclopropane-1-carboxamide (Example 41, 25 mg) as Peak 2. The absolute stereochemistry was not determined.

Prep-SFC method: SFC Prep purification was performed on a Waters THAR SFC-80 instrument using Chiralpak ID (21 mm×250 mm), 5μ, which was operated at 35° C. temperature with a flow rate of 60 mL/min, using 50% C₀₂ in super critical state and 50% of ACN:MeOH (1:1). The instrument was held in isocratic mode for up to 12 min and at an isobaric condition of bar with a wavelength of 240 nm.

Example 40: Chiral 1-(1-((R)-2-((1-oxo-4-(v-tolyl)-1,2-dihydroisoquinolin-7-yl)oxy)propanoyl)piperidin-3-yl)cyclopropane-1-carboxamide (Peak 1): LCMS (ESI) Calcd. for C₂₈H₃₁N₃O₄: 474, found [M+H]⁺=474: HPLC chiral purity: 100% de. ¹H NMR (400 MHz, DMSO-d₆ at 100° C.): δ_{H} 10.98 (br s, 1H), 7.67 (s, 1H), 7.34 (d, 2H), 7.29-7.18 (m, 3H), 6.93 (d, 1H), 6.84 (s, 1H), 6.55 (br s, 2H), 5.31 (d, 1H), 4.18 (br s, 4H), 2.06 (s, 3H), 1.73-1.70 (m, 3H), 1.50 (d, 3H), 1.39-1.37 (m, 2H), 0.92-0.91 (m, 1H), 0.80 (br s, 1H), 0.65 (br s, 2H).

Example 41: Chiral 1-(1-((R)-2-((1-oxo-4-(o-tolyl)-1,2-dihydroisoquinolin-7-yl)oxy)propanoyl)piperidin-3-yl)cyclopropane-1-carboxamide (Peak 2): LCMS (ESI) Calcd. for C₂₈H₃₁N₃O₄: 474, found [M+H]⁺=474. HPLC chiral purity: 100% de. ¹H NMR (400 MHz, DMSO-d₆ at 100° C.): 6j 10.96 (br s, 1H), 7.69 (s, 1H), 7.34 (d, 2H), 7.29-7.18 (m, 3H), 6.93 (d, 1H), 6.83 (s, 1H), 6.53 (br s, 2H), 5.28 (q, 1H), 4.23 (br s, 4H), 2.07 (s, 3H), 1.78-1.60 (m, 3H), 1.50 (d, 3H), 1.39-1.27 (m, 2H), 0.92-0.89 (m, 1H), 0.83-0.81 (m, 1H), 0.65 (br s, 2H).

### Example 42: Synthesis of 2-((4-(2-(2-hydroxyethyl)phenyl)-1-oxo-1,2-dihydroisoquinolin-7-yl)oxy)acetonitrile

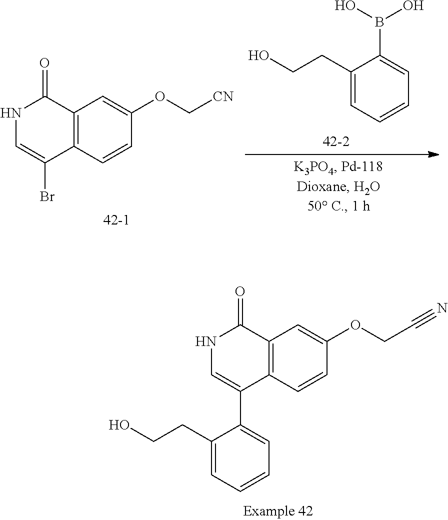 [SMILES: N#CCOc1ccc2c(Br)c[nH]c(=O)c2c1 ; OCCc1ccccc1B(O)O ; N#CCOc1ccc2c(-c3ccccc3CCO)c[nH]c(=O)c2c1]

To a stirred solution of 2-((4-bromo-1-oxo-1,2-dihydroisoquinolin-7-yl)oxy)acetonitrile (42-1, 100 mg, 0.4 mmol) in 1,4-dioxane (4 mL) and water (1 mL) mixture, K₃PO₄ (150 mg, 0.7 mmol) and (2-(2-hydroxyethyl)phenyl)boronic acid (42-2, 95 mg, 0.6 mmol) were added. The reaction mixture was degassed with nitrogen gas for 10 min. Into this purged reaction mixture [1,1′-bis(di-tert-butylphosphino)ferrocene]dichloropalladium(II) (25 mg, 0.03 mmol) was added and the reaction mixture was heated at 50° C. for 1 h. After completion, the reaction mixture was filtered through a celite bed and the bed was washed with ethyl acetate. The combined filtrate was washed with water, brine, dried over anhydrous Na₂SO₄ and concentrated under reduced pressure. The product was purified by reverse phase prep HPLC purification method and lyophilized to afford 2-((4-(2-(2-hydroxyethyl)phenyl)-1-oxo-1,2-dihydroisoquinolin-7-yl)oxy)acetonitrile (Example 42, 50 mg). LCMS (ESI) Calcd. for C₁₉H₁₆N₂O₃: 320, found [M+H]⁺=321. ¹H NMR (400 MHz, DMSO-d₆): δ_{H} 11.48 (br s, 1H), 7.86 (d, 1H), 7.41-7.35 (m, 4H), 7.31-7.27 (m, 1H), 7.19-7.17 (m, 1H), 6.98-6.94 (m, 1H), 5.32 (s, 2H), 4.52-4.49 (m, 1H), 3.42-3.30 (m, 2H), 2.62-2.32 (m, 2H).

### Example 43-44: Synthesis of chiral analogs of 2-methyl-2-(1-((R)-2-((1-oxo-4-(o-tolyl)-1,2-dihydroisoquinolin-7-yl)oxy)propanoyl)piperidin-3-yl)propanoic acid

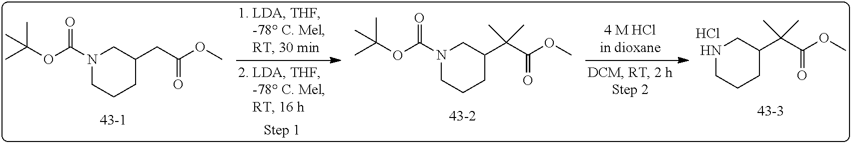 [SMILES: COC(=O)CC1CCCN(C(=O)OC(C)(C)C)C1 ; COC(=O)C(C)(C)C1CCCN(C(=O)OC(C)(C)C)C1 ; Cl ; COC(=O)C(C)(C)C1CCCNC1]

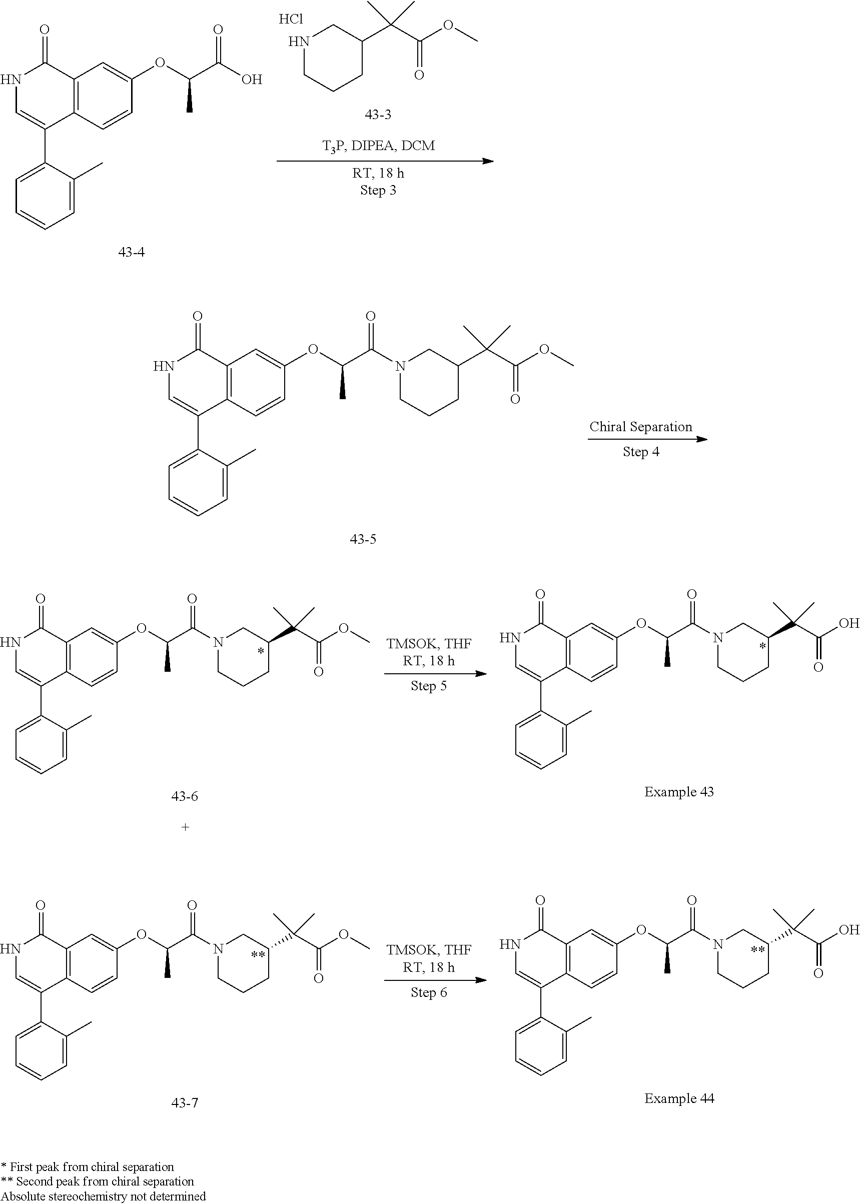

Synthesis of tert-butyl 3-(1-methoxy-2-methyl-1-oxopropan-2-yl)piperidine-1-carboxylate, 43-2 [Step 1]: To a freshly prepared lithium diisopropyl amide solution [prepared from diisopropyl amine (0.6 mL, 4.3 mmol) and n-butyllithium (2.5M in hexane) (1.7 mL, 4.3 mmol) at −78° C. in THF (10 mL)] was added dropwise a THF (5 mL) solution of tert-butyl 3-(2-methoxy-2-oxoethyl)piperidine-1-carboxylate (43-1, 1.0 g, 3.9 mmol) at −78° C. The mixture was stirred at −78° C. for 15 minutes. Methyl iodide (0.3 mL, 4.3 mmol) was added in to the mixture and allowed to warm to ambient temperature and stirred over a period of 30 minutes. Another batch of freshly prepared lithium diisopropyl amide solution [prepared from diisopropyl amine (0.6 mL, 4.3 mmol) and n-butyllithium (2.5M in hexane) (1.7 mL, 4.3 mmol) in dry THF (10 mL) at −78° C.] was added dropwise to the reaction mixture and stirred at −78° C. for 15 min. Methyl iodide (0.3 mL, 4.3 mmol) was added in to the mixture and allowed to warm to ambient temperature and stirred for 16 h. After completion, the reaction mixture was quenched with 10 ml of 0.1M HCl and extracted with tert-butyl methyl ether (twice). The combined organic extract was washed with brine, dried over Na₂SO₄ and concentrated under reduced pressure to afford tert-butyl 3-(1-methoxy-2-methyl-1-oxopropan-2-yl)piperidine-1-carboxylate (43-2, 950 mg).

Synthesis of methyl 2-methyl-2-(piperidin-3-yl)propanoate hydrochloride, 43-3 [Step 2]: A stirred solution of tert-butyl 3-(2-methoxy-1,1-dimethyl-2-oxo-ethyl)piperidine-1-carboxylate (43-2, 450 mg, 1.6 mmol) in dichloromethane (20 mL) was cooled to 0° C. 4M HCl in dioxane (4.0 mL, 15.8 mmol) was added dropwise. The mixture was gradually warmed to ambient temperature and stirred for 2 h. After completion, the mixture was concentrated under reduced pressure, washed with diethyl ether and dried under reduced pressure to afford methyl 2-methyl-2-(piperidin-3-yl)propanoate hydrochloride (43-3, 310 mg).

Synthesis of methyl 2-methyl-2-(1-((R)-2-((1-oxo-4-(o-tolyl)-1,2-dihydroisoquinolin-7-yl)oxy)propanoyl)piperidin-3-yl)propanoate, 43-5 [Step 3]: To a stirred solution of (R)-2-((1-oxo-4-(o-tolyl)-1,2-dihydroisoquinolin-7-yl)oxy)propanoic acid (43-4, 300 mg, 0.9 mmol) and methyl 2-methyl-2-(piperidin-3-yl)propanoate hydrochloride (43-3, 310 mg, 1.4 mmol) in dichloromethane (20 mL), N,N-diisopropylethylamine (0.8 mL, 4.6 mmol) was added and the mixture was cooled to 0° C. Into the mixture, T₃P (0.8 mL, 1.4 mmol, 50% in ethyl acetate) was added dropwise at ice cold condition and the mixture was stirred at ambient temperature for 18 h. After completion, the reaction mixture was diluted water and extracted with dichloromethane (twice). The combined organic extract was dried over anhydrous Na₂SO₄ and concentrated under reduced pressure. The product was purified by reverse phase prep HPLC to afford methyl 2-methyl-2-(1-((R)-2-((1-oxo-4-(o-tolyl)-1,2-dihydroisoquinolin-7-yl)oxy)propanoyl)piperidin-3-yl)propanoate (43-5, 180 mg). LCMS (ESI) Calcd. for C₂₉H₃₄N₂O₅: 490; found [M+H]⁺=491. ¹H NMR (400 MHz, DMSO-d₆): δ_{H} 11.34 (s, 1H), 7.66-7.54 (m, 1H), 7.34 (br s, 2H), 7.28-7.23 (m, 2H), 7.19-7.18 (m, 1H), 6.89 (br s, 2H), 5.42-5.25 (m, 1H), 4.37 (br s, 1H), 4.10-4.01 (m, 1H), 3.59 (s, 3H), 3.17-3.16 (m, 1H), 2.92-2.86 (m, 1H), 2.29 (t, 1H), 2.05 (s, 3H), 1.73-1.62 (m, 3H), 1.46-1.42 (m, 3H), 1.32-1.23 (m, 1H), 1.11-1.03 (m, 6H).

Synthesis of chiral methyl 2-methyl-2-(1-((R)-2-((1-oxo-4-(o-tolyl)-1,2-dihydroisoquinolin-7-yl)oxy)propanoyl)piperidin-3-yl)propanoate, 43-6 and 43-7 [Step 4]: The racemic methyl 2-methyl-2-(1-((R)-2-((1-oxo-4-(o-tolyl)-1,2-dihydroisoquinolin-7-yl)oxy)propanoyl)piperidin-3-yl)propanoate (43-5, 170 mg, 0.3 mmol) was used for HPLC chiral SFC separation to afford the first product as chiral methyl 2-methyl-2-(1-((R)-2-(1-oxo-4-(o-tolyl)-1,2-dihydroisoquinolin-7-yl)oxy)propanoyl)piperidin-3-yl)propanoate (43-6, 60 mg) as Peak 1 and the second product as chiral methyl 2-methyl-2-(1-((R)-2-((1-oxo-4-(o-tolyl)-1,2-dihydroisoquinolin-7-yl)oxy)propanoyl)piperidin-3-yl)propanoate (43-7, 65 mg) as Peak 2. The absolute stereochemistry for these compounds was not determined.

Chiral HPLC prep-Method (SFC): SFC prep purification was performed on a Waters THAR SFC-80 instrument using Chiralpak ID (21 mm×250 mm), 5μ, which was operated at 35° C. temperature with a flow rate of 70 mL/min. The mobile phase was a mixture of 70% CO₂ in super critical state and 30% of (0.3% IPAmine in MeOH). The instrument was held in isocratic mode for up to 15 min and at an isobaric condition of 110 bar with a wavelength of 220 nm.

43-6: Chiral methyl 2-methyl-2-(1-((R)-2-((1-oxo-4-(o-tolyl)-1,2-dihydroisoquinolin-7-yl)oxy)propanoyl)piperidin-3-yl)propanoate (Peak 1): LCMS (ESI) Calcd. for C₂₉H₃₄N₂O₅: 490, found [M+H]⁺=491: Chiral purity: 100% de.

43-7: Chiral methyl 2-methyl-2-(1-((R)-2-((1-oxo-4-(o-tolyl)-1,2-dihydroisoquinolin-7-yl)oxy)propanoyl)piperidin-3-yl)propanoate (Peak 2): LCMS (ESI) Calcd. for C₂₉H₃₄N₂O₅: 490, found [M+H]⁺=491; Chiral purity: 95.96% de.

Synthesis of 2-methyl-2-(1-((R)-2-((1-oxo-4-(o-tolyl)-1,2-dihydroisoquinolin-7-yl)oxy)propanoyl)piperidin-3-yl)propanoic acid, Example 43 [Step 5]: To a stirred solution of chiral methyl 2-methyl-2-(1-((R)-2-((1-oxo-4-(o-tolyl)-1,2-dihydroisoquinolin-7-yl)oxy)propanoyl)piperidin-3-yl)propanoate (43-6, 60 mg, 0.12 mmol) in THF (6 mL), potassium trimethylsilanolate (50 mg, 0.4 mmol) was added. The reaction mixture was stirred at ambient temperature for 18 h. After completion, the reaction mixture was concentrated under reduced pressure. The residue was acidified with 2N HCl to pH=4 and extracted with dichloromethane (twice). The combined organic extract was washed with brine, dried over anhydrous Na₂SO₄ and concentrated under reduced pressure. The product was purified by reverse phase prep HPLC to afford 2-methyl-2-(1-((R)-2-((1-oxo-4-(o-tolyl)-1,2-dihydroisoquinolin-7-yl)oxy)propanoyl)piperidin-3-yl)propanoic acid (Example 43, 30 mg). LCMS (ESI) Calcd. for C₂H₃₂N₂O₅: 476, found [M+H]⁺=477. ¹H NMR (400 MHz, DMSO-d₆): δ_{H} 11.05 (br s, 1H), 7.69 (s, 1H), 7.34 (br s, 2H), 7.26-7.26 (m, 1H), 7.23-7.18 (m, 2H), 6.92 (d, 1H), 6.84 (s, 1H), 5.29 (br s, 1H), 4.27 (br s, 2H), 3.02-2.84 (m, 1H), 2.44-2.07 (m, 2H), 2.06 (s, 3H), 1.76-1.73 (m, 2H), 1.50 (d, 3H), 1.35-1.29 (m, 2H), 1.08 (s, 6H). The absolute stereochemistry was not determined.

Synthesis of 2-methyl-2-(1-((R)-2-((1-oxo-4-(o-tolyl)-1,2-dihydroisoquinolin-7-yl)oxy)propanoyl)piperidin-3-yl)propanoic acid, Example 44 [Step 6]: To a stirred solution of methyl 2-methyl-2-(1-((R)-2-((1-oxo-4-(o-tolyl)-1,2-dihydroisoquinolin-7-yl)oxy)propanoyl)piperidin-3-yl)propanoate (43-7, 65 mg, 0.13 mmol) in THF (10 mL), potassium trimethylsilanolate (50 mg, 0.4 mmol) was added. The reaction mixture was stirred at ambient temperature for 18 h. The reaction mixture was concentrated under reduced pressure. The residue was acidified with 2N HCl to pH=5 and was extracted with dichloromethane (twice). The combined organic extract was washed with brine, dried over anhydrous Na₂SO₄ and concentrated under reduced pressure. The product was purified by reverse phase prep HPLC to afford 2-methyl-2-(1-((R)-2-((1-oxo-4-(o-tolyl)-1,2-dihydroisoquinolin-7-yl)oxy)propanoyl)piperidin-3-yl)propanoic acid (Example 44, 25 mg). LCMS (ESI) Calcd. for C₂₈H₃₂N₂O₅: 476, found [M+H]⁺=477. ¹H NMR (400 MHz, DMSO-d₆): δ_{H} 11.09 (br s, 1H), 7.68 (s, 1H), 7.35-7.18 (m, 5H), 6.92 (d, 1H), 6.84 (s, 1H), 5.30 (br s, 1H), 4.44-4.35 (m, 1H), 4.06-4.03 (m, 1H), 2.94-2.89 (m, 1H), 2.34-2.26 (m, 1H), 2.07 (s, 3H), 1.76-1.72 (m, 2H), 1.65-1.59 (m, 1H), 1.48 (d, 3H), 1.32-1.29 (m, 2H), 1.08 (s, 6H). The absolute stereochemistry was not determined.

### Example 45-46: Synthesis of chiral analogs of 2-methyl-2-(1-((R)-2-((1-oxo-4-(o-tolyl)-1,2-dihydroisoquinolin-7-yl)oxy)propanoyl)piperidin-3-yl)propanamide

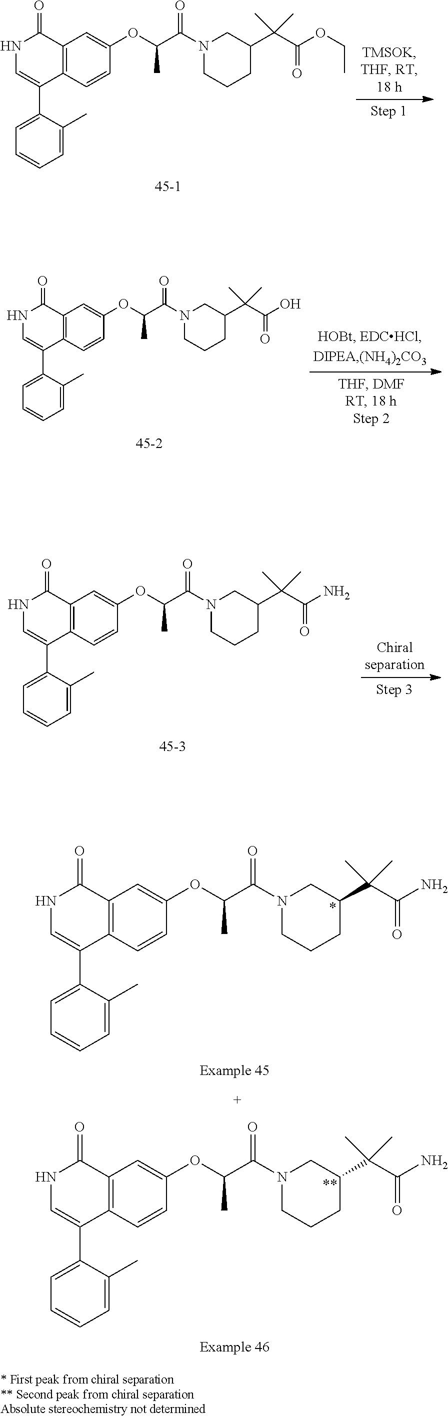 [SMILES: CCOC(=O)C(C)(C)C1CCCN(C(=O)[C@@H](C)Oc2ccc3c(-c4ccccc4C)c[nH]c(=O)c3c2)C1 ; Cc1ccccc1-c1c[nH]c(=O)c2cc(O[C@H](C)C(=O)N3CCCC(C(C)(C)C(=O)O)C3)ccc12 ; Cc1ccccc1-c1c[nH]c(=O)c2cc(O[C@H](C)C(=O)N3CCCC(C(C)(C)C(N)=O)C3)ccc12 ; Cc1ccccc1-c1c[nH]c(=O)c2cc(O[C@H](C)C(=O)N3CCC[C@H](C(C)(C)C(N)=O)C3)ccc12 ; Cc1ccccc1-c1c[nH]c(=O)c2cc(O[C@H](C)C(=O)N3CCC[C@@H](C(C)(C)C(N)=O)C3)ccc12]

Synthesis of 2-methyl-2-(1-((R)-2-((1-oxo-4-(o-tolyl)-1,2-dihydroisoquinolin-7-yl)oxy)propanoyl)piperidin-3-yl)propanoic acid, 45-2 [Step 1]: To a stirred solution of methyl 2-methyl-2-(1-((R)-2-((1-oxo-4-(o-tolyl)-1,2-dihydroisoquinolin-7-yl)oxy)propanoyl)piperidin-3-yl)propanoate (45-1, 340 mg, 0.5 mmol) in THF (20 mL), potassium trimethylsilanolate (205 mg, 1.6 mmol) was added. The mixture was stirred at ambient temperature for 18 h. The reaction mixture was concentrated under reduced pressure. The residue was acidified with 2N HCl to pH=5 and extracted with dichloromethane (twice). The combined organic extract was washed with brine, dried over anhydrous Na₂SO₄ and concentrated under reduced pressure to afford 2-methyl-2-(1-((R)-2-((1-oxo-4-(o-tolyl)-1,2-dihydroisoquinolin-7-yl)oxy)propanoyl)piperidin-3-yl)propanoic acid (45-2, 310 mg). LCMS (ESI) Calcd. for C₂₈H₃₂N₂O₅: 476, found [M+H]⁺=477.

Synthesis of 2-methyl-2-(1-((R)-2-((1-oxo-4-(o-tolyl)-1,2-dihydroisoquinolin-7-yl)oxy)propanoyl)piperidin-3-yl)propanamide, 45-3 [Step 2]: To a stirred solution of 2-methyl-2-(1-((R)-2-((1-oxo-4-(o-tolyl)-1,2-dihydroisoquinolin-7-yl)oxy)propanoyl)piperidin-3-yl)propanoic acid (45-2, 300 mg, 0.5 mmol) in THF (6 mL) and DMF (3 mL), hydroxybenzotriazole (HOBt) (95 mg, 0.7 mmol), 1-(3-dimethylaminopropyl)-3-ethylcarbodiimide hydrochloride (135 mg, 0.7 mmol) and N,N-diisopropylethylamine (0.4 mL, 2.3 mmol) were added and the mixture was stirred for 5 min. Ammonium carbonate (220 mg, 2.3 mmol) was added to the above mixture and stirred at ambient temperature for 18 h. After completion, the reaction mixture was diluted with water and extracted with dichloromethane (twice). The combined extract was washed with aqueous NaHCO₃ solution and brine, dried over anhydrous Na₂SO₄ and concentrated under reduced pressure. The product was purified by reverse phase prep HPLC to afford 2-methyl-2-(1-((R)-2-((1-oxo-4-(o-tolyl)-1,2-dihydroisoquinolin-7-yl)oxy)propanoyl)piperidin-3-yl)propanamide (45-3, 100 mg). LCMS (ESI) Calcd. for C₂₈H₃₃N₃O₄: 475, found [M+H]⁺=476.

Synthesis of chiral 2-methyl-2-(1-((R)-2-((1-oxo-4-(o-tolyl)-1,2-dihydroisoquinolin-7-yl)oxy)propanoyl)piperidin-3-yl)propanamide, Example 45 and Example 46 [Step 3]: The racemic 2-methyl-2-(1-((R)-2-((1-oxo-4-(o-tolyl)-1,2-dihydroisoquinolin-7-yl)oxy)propanoyl)piperidin-3-yl)propanamide (45-3, 100 mg, 0.2 mmol) was used for normal phase chiral HPLC separation to afford the first product as chiral 2-methyl-2-(1-((R)-2-((1-oxo-4-(o-tolyl)-1,2-dihydroisoquinolin-7-yl)oxy)propanoyl)piperidin-3-yl)propanamide (Example 45, 25 mg) as Peak 1 and the second product as chiral 2-methyl-2-(1-((R)-2-((1-oxo-4-(o-tolyl)-1,2-dihydroisoquinolin-7-yl)oxy)propanoyl)piperidin-3-yl)propanamide (Example 46, 40 mg) as Peak 2. The absolute stereochemistry of these compounds were not determined.

Chiral HPLC normal phase separation method: Chiral separation was performed on an Agilent 1200 series instrument using CHIRALPAK IG (250×21 mm), 5μ, which was operated at ambient temperature with a flow rate of 21.0 mL/min. The mobile phase was a mixture of 60% hexane, 20% EtOH, and 20% DCM. The instrument was held in isocratic mode for up to 30 min with a wavelength of 282 nm.

Example 45: Chiral 2-methyl-2-(1-((R)-2-((1-oxo-4-(o-tolyl)-1,2-dihydroisoquinolin-7-yl)oxy)propanoyl)piperidin-3-yl)propanamide (Peak 1): LCMS (ESI) Calcd. for C₂₈H₃₃N₃O₄: 475, found [M+H]⁺=476. ¹H NMR (400 MHz, DMSO-d₆): δ_{H} 11.03 (br s, 1H), 7.69 (s, 1H), 7.34 (br s, 2H), 7.28-7.18 (m, 3H), 6.94 (d, 1H), 6.85 (s, 1H), 6.69 (br s, 2H), 5.29 (br s, 1H), 4.26 (br s, 2H), 3.01-2.88 (m, 1H), 2.07 (s, 3H), 1.79-1.65 (m, 3H), 1.50 (d, 3H), 1.34-1.28 (m, 3H), 1.09 (s, 6H).

Example 46: Chiral 2-methyl-2-(1-((R)-2-((1-oxo-4-(o-tolyl)-1,2-dihydroisoquinolin-7-yl)oxy)propanoyl)piperidin-3-yl)propanamide (Peak 2): LCMS (ESI) Calcd. for C₂₈H₃₃N₃O₄: 475, found [M+H]⁺=476. ¹H NMR (400 MHz, DMSO-d₆): δ_{H} 10.99 (br s, 1H), 7.70 (s, 1H), 7.34 (br s, 2H), 7.28-7.18 (m, 3H), 6.94 (d, 1H), 6.84 (s, 1H), 6.63 (br s, 2H), 5.27 (br s, 11H), 4.19 (br s, 2H), 2.92-2.87 (m, 1H), 2.07 (s, 3H), 1.79-1.65 (m, 4H), 1.49 (d, 3H), 1.33-1.27 (m, 2H), 1.06 (s, 6H).

### Example 47: Synthesis of 2-((4-(2-(methoxymethyl)phenyl)-1-oxo-1,2-dihydroisoquinolin-7-yl)oxy)acetonitrile

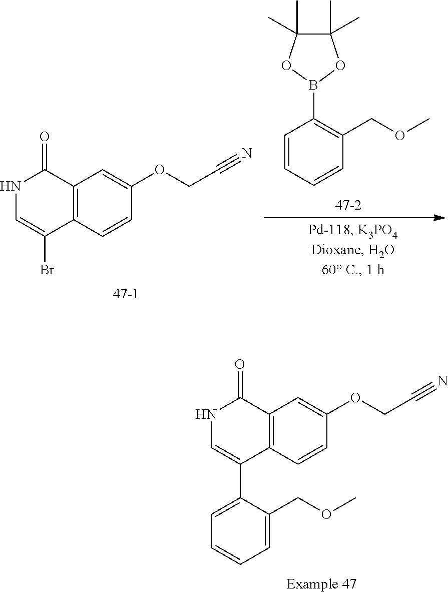

To a stirred solution of 2-((4-bromo-1-oxo-1,2-dihydroisoquinolin-7-yl)oxy)acetonitrile (47-1, 100 mg, 0.4 mmol) and 2-(2-(methoxymethyl)phenyl)-4,4,5,5-tetramethyl-1,3,2-dioxaborolane (47-2, 135 mg, 0.5 mmol) in 1,4-dioxane (6 mL) and water (1 mL) mixture, K₃PO₄ (150 mg, 0.7 mmol) was added. The reaction mixture was purged with nitrogen gas for 10 min and [1,1′-bis-di-tert-butylphosphino)ferrocene]dichloropalladium(II) (25 mg, 0.04 mmol) was added. The reaction mixture was heated at 60° C. for 1 h. After completion, the mixture was diluted with ethyl acetate and washed with brine. The organic extract was dried over anhydrous Na₂SO₄, filtered and concentrated under reduced pressure. The product was purified by reverse phase prep HPLC to afford 2-((4-(2-(methoxymethyl)phenyl)-1-oxo-1,2-dihydroisoquinolin-7-yl)oxy)acetonitrile (Example 47, 55 mg). LCMS (ESI) Calcd. for C₁₉H₁₆N₂O₃: 320, found [M+H]⁺=321. ¹H NMR (400 MHz, DMSO-d₆): δ_{H} 11.11 (br s, 1H), 7.90 (s, 1H), 7.55 (d, 1H), 7.48-7.39 (m, 2H), 7.34 (d, 1H), 7.25 (d, 1H), 7.03 (d, 1H), 6.93 (s, 1H), 5.23 (s, 2H), 4.19 (s, 2H), 3.14 (s, 3H).

### Example 48: Synthesis of 2-(7-(cyanomethoxy)-1-oxo-1,2-dihydroisoquinolin-4-yl)-3-methylbenzonitrile

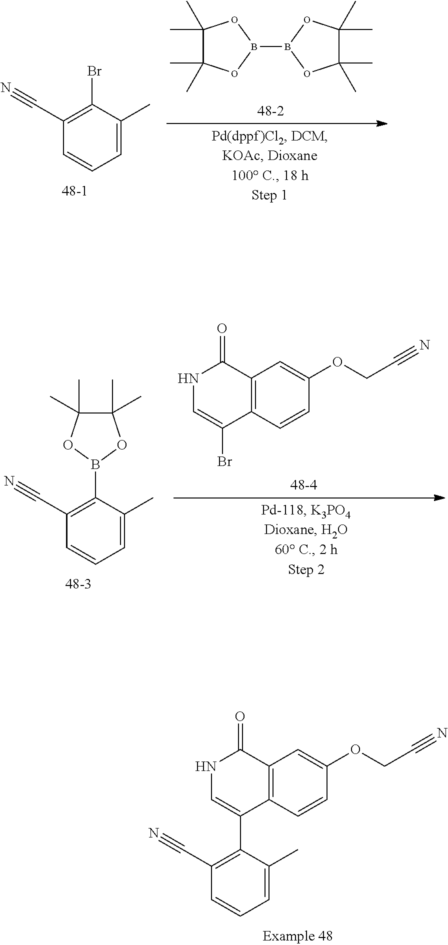

Synthesis of 3-methyl-2-(4,4,5,5-tetramethyl-1,3,2-dioxaborolan-2-yl)benzonitrile, 48-2 [Step 1]: To a stirred solution of dry potassium acetate (300 mg, 3.1 mmol) in 1,4-dioxane (10 mL), 2-bromo-3-methylbenzonitrile (48-1, 200 mg, 1.0 mmol) and 4,4,4′,4′,5,5,5′,5′-octamethyl-2,2′-bi(1,3,2-dioxaborolane) (48-2, 520 mg, 2.0 mmol) was added and the reaction mixture was purged Argon gas for 5 min. [1,1′-Bis(diphenylphosphino)ferrocene]dichloropalladium(II), complex with dichloromethane (85 mg, 0.1 mmol) was added into the reaction mixture and heated at 100° C. for 18 h. After completion, the reaction mixture was filtered through celite bed. The bed was washed with dioxane and concentrated under reduced pressure to afford 3-methyl-2-(4,4,5,5-tetramethyl-1,3,2-dioxaborolan-2-yl)benzonitrile (48-3, 600 mg). LCMS (ESI) Calcd. for C₁₄H₁₈BNO₂: 243, found [M+H]⁺=244.

Synthesis of 2-(7-(cyanomethoxy)-1-oxo-1,2-dihydroisoquinolin-4-yl)-3-methylbenzonitrile, Example 48 [Step 2]: To a stirred solution of 2-((4-bromo-1-oxo-1,2-dihydroisoquinolin-7-yl)oxy)acetonitrile (48-4, 100 mg, 0.4 mmol) and 3-methyl-2-(4,4,5,5-tetramethyl-1,3,2-dioxaborolan-2-yl)benzonitrile (48-3, 525 mg, 0.5 mmol) in 1,4-dioxane (6 mL) and water (1 mL) mixture, K₃PO₄ (150 mg, 0.7 mmol) was added. Nitrogen gas was purged into the reaction mixture for 10 min and [1,1′-bis(di-tert-butylphosphino)ferrocene]dichloropalladium(II) (25 mg, 0.04 mmol) was added and stirred the mixture at 60° C. for 2 h. The reaction mixture was diluted with ethyl acetate and washed with brine. The organic extract was dried over anhydrous Na₂SO₄, filtered and concentrated under reduced pressure. The product was purified by reverse phase HPLC prep chromatography to afford 2-(7-(cyanomethoxy)-1-oxo-1,2-dihydroisoquinolin-4-yl)-3-methylbenzonitrile (Example 48, 27 mg). LCMS (ESI) Calcd. for C₁₉H₁₃N₃O₂: 315, found [M+H]⁺=316. ¹HNMR (400 MHz, DMSO-d₆) 11.69 (br s, 1H), 7.88 (d, 1H), 7.82 (d, 1H), 7.72 (d, 1H), 7.57 (t, 1H), 7.38 (dd, 1H), 7.19 (s, 1H), 6.86 (d, 1H), 5.34 (s, 2H), 2.08 (s, 3H).

### Example 49: Synthesis of 2-((1-oxo-4-(2-(trifluoromethoxy)phenyl)-1,2-dihydroisoquinolin-7-yl)oxy)acetonitrile

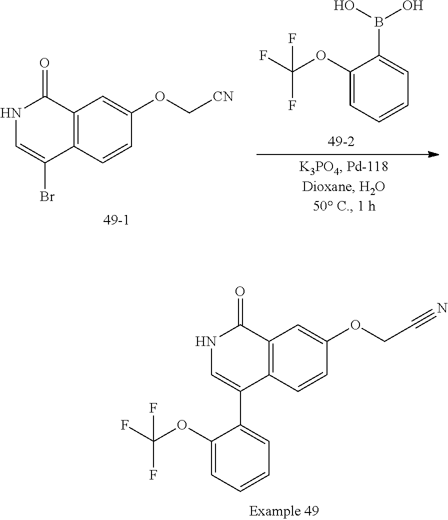 [SMILES: N#CCOc1ccc2c(Br)c[nH]c(=O)c2c1 ; OB(O)c1ccccc1OC(F)(F)F ; N#CCOc1ccc2c(-c3ccccc3OC(F)(F)F)c[nH]c(=O)c2c1]

To a stirred solution of 2-((4-bromo-1-oxo-1,2-dihydroisoquinolin-7-yl)oxy)acetonitrile (49-1, 75 mg, 0.7 mmol) in 1,4-dioxane (3 mL) and water (0.5 mL) mixture, K₃PO₄ (114 mg, 0.5 mmol) and (2-(trifluoromethoxy)phenyl)boronic acid (49-2, 85 mg, 0.4 mmol) were added and the reaction mixture was purged with Argon gas for 5 min. In to this purged reaction mixture [1,1′-bis(di-tert-butylphosphino)ferrocene]dichloropalladium(II) (18 mg, 0.02 mmol) was added and the reaction mixture was heated at 60° C. for 1 h. After completion, the reaction mixture was filtered through celite bed and the bed was washed with ethyl acetate. The combined filtrate was washed with water and brine, dried over anhydrous Na₂SO₄ and concentrated under reduced pressure. The product was purified by reverse phase prep HPLC purification method and lyophilized to afford 2-((1-oxo-4-(2-(trifluoromethoxy)phenyl)-1,2-dihydroisoquinolin-7-yl)oxy)acetonitrile (Example 49, 45 mg). LCMS (ESI) Calcd. for C₁₈H₁₁F₃N₂O₃: 360, found [M+H]⁺=361. ¹H NMR (400 MHz, DMSO-d₆): δ_{H} 11.61-11.59 (m, 1H), 7.85-7.84 (m, 1H), 7.63-7.58 (m, 1H), 7.53-7.50 (m, 3H), 7.41-7.38 (m, 1H), 7.08 (d, 2H), 5.33 (s, 2H).

### Example 50-51: Synthesis of chiral analogs of 2-(1-((R)-2-((4-(2-chloro-4-fluorophenyl)-1-oxo-1,2-dihydroisoquinolin-7-yl)oxy)propanoyl)piperidin-3-yl)-2-methylpropanoic acid

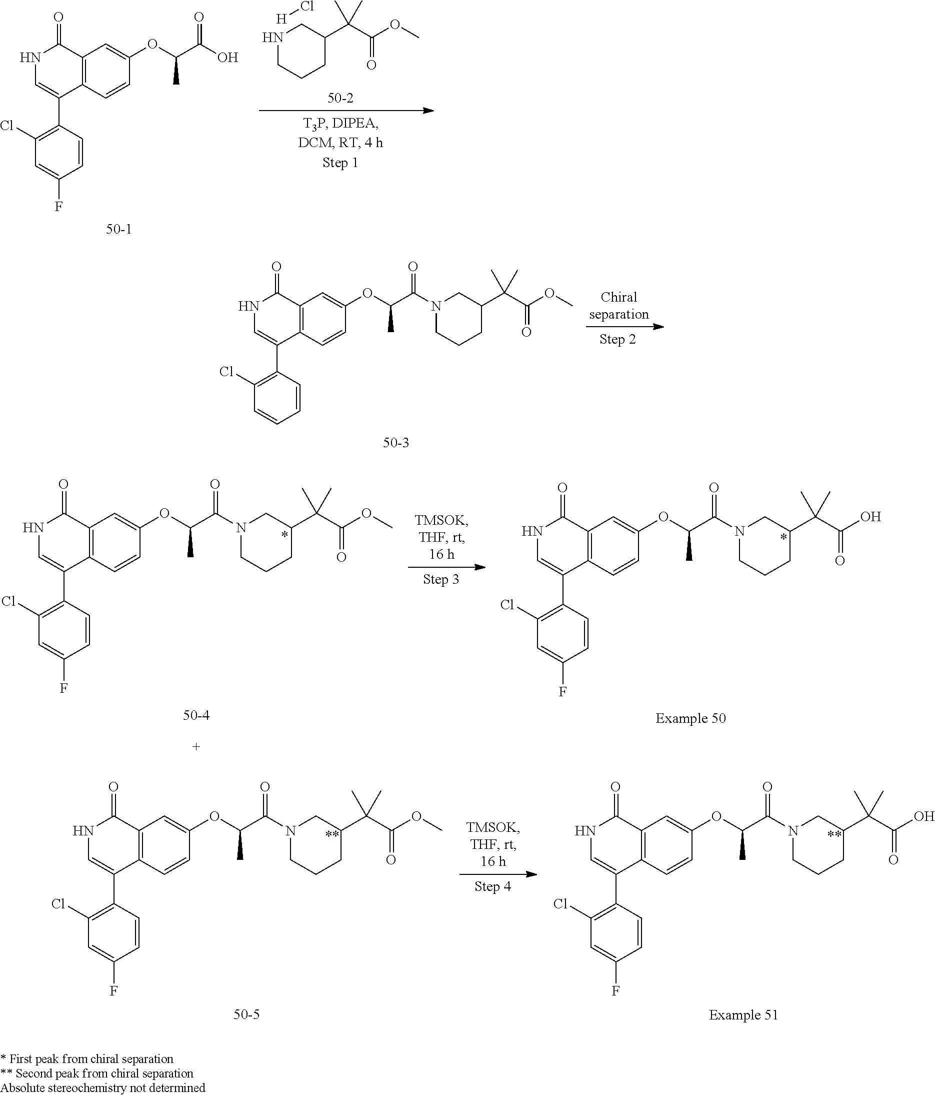

Synthesis of methyl 2-(1-((R)-2-((4-(2-chloro-4-fluorophenyl)-1-oxo-1,2-dihydroisoquinolin-7-yl)oxy)propanoyl)piperidin-3-yl)-2-methylpropanoate, 50-3 [Step 1]: To a stirred solution of (R)-2-((4-(2-chloro-4-fluorophenyl)-1-oxo-1,2-dihydroisoquinolin-7-yl)oxy)propanoic acid (50-1, 300 mg, 0.8 mmol) and methyl 2-methyl-2-(piperidin-3-yl)propanoate hydrochloride (50-2, 550 mg, 2.5 mmol) in dichloromethane (2 mL), DIPEA (0.4 mL, 2.5 mmol) was added followed by T₃P (1.0 mL, 1.7 mmol, 50% in ethyl acetate) and stirred at ambient temperature for 4 h. The reaction mixture was diluted with dichloromethane and washed with water (twice). The organic extract was collected, dried with anhydrous Na₂SO₄ and concentrated under reduced pressure. The product was purified by reverse phase prep-HPLC to afford methyl 2-(1-((R)-2-((4-(2-chloro-4-fluorophenyl)-1-oxo-1,2-dihydroisoquinolin-7-yl)oxy)propanoyl)piperidin-3-yl)-2-methylpropanoate (50-3, 260 mg). LCMS (ESI) Calcd. for C₂₁H₃₀ClFN₂O₅: 528, found [M+H]⁺=529.

Synthesis of chiral methyl 2-(1-((R)-2-((4-(2-chloro-4-fluorophenyl)-1-oxo-1,2-dihydroisoquinolin-7-yl)oxy)propanoyl)piperidin-3-yl)-2-methylpropanoate, 50-4 and 50-5 [Step 2]: Racemic methyl 2-(1-((R)-2-((4-(2-chloro-4-fluorophenyl)-1-oxo-1,2-dihydroisoquinolin-7-yl)oxy)propanoyl)piperidin-3-yl)-2-methylpropanoate (50-3, 260 mg) was purified by prep-SFC purification and lyophilized to afford the first product as chiral methyl 2-(1-((R)-2-((4-(2-chloro-4-fluorophenyl)-1-oxo-1,2-dihydroisoquinolin-7-yl)oxy)propanoyl)piperidin-3-yl)-2-methylpropanoate (50-4, 130 mg) as Peak 1 and the second product as chiral methyl 2-(1-((R)-2-((4-(2-chloro-4-fluorophenyl)-1-oxo-1,2-dihydroisoquinolin-7-yl)oxy)propanoyl)piperidin-3-yl)-2-methylpropanoate (50-5, 130 mg) as Peak 2. The absolute stereochemistries for these compounds were not determined.

Prep-SFC method: SFC Prep purification was performed using a PIC SOLUTION-175 instrument and Chiralcel OX-H (21.0 mm×250 mm), 5μ, which was operated at 35° C. temperature with a flow rate of 50 mL/min. The mobile phase used was a mixture of 65% CO₂ in super critical state and 35% of 0.1% IPAmine in MeOH:CAN (1:1). The instrument was held in isocratic mode for up to 12 min using and at an isobaric condition of 120 bar with a wavelength of 284 nm.

50-4: Chiral methyl 2-(1-((R)-2-((4-(2-chloro-4-fluorophenyl)-1-oxo-1,2-dihydroisoquinolin-7-yl)oxy)propanoyl)piperidin-3-yl)-2-methylpropanoate (Peak 1): LCMS (ESI) Calcd. for C₂₈H₃₀ClFN₂O₅: 528, [M+H]⁺=529.

50-4: Chiral methyl 2-(1-((R)-2-((4-(2-chloro-4-fluorophenyl)-1-oxo-1,2-dihydroisoquinolin-7-yl)oxy)propanoyl)piperidin-3-yl)-2-methylpropanoate (Peak 2): LCMS (ESI) Calcd. for C₂₈H₃₀ClFN₂O₅: 528, [M+H]⁺=529.

Synthesis of 2-(1-((R)-2-((4-(2-chloro-4-fluorophenyl)-1-oxo-1,2-dihydroisoquinolin-7-yl)oxy)propanoyl)piperidin-3-yl)-2-methylpropanoic acid, Example 50 [Step 3]: To a stirred solution of chiral methyl 2-(1-((R)-2-((4-(2-chloro-4-fluorophenyl)-1-oxo-1,2-dihydroisoquinolin-7-yl)oxy)propanoyl)piperidin-3-yl)-2-methylpropanoate (50-4, 130 mg, 0.2 mmol) in THF (5 mL), potassium trimethylsilanolate (95 mg, 0.7 mmol) was added and stirred at ambient temperature for 16 h. The reaction mixture was quenched with 1N HCl solution to adjust to pH 3 and extracted with ethyl acetate (twice). The combined organic extract was dried over anhydrous Na₂SO₄ and concentrated under reduced pressure. The product was purified by reverse phase prep-HPLC and lyophilized to afford 2-(1-((R)-2-((4-(2-chloro-4-fluorophenyl)-1-oxo-1,2-dihydroisoquinolin-7-yl)oxy)propanoyl)piperidin-3-yl)-2-methylpropanoic acid (Example 50, 70 mg). LCMS (ESI) Calcd. for C₂₇H₂₈ClFN₂O₅: 515, found [M+H]⁺=515. ¹H NMR (400 MHz, DMSO-d₆ at 100° C.): δ_{H} 11.00 (br s, 2H), 7.68 (s, 1H), 7.51 (d, 1H), 7.47-7.44 (m, 1H), 7.32-7.23 (m, 2H), 6.96 (d, 1H), 6.93 (s, 1H), 5.29 (br s, 11H), 4.23-4.07 (br s, 3H), 2.53 (br s, 1H), 1.85-1.73 (m, 3H), 1.50 (d, 3H), 1.35-1.19 (m, 2H), 1.09 (s, 6H). The absolute stereochemistry was not determined.

Synthesis of 2-(1-((R)-2-((4-(2-chloro-4-fluorophenyl)-1-oxo-1,2-dihydroisoquinolin-7-yl)oxy)propanoyl)piperidin-3-yl)-2-methylpropanoic acid, Example 51 [Step 4]: To a stirred solution of chiral methyl 2-(1-((R)-2-((4-(2-chloro-4-fluorophenyl)-1-oxo-1,2-dihydroisoquinolin-7-yl)oxy)propanoyl)piperidin-3-yl)-2-methylpropanoate (50-5, 130 mg, 0.2 mmol) in THF (5 mL), potassium trimethylsilanolate (95 mg, 0.7 mmol) was added and stirred at ambient temperature for 16 h. The reaction mixture was quenched with 1N HCl solution to adjust to pH 3 and extracted with ethyl acetate. The organic extract was collected, dried over anhydrous Na₂SO₄ and concentrated under reduced pressure. The product was purified by reverse phase prep-HPLC and lyophilized to afford 2-(1-((R)-2-((4-(2-chloro-4-fluorophenyl)-1-oxo-1,2-dihydroisoquinolin-7-yl)oxy)propanoyl)piperidin-3-yl)-2-methylpropanoic acid (Example 51, 55 mg). LCMS (ESI) Calcd. for C₂₇H₂₈ClFN₂O₅: 515, found [M+H]⁺=515. ¹H NMR (400 MHz, DMSO-d₆ at 100° C.): δ_{H} 11.00 (br s, 2H), 7.68 (s, 1H), 7.52 (d, 1H), 7.48-7.44 (m, 1H), 7.32-7.23 (m, 2H), 6.96 (d, 1H), 6.94 (s, 1H), 5.30 (br s, 1H), 4.26-4.07 (br s, 3H), 2.54 (br s, 1H), 1.76-1.72 (m, 2H), 1.62-1.59 (m, 1H), 1.50 (d, 3H), 1.32-1.29 (m, 2H), 1.09 (s, 6H). The absolute stereochemistry was not determined.

### Example 52-53: Synthesis of chiral analogs of 3-ethyl-1-((R)-2-((1-oxo-4-(o-tolyl)-1,2-dihydroisoquinolin-7-yl)oxy)propanoyl)piperidine-3-carboxylic acid

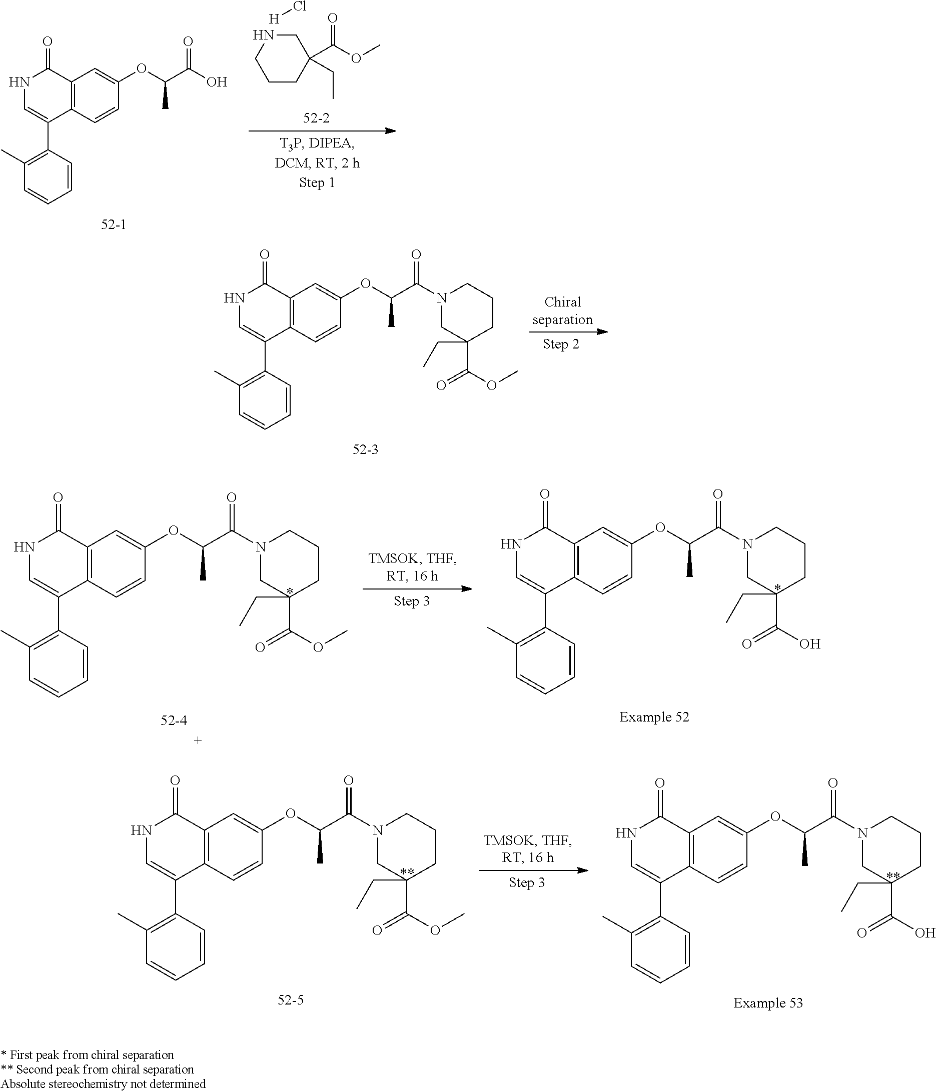

Synthesis of methyl 3-ethyl-1-((R)-2-((1-oxo-4-(o-tolyl)-1,2-dihydroisoquinolin-7-yl)oxy)propanoyl)piperidine-3-carboxylate. 52-3 [Step 1]: To a stirred solution of (R)-2-((1-oxo-4-(o-tolyl)-1,2-dihydroisoquinolin-7-yl)oxy)propanoic acid (52-1, 190 mg, 0.6 mmol) in dichloromethane (15 mL), methyl 3-ethylpiperidine-3-carboxylate hydrochloride (52-2, 185 mg, 0.9 mmol) was added followed by DIPEA (0.4 mL, 2.4 mmol) at 0° C. After 20 min, T₃P (0.5 mL, 0.9 mmol, 50% in ethyl acetate) was added to the reaction mixture at 0° C. and stirred at ambient temperature for 3 h. The reaction mixture was diluted with ice water and extracted with dichloromethane (twice). The combined organic extracts was washed with brine, dried over anhydrous Na₂SO₄ and concentrated under reduced pressure to afford methyl 3-ethyl-1-((R)-2-((1-oxo-4-(o-tolyl)-1,2-dihydroisoquinolin-7-yl)oxy)propanoyl)piperidine-3-carboxylate (52-3, 270 mg). LCMS (ESI) Calcd. for C₂₈H₃₂N₂O₅: 477, found [M+H]⁺=478.

Synthesis of chiral methyl 3-ethyl-1-((R)-2-((1-oxo-4-(o-tolyl)-1,2-dihydroisoquinolin-7-yl)oxy)propanoyl)piperidine-3-carboxylate, 52-4 and 52-5 [Step 2]: Racemic methyl 3-ethyl-1-((R)-2-((1-oxo-4-(o-tolyl)-1,2-dihydroisoquinolin-7-yl)oxy)propanoyl)piperidine-3-carboxylate (52-3, 270 mg) was purified by prep-SFC purification and lyophilized to afford the first product as chiral methyl 3-ethyl-1-((R)-2-((1-oxo-4-(o-tolyl)-1,2-dihydroisoquinolin-7-yl)oxy)propanoyl)piperidine-3-carboxylate (524.90 mg) as Peak 1 and the second product as chiral methyl 3-ethyl-1-((R)-2-((1-oxo-4-(o-tolyl)-1,2-dihydroisoquinolin-7-yl)oxy)propanoyl)piperidine-3-carboxylate (52-5, 110 mg) as Peak 2. The absolute stereochemistry were not determined and was arbitrarily assigned.

Prep-SFC method: SFC Prep purification was performed on a THAR SFC-80 instrument by using Reflect (R,R)Whelk-01 (21.1 mm×250 mm), 5μ, which was operated at 35° C. temperature with a flow rate of 60 gm/min, using 55% CO₂ in supercritical state and 45% of (100% IPA). The instrument was held in isocratic mode for up to 13 min and at an isobaric condition of 120 bar with a wavelength of 230 nm.

52-4: Chiral methyl 3-ethyl-1-((R)-2-((1-oxo-4-(o-tolyl)-1,2-dihydroisoquinolin-7-yl)oxy)propanoyl)piperidine-3-carboxylate (Peak 1): LCMS (ESI) Calcd. for C₂₈H₃₂N₂O₅: 477, [M+H]⁺=477.

52-5: Chiral methyl 3-ethyl-1-((R)-2-((1-oxo-4-(o-tolyl)-1,2-dihydroisoquinolin-7-yl)oxy)propanoyl)piperidine-3-carboxylate (Peak 2): LCMS (ESI) Calcd. for C₂NH₃₂N₂O₅: 477, [M+H]⁺=477.

Synthesis of 3-ethyl-1-((R)-2-((1-oxo-4-(o-tolyl)-1,2-dihydroisoquinolin-7-yl)oxy)propanoyl)piperidine-3-carboxylic acid, Example 52 [Step 3]: To a stirred solution of chiral methyl 3-ethyl-1-((R)-2-((1-oxo-4-(o-tolyl)-1,2-dihydroisoquinolin-7-yl)oxy)propanoyl)piperidine-3-carboxylate (524, 75 mg, 0.2 mmol) in THF (5 mL), potassium trimethylsilanolate (80 mg, 0.6 mmol) was added and stirred at ambient temperature for 16 h. The reaction mixture was evaporated under reduced pressure, diluted with water and acidified with 1N HCl solution to maintain pH˜3. The mixture was extracted with 10% methanol in dichloromethane (twice). The combined organic extract was dried over anhydrous Na₂SO₄ and concentrated under reduced pressure. The product was purified by reverse phase prep-HPLC and lyophilized to afford 3-ethyl-1-((R)-2-((1-oxo-4-(o-tolyl)-1,2-dihydroisoquinolin-7-yl)oxy)propanoyl)piperidine-3-carboxylic acid (Example 52.30 mg). LCMS (ESI) Calcd. for C₂₇H₃₀N₂O₅: 463, found [M−H]⁻=461. ¹H NMR (400 MHz, DMSO-d₆): δ_{H} 11.32 (br s, 1H), 7.63 (s, 1H), 7.35-7.18 (m, 5H), 6.89 (t, 2H), 5.56-5.30 (m, 1H), 4.00-3.67 (m, 2H), 3.10-3.05 (m, 1H), 2.89 (br s, 1H), 2.03 (s, 3H), 1.76-1.21 (m, 10H), 0.76-0.72 (m, 3H). The absolute stereochemistry was not determined.

Synthesis of 3-ethyl-1-((R)-2-((1-oxo-4-(o-tolyl)-1,2-dihydroisoquinolin-7-yl)oxy)propanoyl)piperidine-3-carboxylic acid, Example 53 [Step 4]: To a stirred solution of chiral methyl 3-ethyl-1-((R)-2-((1-oxo-4-(o-tolyl)-1,2-dihydroisoquinolin-7-yl)oxy)propanoyl)piperidine-3-carboxylate (52-5, 95 mg, 0.2 mmol) in THF (6 mL), potassium trimethylsilanolate (100 mg, 0.8 mmol) was added and stirred at ambient temperature for 16 h. The reaction mixture was evaporated under reduced pressure, diluted with water and acidified with 1N HCl solution to maintain pH˜3. The mixture was extracted with 10% methanol in dichloromethane (twice). The combined organic extract was dried over anhydrous Na₂SO₄ and concentrated under reduced pressure. The product was purified by reverse phase prep-HPLC and lyophilized to afford 3-ethyl-1-((R)-2-((1-oxo-4-(o-tolyl)-1,2-dihydroisoquinolin-7-yl)oxy)propanoyl)piperidine-3-carboxylic acid (Example 53, 40 mg). LCMS (ESI) Calcd. for C₂₇H₃₀N₂O₅: 463, found [M−H]⁻=461. ¹H NMR (400 MHz, DMSO-d₆): δ_{H} 11.32 (br s, 1H), 7.63 (d, 1H), 7.35-7.18 (m, 5H), 6.89 (d, 2H), 5.51-5.46 (m, 1H), 4.15 (t, 1H), 3.71 (t, 1H), 2.96 (d, 1H), 2.67-2.53 (m, 1H), 2.13 (d, 1H), 2.03 (s, 3H), 1.80 (s, 2H), 1.64-1.26 (m, 7H), 0.85-0.65 (m, 3H). The absolute stereochemistry was not determined.

### Example 54-55: Synthesis of chiral analogs of (R)-7-((1-(4-acetylpiperazin-1-yl)propan-2-yl)oxy)-4-(o-tolyl)isoquinolin-1(2H)-one

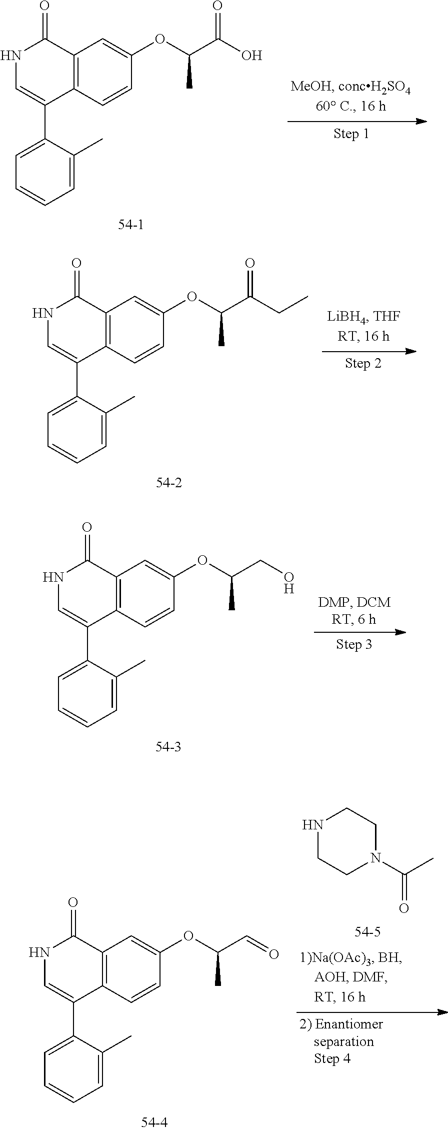

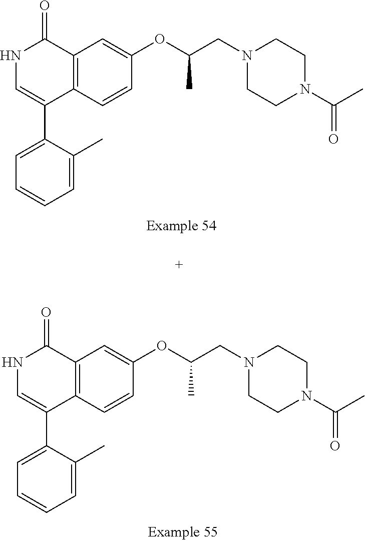 [SMILES: CC(=O)N1CCN(C[C@@H](C)Oc2ccc3c(-c4ccccc4C)c[nH]c(=O)c3c2)CC1 ; CC(=O)N1CCN(C[C@H](C)Oc2ccc3c(-c4ccccc4C)c[nH]c(=O)c3c2)CC1]

Synthesis methyl (R)-2-((1-oxo-4-(o-tolyl)-1,2-dihydroisoquinolin-7-yl)oxy)propanoate, 54-2 [Step 1]: To a stirred solution of (2R)-2-[[4-(o-tolyl)-1-oxo-2H-isoquinolin-7-yl]oxy]propanoic acid (54-1, 2.5 g, 7.73 mmol) in methanol (40 mL) was added conc. sulfuric acid (0.041 mL, 0.77 mmol) at 25° C. The resulting reaction mixture was heated at 60° C. for 16 h and the volatiles were evaporated. The residue was diluted with ethyl acetate and washed with saturated solution of aqueous sodium bicarbonate followed by brine wash. The combined organic extracts were dried over anhydrous sodium sulfate, filtered and concentrated under reduced pressure to give methyl (R)-2-((1-oxo-4-(o-tolyl)-1,2-dihydroisoquinolin-7-yl)oxy)propanoate (54-2, 1.4 g). The product was carried to next step without further purification. LCMS (ESI) Calcd. for C₂₀H₁₉NO₄: 337, found [M+H]⁺=338.

Synthesis (R)-7-((1-hydroxypropan-2-yl)oxy)-4-(o-tolyl)isoquinolin-1(2H)-one, 54-3 [Step 2]: To a stirred solution of methyl (R)-2-((1-oxo-4-(o-tolyl)-1,2-dihydroisoquinolin-7-yl)oxy)propanoate (54-2, 700 mg) in tetrahydrofuran (8 mL) was added lithium borohydride (2M, 7.0 mL) at 0° C. The reaction mixture was allowed to stir at ambient temperature for 16 h, quenched with saturated solution of aqueous ammonium chloride under ice cold condition, diluted with ethyl acetate and washed with water followed by brine wash. The combined organic extracts were dried over anhydrous sodium sulfate, filtered and concentrated under reduced pressure to afford (R)-7-((1-hydroxypropan-2-yl)oxy)-4-(o-tolyl)isoquinolin-1(2H)-one (54-3, 620 mg). The product was carried to next step without further purification. LCMS (ESI) Calcd. for C₁₉H₁₉NO₃: 309, found [M+H]⁺=310.

Synthesis (R)-2-((1-oxo-4-(o-tolyl)-1,2-dihydroisoquinolin-7-yl)oxy)propanal, 54-4 [Step 3]: To a stirred solution of (R)-7-((1-hydroxypropan-2-yl)oxy)-4-(o-tolyl)isoquinolin-1(2H)-one (54-3, 200 mg, 0.64 mmol) in dichloromethane (10 mL) was added Dess-Martin periodinane (412 mg, 0.96 mmol) at 0° C. The reaction mixture was stirred at ambient temperature for 6 h, diluted with dichloromethane, washed with saturated solution of aqueous sodium thiosulfate and saturated solution of aqueous sodium bicarbonate. The combined organic extracts were dried over anhydrous sodium sulfate, filtered and concentrated under reduced pressure to give (R)-2-((1-oxo-4-(o-tolyl)-1,2-dihydroisoquinolin-7-yl)oxy)propanal (54-4, 100 mg). The product was carried to next step without further purification. LCMS (ESI) Calcd. for C₁₉H₁₇NO₃: 307, found [M+H]⁺=308.

Synthesis of (R)-7-((1-(4-acetylpiperazin-1-yl)propan-2-yl)oxy)-4-(o-tolyl)isoquinolin-1(2H)-one and (S)-7-((1-(4-acetylpiperazin-1-yl)propan-2-yl)oxy)-4-(o-tolyl)isoquinolin-1(2H)-one, Example 54 and Example 55 [Step 4]: To a stirred solution of (R)-2-((1-oxo-4-(o-tolyl)-1,2-dihydroisoquinolin-7-yl)oxy)propanal (54-4, 200 mg, 0.651 mmol) in dimethylformamide (5 mL) were added 1-piperazin-1-ylethanone (54-5, 83 mg, 0.651 mmol) and acetic acid (0.41 mL, 7.16 mmol) at 25° C. The resulting reaction mixture was allowed to stir for 30 min and sodium tri acetoxy borohydride (414 mg, 1.95 mmol) was added at 0° C. The resulting reaction mixture was allowed to stir at ambient temperature for 16 h and diluted with ethyl acetate. The organic phase was washed with ice-cold water followed by saturated solution of aqueous sodium bicarbonate. The combined organic extracts were dried over anhydrous sodium sulfate, filtered and concentrated under reduced pressure. The product was purified to give a mixture of (R)-7-((1-(4-acetylpiperazin-1-yl)propan-2-yl)oxy)-4-(o-tolyl)isoquinolin-1(2H)-one and (S)-7-((1-(4-acetylpiperazin-1-yl)propan-2-yl)oxy)-4-(o-tolyl)isoquinolin-1(2H)-one (90 mg, 4:1). The enantiomers were separated by normal phase chiral preparative HPLC to give Peak 1 as (R)-7-((1-(4-acetylpiperazin-1-yl)propan-2-yl)oxy)-4-(o-tolyl)isoquinolin-1(2H)-one (Example 54, 26 mg) and Peak 2 as (S)-7-((1-(4-acetylpiperazin-1-yl)propan-2-yl)oxy)-4-(o-tolyl)isoquinolin-1(2H)-one (Example 55, 10 mg).

Example 54: (R)-7-((1-(4-acetylpiperazin-1-yl)propan-2-yl)oxy)-4-(o-tolyl)isoquinolin-1(2H)-one (Peak 1): LCMS (ESI) Calcd. for C₂₅H₂₉N₃O₃: 419, found [M+H]⁺=420. ¹H NMR (400 MHz, DMSO-d₆): δ_{H} 11.36-11.34 (m, 1H), 7.75-7.71 (m, 1H), 7.35-7.34 (m, 2H), 7.30-7.25 (m, 2H), 7.19 (d, 1H), 6.91-6.86 (m, 2H), 4.77-4.76 (m, 1H), 3.37 (br s, 4H), 2.67-2.62 (m, 1H), 2.45-2.41 (m, 4H), 2.04 (s, 3H), 1.95 (s, 3H), 1.28 (d, 3H).

Example 55: (S)-7-((1-(4-acetylpiperazin-1-yl)propan-2-yl)oxy)-4-(o-tolyl)isoquinolin-1(2H)-one (Peak 2): LCMS (ESI) Calcd. for C₂₅H₂₉N₃O₃: 419, found [M+H]⁺=420. ¹H NMR (400 MHz, DMSO-d₆): δ_{H} 11.37-11.35 (m, 1H), 7.75 (s, 1H), 7.35-7.34 (m, 2H), 7.27-7.25 (m, 2H), 7.19 (d, 1H), 6.91-6.86 (m, 2H), 4.77-4.75 (m, 1H), 3.37 (br s, 4H), 2.67-2.62 (m, 1H), 2.45-2.41 (m, 4H), 2.04 (s, 3H), 1.95 (s, 3H), 1.28 (d, 3H).

Chiral HPLC Prep-Method: Chiral separation was performed using an Agilent 1200 series instrument and a Chiralpak IG (250×21 mm), 5μ, which was operated at 35° C. temperature with a flow rate of 21.0 mL/min. The mobile phase used was a mixture of 60% hexane, 20% dichloromethane, and 20% ethyl alcohol. The instrument was held in isocratic mode for up to 25 min with a wavelength of 220 nm.

### Example 56. Synthesis of (R)-4-(2-((4-(4-fluoro-2-methylphenyl)-1-oxo-1,2-dihydroisoquinolin-7-yl)oxy)propanoyl)piperazine-1-carboxamide

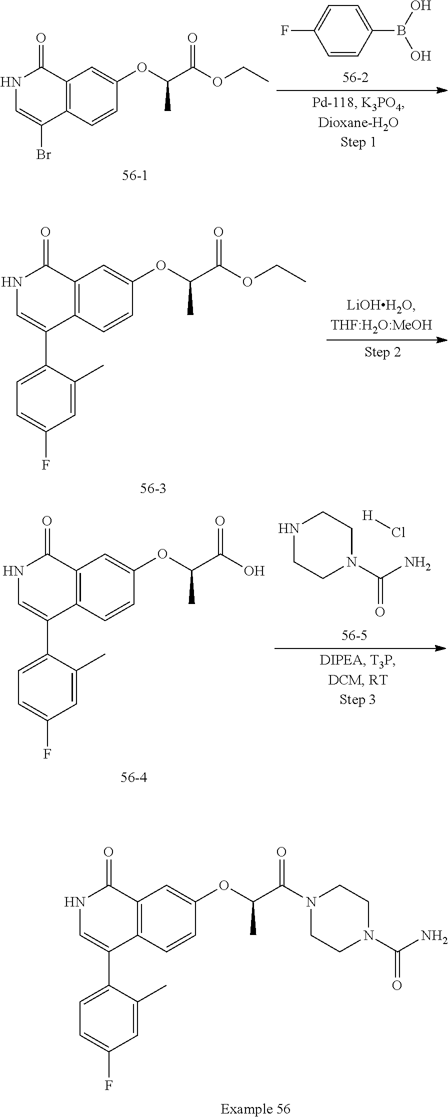

Synthesis of ethyl (R)-2-((4-(4-fluoro-2-methylphenyl)-1-oxo-1,2-dihydroisoquinolin-7-yl)oxy)propanoate, 56-3 [Step 1]: To a stirred solution of ethyl (R)-2-((4-bromo-1-oxo-1,2-dihydroisoquinolin-7-yl)oxy)propanoate (56-1, 300 mg, 0.9 mmol) and (4-fluoro-2-methylphenyl)boronic acid (56-2, 205 mg, 1.3 mmol) in 1,4-dioxane (20 mL) and water (5 mL) mixture, K₃PO₄ (470 mg, 2.2 mmol) was added at ambient temperature. The reaction mixture was degassed with nitrogen gas for 10 min. PdCl₂(dtbpf) (55 mg, 0.1 mmol) was added to it and the reaction mass was heated at 100° C. for 16 h. After completion, the reaction mixture was filtered through celite bed and the bed was washed with ethyl acetate (twice). The combined filtrate was evaporated under reduced pressure. The reduced mass was diluted with water and extracted with ethyl acetate (twice). The combined organic extract was washed with brine, dried over anhydrous sodium sulphate and concentrated under reduced pressure to afford ethyl (R)-2-((4-(4-fluoro-2-methylphenyl)-1-oxo-1,2-dihydroisoquinolin-7-yl)oxy)propanoate (56-3, 150 mg). LCMS (ESI) Calcd. for C₂₁H₂₀FNO₄: 369, found [M+H]⁺=370.

Synthesis of (R)-2-((4-(4-fluoro-2-methylphenyl)-1-oxo-1,2-dihydroisoquinolin-7-yl)oxy)propanoic acid, 56-4 [Step 2]: To a stirred solution of ethyl (R)-2-((4-(4-fluoro-2-methylphenyl)-1-oxo-1,2-dihydroisoquinolin-7-yl)oxy)propanoate (56-3, 0.3 g, 0.8 mmol) in THF (8 mL), methanol (2 mL) and water (4 mL) mixture, LiOH·H₂O (50 mg, 1.2 mmol) was added portion wise at ambient temperature and stirred at ambient temperature for 16 h. After completion, the reaction mixture was concentrated under reduced pressure. The reduced mass was diluted with water and washed with ethyl acetate (twice). The organic layer was discarded and the aqueous extract was acidified using 1N HCl solution to maintain pH˜3. It was extracted with 10% methanol in dichloromethane (twice). The combined organic extract was washed with brine, dried over anhydrous sodium sulphate and concentrated under reduced pressure to afford (R)-2-((4-(4-fluoro-2-methylphenyl)-1-oxo-1,2-dihydroisoquinolin-7-yl)oxy)propanoic acid (56-4, 200 mg). LCMS (ESI) Calcd. for C₁₉H₁₆FNO₄: 341, found [M+H]⁺=342.

Synthesis of (R)-4-(2-((4-(4-fluoro-2-methylphenyl)-1-oxo-1,2-dihydroisoquinolin-7-yl)oxy)propanoyl)piperazine-1-carboxamide, Example 56 [Step 3]: To a stirred solution of (R)-2-((4-(4-fluoro-2-methylphenyl)-1-oxo-1,2-dihydroisoquinolin-7-yl)oxy)propanoic acid (56-4, 310 mg, 0.9 mmol) in dichloromethane (3 mL), piperazine-1-carboxamide hydrochloride (56-5, 225 mg, 1.4 mmol) was added followed by DIPEA (0.6 mL, 3.6 mmol) at 0° C. After 10 min, T₃P (0.8 mL, 1.4 mmol, 50% in ethyl acetate) was added to the reaction mixture at 0° C. and stirred at ambient temperature for 2 h. After completion, the reaction mixture was quenched with ice water and extracted with dichloromethane (twice). The combined organic extract was washed with brine, dried over anhydrous sodium sulphate and concentrated under reduced pressure. The product was purified by reverse phase prep HPLC and lyophilized to afford (R)-4-(2-((4-(4-fluoro-2-methylphenyl)-1-oxo-1,2-dihydroisoquinolin-7-yl)oxy)propanoyl)piperazine-1-carboxamide (Example 56, 120 mg). LCMS (ESI) Calcd. for C₂₄H₂₅FN₄O₄: 452, found [M+H]⁺=453. ¹H NMR (400 MHz, DMSO-d₆) δ_{H} 11.33 (br s, 1H), 7.61-7.59 (m, 1H), 7.24-7.20 (m, 3H), 7.12-7.08 (m, 1H), 6.90-6.88 (m, 2H), 6.90 (s, 2H), 5.49-5.46 (m, 1H), 3.77-3.64 (m, 2H), 3.50-3.38 (m, 4H), 3.18-3.11 (m, 2H), 2.03 (s, 3H), 1.45 (d, 3H).

### Example 57: Synthesis of (R)-4-(2-((4-(3-fluoro-2-methylphenyl)-1-oxo-1,2-dihydroisoquinolin-7-yl)oxy)propanoyl)piperazine-1-carboxamide

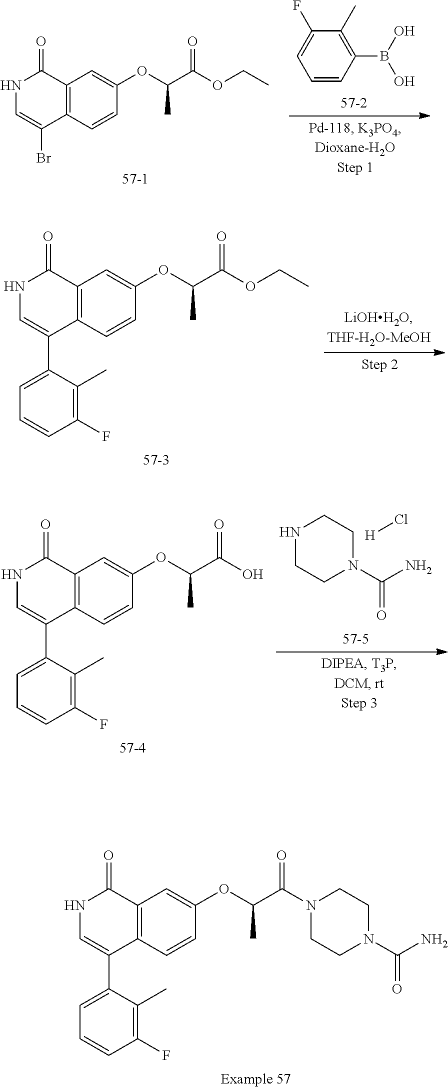

Synthesis of ethyl (R)-2-((4-(3-fluoro-2-methylphenyl)-1-oxo-1,2-dihydroisoquinolin-7-yl)oxy)propanoate, 57-3 [Step 1]: To a stirred solution of ethyl (R)-2-((4-bromo-1-oxo-1,2-dihydroisoquinolin-7-yl)oxy)propanoate (57-1, 300 mg, 0.9 mmol) and (3-fluoro-2-methylphenyl)boronic acid (57-2, 205 mg, 1.3 mmol) in 1,4-dioxane (20 mL) and water (5 mL) mixture, K₃PO₄ (470 mg, 2.2 mmol) was added at ambient temperature. The reaction mixture was degassed with nitrogen gas for 10 min. PdCl₂(dtbpf) (55 mg, 0.1 mmol) was added to it and the reaction mass was stirred at 100° C. for 16 h. After completion, the reaction mixture was filtered through celite bed and bed was washed with ethyl acetate (twice). Combined filtrate was evaporated under reduced pressure. The reduced mass was diluted with water and washed with ethyl acetate (twice). Combined organic extracts was washed with brine, dried over anhydrous sodium sulphate and concentrated under reduced pressure to afford ethyl (R)-2-((4-(3-fluoro-2-methylphenyl)-1-oxo-1,2-dihydroisoquinolin-7-yl)oxy)propanoate (57-3, 320 mg). LCMS (ESI) Calcd. for C₂₁H₂₀FNO₄: 369, found [M+H]⁺=370.

Synthesis of (R)-2-((4-(3-fluoro-2-methylphenyl)-1-oxo-1,2-dihydroisoquinolin-7-yl)oxy)propanoic acid, 57-4 [Step 2]: To a stirred solution of ethyl (R)-2-((4-(3-fluoro-2-methylphenyl)-1-oxo-1,2-dihydroisoquinolin-7-yl)oxy)propanoate (57-3, 0.30 g, 0.8 mmol) in THF (8 mL), methanol (2 mL) and water (4 mL) mixture, LiOH·H₂O (50 mg, 1.2 mmol) was added portion wise at ambient temperature and stirred at ambient temperature for 16 h. After completion, the reaction mixture was concentrated under reduced pressure. The reduced mass was diluted with water and washed with ethyl acetate (twice). The organic layer was discarded and the aqueous extract was acidified using 1N HCl solution to maintain pH˜3. It was extracted with 10% methanol in dichloromethane (twice). Combined organic extracts was washed with brine, dried over anhydrous sodium sulphate and concentrated under reduced pressure to afford (R)-2-((4-(3-fluoro-2-methylphenyl)-1-oxo-1,2-dihydroisoquinolin-7-yl)oxy)propanoic acid (57-4, 210 mg). LCMS (ESI) Calcd. for C₁₉H₁₆FNO₄: 341, found [M+H]⁺=342.

Synthesis of (R)-4-(2-((4-(3-fluoro-2-methylphenyl)-1-oxo-1,2-dihydroisoquinolin-7-yl)oxy)propanoyl)piperazine-1-carboxamide, Example 57 [Step 3]: To a stirred solution (R)-2-((4-(3-fluoro-2-methylphenyl)-1-oxo-1,2-dihydroisoquinolin-7-yl)oxy)propanoic acid (57-4, 310 mg, 0.9 mmol) in dichloromethane (3 mL), piperazine-1-carboxamide hydrochloride (57-5, 230 mg, 1.4 mmol) was added followed by DIPEA (0.7 mL, 3.6 mmol) at 0° C. After 10 min, T₃P (0.8 mL, 1.4 mmol, 50% in ethyl acetate) was added to the reaction mixture at 0° C. and stirred at ambient temperature for 2 h. After completion, the reaction mixture was quenched with ice water and extracted with dichloromethane (twice). Combined organic extracts was washed with brine, dried over anhydrous sodium sulphate and concentrated under reduced pressure. The product was purified by reverse phase HPLC and lyophilized to afford (R)-4-(2-((4-(3-fluoro-2-methylphenyl)-1-oxo-1,2-dihydroisoquinolin-7-yl)oxy)propanoyl)piperazine-1-carboxamide (Example 57, 170 mg). LCMS (ESI) Calcd. for C₂₄H₂₅FN₄O₄: 452, found [M+H]⁺=453. ¹H NMR (400 MHz, DMSO-d₆): δ_{H} 11.37 (br s, 1H), 7.61-7.59 (m, 1H), 7.33-7.29 (m, 1H), 7.26-7.22 (m, 2H), 7.07 (d, 1H), 6.94-6.92 (m, 2H), 6.08 (s, 2H), 5.49-5.46 (m, 1H), 3.77-3.63 (m, 2H), 3.46-3.30 (m, 4H), 3.18-3.11 (m, 2H), 1.94 (s, 3H), 1.45 (d, 3H).

### Example 60-62: Synthesis (R)-N-(2-amino-2-oxoethyl)-2-((1-oxo-4-(o-tolyl)-1,2-dihydroisoquinolin-7-yl)oxy)propanamide, (R)-N-(2-amino-2-oxoethyl)-N-methyl-2-((1-oxo-4-(o-tolyl)-1,2-dihydroisoquinolin-7-yl)oxy)propanamide, and (R)-1-(2-((1-oxo-4-(o-tolyl)-1,2-dihydroisoquinolin-7-yl)oxy)propanamido)cyclopropane-1-carboxamide

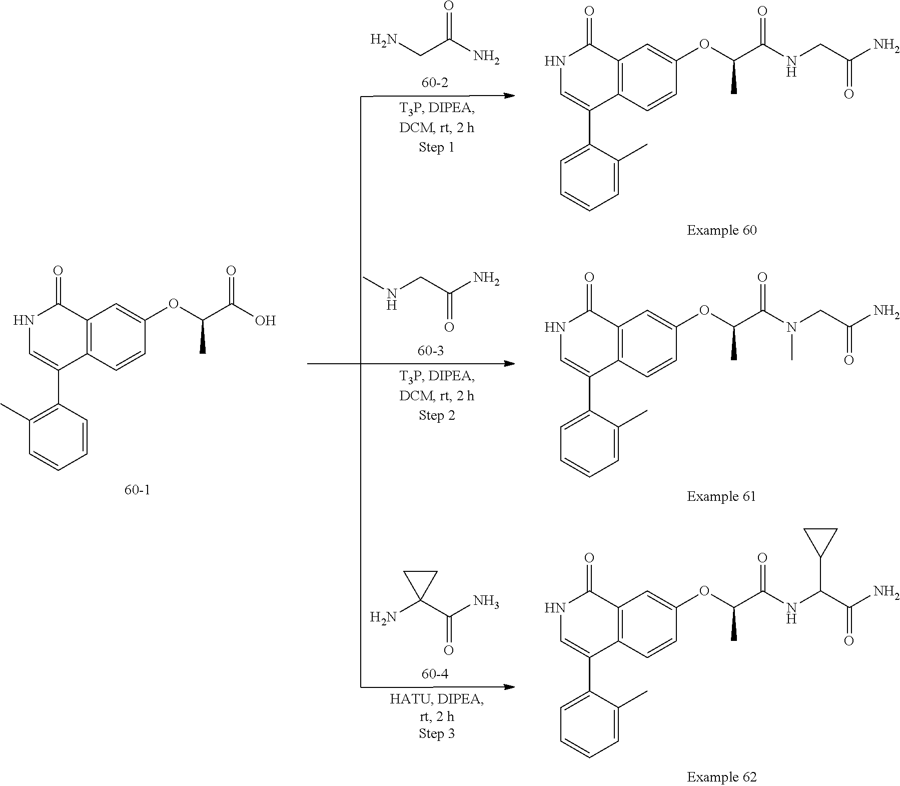

Synthesis of (R)-N-(2-amino-2-oxoethyl)-2-((1-oxo-4-(o-tolyl)-1,2-dihydroisoquinolin-7-yl)oxy)propanamide, Example 60 [Step 1]: To a stirred solution of (R)-2-((1-oxo-4-(o-tolyl)-1,2-dihydroisoquinolin-7-yl)oxy)propanoic acid (60-1, 100 mg, 0.3 mmol) and 2-aminoacetamide (60-2, 45 mg, 0.6 mmol) in dichloromethane (4 mL), DIPEA (0.2 mL, 0.9 mmol) was added followed by T₃P (50% in ethyl acetate) (0.4 mL, 0.6 mmol) dropwise at 0° C. and stirred at ambient temperature for 2 h. The reaction mixture was diluted with dichloromethane and washed with water (twice). The organic extract was collected, dried with anhydrous Na₂SO₄ and concentrated under reduced pressure. The product was purified by reverse phase prep-HPLC and lyophilized to afford (R)-N-(2-amino-2-oxoethyl)-2-((1-oxo-4-(o-tolyl)-1,2-dihydroisoquinolin-7-yl)oxy)propanamide (Example 60, 20 mg). LCMS (ESI) Calcd. for C₂₁H₂₁N₃O₄: 379; found [M+H]⁺=380. ¹H NMR (400 MHz, DMSO-d₆): δ_{H} 11.36 (d, 1H), 8.32-8.28 (m, 1H), 7.69 (s, 1H), 7.35-7.18 (m, 6H), 7.04 (s, 1H), 6.92-6.88 (m, 2H), 4.93 (q, 1H), 3.66 (d, 2H), 2.03 (s, 3H), 1.47 (d, 3H).

Synthesis of (R)-N-(2-amino-2-oxoethyl)-N-methyl-2-((1-oxo-4-(o-tolyl)-1,2-dihydroisoquinolin-7-yl)oxy)propanamide, Example 61 [Step 2]: To a stirred solution of (R)-2-((1-oxo-4-(o-tolyl)-1,2-dihydroisoquinolin-7-yl)oxy)propanoic acid (60-1, 100 mg, 0.3 mmol) and 2-(methylamino)acetamide hydrochloride (60-3, 60 mg, 0.5 mmol) in dichloromethane (4 mL), DIPEA (0.2 mL, 0.9 mmol) was added followed by TP (0.4 mL, 0.6 mmol, 50% in ethyl acetate) dropwise at 0° C. and stirred at ambient temperature for 2 h. The reaction mixture was diluted with dichloromethane and washed with water (twice). The organic extract was collected, dried with anhydrous Na₂SO₄ and concentrated under reduced pressure. The product was purified by reverse phase prep-HPLC and lyophilized to afford (R)-N-(2-amino-2-oxoethyl)-N-methyl-2-((1-oxo-4-(o-tolyl)-1,2-dihydroisoquinolin-7-yl)oxy)propanamide (Example 61, 20 mg). LCMS (ESI) Calcd. for C₂₂H₂₃N₃O₄: 393; found [M+H]⁺=394. ¹H NMR (400 MHz, DMSO-d₆ at 100° C.) δ_{H} 10.96 (br s, 1H), 7.71 (s, 1H), 7.34 (d, 2H), 7.29-7.24 (m, 2H), 7.18 (d, 1H), 6.92 (d, 1H), 6.83 (br s, 2H), 5.29 (br s, 1H), 4.02-3.93 (m, 2H), 3.15 (br s, 2H), 2.07 (s, 3H), 1.50 (d, 3H).

Synthesis of (R)-1-(2-((1-oxo-4-(o-tolyl)-1,2-dihydroisoquinolin-7-yl)oxy)propanamido)cyclopropane-1-carboxamide, Example 62 [Step 3]: To a stirred solution of (R)-2-((1-oxo-4-(o-tolyl)-1,2-dihydroisoquinolin-7-yl)oxy)propanoic acid (60-1, 120 mg, 0.4 mmol) and 1-aminocyclopropane-1-carboxamide (60-4, 45 mg, 0.4 mmol) in DMF (2 mL), HATU (170 mg, 0.4 mmol) was added followed by DIPEA (0.2 mL, 1.1 mmol) at 0° C. and stirred at ambient temperature for 2 h. The reaction mixture was diluted with ethyl acetate and washed with cold water (thrice). The organic extract was collected, washed with brine, dried over anhydrous Na₂SO₄ and concentrated under reduced pressure. The product was purified by reverse phase prep HPLC and isolated compound was lyophilized to afford (R)-1-(2-((1-oxo-4-(o-tolyl)-1,2-dihydroisoquinolin-7-yl)oxy)propanamido)cyclopropane-1-carboxamide (Example 62, 55 mg). LCMS (ESI) Calcd. for C₂₃H₂₃N₃O₄: =405, found [M+H]⁺=406. ¹H NMR (400 MHz, DMSO-d₆) δ_{H} 11.33 (br s, 1H), 8.32 (s, 1H), 7.66 (s, 1H), 7.35 (br s, 2H), 7.29 (d, 2H), 7.19 (d, 1H), 7.09 (br s, 1H), 6.92-6.89 (m, 2H), 6.80 (br s, 1H), 4.85 (d, 1H), 2.03 (s, 3H), 1.47 (d, 3H), 1.22 (s, 2H), 0.91-0.77 (m, 2H).

### Example 63. Synthesis of 2-((4-(2-(2-methoxyethyl)phenyl)-1-oxo-1,2-dihydroisoquinolin-7-yl)oxy)acetonitrile

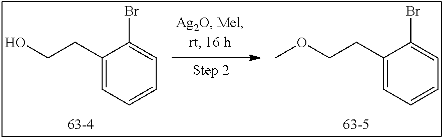

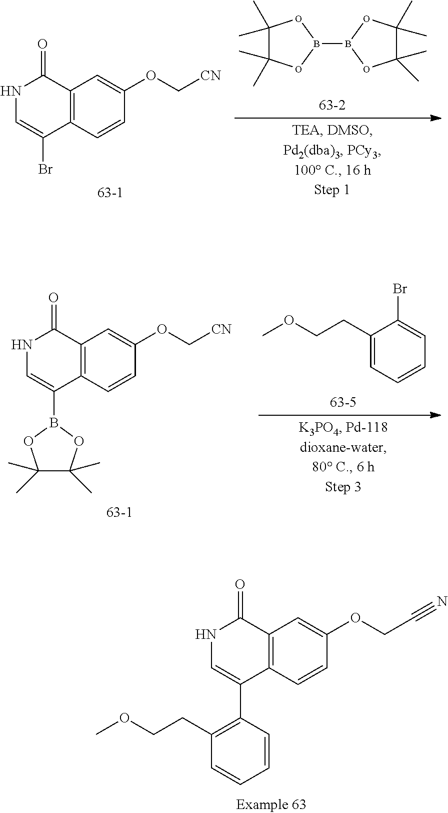

Synthesis of 2-((1-oxo-4-(4,4,5,5-tetramethyl-1,3,2-dioxaborolan-2-yl)-1,2-dihydroisoquinolin-7-yl)oxy)acetonitrile 63-3 [Step 1]: A stirred solution of 2-((4-bromo-1-oxo-1,2-dihydroisoquinolin-7-yl)oxy)acetonitrile (63-1, 200 mg, 0.7 mmol) and bis(pinacolato)diboron (63-2, 730 mg, 2.9 mmol) in DMSO (5 mL) was purged Argon gas for 5 min. Tris(dibenzylideneacetone)dipalladium (65 mg, 0.1 mmol), tricyclohexylphosphine (40 mg, 0.1 mmol) and triethylamine (1.0 mL, 7.2 mmol) were added into the reaction mixture and stirred at 110° C. for 18 h. The reaction mixture was passed through a celite bed, and washed with ethyl acetate. The combined filtrate was concentrated under reduced pressure to afford 2-((1-oxo-4-(4,4,5,5-tetramethyl-1,3,2-dioxaborolan-2-yl)-1,2-dihydroisoquinolin-7-yl)oxy)acetonitrile (63-3, 80 mg). LCMS (ESI) Calcd. for Cl₇H₁₉BN₂O₄: 326, found [M+H]⁺=327.

Synthesis of 1-bromo-2-(2-methoxyethyl)benzene, 63-5 [Step 2]: To a solution of 2-(2-bromophenyl)ethan-1-ol (64-3, 500 mg, 2.5 mmol) in methyl iodide (3.7 mL, 60.0 mmol), silver oxide (1.2 g, 5.0 mmol) was added and stirred at ambient temperature for 16 h. After completion, the reaction mixture was filtered, concentrated under reduced pressure to afford 1-bromo-2-(2-methoxyethyl)benzene (63-5, 520 mg). ¹H NMR (400 MHz, DMSO-d₆) δ_{H} 7.61-7.55 (m, 1H), 7.37-7.28 (m, 2H), 7.19-7.12 (m, 1H), 3.54-3.50 (m, 2H), 3.32-3.24 (m, 3H), 2.95-2.87 (m, 2H).

Synthesis of 2-((4-(2-(2-methoxyethyl)phenyl)-1-oxo-1,2-dihydroisoquinolin-7-yl)oxy)acetonitrile, Example 63 [Step 3]: A solution of 2-((1-oxo-4-(4,4,5,5-tetramethyl-1,3,2-dioxaborolan-2-yl)-1,2-dihydroisoquinolin-7-yl)oxy)acetonitrile (63-3, 200 mg, 0.6 mmol), 1-bromo-2-(2-methoxyethyl)benzene (63-5, 190 mg, 0.9 mmol) and K₃PO₄ (250 mg, 1.2 mmol) in 1,4-dioxane (3 mL) and water (0.5 mL) mixture was purged with Argon gas for 10 min. Into this purged reaction mixture [1,1′-bis(di-tert-butylphosphino)ferrocene]dichloropalladium(II) (38 mg, 0.06 mmol) was added and heated at 60° C. for 4 h. After completion, the reaction mixture was filtered through celite bed and the filtrate was diluted with ethyl acetate and washed with water.

Combined organic extract was washed with brine, dried over anhydrous Na₂SO₄ and concentrated under reduced pressure. The product was purified by reverse phase prep HPLC purification method and lyophilized to afford 2-((4-(2-(2-methoxyethyl)phenyl)-1-oxo-1,2-dihydroisoquinolin-7-yl)oxy)acetonitrile (Example 63, 50 mg). LCMS (ESI) Calcd. for C₂₀H₁₈N₂O₃: 334, found [M+H]⁺=335. ¹H NMR (400 MHz, DMSO-d₆) δ_{H} 11.48-11.47 (br s, 1H), 7.86-7.85 (m, 11H), 7.43-7.35 (m, 3H), 7.33-7.29 (m, 1H), 7.20-7.18 (m, 1H), 6.98-6.94 (m, 2H), 5.32 (s, 2H), 3.35-3.29 (m, 2H), 3.05 (s, 3H), 2.69-2.49 (m, 2H).

### Example 64: Synthesis of 7-(((R)-1-((R)-3-(dimethylamino)pyrrolidin-1-yl)-1-oxopropan-2-yl)oxy)-4-(o-tolyl)isoquinolin-1(21)-one

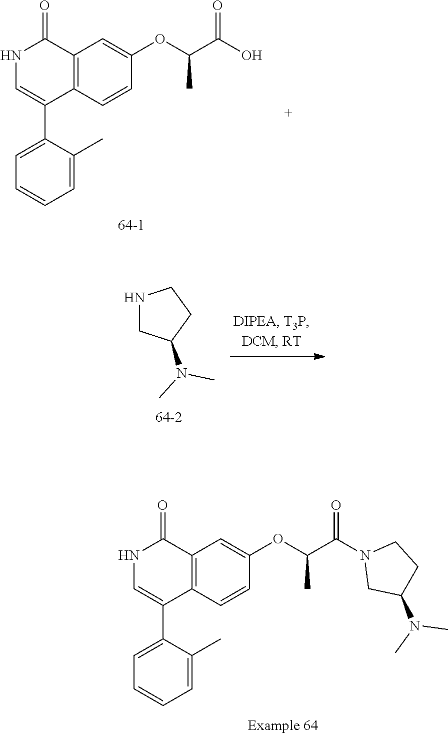

To a stirred solution of (R)-2-((1-oxo-4-(o-tolyl)-1,2-dihydroisoquinolin-7-yl)oxy)propanoic acid (64-1, 120 mg, 0.37 mmol) in dichloromethane (10 mL) was added (R)-N,N-dimethylpyrrolidin-3-amine (64-2, 70 mg, 0.6 mmol) followed by DIPEA (0.3 mL, 1.5 mmol) at 0° C. After 10 min T₃P (0.4 mL, 0.6 mmol, 50% in ethyl acetate) was added to the reaction mixture at 0° C. and stirred at ambient temperature for 2 h. After completion, reaction mixture was quenched with ice water and extracted with dichloromethane (twice). Combined organic extracts was washed with brine, dried over anhydrous sodium sulphate and concentrated under reduced pressure. The product was purified by reverse phase prep HPLC and lyophilized to afford 7-(((R)-1-((R)-3-(dimethylamino)pyrrolidin-1-yl)-1-oxopropan-2-yl)oxy)-4-(0-tolyl)isoquinolin-1(2H)-one (Example 64, 50 mg). LCMS (ESI) Calcd. for C₂₅H₂₉N₃O₃: 420, found [M+H]⁺=420. ¹H NMR (400 MHz, DMSO-d₆) δ_{H} 10.92 (br s, 1H), 7.67 (d, 1H), 7.34-7.33 (m, 2H), 7.30-7.25 (m, 1H), 7.22-7.17 (m, 2H), 6.93 (d, 1H), 6.83 (s, 1H), 5.13-5.09 (q, 1H), 3.86 (br s, 1H), 3.53-3.49 (m, 2H), 3.31-3.20 (m, 2H), 2.18 (s, 6H), 2.06-2.01 (s, 3H), 1.84 (br s, 1H), 1.67-1.65 (m, 1H), 1.49 (d, 3H).

### Example 65: Synthesis of 7-(((R)-1-((S)-3-(dimethylamino)pyrrolidin-1-yl)-1-oxopropan-2-yl)oxy)-4-(o-tolyl)isoquinolin-1(2H)-one

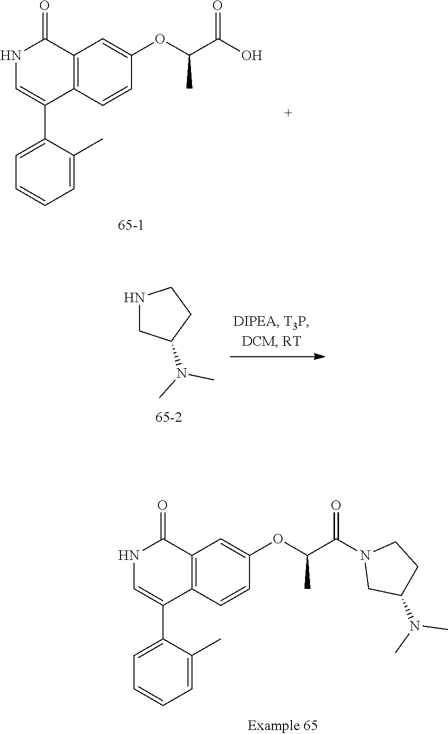

To a stirred solution of (R)-2-((1-oxo-4-(o-tolyl)-1,2-dihydroisoquinolin-7-yl)oxy)propanoic acid (65-1, 120 mg, 0.37 mmol) in dichloromethane (10 mL) was added (S)-N,N-dimethylpyrrolidin-3-amine (65-2, 70 mg, 0.6 mmol) followed by DIPEA (0.3 mL, 1.5 mmol) at 0° C. After 10 min T₃P (0.4 mL, 0.6 mmol, 50% in ethyl acetate) was added to the reaction mixture at 0° C. and stirred at ambient temperature for 2 h. After completion, reaction mixture was quenched with ice water and extracted with dichloromethane (twice). Combined organic extracts was washed with brine, dried over anhydrous sodium sulphate and concentrated under reduced pressure. The product was purified by reverse phase prep HPLC and lyophilized to afford 7-(((R)-1-((S)-3-(dimethylamino)pyrrolidin-1-yl)-1-oxopropan-2-yl)oxy)-4-(o-tolyl)isoquinolin-1(2H)-one (Example 65, 50 mg). LCMS (ESI) Calcd. for C₂₅H₂₉N₃O₃: 420, found [M+H]⁺=420. ¹H NMR (400 MHz, DMSO-d₆) δ_{H} 10.95 (br s, 1H), 7.68 (d, 1H), 7.34-7.33 (m, 2H), 7.29-7.17 (m, 3H), 6.93 (d, 1H), 6.83 (s, 1H), 5.13-5.08 (q, 1H), 3.61-3.13 (m, 4H), 2.80 (br s, 1H), 2.18 (s, 6H), 2.06 (s, 3H), 1.78 (br s, 2H), 1.48 (d, 3H).

### Example 66-67: Synthesis of chiral analogs of 1-((R)-2-((1-oxo-4-(o-tolyl)-1,2-dihydroisoquinolin-7-yl)oxy)propanoyl)-3-propylpiperidine-3-carboxylic acid

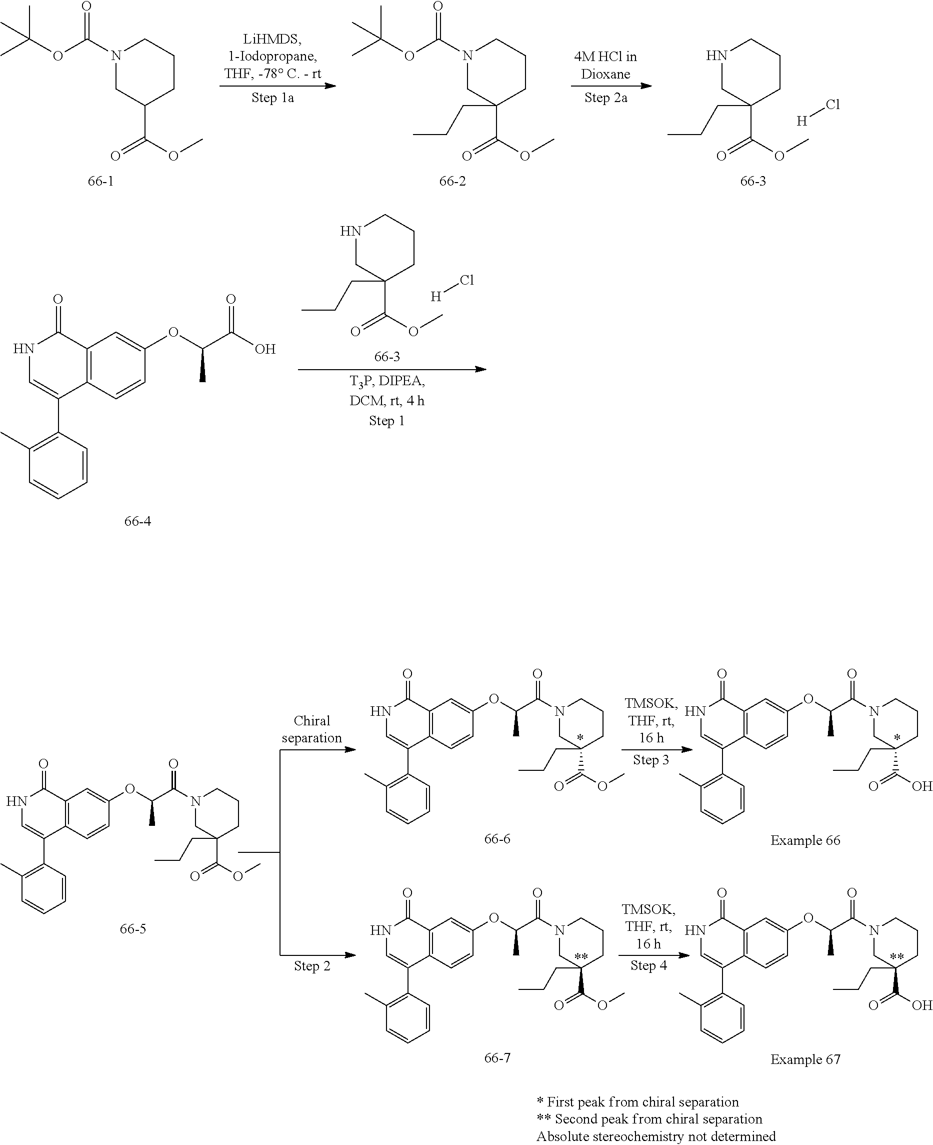

Synthesis of 1-(tert-butyl) 3-methyl 3-propylpiperidine-1,3-dicarboxylate, 66-2 [Step 1a]: To a stirred solution of 1-tert-butyl) 3-methyl piperidine-1,3-dicarboxylate (66-1, 500 mg, 2. mmol) in tetrahydrofuran (10 mL), LiHMDS (13 mL, 12 mmol, 1M in THF) was added dropwise at −78° C. and stirred at same temperature for another 20 min. 1-iodopropane (0.3 mL, 3.0 mmol) was added to the reaction mixture at −78° C. and stirred at ambient temperature for 4 h. After completion, the reaction mixture was quenched with saturated aqueous NH₄Cl solution and extracted with ethyl acetate (twice). Combined organic extract was washed with brine solution, dried over anhydrous sodium sulphate, concentrated under reduced pressure. The product was purified by column chromatography to afford 1-(tert-butyl) 3-methyl 3-propylpiperidine-1,3-dicarboxylate (66-2, 400 mg). ¹H NMR (400 MHz, DMSO-d₆) δ_{H} 3.70 (d, 1H), 3.59 (s, 3H), 3.13 (br s, 2H), 1.93-1.90 (m, 1H), 1.44-1.42 (m, 3H), 1.37 (s, 9H), 1.16-1.10 (q, 2H), 0.82 (t, 3H).

Synthesis of methyl 3-propylpiperidine-3-carboxylate hydrochloride, 66-3 [Step 2a]: To a stirred solution of 1-tert-butyl) 3-methyl 3-propylpiperidine-1,3-dicarboxylate (66-2, 200 mg, 0.7 mmol) in 1,4-dioxane (2 mL), 4M HCl in dioxane (2.0 mL, 7.0 mmol) was added dropwise at 0° C. and stirred at ambient temperature for 16 h. After completion, the reaction mixture was concentrated under reduced pressure to afford methyl 3-propylpiperidine-3-carboxylate hydrochloride (66-3, 200 mg). LCMS (ESI) Calcd. for C₁₀H₁₉NO₂: 185, found [M+H]⁺=186.

Synthesis of methyl 1-((R)-2-((1-oxo-4-(o-tolyl)-1,2-dihydroisoquinolin-7-yl)oxy)propanoyl)-3-propylpiperidine-3-carboxylate, 66-5 [Step 1]: To a stirred solution of (R)-2-((1-oxo-4-(o-tolyl)-1,2-dihydroisoquinolin-7-yl)oxy)propanoic acid (66-4, 500 mg, 1.6 mmol) and methyl 3-propylpiperidine-3-carboxylate hydrochloride (66-3, 520 mg, 2.3 mmol) in dichloromethane (2 mL), DIPEA (1.1 mL, 6.2 mmol) was added followed by T₃P (1.4 mL, 2.3 mmol, 50% in ethyl acetate) and stirred at ambient temperature for 4 h. After completion, the reaction mixture was diluted with dichloromethane and washed with water (twice). The combined organic extract was collected, dried with anhydrous Na₂SO₄ and concentrated under reduced pressure to afford methyl 1-((R)-2-((1-oxo-4-(o-tolyl)-1,2-dihydroisoquinolin-7-yl)oxy)propanoyl)-3-propylpiperidine-3-carboxylate (66-5, 470 mg). LCMS (ESI) Calcd. for C₂₉H₃₄N₂O₅: 491, found [M+H]⁺=491.

Synthesis of chiral methyl 1-((R)-2-((1-oxo-4-(o-tolyl)-1,2-dihydroisoquinolin-7-yl)oxy)propanoyl)-3-propylpiperidine-3-carboxylate, 66-6 and 66-7 [Step 2]: Racemic methyl 1-((R)-2-((1-oxo-4-(0-tolyl)-1,2-dihydroisoquinolin-7-yl)oxy)propanoyl)-3-propylpiperidine-3-carboxylate (66-5, 470 mg) was purified by Prep-SFC purification and lyophilized to afford the first product as chiral methyl 1-((R)-2-((1-oxo-4-(o-tolyl)-1,2-dihydroisoquinolin-7-yl)oxy)propanoyl)-3-propylpiperidine-3-carboxylate (66-6, 170 mg) as Peak 1 and the second product as chiral methyl 1-((R)-2-((1-oxo-4-(o-tolyl)-1,2-dihydroisoquinolin-7-yl)oxy)propanoyl)-3-propylpiperidine-3-carboxylate (66-7, 150 mg) as Peak 2. The absolute stereochemistry were not determined.

Normal Phase Prep Method: Chiral separation was performed using an Agilent 1200 series instrument and a CHIRALPAK IC (250×21 mm), 5μ, which was operated at ambient temperature with a flow rate of 21.0 mL/min. The mobile phase was a mixture of 70% hexane and 30% ethanol. the instrument was held in isocratic mode for up to 25 min at a wavelength of nm.

66-6: Chiral methyl 1-((R)-2-((1-oxo-4-(o-tolyl)-1,2-dihydroisoquinolin-7-yl)oxy)propanoyl)-3-propylpiperidine-3-carboxylate (Peak 1): LCMS (ESI) Calcd. for C₂₉H₃₄N₂O₅: 491, [M+H]⁺=491. Chiral-HPLC purity: 100% de.

66-7: Chiral methyl 1-((R)-2-((1-oxo-4-(o-tolyl)-1,2-dihydroisoquinolin-7-yl)oxy)propanoyl)-3-propylpiperidine-3-carboxylate (Peak 2): LCMS (ESI) Calcd. for C₂₉H₃₄N₂O₅: 491, [M+H]⁺=491. Chiral-HPLC purity: 100% de.

Synthesis of 1-((R)-2-((1-oxo-4-(o-tolyl)-1,2-dihydroisoquinolin-7-yl)oxy)propanoyl)-3-propylpiperidine-3-carboxylic acid, Example 66 [Step 3]: To a stirred solution of chiral methyl 1-((R)-2-((1-oxo-4-(o-tolyl)-1,2-dihydroisoquinolin-7-yl)oxy)propanoyl)-3-propylpiperidine-3-carboxylate (66-6, 200 mg, 0.4 mmol) in tetrahydrofuran (6 mL), potassium trimethylsilanolate (210 mg, 1.6 mmol) was added and stirred at ambient temperature for 16 h. The reaction mixture was quenched with 1N HCl solution to adjust the pH˜3 and extracted with ethyl acetate (twice). The organic extract was collected, dried over anhydrous Na₂SO₄ and concentrated under reduced pressure. The product was purified by reverse phase prep-HPLC and lyophilized to afford 1-((R)-2-((1-oxo-4-(o-tolyl)-1,2-dihydroisoquinolin-7-yl)oxy)propanoyl)-3-propylpiperidine-3-carboxylic acid (Example 66, 120 mg). LCMS (ESI) Calcd. for C₂₈H₃₂N₂O₅: 477, found [M+H]⁺=477. ¹H NMR (400 MHz, DMSO-d₆ at 100° C.) δ_{H} 10.89 (br s, 1H), 7.71 (s, 1H), 7.34-7.33 (m, 2H), 7.29-7.17 (m, 3H), 6.93 (d, 1H), 6.84 (s, 1H), 5.37 (br s, 1H), 3.88-3.78 (m, 2H), 2.06 (s, 3H), 1.96 (br s, 2H), 1.49 (d, 6H), 1.35 (br s, 3H), 1.54 (br s, 2H), 0.75 (m, 3H). Absolute stereochemistry was not determined.

Synthesis of 1-((R)-2-((1-oxo-4-(o-tolyl)-1,2-dihydroisoquinolin-7-yl)oxy)propanoyl)-3-propylpiperidine-3-carboxylic acid, Example 67 [Step 4]: To a stirred solution of chiral methyl 1-((R)-2-((1-oxo-4-(o-tolyl)-1,2-dihydroisoquinolin-7-yl)oxy)propanoyl)-3-propylpiperidine-3-carboxylate (66-7, 150 mg, 0.3 mmol) in tetrahydrofuran (6 mL), potassium trimethylsilanolate (390 mg, 3.4 mmol) was added and stirred at ambient temperature for 16 h. The reaction mixture was quenched with 1N HCl solution to adjust the pH˜3 and extracted with ethyl acetate (twice). The combined organic extract was collected, dried over anhydrous Na₂SO₄ and concentrated under reduced pressure. The product was purified by reverse phase prep-HPLC and lyophilized to afford 1-((R)-2-((1-oxo-4-(o-tolyl)-1,2-dihydroisoquinolin-7-yl)oxy)propanoyl)-3-propylpiperidine-3-carboxylic acid (Example 67, 50 mg). LCMS (ESI) Calcd. for C₂₈H₃₂N₂O₅: 477, found [M+H]⁺=477. ¹H NMR (400 MHz, DMSO-d₆ at 100° C.) δ_{H} 7.70 (br s, 1H), 7.33-7.17 (m, 5H), 6.93 (d, 1H), 6.84 (s, 1H), 5.43 (q, 1H), 2.06 (s, 3H), 1.70 (br s, 1H), 1.52-1.21 (m, 10H), 0.75 (m, 3H). The absolute stereochemistry was not determined.

### Example 68: Synthesis of (R)-7-((1-(4-(methylamino)piperidin-1-yl)-1-oxopropan-2-yl)oxy)-4-(o-tolyl)isoquinolin-1(2H)-one

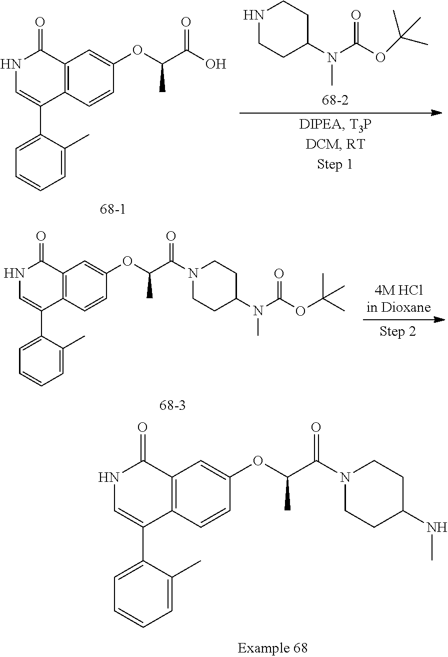 [SMILES: Cc1ccccc1-c1c[nH]c(=O)c2cc(O[C@H](C)C(=O)O)ccc12 ; CN(C(=O)OC(C)(C)C)C1CCNCC1 ; Cc1ccccc1-c1c[nH]c(=O)c2cc(O[C@H](C)C(=O)N3CCC(N(C)C(=O)OC(C)(C)C)CC3)ccc12 ; CNC1CCN(C(=O)[C@@H](C)Oc2ccc3c(-c4ccccc4C)c[nH]c(=O)c3c2)CC1]

Synthesis of tert-butyl (R)-methyl(1-(2-((1-oxo-4-(o-tolyl)-1,2-dihydroisoquinolin-7-yl)oxy)propanoyl)piperidin-4-yl)carbamate, 68-3 [Step 1]: To a stirred solution of (R)-2-((1-oxo-4-(o-tolyl)-1,2-dihydroisoquinolin-7-yl)oxy)propanoic acid (68-1, 120 mg, 0.4 mmol) in dichloromethane (10 mL), tert-butyl methyl(piperidin-4-yl)carbamate (68-2, 120 mg, 0.6 mmol) was added followed by DIPEA (0.3 mL, 1.5 mmol) at 0° C. After 10 min, T₃P (0.4 mL, 0.6 mmol, 50% in ethyl acetate) was added to the reaction mixture at 0° C. and stirred at ambient temperature for 2 h. After completion, the reaction mixture was quenched with ice water and extracted with dichloromethane (twice). Combined organic extracts was washed with brine, dried over anhydrous sodium sulphate and concentrated under reduced pressure to afford tert-butyl (R)-methyl(1-(2-((1-oxo-4-(o-tolyl)-1,2-dihydroisoquinolin-7-yl)oxy)propanoyl)piperidin-4-yl)carbamate (68-3, 100 mg). LCMS (ESI) Calcd. for C₃₀H₃₇N₃O₅: 520, found [M+H]⁺=520.

Synthesis of (R)-7-((1-(4-(methylamino)piperidin-1-yl)-1-oxopropan-2-yl)oxy)-4-(o-tolyl)isoquinolin-1(2H)-one, Example 68 [Step 2]: To a stirred solution of tert-butyl (R)-methyl(1-(2-((1-oxo-4-(o-tolyl)-1,2-dihydroisoquinolin-7-yl)oxy)propanoyl)piperidin-4-yl)carbamate (68-3, 170 mg, 0.3 mmol) in 1,4-dioxane (2 mL), 4M HCl in dioxane (2.0 mL, 3.3 mmol) was added dropwise at 0° C. and stirred at ambient temperature for 16 h. After completion, the reaction mixture was concentrated under reduced pressure. The product was purified by reverse phase prep HPLC and lyophilized to afford (R)-7-((1-(4-(methylamino)piperidin-1-yl)-1-oxopropan-2-yl)oxy)-4-(o-tolyl)isoquinolin-1(2H)-one (Example 68, 40 mg). LCMS (ESI) Calcd. for C₂₅H₂₉N₃O₃: 420, found [M+H]⁺=420. ¹H NMR (400 MHz, DMSO-d₆) δ_{H} 11.00 (br s, 1H), 7.69 (s, 1H), 7.32 (d, 2H), 7.29-7.17 (m, 3H), 6.93 (d, 1H), 6.83 (s, 1H), 5.32-5.27 (q, 1H), 4.04-3.96 (m, 2H), 3.09 (br s, 1H), 2.67-2.57 (m, 1H), 2.31 (s, 3H), 2.07 (s, 3H), 1.83 (br s, 2H), 150 (d, 3H), 1.32-1.17 (m, 2H).

### Example 69: (R)-2-(2-(methyl(1-oxo-4-(o-tolyl)-1,2-dihydroisoquinolin-7-yl)amino)acetamido)propanamide

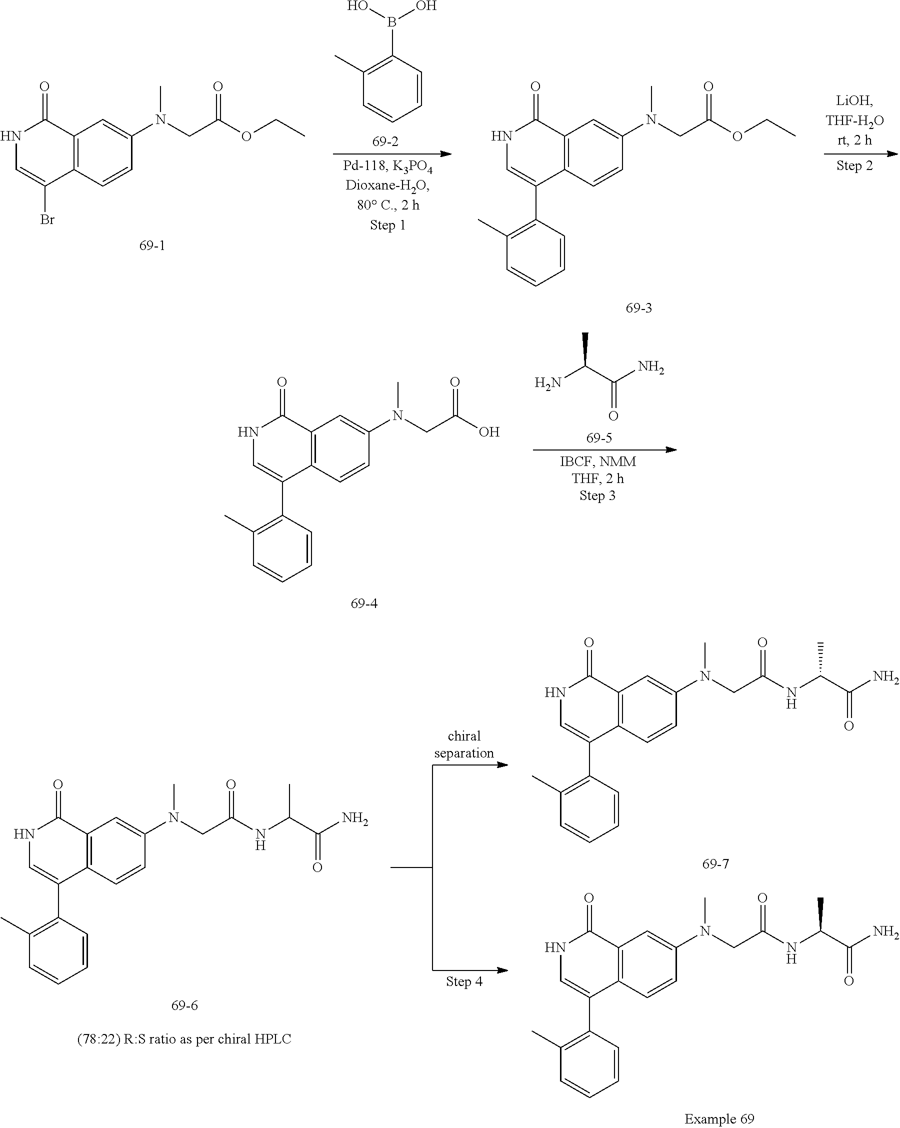

Synthesis of ethyl N-methyl-N-(1-oxo-4-(o-tolyl)-1,2-dihydroisoquinolin-7-yl)glycinate, 69-3 [Step 1]: To a stirred solution of ethyl N-(4-bromo-1-oxo-1,2-dihydroisoquinolin-7-yl)-N-methylglycinate (69-1, 280 mg, 0.8 mmol) in 1,4-dioxane (4 mL) and water (1 mL) mixture, K₃PO₄ (435 mg, 2 mmol) and o-tolylboronic acid (165 mg, 1.2 mmol) were added. The reaction mixture was degassed with nitrogen gas for 10 min. Pd-118 (55 mg, 0.1 mmol) was added to the reaction mixture. The reaction mixture was heated at 60° C. for 1 h. After the completion of the reaction, it was filtered through celite bed. The filtrate was diluted with water and extracted with ethyl acetate. Combined organic extract was washed with brine, dried over anhydrous Na₂SO₄ and concentrated under reduced pressure. The product was purified by flash chromatography to afford ethyl N-methyl-N-(1-oxo-4-(o-tolyl)-1,2-dihydroisoquinolin-7-yl)glycinate (69-3, 235 mg). LCMS (ESI) Calcd. C₂₁H₂₂N₂O₃: 350, found [M+H]⁺=351.

Synthesis of N-methyl-N-(1-oxo-4-(o-tolyl)-1,2-dihydroisoquinolin-7-yl)glycine, 69-4 [Step 2]: To a stirred solution of ethyl N-methyl-N-(1-oxo-4-(o-tolyl)-1,2-dihydroisoquinolin-7-yl)glycinate (69-3, 233 mg, 0.6 mmol) in tetrahydrofuran (4 mL), an aqueous solution (1 mL) of LiOH·H₂O (80 mg, 1.9 mmol) was added and stirred for 1 h. After completion, the reaction mixture with diluted with minimum amount of water, acidified with 1N HCL to pH˜5 and extracted with ethyl acetate (twice). Combined organic extract was washed with brine, dried over anhydrous Na₂SO₄ and concentrated under reduced pressure to afford N-methyl-N-(1-oxo-4-(o-tolyl)-1,2-dihydroisoquinolin-7-yl)glycine (69-4, 200 mg). LCMS (ESI) Calcd. For C₁₉H₁₈N₂O₃: 322, found [M+H]⁺=323.

Synthesis of 2-(2-(methyl(1-oxo-4-(o-tolyl)-1,2-dihydroisoquinolin-7-yl)amino)acetamido)propanamide, 69-6 [Step 3]: To a stirred solution of N-methyl-N-(1-oxo-4-(o-tolyl)-1,2-dihydroisoquinolin-7-yl)glycine (69-4, 220 mg) in tetrahydrofuran (4 mL), IBCF (0.1 mL, 0.6 mmol) and NMM (0.2 mL, 0.7 mmol) were added at 0° C. and stirred for 40 min. After that, (S)-2-aminopropanamide (69-5, 90 mg) followed by NMM (0.2 mL, 0.8 mmol) were added into the reaction mixture and stirred for another 1 h. After completion, the volatiles were removed under reduced pressure. The residue was diluted with minimum amount of water and extracted with ethyl acetate (twice). Combined organic extract was washed with brine, dried over anhydrous Na₂SO₄ and concentrated under reduced pressure. The product was purified by reverse phase prep HPLC purification method and lyophilized to afford 2-(2-(methyl(1-oxo-4-(o-tolyl)-1,2-dihydroisoquinolin-7-yl)amino)acetamido)propanamide (69-6, 30 mg). LCMS (ESI) Calcd. For C₂₂H₂₄N₄O₃: 392, found [M+H]⁺=393.

Synthesis of (R)-2-(2-(methyl(1-oxo-4-(o-tolyl)-1,2-dihydroisoquinolin-7-yl)amino)acetamido)propanamide, Example 69 [Step 4]: Racemic mixture of 2-(2-(methyl(1-oxo-4-(o-tolyl)-1,2-dihydroisoquinolin-7-yl)amino)acetamido)propanamide (69-6, 30 mg, 0.1 mmol) was separated by chiral prep HPLC purification method and lyophilized to afford the first product as (R)-2-(2-(methyl(1-oxo-4-(o-tolyl)-1,2-dihydroisoquinolin-7-yl)amino)acetamido)propanamide (Example 69, 16 mg) as Peak 1 and the second product as (S)-2-(2-(methyl(1-oxo-4-(o-tolyl)-1,2-dihydroisoquinolin-7-yl)amino)acetamido)propanamide (69-7, 3 mg) as Peak 2. The absolute stereochemistry was assigned by assuming the major yielding compound was Example 69.

Example 69: (R)-2-(2-(methyl(1-oxo-4-(o-tolyl)-1,2-dihydroisoquinolin-7-yl)amino)acetamido)propanamide (Peak 1): LCMS (ESI) Calcd. for C₂₂H₂₄N₄O₃: 392, found [M−H]⁻=391. ¹H NMR (400 MHz, DMSO-d₆) δ_{H} 11.10 (br s, 1H), 8.03-8.02 (m, 1H), 7.39 (br s, 1H), 7.33-7.25 (m, 3H), 7.18-7.16 (m, 1H), 7.09-7.07 (m, 1H), 7.00 (br s, 1H), 6.80-6.78 (d, 1H), 6.71-6.70 (m, 1H), 4.22-4.00 (m, 3H), 3.49-3.41 (m, 2H), 3.04 (s, 3H), 2.04 (s, 3H), 1.35-1.18 (m, 2H).

Chiral Prep method: Chiral separation was performed using an Agilent 1200 series instrument and a CHLRALPAK IC (250×21 mm), 5μ, which was operated at ambient temperature with a flow rate of 21.0 mL/min. The mobile phase was a mixture of 70% hexane and 30% ethyl alcohol. The instrument was held in isocratic mode for up to 37 min at a wavelength of 310 nm.

### Example 70: Synthesis of 2-((4-(3-fluoro-2-methylphenyl)-1-oxo-1,2-dihydroisoquinolin-7-yl)(methyl)amino)-N-(2-hydroxyethyl)acetamide

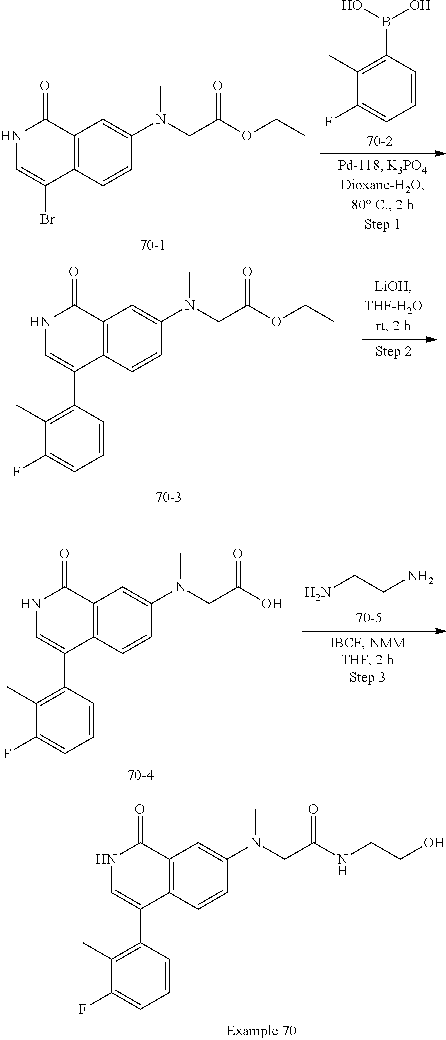

Synthesis of ethyl N-(4-(3-fluoro-2-methylphenyl)-1-oxo-1,2-dihydroisoquinolin-7-yl)-N-methylglycinate, 70-3 [Step 1]: To a stirred solution of ethyl N-(4-bromo-1-oxo-1,2-dihydroisoquinolin-7-yl)-N-methylglycinate (70-1, 270 mg, 0.8 mmol) in 1,4-dioxane (4 mL) and water (1 mL) mixture, K₃PO₄ (420 mg, 2.0 mmol) and (3-fluoro-2-methyl-phenyl)boronic acid (70-2, 185 mg, 1.2 mmol) were added into the reaction mixture. The reaction mixture was degassed with nitrogen gas for 10 min and into the solution Pd-118 (50 mg, 0.1 mmol) was added. The reaction mixture was heated at 60° C. for 1 h. After completion, the reaction mixture was filtered through celite bed. The filtrate was diluted with water and extracted with ethyl acetate (twice). Combined organic extract was washed with brine, dried over anhydrous Na₂SO₄and concentrated under reduced pressure. The product was purified by combiflash column chromatography to afford ethyl N-(4-(3-fluoro-2-methylphenyl)-1-oxo-1,2-dihydroisoquinolin-7-yl)-N-methylglycinate (70-3, 130 mg). LCMS (ESI) Calcd. for C₂₁H₂₁FN₂O₃: 368, found [M+H]⁺=369.

Synthesis of N-(4-(3-fluoro-2-methylphenyl)-1-oxo-1,2-dihydroisoquinolin-7-yl)-N-methylglycine, 70-4 [Step 2]: To a stirred solution of ethyl N-(4-(3-fluoro-2-methylphenyl)-1-oxo-1,2-dihydroisoquinolin-7-yl)-N-methylglycinate (70-3, 130 mg, 0.3 mmol) in tetrahydrofuran (4 mL), an aqueous solution (1 mL) of LiOH·H₂O (45 mg, 1 mmol) was added and allowed to stir at ambient temperature for 1 h. The reaction mixture was diluted with minimum amount of water and acidified to pH˜4-5 with 1N HCl and extracted with ethyl acetate (twice). Combined organic extract was concentrated under reduced pressure to afford N-(4-(3-fluoro-2-methylphenyl)-1-oxo-1,2-dihydroisoquinolin-7-yl)-N-methylglycine (70-4, 115 mg). LCMS (ESI) Calcd. for C₁₉H₁₇FN₂O₃: 340, found [M+H]⁺=341.

Synthesis of 2-((4-(3-fluoro-2-methylphenyl)-1-oxo-1,2-dihydroisoquinolin-7-yl)(methyl)amino)-N-(2-hydroxyethyl)acetamide, Example 70 [Step 3]: To a stirred solution of N-(4-(3-fluoro-2-methylphenyl)-1-oxo-1,2-dihydroisoquinolin-7-yl)-N-methylglycine (70-4, 100 mg, 0.3 mmol) in tetrahydrofuran (3 mL), IBCF (0.03 mL, 0.3 mmol) and NMM (0.1 mL, 0.3 mmol) were added at 0° C. and stirred for 40 min. 2-Aminoethanol hydrochloride (70-5, 45 mg, 0.4 mmol) and NMM (0.1 mL, 0.3 mmol) were added into the reaction mixture and stirred for another 1 h. After completion, the volatiles were removed under reduced pressure. The residue was diluted with minimum amount of water and extracted with ethyl acetate, washed with brine, dried over anhydrous Na₂SO₄and concentrated under reduced pressure. The product was purified by reverse phase prep HPLC purification and lyophilized to afford 2-((4-(3-fluoro-2-methylphenyl)-1-oxo-1,2-dihydroisoquinolin-7-yl)(methyl)amino)-N-(2-hydroxyethyl)acetamide (Example 70, 40 mg). LCMS (ESI) Calcd. for C₂₁H₂₂FN₃O₃: 383, found [M+H]⁺=384. ¹H NMR (400 MHz, DMSO-d₆): δ_{H} 11.17 (br s, 1H), 7.94 (br s, 1H), 7.38 (br s, 1H), 7.33-7.28 (m, 1H), 7.24-7.20 (m, 1H), 7.09-7.04 (m, 2H), 6.85-6.82 (m, 1H), 6.76 (s, 1H), 4.65-4.63 (m, 1H), 3.99 (br s, 2H), 3.37-3.28 (m, 2H), 3.18-3.12 (m, 2H), 3.05 (s, 3H), 1.95 (s, 3H).

### Example 71: Synthesis of 4-(2-(hydroxymethyl)-6-methylphenyl)-7-(2,2,2-trifluoroethoxy)isoquinolin-1(2H)-one

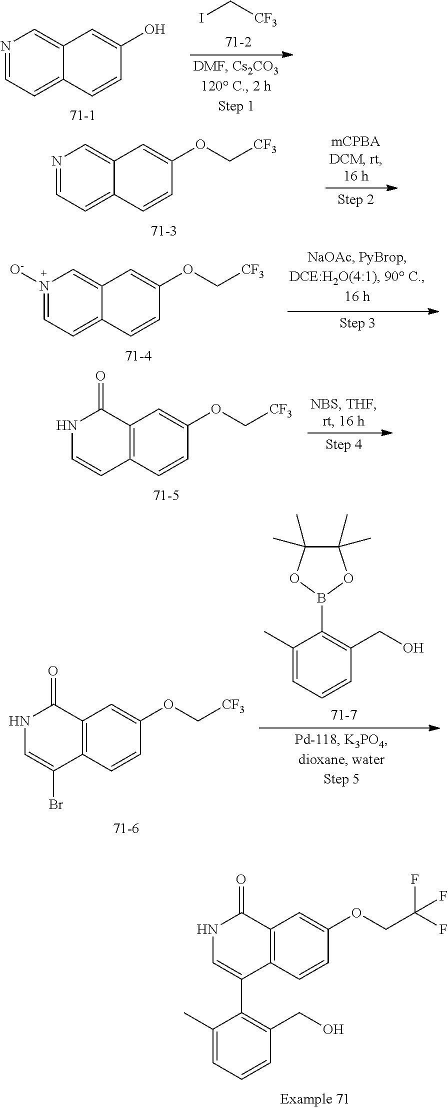

Synthesis of 7-(2,2,2-trifluoroethoxy)isoquinoline, 71-3 [Step 1]: To a stirred solution of isoquinolin-7-ol (71-1, 500 mg, 3.4 mmol) and Cs₂CO₃ (2.2 g, 6.9 mmol) in DMF (10 mL), 1,1,1-trifluoro-2-iodo-ethane (71-2, 1.0 g, 5.8 mmol) was added at ambient temperature and heated at 120° C. for 3 h. After completion, the reaction mixture was diluted with water and extracted with ethyl acetate. The combined organic extract was washed with cold water and brine, dried over anhydrous Na₂SO₄, and concentrated under reduced pressure to afford 7-(2,2,2-trifluoroethoxy)isoquinoline (71-3, 600 mg). LCMS (ESI) Calcd. for C₁₁H₁₈F₃NO: 227, found [M+H]⁺=228.

Synthesis of 7-(2,2,2-trifluoroethoxy)isoquinoline 2-oxide, 71-4 [Step 2]: To a stirred solution of 7-(2,2,2-trifluoroethoxy)isoquinoline (71-3, 1.0 g, 4.4 mmol) in dichloromethane (30 mL), mCPBA (1.6 gm, 6.6 mmol, 70% w/w) was added portion-wise at 0° C. and stirred at ambient temperature for 16 h. After completion, the reaction mixture was quenched with saturated NaHCO₃ solution and extracted with dichloromethane (twice). The combined organic extract was washed with brine, dried over anhydrous Na₂SO₄, concentrated under reduced pressure to afford 7-(2,2,2-trifluoroethoxy)isoquinoline 2-oxide (71-4, 740 mg). LCMS (ESI) Calcd. for C₁₁H₈F₃NO₂: 243, found [M+H]⁺=244.

Synthesis of 7-(2,2,2-trifluoroethoxy)isoquinolin-1(2H)-one, 71-5 [Step 3]: To a suspension of 7-(2,2,2-trifluoroethoxy)isoquinoline 2-oxide (71-4, 1.8 g, 7.4 mmol) in dichloroethane (80 mL) and water (20 mL) mixture, NaOAc (1.2 g, 14.8 mmol) and PyBroP (6.9 g, 14.8 mmol) were added subsequently at ambient temperature and heated at 90° C. for 16 h. The reaction mixture was diluted with water and extracted with dichloromethane (twice). The combined organic extract was, dried over anhydrous Na₂SO₄ and concentrated under reduced pressure. The crude was purified by flash column chromatography to afford 7-(2,2,2-trifluoroethoxy)isoquinolin-1(2H)-one (71-5, 1 g). LCMS (ESI) Calcd. for C₁₁H₈F₃NO₂: 243, found [M+H]⁺=244.

Synthesis of 4-bromo-7-(2,2,2-trifluoroethoxy)isoquinolin-1(2H)-one, 71-6 [Step 4]: To a stirred solution of 7-(2,2,2-trifluoroethoxy)isoquinolin-1(2H)-one (71-5, 500 mg, 2.1 mmol) in tetrahydrofuran (40 mL), N-bromosuccinimide (405 mg, 2.3 mmol) was added portionwise at 0° C. and stirred at ambient temperature for 16 h. After completion, the reaction mixture was diluted with water. The solid was filtered and washed with water and dried over reduced pressure to afford 4-bromo-7-(2,2,2-trifluoroethoxy)isoquinolin-1(2H)-one (71-6, 440 mg). LCMS (ESI) Calcd. for C₁₁H₇BrF₃NO₂: 322, found [M+H]⁺=322.

Synthesis of 4-(2-(hydroxymethyl)-6-methylphenyl)-7-(2,2,2-trifluoroethoxy)isoquinolin-1(2H)-one, Example 71 [Step 5]: A sealed tube was charged with 4-bromo-7-(2,2,2-trifluoroethoxy)isoquinolin-1(2H)-one (71-6, 200 mg, 0.6 mmol), (3-methyl-2-(4,4,5,5-tetramethyl-1,3,2-dioxaborolan-2-yl)phenyl)methanol (71-7, 230 mg, 0.9 mmol) and K₃PO₄ (265 mg, 1.2 mmol) in 1,4-dioxane (6 mL) and water (1 mL). The reaction mixture was purged with Argon gas for 10 min. [1,1′-bis(di-tert-butylphosphino)ferrocene]dichloropalladium(II) (40 mg, 0.06 mmol) was added into the reaction mixture and heated at 80° C. for 16 h. The reaction mixture was cooled to ambient temperature and filtered over celite bed. The filtrate was concentrated under reduced pressure and purified by reverse phase HPLC to afford 4-(2-(hydroxymethyl)-6-methylphenyl)-7-(2,2,2-trifluoroethoxy)isoquinolin-1(2H)-one (Example 71, 35 mg). LCMS (ESI) Calcd. for C₁₉H₁₆F₃NO₃: 363, found [M−H]⁻=362. ¹H NMR (400 MHz, DMSO-d₆) δ_{H} 11.43 (d, 1H), 7.82 (s, 1H), 7.43 (d, 1H), 7.37-7.33 (m, 2H), 7.24 (d, 1H), 6.88 (d, 1H), 6.78 (d, 1H), 4.97-4.88 (m, 3H), 4.24 (q, 1H), 4.01 (q, 1H), 1.95 (s, 3H).

### Example 72: Synthesis of 7-(2,2-difluoroethoxy)-4-(2-(hydroxymethyl)-6-methylphenyl)isoquinolin-1(2H)-one

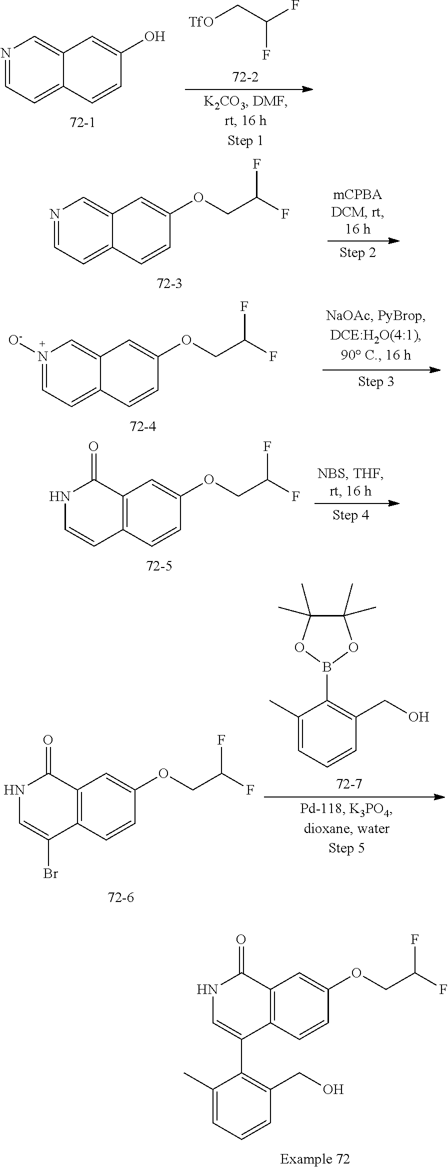

Synthesis of 7-(2,2-difluoroethoxy)isoquinoline, 72-3 [Step 1]: To a stirred solution of isoquinolin-7-ol (72-1, 1.5 g, 10.3 mmol) and K₂CO₃ (6.5 gm, 31.0 mmol) in DMF (30 mL), 1,1-difluoro-2-(trifluoromethylsulfonyl)ethane (72-2, 2.2 mg, 11.4 mmol) was added at ambient temperature and stirred for 16 h. After completion, the reaction mixture was quenched with water and extracted with ethyl acetate (twice). The combined organic extract was washed with cold water, brine, dried over anhydrous Na₂SO₄, and concentrated under reduced pressure to afford 7-(2,2-difluoroethoxy)isoquinoline (72-3, 1.6 g). LCMS (ESI) Calcd. for C₁₁H₉F₂NO: 209, found [M+H]⁺=210.

Synthesis of 7-(2,2-difluoroethoxy)isoquinoline 2-oxide, 72-4 [Step 2]: To a stirred solution of 7-(2,2-difluoroethoxy)isoquinoline (72-3,1.6 gm, 7.6 mmol) in dichloromethane (60 mL), mCPBA (2.8 gm, 11.5 mmol) was added portion wise at 0° C. and stirred at ambient temperature for 16 h. The reaction mixture was quenched with saturated NaHCO₃ solution and extracted with dichloromethane (twice). The combined organic extract was washed with brine, dried over anhydrous Na₂SO₄, and concentrated under reduced pressure to afford 7-(2,2-difluoroethoxy)isoquinoline 2-oxide (72-4, 1.0 g). LCMS (ESI) Calcd. for C₁₁H₉F₂NO₂: 225, found [M+H]⁺=226.

Synthesis of 7-(2,2-difluoroethoxy)isoquinolin-1(2H)-one, 72-5 [Step 3]: To a stirred solution of 7-(2,2-difluoroethoxy)isoquinoline 2-oxide (72-4, 100 mg, 0.4 mmol) in dichloroethane (10 mL) and water (3 mL) mixture, NaOAc (75 mg, 0.8 mmol) and PyBroP (415 mg, 0.8 mmol) were added at ambient temperature and heated at 90° C. for 4 h. The reaction mixture was cooled to ambient temperature and extracted with dichloromethane. The combined organic extract was washed with brine, dried over anhydrous Na₂SO₄ and concentrated under reduced pressure. The product was purified by flash column chromatography to afford 7-(2,2-difluoroethoxy)isoquinolin-1(2H)-one (72-5, 40 mg). LCMS (ESI) Calcd. for C₁₁H₉F₂NO₂: 225, found [M+H]⁺=226.

Synthesis of 4-bromo-7-(2,2-difluoroethoxy)isoquinolin-1(2H)-one, 72-6 [Step 4]: To a stirred solution of 7-(2,2-difluoroethoxy)isoquinolin-1(2H)-one (72-5, 300 mg, 1.3 mmol) in tetrahydrofuran (10 mL), N-bromosuccinimide (260 mg, 1.5 mmol) was added portion wise at 0° C. and stirred at ambient temperature for 4 h. The reaction mixture was diluted with water. The solid was filtered, washed with water and dried under reduced pressure to afford 4-bromo-7-(2,2-difluoroethoxy)isoquinolin-1(2H)-one (72-6, 220 mg). LCMS (ESI) Calcd. for C₁₁H₈BrF₂NO₂: 304, found [M+H]⁺=306.

Synthesis of 7-(2,2-difluoroethoxy)-4-(2-(hydroxymethyl)-6-methylphenyl)isoquinolin-1(2H)-one, Example 72 [Step-5]: A sealed tube was charged with 4-bromo-7-(2,2-difluoroethoxy)isoquinolin-1(2H)-one (72-6, 200 mg, 0.6 mmol), (3-methyl-2-(4,4,5,5-tetramethyl-1,3,2-dioxaborolan-2-yl)phenyl)methanol (72-7, 245 mg, 0.9 mmol) and K₃PO₄ (280 mg, 1.3 mmol) in 1,4-dioxane (6 mL) and water (1 mL). The reaction mixture was purged with Argon gas for 10 min. [1,1′-Bis(di-tert-butylphosphino)ferrocene]dichloropalladium(II) (45 mg, 0.06 mmol) was added into the reaction mixture and heated at 80° C. for 16 h. The reaction mixture was cooled to ambient temperature and filtered over celite bed. The filtrate was concentrated under reduced pressure. The product was purified by reverse phase prep HPLC to afford 7-(2,2-difluoroethoxy)-4-(2-(hydroxymethyl)-6-methylphenyl)isoquinolin-1(2H)-one (Example 72, 16 mg). LCMS (ESI) Calcd. for C₁₉H₁₉F₂NO₃: 345, found [M+H]⁺=346. ¹H NMR (400 MHz, DMSO-d₆) δ_{H} 11.41 (d, 1H), 7.75 (s, 1H), 7.43 (d, 1H), 7.37-7.30 (m, 2H), 7.23 (d, 1H), 6.86 (d, 1H), 6.76 (d, 1H), 6.42 (t, 1H), 4.96 (t, 1H), 4.50 (t, 2H), 4.42 (q, 1H), 4.02 (q, 1H), 1.95 (s, 3H).

### Example 73: Synthesis of (R)-N-((S)-1-amino-1-oxopropan-2-yl)-2-((1-oxo-4-(o-tolyl)-1,2-dihydroisoquinolin-7-yl)oxy)propanamide

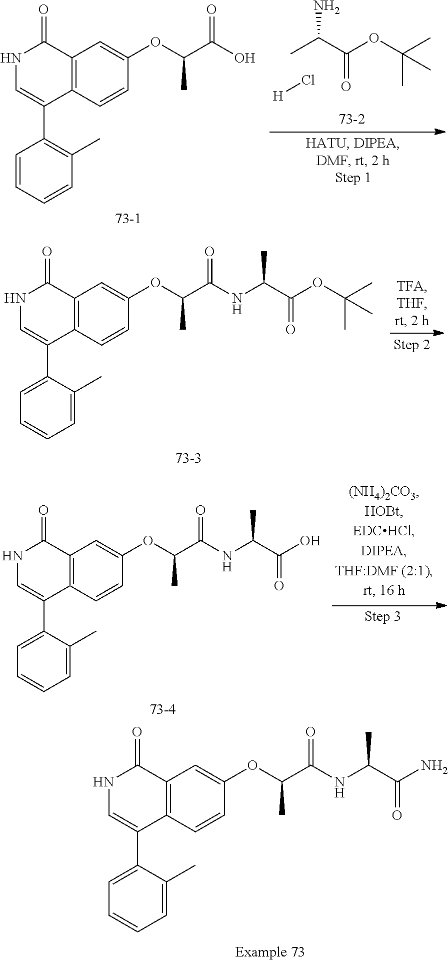

Synthesis of tert-butyl ((R)-2-((1-oxo-4-(o-tolyl)-1,2-dihydroisoquinolin-7-yl)oxy)propanoyl)-L-alaninate, 73-3 [Step 1]: To a stirred solution of (R)-2-((1-oxo-4-(o-tolyl)-1,2-dihydroisoquinolin-7-yl)oxy)propanoic acid (73-1, 170 mg, 0.5 mmol) and tert-butyl L-alaninate hydrochloride (73-2, 115 mg, 0.6 mmol) in dry DMF (3 mL), HATU (240 mg, 0.6 mmol) was added followed by DIPEA (0.3 mL, 1.6 mmol) at 0° C. and stirred at ambient temperature for 2 h. The reaction mixture was diluted with ethyl acetate and washed with ice cold water and brine. The organic extract was collected, dried over anhydrous Na₂SO₄ and concentrated under reduced pressure to afford tert-butyl ((R)-2-((1-oxo-4-(o-tolyl)-1,2-dihydroisoquinolin-7-yl)oxy)propanoyl)-L-alaninate (73-3, 220 mg). LCMS (ESI) Calcd. for C₂₆H₃₀N₂O₅=451, found [M+H]⁺=451.

Synthesis of ((R)-2-((1-oxo-4-(o-tolyl)-1,2-dihydroisoquinolin-7-yl)oxy)propanoyl)-L-alanine, 73-4 [Step 2]: To a stirred solution of tert-butyl ((R)-2-((1-oxo-4-(o-tolyl)-1,2-dihydroisoquinolin-7-yl)oxy)propanoyl)-L-alaninate (73-3, 220 mg, 0.5 mmol) in dichloromethane (5 mL), trifluoroacetic acid (0.9 mL, 12.2 mmol) was added dropwise at 0° C. and stirred at ambient temperature for 2 h. After completion, the reaction mixture was concentrated under reduced pressure to afford ((R)-2-((1-oxo-4-(o-tolyl)-1,2-dihydroisoquinolin-7-yl)oxy)propanoyl)-L-alanine (73-4, 180 mg). LCMS (ESI) Calcd. for C₂₂H₂₂N₂O₅: 394; found [M+H]⁺=395.

Synthesis of (R)-N-((S)-1-amino-1-oxopropan-2-yl)-2-((1-oxo-4-(o-tolyl)-1,2-dihydroisoquinolin-7-yl)oxy)propanamide, Example 73 [Step 3]: To a stirred solution of ((R)-2-((1-oxo-4-(o-tolyl)-1,2-dihydroisoquinolin-7-yl)oxy)propanoyl)-L-alanine (73-4, 220 mg, 0.6 mmol) in THF (4 mL) and DMF (2 mL) mixture, HOBt (115 mg, 0.8 mmol), EDC·HCl (160 mg, 0.8 mmol) and DIPEA (0.4 mL, 2.2 mmol) were added followed by (NH₄)₂CO₃(215 mg, 2.2 mmol) and stirred at ambient temperature for 16 h. After completion, the reaction mixture was diluted with water and extracted with ethyl acetate (twice). The organic layer was collected, washed with brine, dried over anhydrous Na₂SO₄ and, concentrated under reduced pressure. The product was purified by reverse phase prep-HPLC and lyophilized to afford (R)-N-((S)-1-amino-1-oxopropan-2-yl)-2-((1-oxo-4-(o-tolyl)-1,2-dihydroisoquinolin-7-yl)oxy)propanamide (Example 73, 20 mg). LCMS (ESI) Calcd. for C₂₂H₂₃N₃O₄: 393, found [M−H]⁻=392. ¹H NMR (400 MHz, DMSO-d₆): δ_{H} 11.34 (d, 1H), 8.12 (dd, 1H), 7.68 (br s, 1H), 7.35-7.26 (m, 5H), 7.19 (d, 1H), 7.07 (br s, 1H), 6.92-6.89 (m, 2H), 4.91 (q, 1H), 4.21 (q, 1H), 2.03 (s, 3H), 1.46 (d, 3H), 1.17 (d, 3H).

### Example 74. Synthesis of (R)-4-(2-((4-(2-(methyl-d₃)phenyl)-1-oxo-1,2-dihydroisoquinolin-7-yl)oxy)propanoyl)piperazine-1-carboxamide

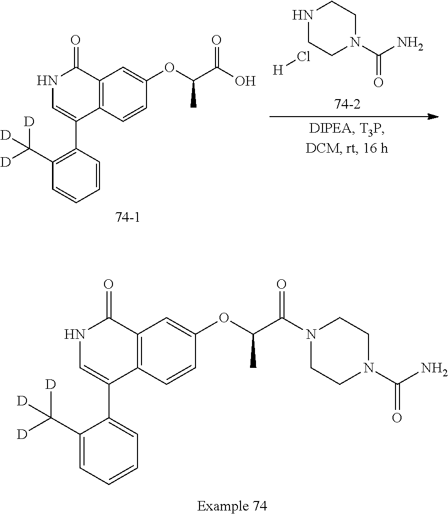

To a stirred solution of (R)-2-((4-(2-(methyl-d₃)phenyl)-1-oxo-1,2-dihydroisoquinolin-7-yl)oxy)propanoic acid (74-1, 270 mg, 0.9 mmol) in dichloromethane (8 mL), piperazine-1-carboxamide hydrochloride (74-2, 205 mg, 1.3 mmol) was added followed by DIPEA (0.6 mL, 3.3 mmol) at 0° C. After 10 min, T₃P (0.8 mL, 1.3 mmol, 50% in ethyl acetate) was added to the reaction mixture at 0° C. and stirred at ambient temperature for 2 h. After completion, the reaction mixture was quenched with ice water and extracted with dichloromethane (twice). The combined organic extract was washed with brine, dried over anhydrous sodium sulphate and concentrated under reduced pressure. The product was purified by reverse phase prep HPLC and lyophilized to afford (R)-4-(2-((4-(2-(methyl-d₃)phenyl)-1-oxo-1,2-dihydroisoquinolin-7-yl)oxy)propanoyl)piperazine-1-carboxamide (Example 74, 150 mg). LCMS (ESI) Calcd. for C₂₄H₂₃D₃N₄O₄: 438, found [M+H]⁺=438. ¹H NMR (400 MHz, DMSO-d₆) δ_{H} 10.94 (br s, 1H), 7.70 (s, 1H), 7.34-7.18 (m, 5H), 6.94 (d, 1H), 6.84 (s, 1H), 5.71 (s, 2H), 5.38-5.33 (q, 1H), 3.54-3.34 (m, 8H), 1.50 (d, 3H).

### Example 75-76: Synthesis of chiral analogs of 7-(((2R)-1-(3-(dimethylamino)azepan-1-yl)-1-oxopropan-2-yl)oxy)-4-(o-tolyl)isoquinolin-1(2H)-one

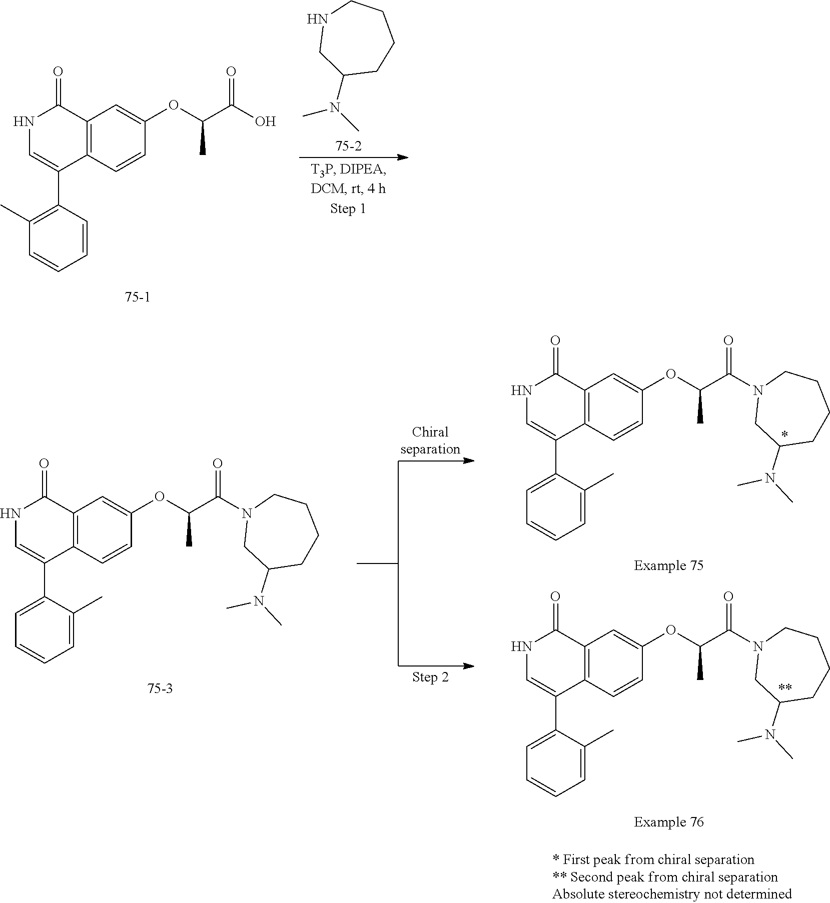

Synthesis of 7-(((2R)-1-(3-(dimethylamino)azepan-1-yl)-1-oxopropan-2-yl)oxy)-4-(o-tolyl)isoquinolin-1(2H)-one, 75-3 [Step 1]: To a stirred solution of (R)-2-((1-oxo-4-(o-tolyl)-1,2-dihydroisoquinolin-7-yl)oxy)propanoic acid (75-1, 220 mg, 0.8 mmol) and N,N-dimethylazepan-3-amine (75-2, 165 mg, 1.2 mmol) in dichloromethane (5 mL), DIPEA (0.6 mL, 3.1 mmol) was added followed by T₃P (0.7 mL, 1.2 mmol, 50% in ethyl acetate) and stirred at ambient temperature for 4 h. After completion, the reaction mixture was diluted with dichloromethane and washed with water (twice). The organic extract was collected, dried with anhydrous Na₂SO₄ and concentrated under reduced pressure to afford 7-(((2R)-1-(3-(dimethylamino)azepan-1-yl)-1-oxopropan-2-yl)oxy)-4-(o-tolyl)isoquinolin-1(2H)-one (75-3, 260 mg). LCMS (ESI) Calcd. for C₂₇H₃₃N₃O₃: 448, found [M+H]⁺=448.

Synthesis of chiral 7-(((2R)-1-(3-(dimethylamino)azepan-1-yl)-1-oxopropan-2-yl)oxy)-4-(o-tolyl)isoquinolin-1(2H)-one, Example 75 and Example 76 [Step 2]: Racemic 7-(((2R)-1-(3-(dimethylamino)azepan-1-yl)-1-oxopropan-2-yl)oxy)-4-(o-tolyl)isoquinolin-1(2H)-one (75-3, 260 mg) was purified by prep-SFC purification and lyophilized to afford the first product as chiral 7-(((2R)-1-(3-(dimethylamino)azepan-1-yl)-1-oxopropan-2-yl)oxy)-4-(o-tolyl)isoquinolin-1(2H)-one (Example 75, 65 mg) as Peak 1 and the second product as chiral 7-(((2R)-1-(3-(dimethylamino)azepan-1-yl)-1-oxopropan-2-yl)oxy)-4-(o-tolyl)isoquinolin-1(2H)-one (Example 76, 42 mg) as Peak 2. The absolute stereochemistry was not determined.

Example 75: Chiral 7-(((2R)-1-(3-(dimethylamino)azepan-1-yl)-1-oxopropan-2-yl)oxy)-4-(o-tolyl)isoquinolin-1(2H)-one (Peak 1): LCMS (ESI) Calcd. for C₂₇H₃₃N₃O₃: 448, [M+H]⁺=448. ¹H NMR (400 MHz, DMSO-d₆ at 100° C.) δ_{H} 11.04 (br s, 1H), 7.70 (s, 1H), 7.34-7.17 (m, 5H), 6.93 (d, 1H), 6.85 (s, 1H), 5.31-5.29 (q, 1H), 3.84 (m, 3H), 3.28 (br s, 2H), 2.81 (br s, 1H), 2.32-2.21 (m, 6H), 2.05 (s, 3H), 1.76 (br s, 3H), 1.52 (s, 4H), 1.26 (br s, 1H).

Example 76: Chiral 7-(((2R)-1-(3-(dimethylamino)azepan-1-yl)-1-oxopropan-2-yl)oxy)-4-(o-tolyl)isoquinolin-1(2H)-one (Peak 2): LCMS (ESI) Calcd. for C₂₇H₃₃N₃O₃: 448, [M+H]⁺=448. ¹H NMR (400 MHz, DMSO-d₆ at 100° C.) δ_{H} 10.98 (br s, 1H), 7.68 (br s, 1H), 7.34-7.18 (m, 5H), 6.92 (d, 1H), 6.84 (s, 1H), 5.30 (br s, 1H), 4.02-4.00 (m, 1H), 3.78 (br s, 2H), 3.39 (br s, 2H), 2.18 (s, 6H), 2.06 (s, 3H), 1.85-1.74 (m, 4H), 1.80 (br s, 5H).

Prep-SFC method: SFC PREP purification was performed using a Pic Solution 175 instrument equipped with a Knauer UV Detector 40D using a Chiralpak-ID column (21.0 mm×250 mm), 5μ, operating at 35° C. temperature and a flow rate of 60 mL/min. The mobile phase used was 55% CO₂ in super critical state and 45% of [0.1% IPAmine in MeOH:ACN(1:1)]. The instrument was run in isocratic mode for up to 10.0 min and at an isobaric condition of 120 bar at a wavelength of 220 nm.

### Example 77: Synthesis of (R)-7-((1-(4-(ethylamino)piperidin-1-yl)-1-oxopropan-2-yl)oxy)-4-(o-tolyl)isoquinolin-1(2H)-one

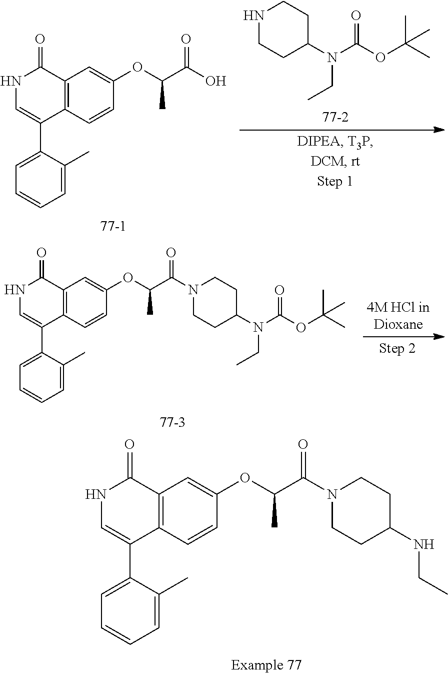 [SMILES: Cc1ccccc1-c1c[nH]c(=O)c2cc(O[C@H](C)C(=O)O)ccc12 ; CCN(C(=O)OC(C)(C)C)C1CCNCC1 ; CCN(C(=O)OC(C)(C)C)C1CCN(C(=O)[C@@H](C)Oc2ccc3c(-c4ccccc4C)c[nH]c(=O)c3c2)CC1 ; CCNC1CCN(C(=O)[C@@H](C)Oc2ccc3c(-c4ccccc4C)c[nH]c(=O)c3c2)CC1]

Synthesis of tert-butyl (R)-ethyl(1-(2-((1-oxo-4-(o-tolyl)-1,2-dihydroisoquinolin-7-yl)oxy)propanoyl)piperidin-4-yl)carbamate, 77-3 [Step 1]: To a stirred solution of (R)-2-((1-oxo-4-(o-tolyl)-1,2-dihydroisoquinolin-7-yl)oxy)propanoic acid (77-1, 120 mg, 0.4 mmol) in dichloromethane (10 mL), tert-butyl ethyl(piperidin-4-yl)carbamate (77-2, 130 mg, 0.6 mmol) was added followed by DIPEA (0.3 mL, 1.5 mmol) at 0° C. After 10 min, T₃P (0.4 mL, 0.6 mmol, 50% in ethyl acetate) was added to the reaction mixture at 0° C. and stirred at ambient temperature for 2 h. After completion, the reaction mixture was quenched with ice water and extracted with dichloromethane (twice). Combined organic extracts was washed with brine, dried over anhydrous sodium sulphate and concentrated under reduced pressure to afford tert-butyl (R)-ethyl(1-(2-((1-oxo-4-(o-tolyl)-1,2-dihydroisoquinolin-7-yl)oxy)propanoyl)piperidin-4-yl)carbamate (77-3, 140 mg). LCMS (ESI) Calcd. for C₃₁H₃₉N₃O₅: 534, found [M+H]⁺=534.

Synthesis of (R)-7-((1-(4-(ethylamino)piperidin-1-yl)-1-oxopropan-2-yl)oxy)-4-(o-tolyl)isoquinolin-1(2H)-one, Example 77 [Step 2]: To a stirred solution of tert-butyl (R)-ethyl(1-(2-((1-oxo-4-(o-tolyl)-1,2-dihydroisoquinolin-7-yl)oxy)propanoyl)piperidin-4-yl)carbamate (77-3, 150 mg, 0.3 mmol) in 1,4-dioxane (2 mL), 4M HCl in dioxane (2.0 mL, 3 mmol) was added dropwise at 0° C. and stirred at ambient temperature for 16 h. After completion, the reaction mixture was concentrated under reduced pressure. The product was purified by reverse phase prep HPLC and lyophilized to afford (R)-7-((1-(4-(ethylamino)piperidin-1-yl)-1-oxopropan-2-yl)oxy)-4-(o-tolyl)isoquinolin-1(2H)-one (Example 77, 70 mg). LCMS (ESI) Calcd. for C₂₆H₃₁N₃O₃: 433, found [M+H]⁺=434. ¹H NMR (400 MHz, DMSO-d₆) δ_{H} 11.31 (br s, 1H), 7.67 (s, 1H), 7.33-7.18 (m, 5H), 6.92 (d, 1H), 6.83 (s, 1H), 5.32-5.30 (q, 1H), 4.06-3.97 (m, 2H), 2.70-2.58 (m, 3H), 2.06 (s, 3H), 1.91-1.78 (m, 5H), 1.48 (d, 3H), 1.21-1.18 (m, 2H), 1.02 (q, 3H).

### Example 78: Synthesis of (R)-N-methyl-N-(2-((1-oxo-4-(o-tolyl)-1,2-dihydroisoquinolin-7-yl)oxy)propanoyl)glycine

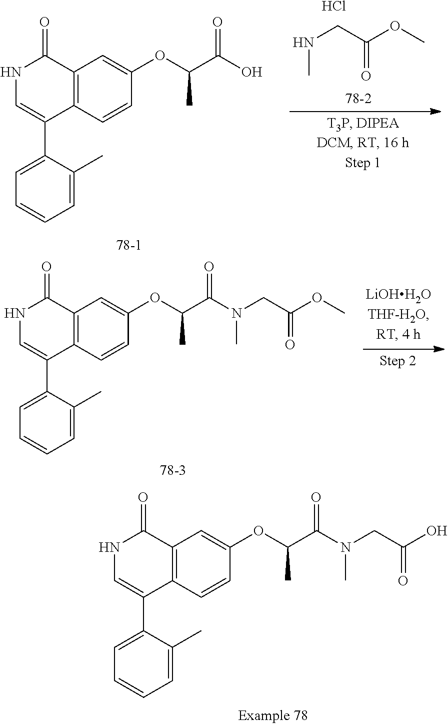

Synthesis of methyl (R)-N-methyl-N-(2-((1-oxo-4-(o-tolyl)-1,2-dihydroisoquinolin-7-yl)oxy)propanoyl)glycinate, 78-3 [Step 1]: To a stirred solution of (R)-2-((1-oxo-4-(0-tolyl)-1,2-dihydroisoquinolin-7-yl)oxy)propanoic acid (78-1, 250 mg, 0.8 mmol) in dichloromethane (5 mL) was added methyl methylglycinate hydrochloride (78-2, 160 mg, 1.2 mmol) and DIPEA (0.40 mL, 2.3 mmol). The reaction mixture was cooled to 0° C. Into this cold reaction mixture, T₃P (0.7 mL, 1.2 mmol, 50% in ethyl acetate) was added and the reaction mixture was allowed to warm up to ambient temperature and stirred for 16 h. After completion, the reaction mixture was quenched with water and extracted with dichloromethane. The combined organic extract was washed with water, brine, dried over anhydrous Na₂SO₄ and concentrated under reduced pressure to afford methyl (R)-N-methyl-N-(2-((1-oxo-4-(o-tolyl)-1,2-dihydroisoquinolin-7-yl)oxy)propanoyl)glycinate (78-3, 160 mg). LCMS (ESI) Calcd. for C₂₃H₂₄N₂O₅: 408, found [M+H]⁺=409.

Synthesis of (R)-N-methyl-N-(2-((1-oxo-4-(o-tolyl)-1,2-dihydroisoquinolin-7-yl)oxy)propanoyl)glycine, Example 78 [Step 2]: To a stirred solution of methyl (R)-N-methyl-N-(2-((1-oxo-4-(o-tolyl)-1,2-dihydroisoquinolin-7-yl)oxy)propanoyl)glycinate (78-3, 160 mg, 0.4 mmol) in THF (4 mL), an aqueous solution (1 mL) of LiOH·H₂O (50 mg, 1.2 mmol) was added at cold condition. The reaction mixture was allowed to warm up to ambient temperature and stirred for 2 h. The reaction mixture was acidified with 1N HCl to pH=5, extracted with ethyl acetate (twice). The combined organic phase was washed with brine, dried over anhydrous Na₂SO₄, concentrated under reduced pressure. The product was purified under reverse phase prep chromatography and lyophilized to afford (R)-N-methyl-N-(2-((1-oxo-4-(0-tolyl)-1,2-dihydroisoquinolin-7-yl)oxy)propanoyl)glycine (Example 78, 64 mg). LCMS (ESI) Calcd. for C₂₂H₂₂N₂O₅: 394, found [M+H]⁺=395. ¹H NMR (400 MHz, DMSO-d₆) δ_{H} 12.75-12.70 (m, 1H), 11.34-11.32 (m, 1H), 7.63-7.60 (m, 1H), 7.35-7.33 (m, 2H), 7.29-7.21 (m, 2H), 7.19-7.17 (m, 1H), 6.89-6.84 (m, 2H), 5.43-5.40 (m, 1H), 432-3.88 (m, 2H), 3.16-2.82 (m, 3H), 2.03 (s, 3H), 1.48-1.40 (m, 3H).

### Example 79. Synthesis of 2-((4-(2-(2-hydroxyethyl)-6-methylphenyl)-1-oxo-1,2-dihydroisoquinolin-7-yl)oxy)acetonitrile

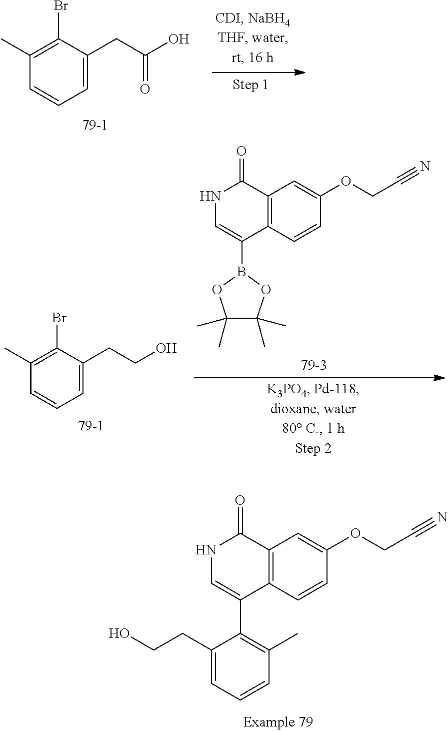

Synthesis of 2-(2-bromo-3-methylphenyl)ethan-1-ol, 2 [Step 1]: To a stirred solution of 2-(2-bromo-3-methylphenyl)acetic acid (79-1, 245 mg, 1.0 mmol) in tetrahydrofuran (3 mL), CDI (190 mg, 1.2 mmol) was added and heated at 40° C. for 1 h. The reaction mixture was cooled to ambient temperature. Then it was poured dropwise into a pre-cooled aqueous solution (1 ml) of NaBH₄ (120 mg, 3.2 mmol) in a round bottom flask. The reaction mixture was allowed to stir for 16 h at ambient temperature. After completion, it was extracted with ethyl acetate (twice). The combined organic extract was washed with water, brine, dried over anhydrous Na₂SO₄ and concentrated under reduced pressure. The product was purified by combi flash column chromatography to afford 2-(2-bromo-3-methylphenyl)ethan-1-ol (79-2, 150 mg). ¹H NMR (400 MHz, DMSO-d₆) δ_{H} 7.19-7.14 (m, 3H), 4.71 (t, 1H), 3.61-3.56 (t, 2H), 2.90-2.86 (t, 2H), 2.35-2.26 (d, 2H).

Synthesis of 2-((4-(2-(2-hydroxyethyl)-6-methylphenyl)-1-oxo-1,2-dihydroisoquinolin-7-yl)oxy)acetonitrile, Example 79 [Step 2]: To a stirred solution of 2(2-bromo-3-methylphenyl)ethan-1-ol (79-2, 100 mg, 0.3 mmol) in 1,4-dioxane (4 mL) and water (1 mL) mixture, 2-((1-oxo-4-(4,4,5,5-tetramethyl-1,3,2-dioxaborolan-2-yl)-1,2-dihydroisoquinolin-7-yl)oxy)acetonitrile (79-3, 100 mg, 0.5 mmol), K₃PO₄ (130 mg, 0.6 mmol) were added and the reaction mixture was purged with Argon gas for 5 min. Into this purged reaction mixture, Pd-118 (20 mg, 0.03 mmol) was added and the reaction mixture was heated at 80° C. for 1 h. The reaction mixture was filtered through celite bed. The filtrate was diluted with ethyl acetate and washed with water, brine, dried over anhydrous Na₂SO₄ and concentrated under reduced pressure. The product was purified by reverse phase prep HPLC purification method and lyophilized to afford 2-((4-(2-(2-hydroxyethyl)-6-methylphenyl)-1-oxo-1,2-dihydroisoquinolin-7-yl)oxy)acetonitrile (Example 79, 8 mg). LCMS (ESI) Calcd. for C₂₀H₁₈N₂O₃: 334, found [M+H]⁺=335. ¹H NMR (400 MHz, DMSO-d₆) δ_{H} 11.48 (br s, 1H), 7.86 (br s, 1H), 7.35-3.33 (m, 1H), 7.29-7.26 (m, 1H), 7.22-7.18 (m, 2H), 6.92 (s, 1H), 6.82-6.79 (m, 1H), 5.32 (s, 2H), 4.48-4.46 (m, 1H), 3.39-3.32 (m, 2H), 2.40-2.32 (m, 2H), 1.94 (s, 3H).

### Example 80: Synthesis of (R)-7-((1-oxo-1-(4-((2,2,2-trifluoroethyl)amino)piperidin-1-yl)propan-2-yl)oxy)-4-(o-tolyl)isoquinolin-1(2H)-one

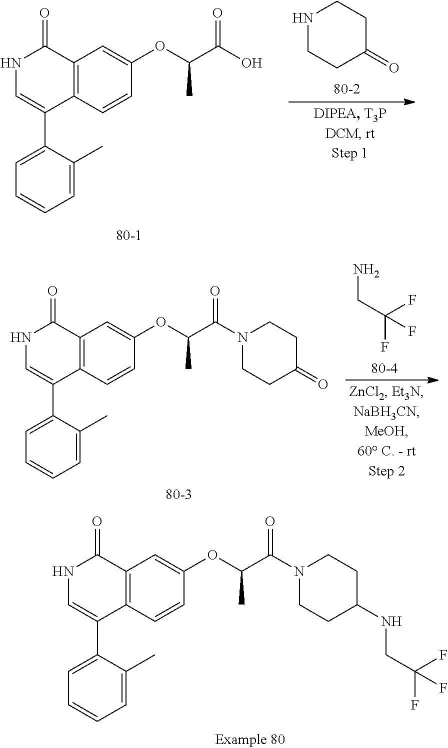 [SMILES: Cc1ccccc1-c1c[nH]c(=O)c2cc(O[C@H](C)C(=O)O)ccc12 ; O=C1CCNCC1 ; Cc1ccccc1-c1c[nH]c(=O)c2cc(O[C@H](C)C(=O)N3CCC(=O)CC3)ccc12 ; NCC(F)(F)F ; Cc1ccccc1-c1c[nH]c(=O)c2cc(O[C@H](C)C(=O)N3CCC(NCC(F)(F)F)CC3)ccc12]

Synthesis of (R)-7-((1-oxo-1-(4-oxopiperidin-1-yl)propan-2-yl)oxy)-4-(o-tolyl)isoquinolin-1(2H)-one, 80-3 [Step 1]: To a stirred solution of (R)-2-((1-oxo-4-(o-tolyl)-1,2-dihydroisoquinolin-7-yl)oxy)propanoic acid (80-1, 100 mg, 0.3 mmol) in dichloromethane (10 mL), piperidin-4-one (80-2, 65 mg, 0.6 mmol) was added followed by DIPEA (0.3 mL, 1.3 mmol) at 0° C. After 10 min, T₃P (0.3 mL, 0.5 mmol, 50% in ethyl acetate) was added to the reaction mixture at 0° C. and stirred at ambient temperature for 2 h. After completion, the reaction mixture was quenched with ice water and extracted with dichloromethane (twice). The combined organic extracts was washed with brine, dried over anhydrous sodium sulphate and concentrated under reduced pressure to afford (R)-7-((1-oxo-1-(4-oxopiperidin-1-yl)propan-2-yl)oxy)-4-(o-tolyl)isoquinolin-1(2H)-one (80-3, 120 mg). LCMS (ESI) Calcd. for C₂₄H₂₄N₂O₄: 404, found [M+H]⁺=405.

Synthesis of (R)-7-((1-oxo-1-(4-((2,2,2-trifluoroethyl)amino)piperidin-1-yl)propan-2-yl)oxy)-4-(o-tolyl)isoquinolin-1(2H)-one, Example 80 [Step 2]: To a stirred solution of (R)-7-((1-oxo-1-(4-oxopiperidin-1-yl)propan-2-yl)oxy)-4-(o-tolyl)isoquinolin-1(2H)-one (80-3, 130 mg, 0.3 mmol) in methanol (6 mL), 2,2,2-trifluoroethanamine (80-4, 35 mg, 0.4 mmol) was added followed by Et₃N (0.15 mL, 1.0 mmol) and ZnCl₂ (5.0 mg, 0.05 mmol) at 0° C. The reaction mixture was then heated at 60° C. for 7 h. NaBH₃CN (60 mg, 1.0 mmol) was added portion wise and the resulting mixture was stirred at ambient temperature for 16 h. After completion, the solvent was evaporated under reduced pressure. The residue was diluted with dichloromethane and washed with water and brine, dried over anhydrous sodium sulfate and concentrated under reduced pressure. The product was purified by reverse phase HPLC and lyophilized to afford (R)-7-((1-oxo-1-(4-((2,2,2-trifluoroethyl)amino)piperidin-1-yl)propan-2-yl)oxy)-4-(o-tolyl)isoquinolin-1(2H)-one (Example 80, 28 mg). LCMS (ESI) Calcd. for C₂₆H₂₈F₃N₃O₃: 488, found [M+H]⁺=488. ¹H NMR (400 MHz, DMSO-d₆): δ_{H} 11.05 (br s, 1H), 7.64 (s, 1H), 7.34-7.19 (m, 5H), 6.92 (d, 1H), 6.84 (s, 1H), 5.35-5.30 (q, 1H), 4.0 (d, 2H), 3.29-3.22 (m, 3H), 2.75 (br s, 1H), 2.15 (br s, 1H), 2.06 (s, 3H), 1.85 (br s, 3H), 1.48 (d, 3H), 1.26-1.19 (m, 2H).

### Example 81: Synthesis of 2-((4-(2-fluoro-6-(2-hydroxyethyl)phenyl)-1-oxo-1,2-dihydroisoquinolin-7-yl)oxy)acetonitrile

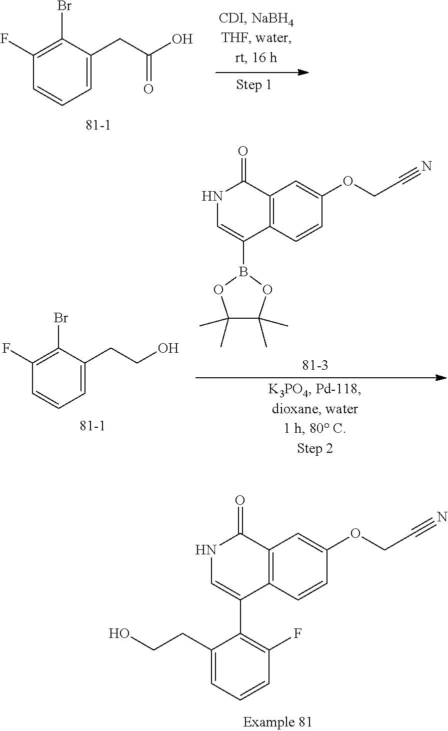

Synthesis of 2-(2-bromo-3-fluorophenyl)ethan-1-ol, 81-2 [Step 1]: To a stirred solution of 2-(2-bromo-3-fluorophenyl)acetic acid (81-1, 250 mg, 1.0 mmol) in tetrahydrofuran (3 mL), CDI (190 mg, 1.2 mmol) was added and heated at 40° C. for 1 h. The reaction mixture was cooled to ambient temperature. It was poured dropwise into a pre-cooled aqueous (1 ml) solution of NaBH₄ (120 mg, 3.2 mmol) in a round bottom flask. The reaction mixture was stirred at ambient temperature for 16 h. After completion, the reaction mixture was extracted with ethyl acetate, washed with water, brine, dried over anhydrous Na₂SO₄ and concentrated under reduced pressure. The product was purified by flash column chromatography to afford 2-(2-bromo-3-fluorophenyl)ethan-1-ol (81-2, 60 mg). ¹H NMR (400 MHz, DMSO-d₆) δ_{H} 7.36-7.31 (m, 1H), 7.22-7.18 (m, 2H), 4.79-4.77 (m, 2H), 3.62-3.58 (m, 2H).

Synthesis of 2-((4-(2-fluoro-6-(2-hydroxyethyl)phenyl)-1-oxo-1,2-dihydroisoquinolin-7-yl)oxy)acetonitrile, Example 81 [Step 2]: To a stirred solution of 2-(2-bromo-3-fluorophenyl)ethan-1-ol (81-2, 100 mg, 0.5 mmol) in 1,4-dioxane (3 mL) and water (0.5 mL), 2-((1-oxo-4-(4,4,5,5-tetramethyl-1,3,2-dioxaborolan-2-yl)-1,2-dihydroisoquinolin-7-yl)oxy)acetonitrile (81-3, 100 mg, 0.3 mmol) and K₃PO₄ (130 mg, 0.6 mmol). The reaction mixture was purged with Argon gas for 5 min. Pd-118 (20 mg, 0.03 mmol) was added into the reaction mixture and stirred at 80° C. for 1 h. The reaction mixture was filtered through celite bed. The filtrate was diluted with ethyl acetate and was washed with water, brine, dried over anhydrous Na₂SO₄ and concentrated under reduced pressure. The product was purified under reverse phase prep HPLC purification method and lyophilized to afford 2-((4-(2-fluoro-6-(2-hydroxyethyl)phenyl)-1-oxo-1,2-dihydroisoquinolin-7-yl)oxy)acetonitrile (Example 81, 20 mg). LCMS (ESI) Calcd. for C₁₉H₁₅FN₂O₃: 338, found [M+H]⁺=339. ¹H NMR (400 MHz, DMSO-d₆) δ_{H} 11.59-11.57 (m, 1H), 7.86-7.85 (m, 1H), 7.46-7.42 (m, 1H), 7.39-7.36 (m, 1H), 7.27 (d, 1H), 7.19-7.14 (m, 1H), 7.08-7.07 (m, 1H), 6.94-6.91 (m, 1H), 5.32 (s, 2H), 4.55-4.53 (m, 1H), 3.41-3.34 (m, 2H), 2.66 (br s, 1H), 2.49-2.44 (m, 1H).

### Example 82: Synthesis of 2-((4-(2-(2-hydroxypropyl)phenyl)-1-oxo-1,2-dihydroisoquinolin-7-yl)oxy)acetamide

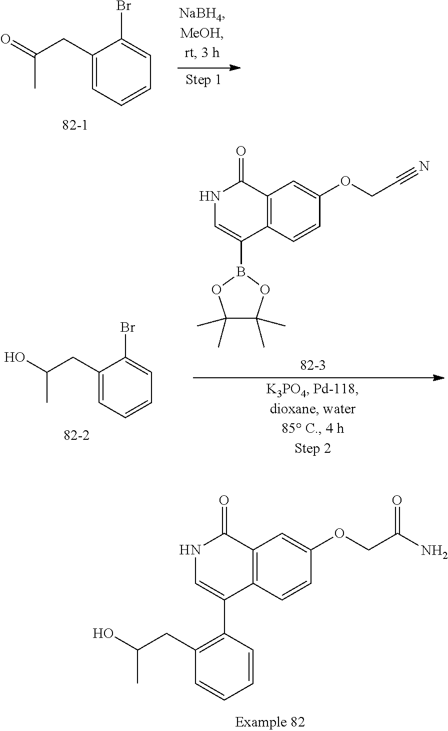 [SMILES: CC(=O)Cc1ccccc1Br ; CC(O)Cc1ccccc1Br ; CC1(C)OB(c2c[nH]c(=O)c3cc(OCC#N)ccc23)OC1(C)C ; CC(O)Cc1ccccc1-c1c[nH]c(=O)c2cc(OCC(N)=O)ccc12]

Synthesis of 1-(2-bromophenyl)propan-2-ol, 82-2 [Step 1]: To a stirred solution of 1-(2-bromophenyl)propan-2-one (82-1, 1.0 g, 4.7 mmol) in methanol (40 mL), sodium borohydride (215 mg, 5.6 mmol) was added portion wise at 0° C. The reaction mixture was stirred at ambient temperature for 3 h. After completion, the reaction mixture was quenched with 10% HCl solution and concentrated under reduced pressure. The reduced mass was diluted with ethyl acetate and washed with brine. The organic extract was dried over anhydrous Na₂SO₄ and concentrated under reduced pressure to afford 1-(2-bromophenyl)propan-2-ol (82-2, 850 mg). ¹H NMR (400 MHz, DMSO-d₆) δ_{H} 7.55 (t, 11H), 7.44-6.95 (m, 3H), 4.64-4.32 (m, 1H), 3.92-3.82 (m, 1H), 2.83-2.60 (m, 2H), 1.09-0.96 (m, 3H).

Synthesis of 2-((4-(2-(2-hydroxypropyl)phenyl)-1-oxo-1,2-dihydroisoquinolin-7-yl)oxy)acetamide, Example 82 [Step 2]: To a stirred solution of 2-((1-oxo-4-(4,4,5,5-tetramethyl-1,3,2-dioxaborolan-2-yl)-1,2-dihydroisoquinolin-7-yl)oxy)acetonitrile (82-3, 100 mg, 0.3 mmol) and 1-(2-bromophenyl)propan-2-ol (82-2, 100 mg, 0.5 mmol) in 1,4-dioxane (4 mL) and water (1 mL), KPO₄ (130 mg, 0.6 mmol) was added. Then nitrogen gas was purged into the reaction mixture for 10 min. [1,1′-Bis(di-tert-butylphosphino)ferrocene]dichloropalladium(II) (20 mg, 0.03 mmol) was added into the mixture and heated at 85° C. for 4 h. After completion, the mixture was diluted with ethyl acetate and washed with brine. The organic extract was dried over anhydrous Na₂SO₄, filtered and concentrated under reduced pressure. The product was purified by reverse phase prep HPLC to afford 2-((4-(2-(2-hydroxypropyl)phenyl)-1-oxo-1,2-dihydroisoquinolin-7-yl)oxy)acetamide (Example 82, 50 mg). LCMS (ESI) Calcd. for C₂₀H₂₀N₂O₄: 352, found [M+H]⁺=353. ¹H NMR (400 MHz, DMSO-d₆) 11.36 (s, 1H), 7.67-7.63 (m, 2H), 7.39-7.36 (m, 3H), 7.32-7.27 (m, 2H), 7.18-7.15 (m, 1H), 6.92-6.89 (m, 2H), 4.55 (s, 2H), 4.43 (d, 1H), 3.80-3.50 (m, 1H), 2.67-2.64 (m, 1H), 2.34-2.32 (m, 1H), 0.86-0.79 (m, 3H).

### Example 83: Synthesis of 2-(7-(cyanomethoxy)-1-oxo-1,2-dihydroisoquinolin-4-yl)-N,N,3-trimethylbenzamide

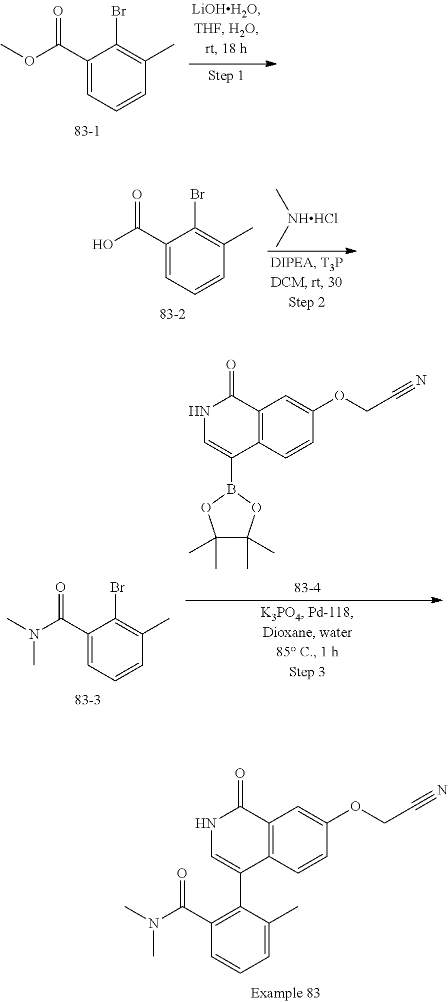

Synthesis of 2-bromo-3-methylbenzoic acid, 83-2 [Step 1]: To a stirred solution of methyl 2-bromo-3-methylbenzoate (83-1, 4.6 g, 20.1 mmol) in THF (50 mL) and water (10 mL), LiOH·H2O (2.5 g, 60.2 mmol) was added at 0° C. The mixture was stirred at ambient temperature for 18 h. The reaction mixture was concentrated under reduced pressure and the residue was acidified with 2N HCl to pH=4. The precipitate observed was filtered, washed with water, hexane and dried under reduced pressure to afford 2-bromo-3-methylbenzoic acid (83-2, 3.8 g). ¹H NMR (400 MHz, DMSO-d₆) δ 13.35 (s, 1H), 7.48-7.42 (m, 2H), 7.34 (t, 1H), 2.39 (s, 3H).

Synthesis of 2-bromo-N,N,3-trimethylbenzamide, 83-3 [Step 2]: To a stirred solution of 2-bromo-3-methylbenzoic acid (83-2, 1.0 g, 4.7 mmol) and N-methylmethanamine hydrochloride (2.7 g, 32.6 mmol) in dichloromethane (30 mL), N,N-Diisopropylethylamine (6.5 mL, 37.2 mmol) was added. The mixture was cooled to 0° C. and stirred for 10 min. T₃P (4.2 mL, 6.9 mmol, 50% in ethyl acetate) was added dropwise at ice cold condition and the mixture was stirred at ambient temperature for 30 h. The reaction mixture was diluted with water and extracted with dichloromethane (twice). The combined organic extract was washed with saturated NaHCO₃ solution, dried over anhydrous Na₂SO₄ and concentrated under reduced pressure. The product was purified by column chromatography to afford 2-bromo-N,N,3-trimethylbenzamide (83-3, 700 mg). LCMS (ESI) Calcd. for C₁₀H₁₂BrNO: 241, found [M+H]⁺=242. ¹H NMR (400 MHz, DMSO-d₆) δ_{H} 7.38-7.32 (m, 2H), 7.10 (d, 1H), 2.99 (s, 3H), 2.74 (s, 3H), 2.38 (s, 3H).

Synthesis of 2-(7-(cyanomethoxy)-1-oxo-1,2-dihydroisoquinolin-4-yl)-N,N,3-trimethylbenzamide. Example 83 [Step 3]: To a stirred solution of 2-((1-oxo-4-(4,4,5,5-tetramethyl-1,3,2-dioxaborolan-2-yl)-1,2-dihydroisoquinolin-7-yl)oxy)acetonitrile (83-4, 150 mg, 0.5 mmol) and 2-bromo-N,N,3-trimethylbenzamide (83-3, 170 mg, 0.7 mmol) in 1,4-dioxane (4 mL) and water (1 mL), K₃PO₄ (195 mg, 0.9 mmol) was added. Then nitrogen gas was purged for 10 min. [1,1′-Bis(di-tert-butylphosphino)ferrocene]dichloropalladium(II) (30 mg, 0.05 mmol) was added into the mixture and heated at 85° C. for 1 h. After completion, the mixture was diluted with ethyl acetate and washed with brine. The organic extract was dried over anhydrous Na₂SO₄, filtered and concentrated under reduced pressure. The product was purified by reverse phase prep HPLC to afford 2-(7-(cyanomethoxy)-1-oxo-1,2-dihydroisoquinolin-4-yl)-N,N,3-trimethylbenzamide (Example 83, 8 mg). LCMS (ESI) Calcd. for C₂₁H₁₉N₃O₃: 361, found [M+H]⁺=362. ¹H NMR (400 MHz, DMSO-d₆) δ_{H} 11.37 (s, 1H), 7.81 (s, 1H), 7.43-7.38 (m, 2H), 7.35 (dd, 1H), 7.17 (d, 1H), 6.98 (br s, 1H), 6.86 (d, 1H), 5.32 (s, 2H), 2.69-2.62 (m, 6H), 1.99 (s, 3H).

### Example 84-85: Synthesis of chiral analogs of 7-(((2R)-1-(4-(dimethylamino)azepan-1-yl)-1-oxopropan-2-yl)oxy)-4-(o-tolyl)isoquinolin-1(2H)-one

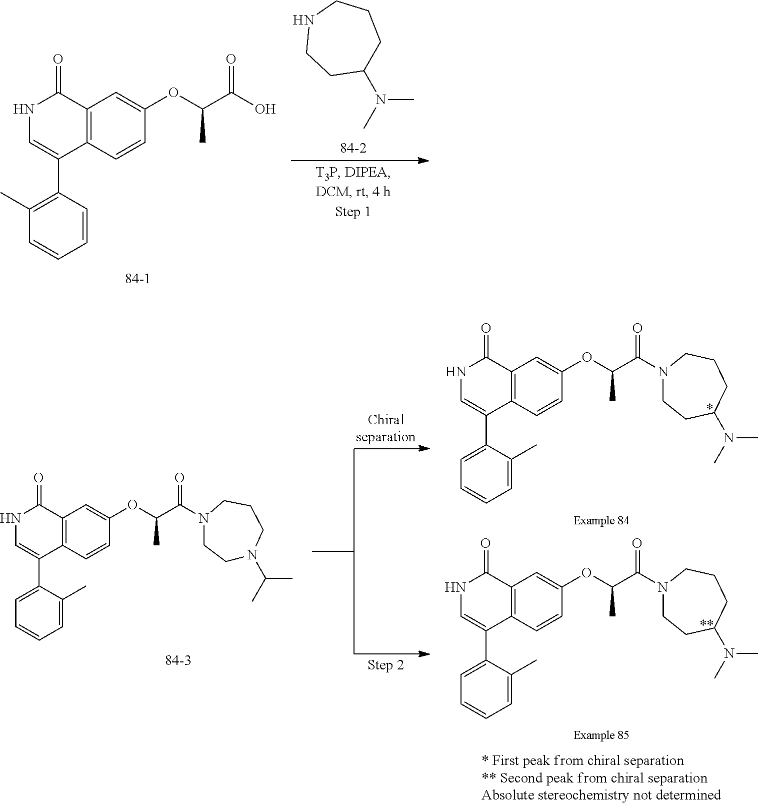

Synthesis of 7-(((2R)-1-(4-(dimethylamino)azepan-1-yl)-1-oxopropan-2-yl)oxy)-4-(o-tolyl)isoquinolin-1(2H)-one, 84-3 [Step 1]: To a stirred solution of (R)-2-((1-oxo-4-(o-tolyl)-1,2-dihydroisoquinolin-7-yl)oxy)propanoic acid (84-1, 250 mg, 0.8 mmol) and N,N-dimethylazepan-4-amine (84-2, 165 mg, 1.2 mmol) in dichloromethane (5 mL), DIPEA (0.6 mL, 3.1 mmol) was added followed by T₃P (0.7 mL, 1.2 mmol, 50% in ethyl acetate) and stirred at ambient temperature for 4 h. After completion, the reaction mixture was diluted with dichloromethane and washed with water (twice). The combined organic extract was collected, dried with anhydrous Na₂SO₄ and concentrated under reduced pressure to afford 7-(((2R)-1-(4-(dimethylamino)azepan-1-yl)-1-oxopropan-2-yl)oxy)-4-(o-tolyl)isoquinolin-1(2H)-one (84-3, 260 mg). LCMS (ESI): Calcd. for C₂₇H₃₃N₃O₃: 448, found [M+H]⁺=448.

Synthesis of chiral 7-(((2R)-1-(4-(dimethylamino)azepan-1-yl)-1-oxopropan-2-yl)oxy)-4-(o-tolyl)isoquinolin-1(2H)-one, Example 84 and Example 85 [Step 2]: Racemic 7-(((2R)-1-(4-(dimethylamino)azepan-1-yl)-1-oxopropan-2-yl)oxy)-4-(o-tolyl)isoquinolin-1(2H)-one (84-3, 260 mg) was purified by prep-SFC purification and lyophilized to afford the first product as chiral 7-(((2R)-1-(4-(dimethylamino)azepan-1-yl)-1-oxopropan-2-yl)oxy)-4-(o-tolyl)isoquinolin-1(2H)-one, (Example 84, 45 mg) as Peak 1 and the second product as chiral 7-(((2R)-1-(4-(dimethylamino)azepan-1-yl)-1-oxopropan-2-yl)oxy)-4-(o-tolyl)isoquinolin-1(2H)-one (Example 85, 32 mg) as Peak 2. The absolute stereochemistry were not determined for these compounds.

Prep-SFC method: SFC PREP PURIFICATION was performed using a Pic Solution instrument equipped with a Knauer UV Detector 40D and a Chiralcel OX-H (21 mm×250 mm), 5μ, operating at 35° C. temperature with a flow rate of 50 ml/min. The mobile phase used with a mixture of 65% CO₂ in super critical state and 35% of (0.3% IPAmine in methanol). The instrument was held in isocratic mode for up to 22.0 minutes and at an isobaric condition of 120 bar at a wavelength of 230 nm.

Example 84: Chiral 7-(((2R)-1-(4-(dimethylamino)azepan-1-yl)-1-oxopropan-2-yl)oxy)-4-(o-tolyl)isoquinolin-1(2H)-one (Peak 1): LCMS (ESI) Calcd. for C₂₇H₃₃N₃O₃: 448, found [M+H]⁺=448. ¹H NMR (400 MHz, DMSO-d₆ at 100° C.) δ_{H} 10.95 (br s, 1H), 7.69 (s, 1H), 7.33-7.17 (m, 5H), 6.92 (d, 1H), 6.82 (s, 1H), 5.29-5.25 (q, 1H), 3.64 (br s, 3H), 3.21 (br s, 1H), 2.13 (s, 6H), 2.06 (s, 3H), 1.84-1.74 (m, 4H), 1.50 (d, 6H).

Example 85: Chiral 7-(((2R)-1-(4-(dimethylamino)azepan-1-yl)-1-oxopropan-2-yl)oxy)-4-(o-tolyl)isoquinolin-1(2H)-one (Peak 2): LCMS (ESI) Calcd. for C₂₇H₃₃N₃O₃: 448, found [M+H]⁺=448. ¹H NMR (400 MHz, DMSO-d₆ at 100° C.) δ_{H} 10.95 (br s, 1H), 7.71 (s, 1H), 7.33-7.16 (m, 5H), 6.92 (d, 1H), 6.82 (s, 1H), 5.22-5.25 (q, 1H), 3.70 (br s, 2H), 3.42 (br s, 4H), 2.10-2.06 (m, 8H), 1.81-1.72 (m, 4H), 1.50 (d, 3H), 1.37 (br s, 2H).

### Example 86 and 87: Synthesis of chiral analogs of 2-((4-(2-(hydroxymethyl)-6-methylphenyl)-1-oxo-1,2-dihydroisoquinolin-7-yl)oxy)propanenitrile

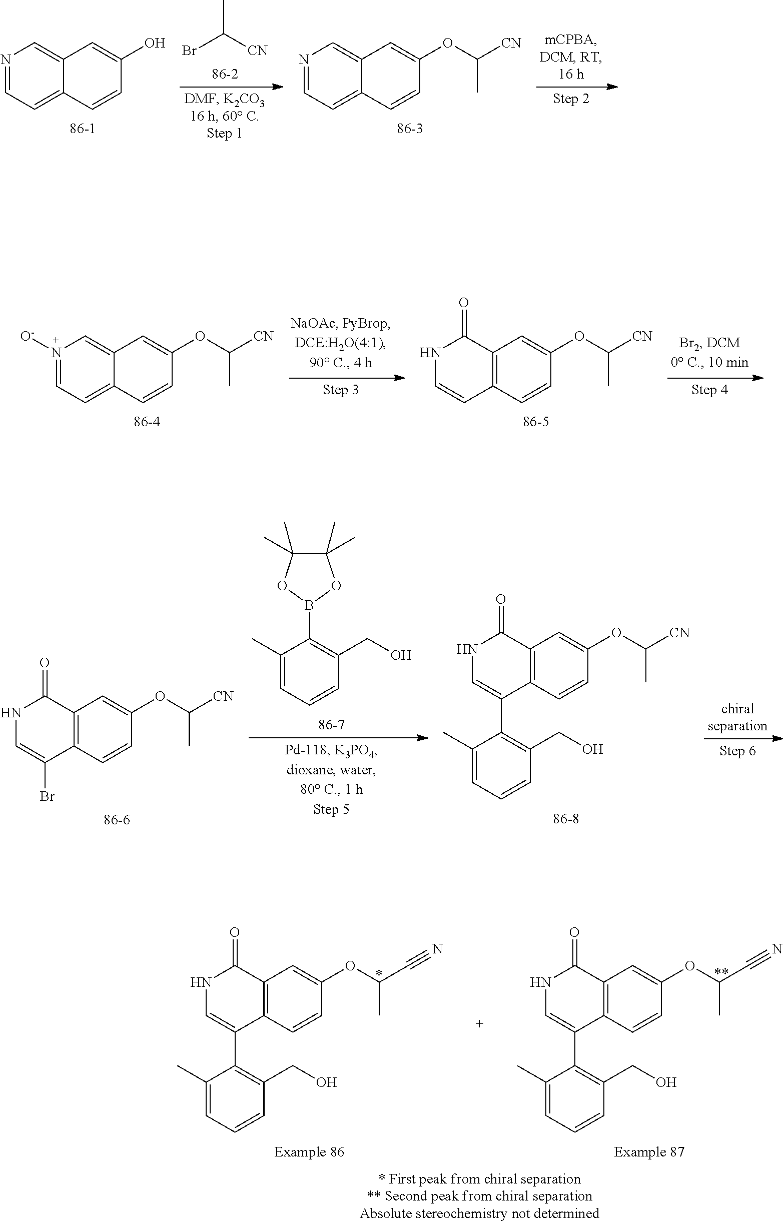

Synthesis of 2-(isoquinolin-7-yloxy)propanenitrile, 86-3 [Step 1]: To a stirred solution of isoquinolin-7-ol (86-1, 1.50 g, 10.3 mmol) and K₂CO₃(6.58 g, 31.0 mmol) in DMF (50 mL), 2-bromopropanenitrile (86-2, 1.8 g, 13.4 mmol) was added and the reaction mixture was heated at 60° C. for 16 h. After completion, the reaction mixture was diluted with water and extracted with ethyl acetate (twice). The combined organic extract was washed with water, brine, dried over anhydrous Na₂SO₄, and concentrated under reduced pressure to afford 2-(7-isoquinolyloxy)propanenitrile (86-3, 1.8 g). LCMS (ESI) Calcd. for C₁₂H₁₀N₂O: 198, found [M+H]⁺=199.

Synthesis of 7-(1-cyanoethoxy)isoquinoline 2-oxide, 86-4 [Step 2]: To a stirred solution of 2-(7-isoquinolyloxy)propanenitrile (86-3, 2 g, 10.1 mmol) in dichloromethane (50 mL), mCPBA (3.7 g, 15.1 mmol) was added portion-wise at 0° C. and stirred at ambient temperature for 16 h. After completion, the reaction mixture was quenched with saturated NaHCO₃ solution and extracted with dichloromethane (twice). The combined organic extract was washed with brine, dried over anhydrous Na₂SO₄, concentrated under reduced pressure to afford 7-(1-cyanoethoxy)isoquinoline 2-oxide (86-4, 1.5 g). LCMS (ESI) Calcd. for C₁₂H₁₀N₂O₂: 214, found [M+H]⁺=215.

Synthesis of 2-((1-oxo-1,2-dihydroisoquinolin-7-yl)oxy)propanenitrile, 86-5 [Step 3]: To a stirred solution of 7-(1-cyanoethoxy)isoquinoline 2-oxide (86-4, 3.5 g, 16.3 mmol) in dichloroethane (80 mL) and water (20 mL) mixture, NaOAc (2.7 g, 32.7 mmol) and PyBroP (15.2 g, 32.7 mmol) were added at ambient temperature and heated at 90° C. for 4 h. After completion, the reaction mixture was cooled to ambient temperature and extracted with dichloromethane (twice). The combined organic extract was washed with brine, dried over anhydrous Na₂SO₄ and concentrated under reduced pressure and the crude was purified by flash column chromatography to afford 2-((1-oxo-1,2-dihydroisoquinolin-7-yl)oxy)propanenitrile (86-5, 2.5 g). LCMS (ESI) Calcd. for C₁₂H₁₀N₂O₂: 214, found [M+H]⁺=215.

Synthesis of 2-((4-bromo-1-oxo-1,2-dihydroisoquinolin-7-yl)oxy)propanenitrile, 86-6 [Step 4]: To a stirred solution of 2-((1-oxo-1,2-dihydroisoquinolin-7-yl)oxy)propanenitrile (86-5, 900 mg, 4.2 mmol) in dichloromethane (40 mL), a solution of bromine (0.3 mL, 4.6 mmol) in dichloromethane (5 mL) was added dropwise at 0° C. and stirred at same temperature for 10 min. After completion, the reaction mixture was quenched with saturated Na₂S₂O. solution and extracted with dichloromethane (twice). The combined organic extract was washed with brine, dried over anhydrous Na₂SO₄ and concentrated under reduced pressure to afford 2-((4-bromo-1-oxo-1,2-dihydroisoquinolin-7-yl)oxy)propanenitrile (86-6, 850 mg). LCMS (ESI) Calcd. for C₁₂H₉BrN₂O₂: 293, found [M+2H]⁻=295.

Synthesis of 2-((4-(2-(hydroxymethyl)-6-methylphenyl)-1-oxo-1,2-dihydroisoquinolin-7-yl)oxy)propanenitrile, 86-8 [Step 5]: A sealed tube was charged with 2-((4-bromo-1-oxo-1,2-dihydroisoquinolin-7-yl)oxy)propanenitrile (86-6, 300 mg, 1.0 mmol), (3-methyl-2-(4,4,5,5-tetramethyl-1,3,2-dioxaborolan-2-yl)phenyl)methanol (7, 380 mg, 1.5 mmol) and K₃PO₄ (435 mg, 2.0 mmol) in 1,4-dioxane (6 mL) and water (1 mL) mixture. The reaction mixture was purged with Argon gas for 10 min. [1,1′-Bis(di-tert-butylphosphino)ferrocene]dichloropalladium(II) (65 mg, 0.1 mmol) was added into the reaction mixture and heated at 80° C. for 1 h. The reaction mixture was cooled to ambient temperature and filtered over celite bed. The filtrate was concentrated under reduced pressure and purified by reverse phase prep HPLC to afford 2-((4-(2-(hydroxymethyl)-6-methylphenyl)-1-oxo-1,2-dihydroisoquinolin-7-yl)oxy)propanenitrile 7 (86-8, 80 mg). LCMS (ESI) Calcd. for C₂₀H₁₈N₂O₃: 334, found [M+H]⁺=335.

Synthesis of chiral 2-((4-(2-(hydroxymethyl)-6-methylphenyl)-1-oxo-1,2-dihydroisoquinolin-7-yl)oxy)propanenitrile, Example 86 and Example 87 [Step 6]: Racemic 2-((4-(2-(hydroxymethyl)-6-methylphenyl)-1-oxo-1,2-dihydroisoquinolin-7-yl)oxy)propanenitrile (86-8, 80 mg) was separated by SFC HPLC chiral purification and lyophilized to afford the first product as chiral 24(4-(2-(hydroxymethyl)-6-methylphenyl)-1-oxo-1,2-dihydroisoquinolin-7-yl)oxy)propanenitrile (Example 86, 20 mg) as Peak 1 and the second product as chiral 2-((4-(2-(hydroxymethyl)-6-methylphenyl)-1-oxo-1,2-dihydroisoquinolin-7-yl)oxy)propanenitrile (Example 87, 21 mg) as Peak 2. The absolute stereochemistry of these compounds were not determined.

Example 86: Chiral 2-((4-(2-(hydroxymethyl)-6-methylphenyl)-1-oxo-1,2-dihydroisoquinolin-7-yl)oxy)propanenitrile, Peak 1: LCMS (ESI) Calcd. for C₂₀H₁₈N₂O₃: 334, found [M+H]⁺=335. ¹H NMR (400 MHz, DMSO-d₆) δ_{H} 11.48 (s, 1H), 7.89 (d, 1H), 7.43 (d, 1H), 7.37-7.32 (m, 2H), 7.24 (d, 1H), 6.91 (d, 1H), 6.81 (d, 1H), 5.63 (q, 1H), 4.96 (t, 1H), 4.27-4.21 (m, 1H), 4.07-4.00 (m, 1H), 1.96 (d, 3H), 1.70 (d, 3H).

Example 87: Chiral 2-((4-(2-(hydroxymethyl)-6-methylphenyl)-1-oxo-1,2-dihydroisoquinolin-7-yl)oxy)propanenitrile, Peak 2: LCMS (ESI) Calcd. for C₂₀H₁₈N₂O₃: 334, found [M+H]⁺=335. ¹H NMR (400 MHz, DMSO-d₆) δ_{H} 11.48 (s, 1H), 7.89 (d, 1H), 7.43 (d, 1H), 7.37-7.32 (m, 2H), 7.24 (d, 1H), 6.91 (d, 1H), 6.81 (d, 1H), 5.63 (q, 1H), 4.96 (t, 1H), 4.27-4.21 (m, 1H), 4.07-4.00 (m, 1H), 1.96 (d, 3H), 1.70 (d, 3H).

SFC chiral HPLC method: Chiral separation was performed using a THAR SFC 80 instrument with a Waters UV Detector 2489 using a (R,R) WHELK-Ol column (21.1 mm×250 mm), 5μ, operating at 35° C. temperature with a flow rate of 60 g/min. The mobile phase used was a mixture of 70% CO₂ in super critical state and 30% of (100% MeOH). The instrument was held in isocratic mode for up to 7.5 minutes and at an isobaric condition of 100 bar at a wavelength of 210 nm.

### Example 88. Synthesis of (R)-N-((R)--amino-1-oxopropan-2-yl)-N-methyl-2-((1-oxo-4-(o-tolyl)-1,2-dihydroisoquinolin-7-yl)oxy)propanamide

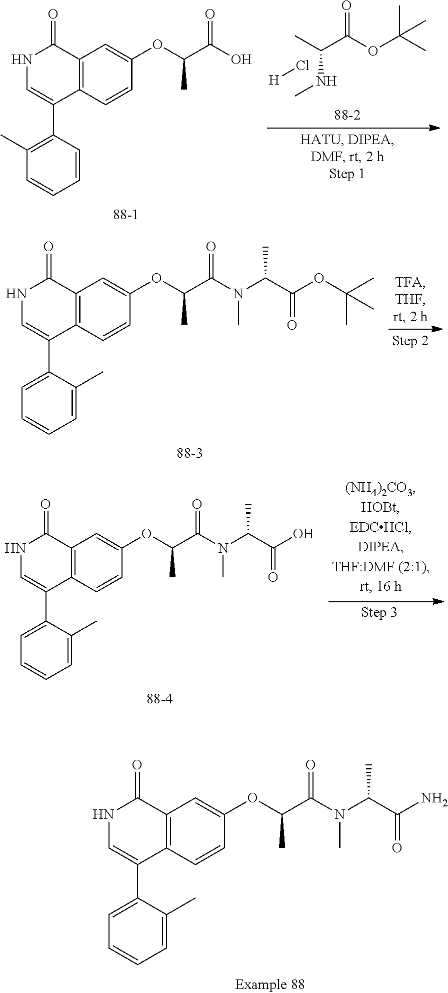

Synthesis of tert-butyl N-methyl-N-((R)-2-((1-oxo-4-(o-tolyl)-1,2-dihydroisoquinolin-7-yl)oxy)propanoyl)-D-alaninate, 88-3 [Step 1]: To a stirred solution of (R)-2-((1-oxo-4-(o-tolyl)-1,2-dihydroisoquinolin-7-yl)oxy)propanoic acid (88-1, 180 mg, 0.6 mmol) and tert-butyl methyl-D-alaninate hydrochloride (88-2, 165 mg, 0.8 mmol) in DMF (2 mL), HATU (255 mg, 0.7 mmol) was added followed by DIPEA (0.3 mL, 1.7 mmol) at 0° C. and stirred at ambient temperature for 2 h. The reaction mixture was diluted with ethyl acetate and washed with ice cold water and brine. The organic extracts was collected, dried over anhydrous Na₂SO₄ and concentrated under reduced pressure to afford tert-butyl N-methyl-N-((R)-2-((1-oxo-4-(o-tolyl)-1,2-dihydroisoquinolin-7-yl)oxy)propanoyl)-D-alaninate (88-3, 230 mg). LCMS (ESI) Calcd. for C₂₇H₃₂N₂O₅: 465, found [M+H]⁺=465.

Synthesis of N-methyl-N-((R)-2-((1-oxo-4-(o-tolyl)-1,2-dihydroisoquinolin-7-yl)oxy)propanoyl)-D-alanine, 88-4 [Step 2]: To a stirred solution of tert-butyl N-methyl-N-((R)-2-((1-oxo-4-(o-tolyl)-1,2-dihydroisoquinolin-7-yl)oxy)propanoyl)-D-alaninate (88-3, 230 mg, 0.5 mmol) in dichloromethane (5 mL), trifluoroacetic acid (1.0 mL, 12.4 mmol) was added and stirred at ambient temperature for 16 h. After completion, the reaction mixture was completely dried under reduced pressure to afford N-methyl-N-((R)-2-((1-oxo-4-(o-tolyl)-1,2-dihydroisoquinolin-7-yl)oxy)propanoyl)-D-alanine (88-4, 190 mg). LCMS (ESI) Calcd. for C₂₃H₂₄N₂O₅: 408, found [M+H]⁺=409.

Synthesis of (R)-N-((R)-1-amino-1-oxopropan-2-yl)-N-methyl-2-((1-oxo-4-(o-tolyl)-1,2-dihydroisoquinolin-7-yl)oxy)propanamide, Example 88 [Step 3]: To a stirred solution of N-methyl-N-((R)-2-((1-oxo-4-(o-tolyl)-1,2-dihydroisoquinolin-7-yl)oxy)propanoyl)-D-alanine (88-4, 180 mg, 0.4 mmol) in THF (6 mL) and DMF (3 mL), HOBt (90 mg, 0.6 mmol), EDC·HCl (125 mg, 0.6 mmol) and DIPEA (0.3 mL, 1.7 mmol) were added followed by (NH₄)₂CO₃ (170 mg, 1.7 mmol) and stirred at ambient temperature for 16 h. After completion, the reaction mixture was diluted with ethyl acetate and washed with ice cold water. The organic extract was collected, washed with brine, dried over anhydrous Na₂SO₄ and concentrated under reduced pressure. The product was purified by reverse phase prep-HPLC and isolated compound was lyophilized to afford (R)-N-((R)-1-amino-1-oxopropan-2-yl)-N-methyl-2-((1-oxo-4-(o-tolyl)-1,2-dihydroisoquinolin-7-yl)oxy)propanamide (Example 88.40 mg). LCMS (ESI) Calcd. for C₂₃H₂₅N₃O₄: 407, found [M+H]⁺=408. ¹H NMR (400 MHz, DMSO-d₆) δ_{H} 11.31 (d, 1H), 7.51 (br s, 1H), 7.34 (br s, 2H), 7.28-7.19 (m, 4H), 7.05 (br s, 1H), 6.90-6.88 (m, 2H), 5.35 (d, 1H), 4.90 (d, 1H), 3.03 (s, 3H), 2.03 (s, 3H), 1.47 (d, 3H), 1.27 (d, 3H).

### Example 89: Synthesis of N-(2-(7-(cyanomethoxy)-1-oxo-1,2-dihydroisoquinolin-4-yl)phenethyl)acetamide

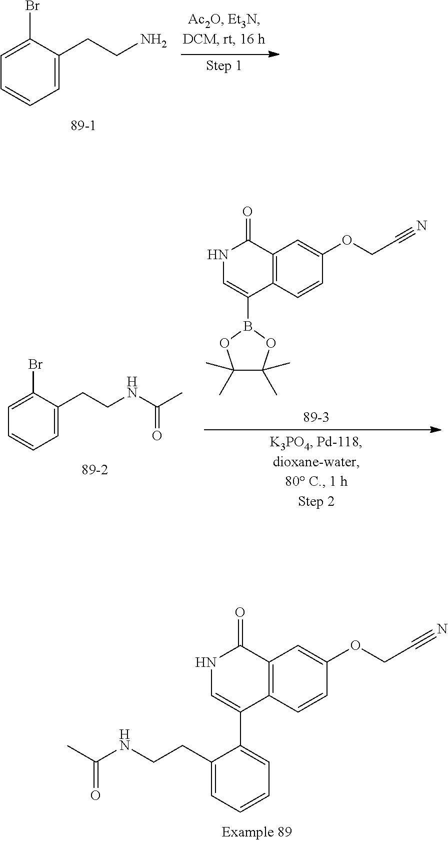

Synthesis of N-(2-bromophenethyl)acetamide, 89-2 [Step 3]: A stirred solution of 2-(2-bromophenyl)ethan-1-amine (89-1, 250 mg, 1.3 mmol) in dichloromethane (4 mL) was cooled to 0° C. and into the cold reaction mixture Et₃N (0.2 mL, 2.5 mmol) and acetic anhydride(0.18 mL, 1.9 mmol) were added. The reaction mixture was allowed to warm up to ambient temperature and stirred for 16 h. After completion, it was quenched with water, extracted with dichloromethane, washed with brine, dried over anhydrous Na₂SO₄ and concentrated under reduced pressure. The product was purified by flash chromatography to afford N-(2-bromophenethyl)acetamide (89-2, 140 mg). LCMS (ESI) Calcd. for C₁₀H₁₂BrNO: 241, found [M+H]⁺=242. ¹H NMR (400 MHz, DMSO-d₆) δ_{H} 7.95 (br s, 1H), 7.59-7.57 (m, 1H), 7.34-7.29 (m, 2H), 7.18-7.14 (m, 1H), 3.32-3.23 (m, 2H), 2.84-2.80 (m, 2H), 1.77 (s, 3H).

Synthesis of N-(2-(7-(cyanomethoxy)-1-oxo-1,2-dihydroisoquinolin-4-yl)phenethyl)acetamide, Example 89 [Step 2]: A stirred solution of 2-((1-oxo-4-(4,4,5,5-tetramethyl-1,3,2-dioxaborolan-2-yl)-1,2-dihydroisoquinolin-7-yl)oxy)acetonitrile (89-3, 300 mg, 0.9 mmol), N-(2-bromophenethyl)acetamide (89-2, 132 mg, 0.5 mmol) and K₃PO₄ (390 mg, 1.8 mmol) in 1,4-dioxane (3 mL) and water (0.5 mL) mixture was purged with Argon gas for 10 min. Into this purged reaction mixture was added [1,1′-bis(di-tert-butylphosphino)ferrocene]dichloropalladium(II) (60 mg, 0.09 mmol) and heated at 80° C. for 1 h. After completion, the reaction mixture was filtered through celite bed. The filtrate was diluted with water and extracted with ethyl acetate (twice). The combined organic extract was washed with brine, dried over anhydrous Na₂SO₄ and concentrated under reduced pressure. The product was purified by reverse phase prep HPLC purification method and lyophilized to afford N-(2-(7-(cyanomethoxy)-1-oxo-1,2-dihydroisoquinolin-4-yl)phenethyl)acetamide (89-3, 60 mg). LCMS (ESI) Calcd. for C₂₁H₁₉N₃O₃: 361, found [M+H]⁺=362. ¹H NMR (400 MHz, DMSO-d₆) δ_{H} 11.48 (br s, 1H), 7.86-7.85 (m, 1H), 7.74-7.71 (m, 1H), 7.41-7.29 (m, 4H), 7.20-7.18 (m, 1H), 6.98-6.94 (m, 2H), 5.32 (m, 2H), 3.11-3.00 (s, 2H), 2.56-2.49 (m, 1H), 2.41-2.37 (m, 1H), 1.65 (s, 3H).

### Example 90-91: Synthesis of chiral analogs of 2-((1-oxo-4-(2-(2,2,2-trifluoro-1-hydroxyethyl)phenyl)-1,2-dihydroisoquinolin-7-yl)oxy)acetonitrile

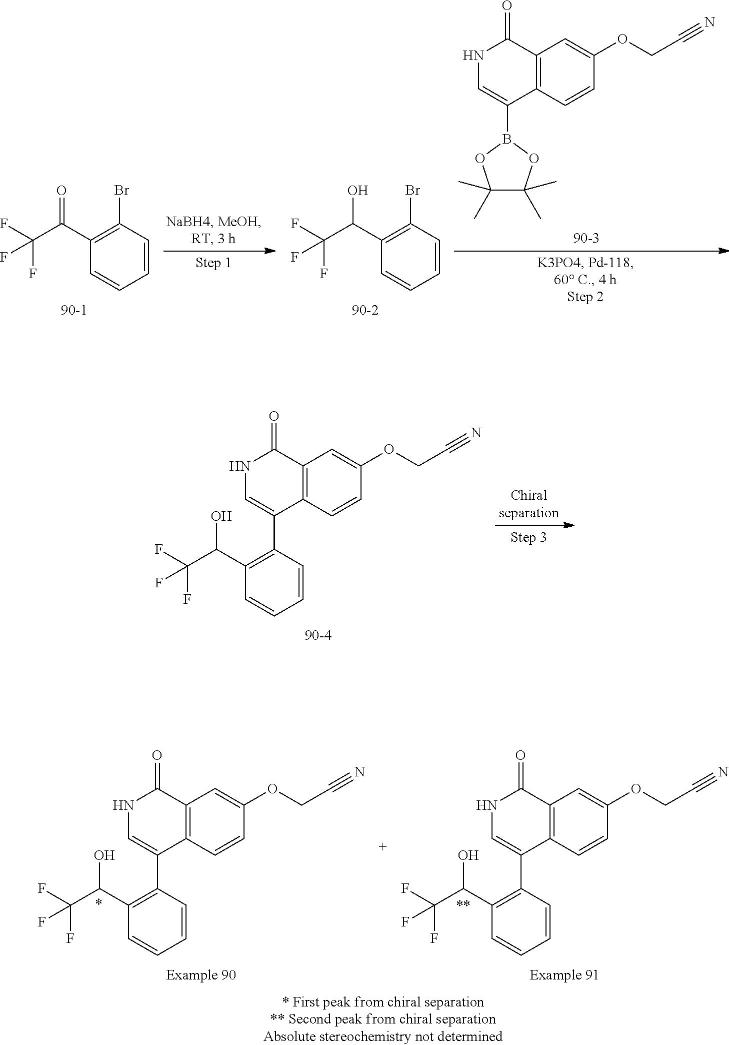

Synthesis of 1-(2-bromophenyl)-2,2,2-trifluoroethan-1-ol, 90-2 [Step 1]: To a stirred solution of 1-(2-bromophenyl)-2,2,2-trifluoroethan-1-one (90-1, 500 mg, 2.0 mmol) in methanol (10 mL) was added sodium borohydride (90 mg, 2.4 mmol) portion wise and the reaction mixture was stirred at ambient temperature for 3 h. After completion, reaction mixture was quenched with 10% HCl solution and concentrated under reduced pressure to afford the crude. The crude material was then diluted with ethyl acetate washed with brine. The organic extract was dried over Na₂SO₄ and concentrated under reduced pressure. The product was purified by flash chromatography to afford 1-(2-bromophenyl)-2,2,2-trifluoroethan-1-ol (90-2, 300 mg). ¹H NMR (400 MHz, DMSO-d₆) δ_{H} NMR (400 MHz, DMSO-d₆) δ_{H} 7.82-7.64 (m, 2H), 7.57-7.37 (m, 1H), 7.35-7.33 (m, 1H), 7.13-7.11 (m, 1H), 5.44-5.39 (m, 1H).

Synthesis of 2-((1-oxo-4-(2-(2,2,2-trifluoro-1-hydroxyethyl)phenyl)-1,2-dihydroisoquinolin-7-yl)oxy)acetonitrile, 90-4 [Step 2]: A stirred solution of 2-((1-oxo-4-(4,4,5,5-tetramethyl-1,3,2-dioxaborolan-2-yl)-1,2-dihydroisoquinolin-7-yl)oxy)acetonitrile (90-3, 200 mg, 0.6 mmol), 1-(2-bromophenyl)-2,2,2-trifluoroethan-1-ol (90-2, 160 mg, 0.6 mmol) and K₃PO₄ (254 mg, 1.2 mmol) in 1,4-dioxane (3 mL) and water (0.5 mL) was purged with Argon for 10 min. Into this purged reaction mixture was added [1,1′-bis(di-tert-butylphosphino)ferrocene]dichloropalladium(II) (40 mg, 0.06 mmol) and heated at 60° C. for 4 h. After completion, the reaction mixture was filtered through celite bed. The filtrate was diluted with water and extracted with ethyl acetate. Combined organic extract was washed with brine, dried over anhydrous Na₂SO₄ and concentrated under reduced pressure. The product was purified by reverse phase prep HPLC purification method and lyophilized to afford 2-((1-oxo-4-(2-(2,2,2-trifluoro-1-hydroxyethyl)phenyl)-1,2-dihydroisoquinolin-7-yl)oxy)acetonitrile (90-4, 70 mg). LCMS (ESI) Calcd. for C₁₉H₁₃F₃N₂O₃: 374, found [M+H]⁺=375.

Synthesis of chiral 2-((1-oxo-4-(2-(2,2,2-trifluoro-1-hydroxyethyl)phenyl)-1,2-dihydroisoquinolin-7-yl)oxy)acetonitrile, Example 90 and Example 91 [Step 3]: Racemic 2-((1-oxo-4-(2-(2,2,2-trifluoro-1-hydroxyethyl)phenyl)-1,2-dihydroisoquinolin-7-yl)oxy)acetonitrile (90-4, 70 mg, 0.2 mmol) was separated by chiral HPLC to afford the second compound as chiral 2-((1-oxo-4-(2-(2,2,2-trifluoro-1-hydroxyethyl)phenyl)-1,2-dihydroisoquinolin-7-yl)oxy)acetonitrile (Example 90, 18 mg) as Peak 2 and the third compound as chiral 2-((1-oxo-4-(2-(2,2,2-trifluoro-1-hydroxyethyl)phenyl)-1,2-dihydroisoquinolin-7-yl)oxy)acetonitrile (Example 91, 20 mg) as Peak 3. The absolute stereochemistry of these compounds was not determined.

Example 90: Chiral 2-((1-oxo-4-(2-(2,2,2-trifluoro-1-hydroxyethyl)phenyl)-1,2-dihydroisoquinolin-7-yl)oxy)acetonitrile, Peak 2: C₁₉H₁₃F₃N₂O₃: 374, found [M+H]⁺=375. ¹H NMR (400 MHz, DMSO-d₆) δ_{H} 11.54-11.52 (br s, 1H), 7.88-7.49 (m, 3H), 7.40-7.27 (m, 3H), 6.97-6.77 (m, 2H), 5.34-5.32 (m, 2H), 4.67-4.65 (m, 2H).

Example 91: Chiral 2-((1-oxo-4-(2-(2,2,2-trifluoro-1-hydroxyethyl)phenyl)-1,2-dihydroisoquinolin-7-yl)oxy)acetonitrile, Peak 3: C₁₉H₁₃F₃N₂O₃: 374, found [M+H]⁺=375. ¹H NMR (400 MHz, DMSO-d₆) δ_{H} 11.54-11.52 (br s, 1H), 7.88-7.49 (m, 3H), 7.40-7.27 (m, 3H), 6.97-6.77 (m, 2H), 5.34-5.32 (m, 2H), 4.67-4.65 (m, 2H).

Chiral Method: Chiral separation was performed on an Agilent 1200 series instrument with a CHIRALPAK IG (250×21 mm), 5μ, which was operated at ambient temperature and a flow rate of 21.0 mL/min. Mobile phase used was a mixture of 80% Hexane, 10% DCM and 10% Ethanol. the instrument was held in isocratic mode for up to 17 min at a wavelength of 282 nm.

### Example 92: Synthesis of 2-((4-(4-fluoro-2-(2-hydroxyethyl)phenyl)-1-oxo-1,2-dihydroisoquinolin-7-yl)oxy)acetonitrile

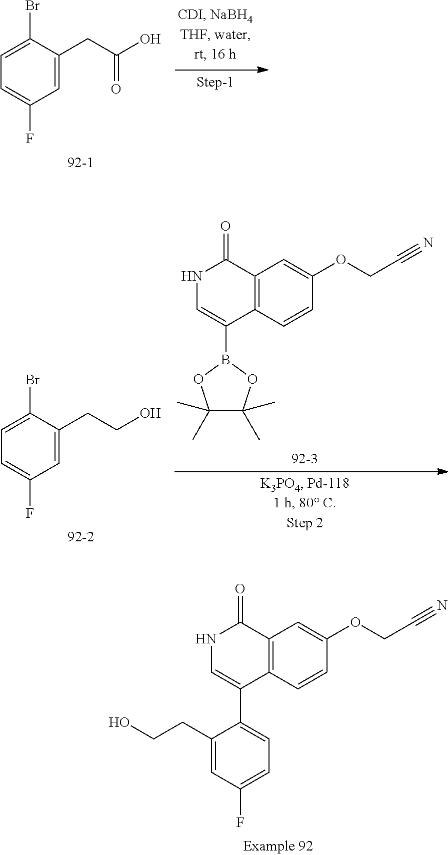

Synthesis of 2-(2-bromo-5-fluorophenyl)ethan-1-ol, 92-2 [Step 1]: To a stirred solution of 2-(2-bromo-5-fluorophenyl)acetic acid (92-1, 250 mg, 1 mmol) in THF (4 mL) was added CDI (191 mg, 1.2 mmol) and the reaction mixture was allowed to stir at 25° C. for 1 h. Then the reaction mixture was allowed to cool to ambient temperature and was added into a pre-cooled water (2 mL) solution of NaBH₄(122 mg, 3.2 mmol) in another round bottom flask. The reaction mixture was allowed to stir at ambient temperature for 16 h. Reaction mixture was quenched with water and extracted with ethyl acetate. Combined organic extract was washed with water, brine, dried over anhydrous Na₂SO₄ and concentrated under reduced pressure. The product was purified by flash chromatography to afford 2-(2-bromo-5-fluorophenyl)ethan-1-ol (92-2, 110 mg). ¹H NMR (400 MHz, DMSO-d₆): δ_{H} 7.62-7.58 (m, 1H), 7.25-7.21 (m, 1H), 7.06-7.01 (m, 1H), 4.78-4.75 (t, 1H), 3.63-3.58 (m, 2H), 2.85-2.82 (m, 2H).

Synthesis of 2-((4-(4-fluoro-2-(2-hydroxyethyl)phenyl)-1-oxo-1,2-dihydroisoquinolin-7-yl)oxy)acetonitrile, Example 92 [Step 2]: To a stirred solution of 2(2-bromo-5-fluorophenyl)ethan-1-ol (92-2, 81 mg, 0.368 mmol) in 1,4-dioxane (3 mL) and water (0.5 mL) was added 2-((1-oxo-4-(4,4,5,5-tetramethyl-1,3,2-dioxaborolan-2-yl)-1,2-dihydroisoquinolin-7-yl)oxy)acetonitrile (92-3, 80 mg, 0.2 mmol) and K₃PO₄ (104 mg, 0.5 mmol). The reaction mixture was purged with argon for 5 min. Then Pd-118 (16 mg, 0.02 mmol) was added in to the reaction mixture and allowed to stir for 1 h at 80° C. Reaction mixture was filtered through a celite bed and washed with ethyl acetate. The filtrate was washed with water, brine, dried over anhydrous Na₂SO₄ and concentrated under reduced pressure. The product was purified under reverse phase prep HPLC purification method and lyophilized to afford 2-((4-(4-fluoro-2-(2-hydroxyethyl)phenyl)-1-oxo-1,2-dihydroisoquinolin-7-yl)oxy)acetonitrile (Example 92, 14 mg). LCMS (ESI) Calcd. for C₁₉H₁₅FN₂O₃: 338, found [M+H]⁺=339. ¹H NMR (400 MHz, DMSO-d₆): δ_{H} 11.49-11.48 (m, 1H), 7.86-7.85 (m, 1H), 7.36-7.35 (m, 1H), 7.27-7.21 (m, 2H), 7.14-7.10 (m, 1H), 6.97-6.94 (m, 2H), 5.32 (s, 2H), 4.60-4.55 (m, 1H), 3.46-3.31 (m, 2H), 2.57-2.39 (m, 1H).

### Example 93: Synthesis of 2-((4-(2-(2-(dimethylamino)ethyl)phenyl)-1-oxo-1,2-dihydroisoquinolin-7-yl)oxy)acetonitrile

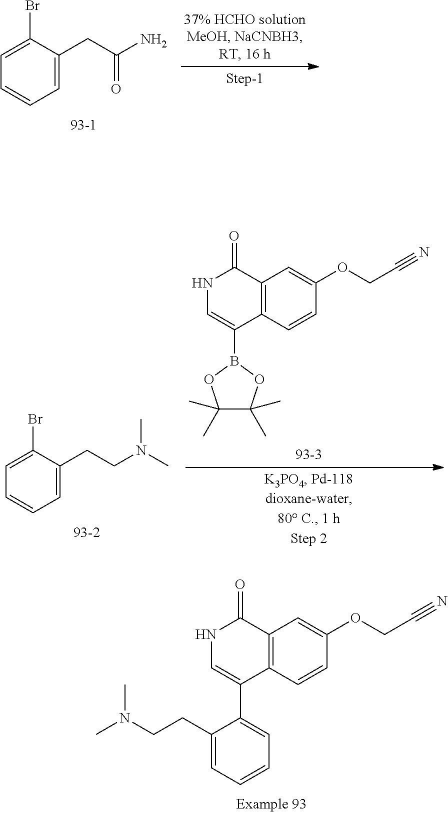

Synthesis of 2-(2-bromophenyl)-N,N-dimethylethan-1-amine, 93-2 [Step 3]: To a stirred solution of 2-(2-bromophenyl)ethan-1-amine (93-1, 500 mg, 2.5 mmol) in methanol (4 mL) was added 37% HCHO solution (0.76 mL, 7.5 mmol), few drops of acetic acid and the reaction mixture was allowed to stir at ambient temperature for 1 h. Then in to this reaction mixture was added NaCNBH₃ (314 mg, 5 mmol) and the reaction mixture was allowed to stir for another 16 h at ambient temperature. Volatiles were removed under reduced pressure. The reaction mass was diluted with water and extracted with ethyl acetate. Combined organic extract was washed with water, brine, dried over anhydrous Na₂SO₄ and concentrated under reduced pressure. The product was purified by flash chromatography to afford 2-(2-bromophenyl)-N, N-dimethylethan-1-amine (93-2, 380 mg). LCMS (ESI) Calcd. for C₁₀H₁₄BrN: 227, found [M+H]⁺=228.

Synthesis of 2-((4-(2-(2-(dimethylamino)ethyl)phenyl)-1-oxo-1,2-dihydroisoquinolin-7-yl)oxy)acetonitrile, Example 93 [Step 2]: To a stirred solution of 2-(2-bromophenyl)-N,N-dimethylethan-1-amine (93-2, 210 mg, 0.9 mmol) in 1,4-dioxane (3 mL) and water (0.5 mL) were added 2-((1-oxo-4-(4,4,5,5-tetramethyl-1,3,2-dioxaborolan-2-yl)-1,2-dihydroisoquinolin-7-yl)oxy)acetonitrile (93-3, 200 mg, 0.6 mmol) and K₃PO₄ (260 mg, 1.2 mmol). The reaction mixture was purged with argon for 5 min. Then Pd-118 (40 mg, 0.06 mmol) was added in to the reaction mixture and allowed to stir for 1 h at 80° C. Reaction mixture was filtered through a celite bed and the bed was washed with ethyl acetate. Filtrate was washed with water, brine, dried over anhydrous Na₂SO₄.and concentrated under reduced pressure. The product was purified under reverse phase prep HPLC purification method and lyophilized to afford 2-((4-(2-(2-(dimethylamino)ethyl)phenyl)-1-oxo-1,2-dihydroisoquinolin-7-yl)oxy)acetonitrile (Example 93, 65 mg). LCMS (ESI) Calcd. for C₂₁H₂₁N₃O₂: 347, found [M+H]⁺=348. ¹H NMR (400 MHz, DMSO-d₆): δ_{H} 11.53 (br s, 1H), 9.32 (br s, 1H), 7.89 (s, 1H), 7.46-7.37 (m, 4H), 7.27-7.25 (d, 1H), 7.06 (s, 1H), 6.95-6.93 (d, 1H), 5.33 (m, 2H), 5.32 (s, 2H), 3.26-3.08 (m, 2H), 2.90-2.87 (m, 2H), 2.63-2.52 (m, 6H).

### Example 94. Synthesis of (R)-3-(2-((1-oxo-4-(o-tolyl)-1,2-dihydroisoquinolin-7-yl)oxy)propanoyl)-3-azabicyclo[3.1.1]heptane-1-carboxylic acid

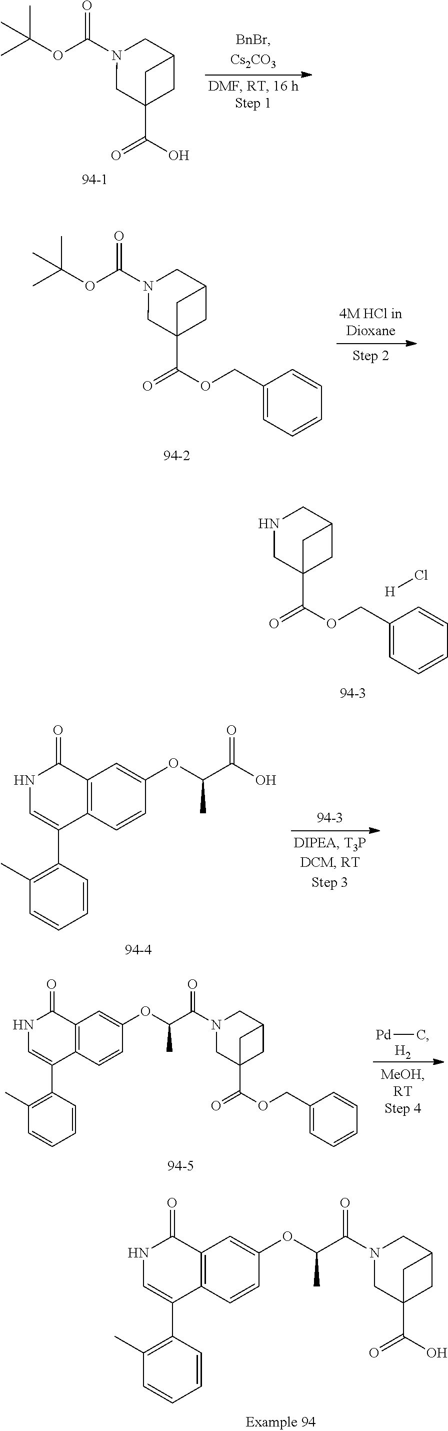

Synthesis of 1-benzyl 3-(tert-butyl) 3-azabicyclo[3.1.1]heptane-1,3-dicarboxylate, 94-[Step 1]: To a stirred solution of 3-(tert-butoxycarbonyl)-3-azabicyclo[3.1.1]heptane-1-carboxylic acid (94-1, 140 mg, 0.6 mmol) in DMF (6 mL) was added cesium carbonate (380 mg, 1.2 mmol) followed by benzyl bromide (0.10 mL, 0.9 mmol) and the resultant mixture was stirred for 16 h at ambient temperature. After completion, the reaction mixture was diluted with water and extracted with ethyl acetate (twice). Combined organic extracts was washed with brine, dried over anhydrous sodium sulphate and concentrated under reduced pressure to afford 1-benzyl 3-(tert-butyl) 3-azabicyclo[3.1.1]heptane-1,3-dicarboxylate (94-2, 150 mg). ¹H NMR (400 MHz, DMSO-d₆): δ_{H} 7.43-7.30 (m, 5H), 5.12 (s, 2H), 4.07 (s, 1H), 4.49-4.47 (m, 1H), 3.43-3.38 (m, 2H), 2.41-2.32 (m, 3H), 1.54-1.52 (m, 2H), 1.49 (s, 9H).

Synthesis of benzyl 3-azabicyclo[3.1.1]heptane-1-carboxylate hydrochloride, 94-3 [Step 2]: To a stirred solution of 1-benzyl 3-(tert-butyl) 3-azabicyclo[3.1.1]heptane-1,3-dicarboxylate (94-2, 100 mg, 0.3 mmol) in 1,4-dioxane (5 mL) was added 4M HCl in dioxane (0.75 mL, 3 mmol) dropwise at ice cold condition. The reaction mixture was stirred at ambient temperature for 12 h. Volatiles were removed under reduced pressure to afford benzyl 3-azabicyclo[3.1.1]heptane-1-carboxylate hydrochloride (94-3, 80 mg). LCMS (ESI) Calcd. for C₁₄H₁₈ClNO₂: 231, found [M+H]⁺=232.

Synthesis of benzyl (R)-3-(2-((1-oxo-4-(o-tolyl)-1,2-dihydroisoquinolin-7-yl)oxy)propanoyl)-3-azabicyclo[3.1.1]heptane-1-carboxylate, 94-5 [Step 3]: To a stirred solution of (R)-2-((1-oxo-4-(o-tolyl)-1,2-dihydroisoquinolin-7-yl)oxy)propanoic acid (94-4, 100 mg, 0.3 mmol) in dichloromethane (10 mL) was added benzyl 3-azabicyclo[3.1.1]heptane-1-carboxylate hydrochloride (94-3, 65 mg, 0.6 mmol) followed by DIPEA (0.3 mL, 1.3 mmol) at 0° C. After 10 min, T₃P (0.3 mL, 0.5 mmol, 50% in ethyl acetate) was added to the reaction mixture at 0° C. and stirred at ambient temperature for 3 h. After completion, reaction mixture was quenched with ice water and extracted with dichloromethane (twice). Combined organic extracts was washed with brine, dried over anhydrous sodium sulphate and concentrated under reduced pressure to afford benzyl (R)-3-(2-((1-oxo-4-(o-tolyl)-1,2-dihydroisoquinolin-7-yl)oxy)propanoyl)-3-azabicyclo[3.1.1]heptane-1-carboxylate (94-5, 140 mg). LCMS (ESI) Calcd. for C₃₃H₃₂N₂O₅: 537, found [M+H]⁺=537.

Synthesis of (R)-3-(2-((1-oxo-4-(o-tolyl)-1,2-dihydroisoquinolin-7-yl)oxy)propanoyl)-3-azabicyclo[3.1.1]heptane-1-carboxylic acid, Example 94 [Step 4]: To the stirred solution of benzyl (R)-3-(2-((1-oxo-4-(o-tolyl)-1,2-dihydroisoquinolin-7-yl)oxy)propanoyl)-3-azabicyclo[3.1.1]heptane-1-carboxylate (94-5, 180 mg, 0.3 mmol) in methanol (15 mL) was added Pd/C (40 mg, 0.04 mmol) and the resultant mixture was stirred under H₂-atmosphere for 16 h at ambient temperature. After completion, the reaction mixture was filtered through celite bed and washed with methanol (twice). Combined filtrate was concentrated under reduced pressure. The product was purified by reverse phase HPLC and lyophilization to afford (R)-3-(2-((1-oxo-4-(o-tolyl)-1,2-dihydroisoquinolin-7-yl)oxy)propanoyl)-3-azabicyclo[3.1.1]heptane-1-carboxylic acid (Example 94, 75 mg). LCMS (ESI) Calcd. for C₂₆H₂₆N₂O₅: 447, found [M+H]⁺=447. ¹H NMR (400 MHz, DMSO-d₆): δ_{H} 10.88 (br s, 1H), 7.62 (s, 1H), 7.33-7.17 (m, 5H), 6.93 (d, 11H), 6.83 (s, 11H), 5.29-5.28 (m, 1H), 4.0-3.90 (m, 2H), 3.78-3.52 (m, 4H), 2.34 (br s, 2H), 2.06 (s, 3H), 1.66 (br s, 1H), 1.53 (d, 4H).

### Example 95: Synthesis of 2-((4-(3-fluoro-2-(2-hydroxyethyl)phenyl)-1-oxo-1,2-dihydroisoquinolin-7-yl)oxy)acetonitrile

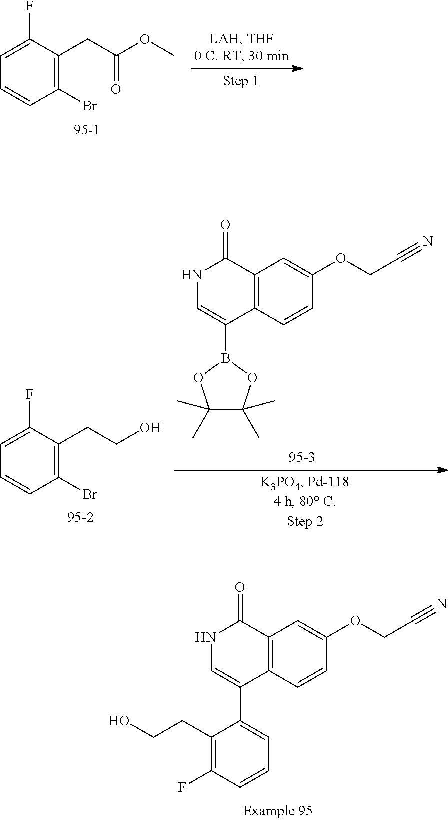

Synthesis of 2-(2-bromo-6-fluorophenyl)ethan-1-ol, 95-2 [Step 1]: A solution of methyl 2-(2-bromo-6-fluorophenyl)acetate (95-1, 200 mg, 0.8 mmol) in THF (4 mL), was cooled at 0° C., and into the solution LiAlH₄ 1M in THF (0.8 mL, 0.81 mmol) was added dropwise into the reaction mixture, and continued to stir at ambient temperature for 30 min. After completion, it quenched with saturated aqueous Na₂SO₄ solution under cold condition and was allowed to stir for 1 h. Then the reaction mixture was filtered through sintered funnel, concentrated under reduced pressure. The product was purified by flash chromatography to afford 2-(2-bromo-6-fluorophenyl)ethan-1-ol (95-2, 160 mg). ¹H NMR (400 MHz, DMSO-d₆) δ_{H} 7.45-7.43 (m, 1H), 7.23-7.20 (m, 2H), 4.86-4.84 (m, 1H), 3.57-3.50 (m, 2H), 2.92-2.89 (m, 2H).

Synthesis of 2-((4-(3-fluoro-2-(2-hydroxyethyl)phenyl)-1-oxo-1,2-dihydroisoquinolin-7-yl)oxy)acetonitrile, Example 95 [Step 2]: A solution of 2-((1-oxo-4-(4,4,5,5-tetramethyl-1,3,2-dioxaborolan-2-yl)-1,2-dihydroisoquinolin-7-yl)oxy)acetonitrile (95-3, 300 mg, 0.9 mmol), 2-(2-bromo-6-fluorophenyl)ethan-1-ol (95-2, 120 mg, 0.6 mmol) and K₃PO₄ (390 mg, 1.8 mmol) in 1,4-Dioxane (3 mL) and Water (0.5 mL) was purged with argon for 10 min. Then into this purged reaction mixture was added [1,1′-bis(di-tert-butylphosphino)ferrocene]dichloropalladium(II) (60 mg, 0.09 mmol) and continued to stir at 80° C. for 1 h. After completion, the reaction mixture was filtered through celite bed; filtrate was diluted with water and extracted with ethyl acetate. Combined organic extract was washed with brine, dried over anhydrous Na₂SO₄ and concentrated under reduced pressure. The product was purified by reverse phase prep HPLC purification method and lyophilized to afford 2-((4-(3-fluoro-2-(2-hydroxyethyl)phenyl)-1-oxo-1,2-dihydroisoquinolin-7-yl)oxy)acetonitrile (Example 95, 25 mg). LCMS (ESI) Calcd. for C₁₉H₁₅N₂O₃: 338, Found [M+H]⁺: 339. ¹H NMR (400 MHz, DMSO-d₆) δ_{H} 11.53-11.51 (br s, 1H), 7.86 (m, 1H), 7.38-7.32 (m, 2H), 7.27-7.23 (m, 1H), 7.07-7.04 (m, 2H), 6.98-6.96 (m, 1H), 5.32 (s, 2H), 4.60-4.57 (m, 1H), 2.73-2.67 (m, 2H), 2.42-2.32 (m, 2H).

### Example 96: Synthesis of 2-((4-(2-(methoxymethyl)-6-methylphenyl)-1-oxo-1,2-dihydroisoquinolin-7-yl)oxy)acetonitrile

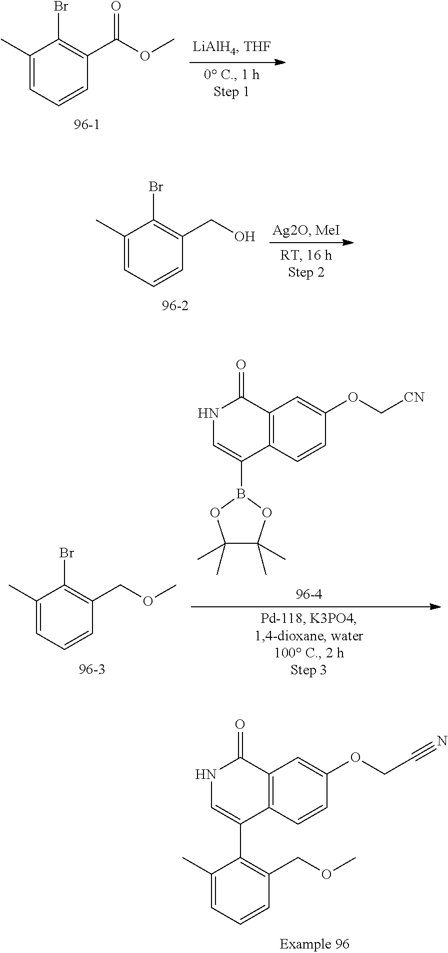

Synthesis of (2-bromo-3-methylphenyl) methanol, 96-2 [Step 1]: To a stirred solution of methyl 2-bromo-3-methylbenzoate (96-1, 150 mg, 0.6 mmol) in THF (2 mL), LiAIH₄ (1M in THF) (0.1 mL, 0.9 mmol) was added dropwise at 0° C. and stirred at same temperature for 1 h. After completion, the reaction mixture was quenched with saturated aqueous solution of NH₄Cl at 0° C. and the solid was filtered through celite bed and washed with ethyl acetate. The filtrate was washed with water, brine, dried over anhydrous Na₂SO₄, and concentrated under reduced pressure to afford (2-bromo-3-methylphenyl) methanol (96-2, 60 mg). ¹H NMR (400 MHz, DMSO-d₆): δ 7.36 (d, 11H), 7.28 (t, 1H), 7.23 (d, 1H), 5.40 (t, 1H), 4.51-4.44 (m, 2H), 2.35 (s, 3H).

Synthesis of 2-bromo-1-(methoxymethyl)-3-methylbenzene, 96-3 [Step 2]: A sealed tube was charged with (2-bromo-3-methylphenyl) methanol (96-2, 600 mg, 2.9 mmol) in iodomethane (4.7 mL, 74.6 mmol), followed by silver oxide (2075 mg, 8.9 mmol) was added and the reaction mixture was stirred at ambient temperature for 16 h. After completion, the reaction mixture was filtered through celite bed and the filtrate was washed with water, brine, dried over anhydrous sodium sulfate, and concentrated under reduced pressure. The product was purified by flash column chromatography to afford 2-bromo-1-(methoxymethyl)-3-methylbenzene (96-3, 250 mg). ¹H NMR (400 MHz, DMSO-d₆): δ 7.28-7.25 (m, 1H), 7.22-7.15 (m, 2H), 4.5 (s, 2H), 3.46 (s, 3H), 2.41 (s, 3H). GCMS Calcd. for C₉H₁₁BrO: 215, found [M−H]⁻=214.

Synthesis of 2-((4-(2-(methoxymethyl)-6-methylphenyl)-1-oxo-1,2-dihydroisoquinolin-7-yl)oxy)acetonitrile, Example 96 [Step 3]: A sealed tube was charged with 2-bromo-1-(methoxymethyl)-3-methylbenzene (96-3, 300 mg, 1.4 mmol), 2-((1-oxo-4-(4,4,5,5-tetramethyl-1,3,2-dioxaborolan-2-yl)-1,2-dihydroisoquinolin-7-yl)oxy)acetonitrile (96-4, 682 mg, 2.0 mmol) and K₃PO₄ (592 mg, 2.8 mmol) in 1,4-dioxane (10 mL and water (2 mL). The reaction mixture was purged with argon for 10 min. [1,1′-bis(di-tert-butylphosphino)ferrocene]dichloropalladium(II) (90.9 mg, 0.1 mmol) was added into the mixture and stirred at 100° C. for 2 h. After completion, the reaction mixture was filtered through celite bed and washed with ethyl acetate. The filtrate was washed with water, brine, dried over anhydrous Na₂SO₄ and concentrated under reduced pressure and purified by reverse phase HPLC to afford 2-((4-(2-(methoxymethyl)-6-methylphenyl)-1-oxo-1,2-dihydroisoquinolin-7-yl)oxy)acetonitrile (Example 96, 20 mg) ¹H NMR (400 MHz, DMSO-d₆): δ 11.49 (d, 1H), 7.86 (s, 1H), 7.36-7.28 (m, 4H), 6.9 (s, 1H), 6.80 (d, 1H), 5.3 (s, 2H), 4.12 (d, 1H), 4.0 (d, 1H), 3.00 (s, 3H), 1.96 (s, 3H). LCMS (ESI) Calcd. for C₂₀H₁₈N₂O₃: 334, found [M+H]⁺=335.

### Example 97: Synthesis of 2-((4-(4-fluoro-2-(hydroxymethyl)-6-methylphenyl)-1-oxo-1,2-dihydroisoquinolin-7-yl)oxy)acetonitrile

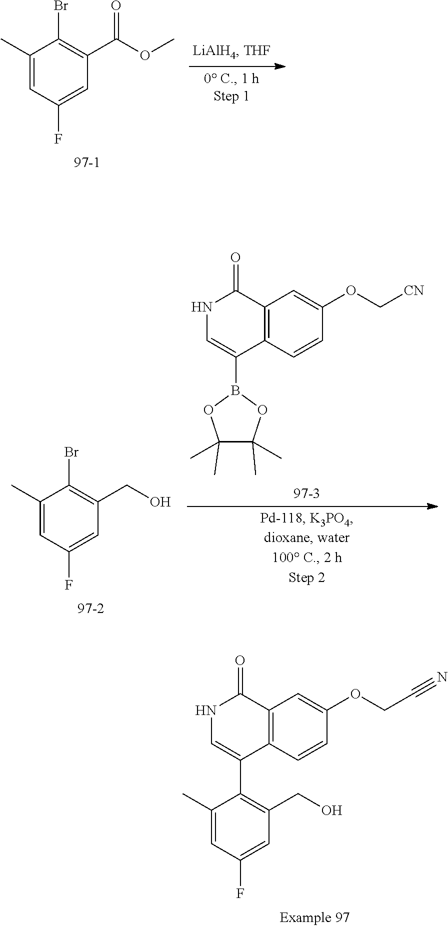

Synthesis of (2-bromo-5-fluoro-3-methylphenyl)methanol, 2 [Step 1]: To a stirred solution of methyl 2-bromo-5-fluoro-3-methyl-benzoate (97-1, 200 mg, 0.8 mmol) in THF (15 mL) LiAlH₄ (1M in THF) (46 mg, 1.2 mmol) was added dropwise at 0° C. and stirred at same temperature for 1 h. After completion, the reaction mixture was quenched with saturated NH₄Cl solution at 0° C. and the solid was filtered and washed with ethyl acetate. The filtrate was washed with water, brine and dried over anhydrous sodium sulfate and concentrated under reduced pressure to afford (2-bromo-5-fluoro-3-methylphenyl)methanol (97-2, 100 mg). GCMS Calcd. for C₈H₈BrFO: 219, found [M−H]⁻=218.

Synthesis of 2-((4-(4-fluoro-2-(hydroxymethyl)-6-methylphenyl)-1-oxo-1,2-dihydroisoquinolin-7-yl)oxy)acetonitrile, Example 97 [Step 2]: (2-bromo-5-fluoro-3-methylphenyl)methanol (97-2, 153 mg, 0.9 mmol), 2-[[1-oxo-4-(4,4,5,5-tetramethyl-1,3,2-dioxaborolan-2-yl)-2H-isoquinolin-7-yl]oxy]acetonitrile (97-3, 341 mg, 1.0 mmol), and K₃PO₄ (296 mg, 1.4 mmol) in 1,4-dioxane (6 mL) and water (1 mL). The reaction mixture was purged with argon for 10 min. [1,1′-Bis(di-tert-butylphosphino)ferrocene]dichloropalladium(II) (45 mg, 0.0697 mmol) was added into the reaction mixture and stirred at 100° C. for 2 h. After completion, the reaction mixture was cooled to ambient temperature and filtered over celite bed. The filtrate was concentrated under reduced pressure and purified by reverse phase HPLC to afford 2-((4-(4-fluoro-2-(hydroxymethyl)-6-methylphenyl)-1-oxo-1,2-dihydroisoquinolin-7-yl)oxy)acetonitrile (Example 97, 11 mg). LCMS (ESI) Calcd. for C₁₉H₁₅FN₂O₃: 338, found [M−H]⁻=339. ¹H NMR (400 MHz, DMSO-d₆): δ_{H} 11.50 (s, 1H), 7.86 (s, 1H), 7.35-7.32 (m, 1H), 7.19 (d, 1H), 7.11 (d, 1H), 6.92 (s, 1H), 6.82 (d, 1H), 5.31 (s, 2H), 5.10 (t, 1H), 4.29-4.24 (m, 1H), 4.01-3.96 (m, 1H), 1.96 (s, 3H).

### Example 98. Synthesis of N-(2-(7-(cyanomethoxy)-1-oxo-1,2-dihydroisoquinolin-4-yl)phenethyl)-N-methylacetamide

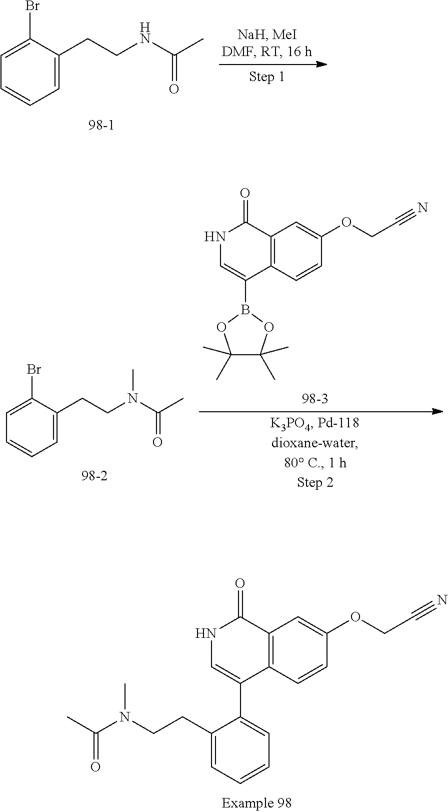

Synthesis of N-(2-bromophenethyl)-N-methylacetamide. 98-2 [Step 3]: To stirred solution of N-(2-bromophenethyl)acetamide (98-1, 100 mg, 0.4 mmol) in DMF (1 mL) was added NaH (33 mg, 0.8 mmol) at 0° C. and the reaction mixture was allowed to stir at the same temperature for 15 min. Then in to this reaction mixture was added Mel (0.13 mL, 2 mmol) and was allowed to warm up to ambient temperature and stirred for 16 h. Reaction mixture was quenched with cold water and extracted with ethyl acetate. Combined organic extract was washed with water, brine, dried over anhydrous Na₂SO₄ and concentrated under reduced pressure. The product was purified by flash column chromatography to afford N-(2-bromophenethyl)-N-methylacetamide (98-2, 80 mg). LCMS (ESI) Calcd. for C₁₁H₁₄BrNO: 255, found [M+H]⁺=256.

Synthesis of N-(2-(7-(cyanomethoxy)-1-oxo-1,2-dihydroisoquinolin-4-yl)phenethyl)-N-methylacetamide, Example 98 [Step 2]: To a stirred solution of 2-((1-oxo-4-(4,4,5,5-tetramethyl-1,3,2-dioxaborolan-2-yl)-1,2-dihydroisoquinolin-7-yl)oxy)acetonitrile (98-3, 150 mg, 0.5 mmol) in 1,4-dioxane (3 mL), water (0.5 mL) were added N-(2-bromophenethyl)-N-methylacetamide (98-2, 177 mg, 0.7 mmol), K₃PO₄ (195 mg, 0.9 mmol) and the reaction mixture was purged with Argon for 5 min. Then in to this reaction mixture was added Pd-118 (30 mg, 0.05 mmol) and the reaction mixture was heated at 80° C. for 1 h. Reaction mixture was filtered through a celite bed and the filtrate was diluted with ethyl acetate. Filtrate was washed with water, brine, dried over anhydrous Na₂SO₄ and concentrated under reduced pressure. The product was purified by reverse phase prep HPLC purification method and lyophilized to afford N-(2-(7-(cyanomethoxy)-1-oxo-11,2-dihydroisoquinolin-4-yl)phenyethyl)-N-methylacetamide (Example 98, 62 mg). LCMS (ESI) Calcd. for C₂₂H₂₁N₃O₃: 375, found [M+H]⁺:376. ¹H NMR (400 MHz, DMSO-d₆): δ_{H} 11.62-11.50 (br s, J H), 7.86 (m, 1H), 7.44-7.31 (m, 4H), 7.24-7.18 (m, 1H), 7.02-6.95 (m, 2H), 5.32 (s, 2H), 3.23-3.16 (m, 3H), 2.71-2.43 (m, 4H₁), 1.80-1.49 (in, 3H).

### Example 99 and 100: Synthesis of chiral analogs of 2-((4-(2-(2-hydroxypropyl)phenyl)-1l-oxo-1,2-dihydroisoquinolin-7-yl)oxy)acetonitrile

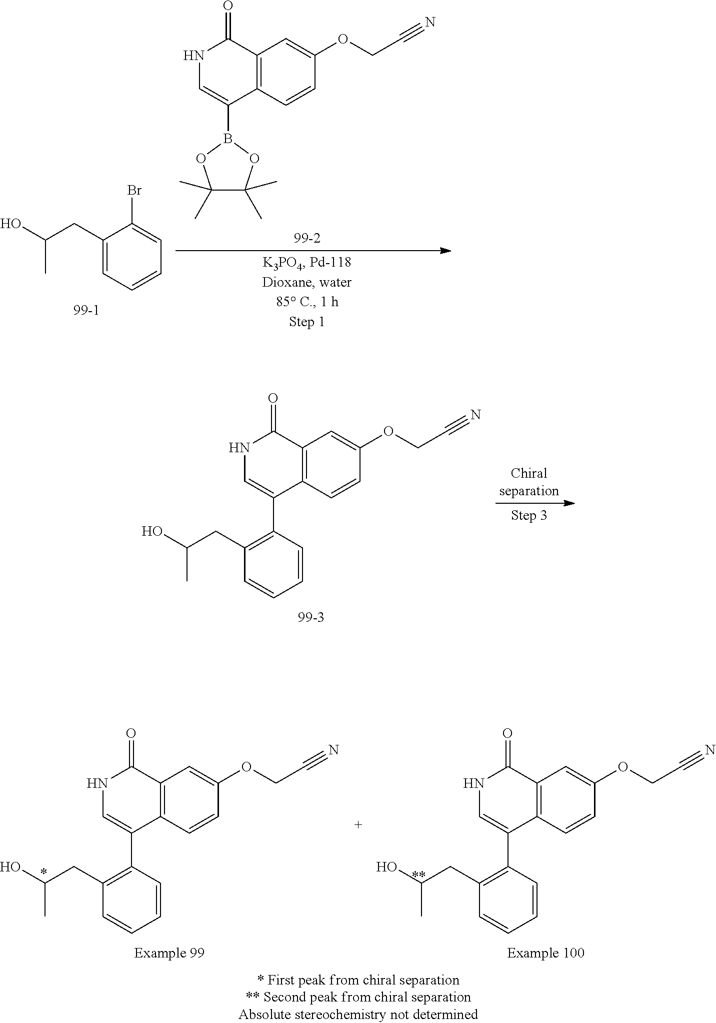

Synthesis of 2-((4-(2-(2-hydroxypropyl)phenyl)-1-oxo-1,2-dihydroisoquinolin-7-yl)oxy)acetonitrile, 99-3 [Step 1]: To a stirred solution of 2-((1-oxo-4-(4,4,5,5-tetramethyl-1,3,2-dioxaborolan-2-yl)-1,2-dihydroisoquinolin-7-yl)oxy)acetonitrile (99-2, 270 mg, 0.6 mmol) and 1-(2-bromophenyl)propan-2-ol (99-1, 200 mg, 0.9 mmol) in 1,4-dioxane (4 mL) and water (1 mL) was added K₃PO₄ (260 mg, 1.2 mmol). Then N₂ was purged for 10 min, [1,1′-bis(di-tert-butylphosphino)ferrocene]dichloropalladium(II) (40 mg, 0.06 mmol) was added into the mixture and stirred at 85° C. for 1 h. After completion, the mixture was diluted with ethyl acetate and washed with brine. The organic extract was dried over anhydrous Na₂SO₄, filtered and concentrated under reduced pressure. The product was purified by reverse phase HPLC preparative chromatography to afford 2-((4-(2-(2-hydroxypropyl)phenyl)-1-oxo-1,2-dihydroisoquinolin-7-yl)oxy)acetonitrile (99-3, 85 mg). LCMS (ESI) Calcd. for C₂₀H₁₈N₂O₃=334, found [M+H]⁺=335. ¹H NMR (400 MHz, DMSO-d₆)=11.45 (br s, 1H), 7.86 (s, 1H), 7.39-7.35 (m, 3H), 7.29 (t, 11H), 7.19-7.17 (m, 1H), 6.96 (d, 2H), 5.32 (s, 2H), 4.42 (t, 1H), 3.69-3.59 (m, 1H), 2.72-2.24 (m, 2H), 0.86-0.80 (m, 3H).

Synthesis of chiral 2-((4-(2-(2-hydroxypropyl)phenyl)-1-oxo-1,2-dihydroisoquinolin-7-yl)oxy)acetonitrile, Example 99 and Example 100 [Step 2]: The racemic 2-((4-(2-(2-hydroxypropyl)phenyl)-1-oxo-1,2-dihydroisoquinolin-7-yl)oxy)acetonitrile (99-3, 85 mg, 0.3 mmol) underwent SFC chiral HPLC separation to afford the first product as 2-((4-(2-(2-hydroxypropyl)phenyl)-1-oxo-1,2-dihydroisoquinolin-7-yl)oxy)acetonitrile, Peak 1 (Example 99, 13 mg), the second product as 2-((4-(2-(2-hydroxypropyl)phenyl)-1-oxo-1,2-dihydroisoquinolin-7-yl)oxy)acetonitrile, Peak 2 (Example 100, 35 mg). The absolute stereochemistry of these compounds was not determined.

Chiral HPLC SFC method: Chiral HPLC SFC was performed using a Pic Solution 175 instrument equipped with a Knauer UV Detector 40D and a Chiralpak IG (30.0 mm×250 mm), 5μ, which was operated at a temperature of 35° C. with a flow rate of 100 mL/min, The mobile phase used was a mixture of 65% CO₂ in super critical state and 35% of (100% MeOH). The instrument was held in isocratic mode for up to 13.0 min and with isobaric conditions of 100 bar at a wavelength of 220 nm.

Example 99: 2-((4-(2-(2-hydroxypropyl)phenyl)-1-oxo-1,2-dihydroisoquinolin-7-yl)oxy)acetonitrile, Peak 1: LCMS (ESI) Calcd. for C₂₀H₁₈N₂O₃=334, found [M+H]⁺=335; Chiral purity: 100% de. ¹H NMR (400 MHz, DMSO-d₆): δ 11.10 (br s, 1H), 7.89 (s, 1H), 7.43 (d, 1H), 7.39-7.33 (m, 2H), 7.29 (t, 1H), 7.18 (d, 1H), 7.00 (d, 1H), 6.91 (d, 1H), 5.24 (s, 2H), 4.06 (br s, 1H), 3.76-3.67 (m, 1H), 2.69-2.65 (m, 1H), 2.35-2.29 (m, 1H), 0.90-0.86 (m, 3H).

Example 100: 2-((4-(2-(2-hydroxypropyl)phenyl)-1-oxo-1,2-dihydroisoquinolin-7-yl)oxy)acetonitrile, Peak 2: LCMS (ESI) Calcd. for C₂₀H₁₈N₂O₃=334, found [M+H]⁺=335; Chiral purity: 80% de. ¹H NMR (400 MHz, DMSO-d₆): δ 11.11 (br s, 1H), 7.89 (s, 1H), 7.43 (d, 1H), 7.39-7.33 (m, 2H), 7.29 (t, 1H), 7.18 (d, 1H), 7.00 (d, 1H), 6.91 (d, 1H), 5.24 (s, 2H), 4.07 (br s, 1H), 3.76-3.67 (m, 1H), 2.69-2.65 (m, 1H), 2.35-2.29 (m, 1H), 0.90-0.86 (m, 3H).

### Example 101: Synthesis of 2-((4-(3-fluoro-6-(hydroxymethyl)-2-methylphenyl)-1-oxo-1,2-dihydroisoquinolin-7-yl)oxy)acetonitrile

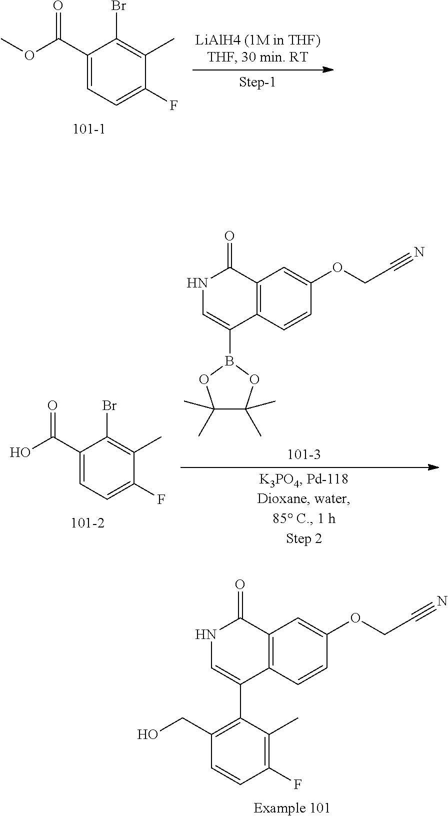

Synthesis of (2-bromo-4-fluoro-3-methylphenyl)methanol, 101-2 [Step 1]: A stirred solution of methyl 2-bromo-4-fluoro-3-methylbenzoate (101-1, 500 mg, 2.0 mmol) in THF (20 mL) was cooled to 0° C. and LiAlH₄ (1M in THF) (4 mL, 4.05 mmol) was added dropwise at ice cold condition. The reaction was stirred at ambient temperature for 30 minutes. The reaction mixture was quenched with saturated Na₂SO₄ solution followed by 15% NaOH. The mixture was passed through celite bed and washed with ethyl acetate. The organic extract was washed with brine, dried over anhydrous Na₂SO₄ and concentrated under reduced pressure to afford (2-bromo-4-fluoro-3-methylphenyl)methanol (101-2, 400 mg). NMR of the crude mixture showed the formation of desired product along with desbromo product.

Synthesis of 2-((4-(3-fluoro-6-(hydroxymethyl)-2-methylphenyl)-1-oxo-1,2-dihydroisoquinolin-7-yl)oxy)acetonitrile, Example 101 [Step 2]: To a stirred solution of 2-((1-oxo-4-(4,4,5,5-tetramethyl-1,3,2-dioxaborolan-2-yl)-1,2-dihydroisoquinolin-7-yl)oxy)acetonitrile (101-3, 150 mg, 0.5 mmol) and (2-bromo-4-fluoro-3-methylphenyl)methanol (101-2, 300 mg, 0.7 mmol) in 1,4-dioxane (4 mL) and water (1 mL) was added K₃PO₄ (195 mg, 0.9 mmol). Then N₂ was purged for 10 min, [1,1′-bis(di-tert-butylphosphino)ferrocene]dichloropalladium(II) (30 mg, 0.05 mmol) was added into the mixture and stirred at 85° C. for 1 h. After completion, the mixture was diluted with ethyl acetate and washed with brine. The organic extract was dried over anhydrous Na₂SO₄, filtered and concentrated under reduced pressure. The product was purified by reverse phase HPLC preparative chromatography to afford 2-((4-(3-fluoro-6-(hydroxymethyl)-2-methylphenyl)-1-oxo-1,2-dihydroisoquinolin-7-yl)oxy)acetonitrile (Example 101, 3 mg). LCMS (ESI) Calcd. for C₁₉H₁₅FN₂O₃=338, found [M+H]⁺=339. ¹H NMR (400 MHz, DMSO-d₆): 11.53 (br s, 1H), 7.85 (s, 1H), 7.34-7.32 (m, 2H), 7.19 (t, 1H), 6.97 (s, 1H), 6.83 (d, 1H), 5.32 (s, 2H), 4.78 (br s, 1H), 4.36-4.34 (m, 1H), 3.96-3.93 (m, 1H), 1.92 (s, 3H).

### Biological/Biochemical Evaluation

### General Protocol for In Vitro Analysis of Compounds

The inhibitory activity of the compounds of the present invention against POLRMT were determined by assays based on Bergbrede, T., et al., “An adaptable high-throughput technology enabling the identification of specific transcription modulators,” SLAC Discov., 22, 378-386 (2017).

The ability of some compounds of the present invention to inhibit POLRMT were determined in a homogeneous TR-FRET Assay using high-throughput screening in a 384-well plate format. This method is used to monitor the activity of mitochondrial transcription through measurement of its product, a 407 bp long RNA transcript. Detection of the product is facilitated by hybridization of two DNA-oligonucleotide probes to specific and adjacent sequences within the RNA product sequence. Upon annealing of the probes, two fluorophores are coupled directly to an acceptor nucleotide probe (ATTO647, 5′), or introduced via a coupled streptavidin with a biotinylated donor nucleotide probe (Europium cryptate) that is brought into sufficient proximity to serve as a fluorescence-donor-acceptor pair. Thus, a FRET signal at 665 nm is generated upon excitation at 340 nm.

Proteins used as transcription factors (POLRMT: NP_005026.3, TFAM: NP_003192.1, TFB2M: NP_071761.1) are diluted from their stocks to working concentrations of 1 μM, 20 μM and 4 μM respectively, in a dilution buffer containing 20 mM Tris-HCl (pH 8.0), 200 mM NaCl, 10% (v/v) glycerol, 1 mM Dithiothreitol (DTT), 0.5 mM EDTA.

DNA template is a pUC18 plasmid with the mitochondrial light strand promotor sequence (1-477) cloned between HindIII and BamHI sites. The DNA template is restriction linearized proximal to the promotor 3′-end (pUC-LSP).

The reaction mixture (10 uL) containing 7.5 nM POLRMT, 15 nM of TFB2M, 30 nM of TFAM, 0.5 nM of DNA template and 500 μM nucleotide triphosphate mix (NTPs) in a reaction buffer (containing 10 mM Tris-HCl (pH 7.5), 10 mM MgCl₂, 40 mM NaCl, 10 mM DTT, 0.005% (w/v) Tween-20, 160 units/ml Rnase inhibitor and 0.1 mg/mL BSA) are dispensed to compounds in microplates, using a Thermo Multidrop® dispenser, and incubated at 37° C. in a VWR INCU-Line incubator for 60 minutes after mixing. No nucleotide triphosphate mix is added to negative control samples. Microplates with compounds to be tested in the assay are prepared from 10 mM compound stocks in 100% DMSO, equal amounts of DMSO without any compound are added to positive control and negative control samples.

During the incubation, a mix of the detection reagents is prepared in a buffer such that the enzymatic reaction is terminated due to chelating of Mg-ions and increased ionic strength, containing 50 mM Tris-HCl (pH 7.5), 700 mM NaCl, 20 mM EDTA, and 0.01% (w/v) Tween-20. Europium-streptavidin is pre-incubated with a 200-fold molar excess of a random sequence oligonucleotide to block unspecific binding of oligo, for two hours at ambient temperature in the dark. Afterwards, the blocked Europium-streptavidin is kept on ice until use.

At the end of the enzymatic reaction time, 5 μL detection oligo mix in the detection buffer is added, and assay plates are mixed and kept at ambient temperature for one hour, protected from light. The concentration of the Acceptor nucleotide oligo (e.g., ATTO647N-5′-ACAAAGAACCCTAACACCAG-3′ (SEQ ID NO: 1)) and Donor nucleotide oligo (e.g., bio-5′-AACACATCTCT(-bio)GCCAAACCCCA-bio-3′ (SEQ ID NO: 2)) in each assay well is 1 nM, and 3 nM, respectively.

After incubation with oligo mix, 5 μL of pre-blocked Europium-streptavidin reagent is dispensed to each assay well, assay plates are again mixed and kept at ambient temperature for one hour, protected from light.

The generated signal is measured with BMG Pherastar microtiter plate reader with a TRF light unit, using excitation at 340 nm, an integration time of 200 μs, and a delay time of 100 μs, before detection at 620 nm and 665 nm. The ratio of donor- and acceptor-fluorescence is used as a measure of the generated transcript product (i.e. enzymatic activity).

The IC₅₀ values are summarized in Table 1.

TABLE 1
IC₅₀ values for isoquinolinone compounds.

| Examples | IC₅₀ (μM) |
|---|---|
| Example 1 | 0.06* |
| Example 2 | 0.05* |
| Example 3 | 0.3 |
| Example 4 | 0.1 |
| Example 5 | 8 |
| Example 6 | 1 |
| Example 7 | 0.2 |
| Example 8 | 93 |
| Example 9 | 0.2 |
| Example 11 | 62 |
| Example 12 | 0.06* |
| Example 13 | 0.07* |
| Example 14 | 0.1* |
| Example 15 | 0.06* |
| Example 16 | 0.1* |
| Example 17 | 2* |
| Example 18 | 0.2* |
| Example 19 | 2* |
| Example 20 | 0.1* |
| Example 21 | 0.2* |
| Example 22 | 2* |
| Example 23 | 0.3* |
| Example 24 | 37* |
| Example 25 | >100* |
| Example 26 | 43* |
| Example 27 | 0.1* |
| Example 28 | 0.1* |
| Example 29 | 0.1* |
| Example 30 | 0.07* |
| Example 31 | 0.3* |
| Example 32 | 0.2* |
| Example 33 | 0.3* |
| Example 34 | 0.03* |
| Example 35 | 0.03* |
| Example 36 | 0.08* |
| Example 37 | 0.05* |
| Example 38 | 0.009 |
| Example 39 | 0.04 |
| Example 40 | 0.02 |
| Example 41 | 0.04 |
| Example 42 | 0.3 |
| Example 43 | 0.02 |
| Example 44 | 0.06 |
| Example 45 | 0.03 |
| Example 46 | 0.04 |
| Example 47 | 18 |
| Example 48 | 0.5 |
| Example 49 | >100 |
| Example 50 | 0.06 |
| Example 51 | 0.3 |
| Example 52 | 0.02 |
| Example 53 | 0.04 |
| Example 54 | 4 |
| Example 55 | 37 |
| Example 56 | 0.03 |
| Example 57 | 0.02 |
| Example 60 | 0.1 |
| Example 61 | 0.1 |
| Example 62 | 0.1 |
| Example 63 | >100 |
| Example 64 | 0.2 |
| Example 65 | 0.1 |
| Example 66 | 0.1 |
| Example 67 | 0.04 |
| Example 68 | 0.2 |
| Example 69 | 3 |
| Example 70 | 1 |
| Example 71 | 0.08 |
| Example 72 | 0.1 |
| Example 73 | 0.1 |
| Example 74 | 0.08 |
| Example 75 | 3 |
| Example 76 | 2 |
| Example 77 | 1 |
| Example 78 | 1* |

*aggregation of data sets

### General Protocol for In Vivo AML (Acute Myeloid Leukemia) Efficacy Experiment

### Determination of Maximum Tolerated Dose of Test Compound

Immunocompromised mice (6-10-week-old, female NSG mice, strain NOD. Cg-Prkdc^{scid} I12rg^{m1wjl}/Szj, Jackson Laboratories) are treated orally with test compound ranging from 75 to 150 mg/kg, once or twice per day for the duration of 14 days. Total body weight is measured, and the general condition of mice is monitored routinely. Mice with severe symptoms and moribund are excluded from study. Submental blood collection method (no anesthesia) is used for all samplings. Plasma levels of test compound are determined at intervals ranging from 0.5 to 4 hours post first and last doses in all dosing groups. From these data pharmacokinetic analysis are conducted.

### In Vivo Efficacy Study in AML Mouse Model

MV4-11 AML cell lines (ATCC) are labelled with luciferase tag by viral transduction procedure (MV4-11-luc).

For an AML cell line xenograft efficacy experiment, female NSG mice are given intravenously ˜1×10⁷ MV4-11-luc cells. Mice are flux sorted and randomized into treatment groups 14 days post transplantation. Mice are then treated with vehicle (50 mM Na₂HPO₄), or test compound at a tolerable dose determined from the above study, once or twice per day for 21 days. Tumor progression/regression is monitored by imaging of mice using luciferin as a substrate (150 mg/kg). Images are taken on a total of 9 time points i.e., one flux sort and once weekly to end date (8 time points). Imaging is performed under anesthesia and using in vivo imaging equipment IVIS. The treatment efficacy is also measured based on proportion of human AML cells, determined by flow cytometry analysis of viable human CD45 positive cell population in peripheral blood of mice one week post last dose. Plasma levels of test compound are determined at intervals ranging from 0.5 to 4 hours post last dose. Animals are monitored individually, and total body weight is measured routinely. The endpoint of the experiment is moribundity. In addition, mice demonstrating tumor-associated symptoms including impairment of hind limb function, ocular proptosis, and weight loss are considered for euthanasia. The remaining mice are euthanized on day 60 of the study.

1. A compound, a prodrug thereof, or a pharmaceutically acceptable salt thereof, represented by formula (I): 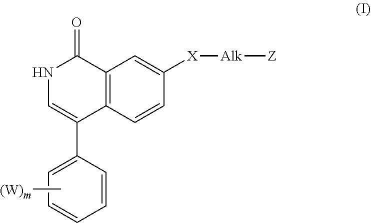
wherein:
W is selected from the group consisting of fluoro, chloro, trifluoromethyl, difluoromethyl, cyano, hydroxy O-Alk, trifluoronethoxv, C(O)NRR, SO₂R, and Alk, wherein the Alk is optionally substituted with one or more deuteriurm or fluoro, hydroxy, O—(C₃-C₆) cycloalkyl, trifluoromethyl, NRR, NRC(O)R, or alkoxy that is optionally substituted with one or more deuterium, wherein at least one W is located at the ortho position;
X is O or NR;
Alk is C₁-C₆ alkyl;
Z is: 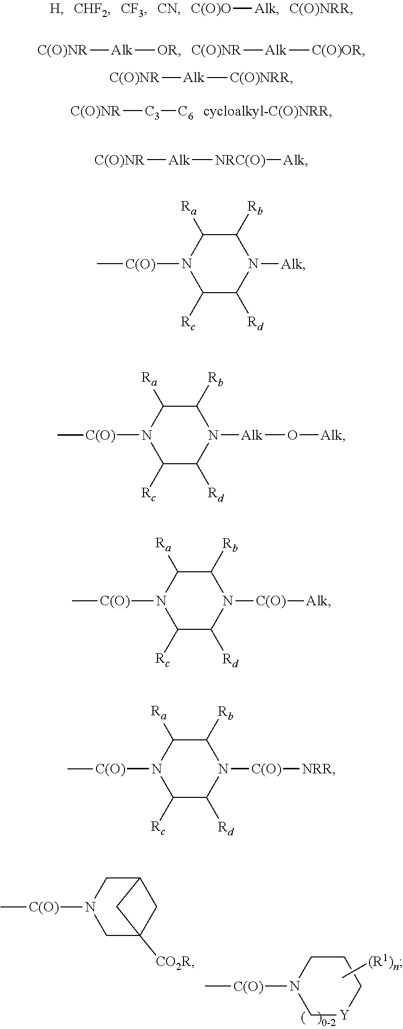
Y is O or CH₂;
R is H or C₁-C₆ alkyl optionally substituted with one or more fluoro groups;
Rₐ-R_{d} are independently selected from the group consisting of H, ═O, C(O)OR, and C(O)NRR, wherein either Rₐ or R_{b} optionally forms a 4- to 6-membered cyclic ring with either of R_{c} or R_{d};
R¹ is Alk, C₃-C₆ cycloalkyl, F, NRR, CO₂, C(O)NRR, NRC(O)R, or NRC(O)NRR, wherein Alk is optionally substituted with one or more deuterium or F, CO₂H, or C(O)NRR, and wherein C₃-C₆ cycloalkyl is substituted with CO₂H or C(O)NRR;
m is 1-5; and
n is 0-4.

2. The compound according to claim 1, a prodrug thereof, or a pharmaceutically acceptable salt thereof, wherein:
W is selected from the group consisting of fluoro, chloro, cyano, trifluoromethoxy, C(O)NRR, and Alk, wherein the Alk is optionally substituted with one or more deuterium or fluoro, hydroxy, NRR, NRC(O)R, or methoxy that is optionally substituted with one or more deuterium, wherein at least one W is located at the ortho position;
X is O or NR;
Alk is C₁-C₃ alkyl;
Z is: 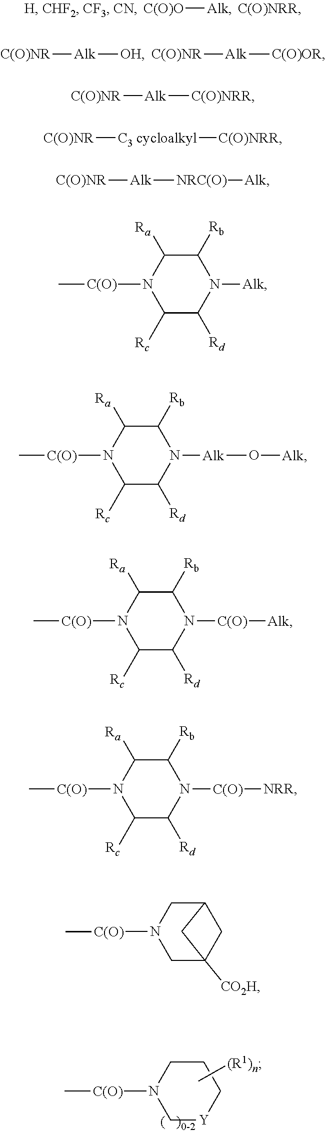
Y is O or CH₂;
R is H or C₁-C₂ alkyl optionally substituted with one or more fluoro groups;
Rₐ-R_{d} are independently selected from the group consisting of H and ═O, wherein either Rₐ or R_{b} optionally forms a 4- or 5-membered cyclic ring with either of R_{c} or Rₐ;
R¹ is Alk, C₃-C₄ cycloalkyl, F, NRR, CO₂H C(O)NRR, NRC(O)R, or NRC(O)NRR, wherein Alk is optionally substituted with one or more deuterium, CO₂H, or C(O)NRR, and wherein C₃-C₄ cycloalkyl is substituted with CO₂H or C(O)NRR;
m is 1-3; and
n is 1-4.

3. The compound according to claim 1, a prodrug thereof, or a pharmaceutically acceptable salt thereof, wherein:
W is selected from the group consisting of fluoro, chloro, cyano, and Alk, wherein the Alk is optionally substituted with hydroxy or one or more deuterium, wherein at least one W is located at the ortho position,
X is O or NR;
Alk is C₁-C₃ alkyl;
Z is: 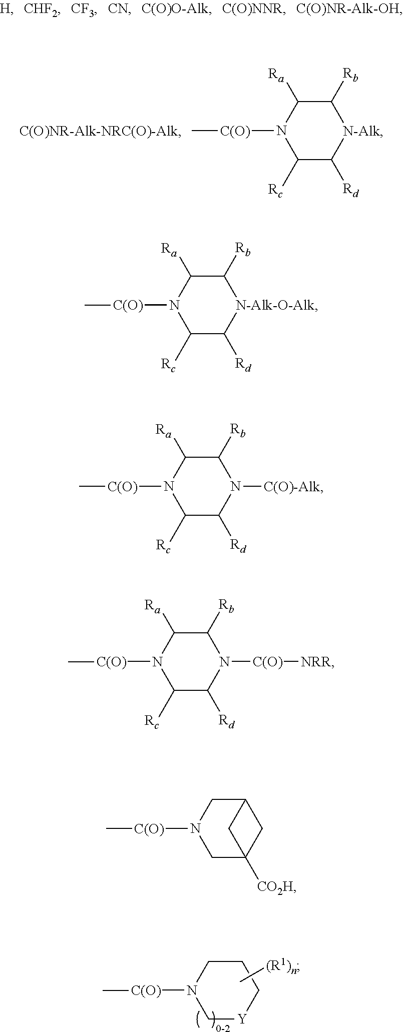
Y is CH₂;
R is H or C₁-C₂ alkyl;
Rₐ-R_{d} is H or either Rₐ or R_{b} optionally forms a 4- or 5-membered cyclic ring with either of R_{c} or R_{d};
R¹ is C₁-C₃ alkyl, F, CO₂H, C(O)NRR, or NRC(O)R;
m is 1-3; and
n is 1-4.

4. The compound according to claim 1, a prodrug thereof, or a pharmaceutically acceptable salt thereof, wherein:
W is selected from the group consisting of fluoro, chloro, and C₁ alkyl, wherein the C₁ alkyl is optionally substituted with one or more deuterium, wherein at least one W is located at the ortho position;
X is O or NR;
Alk is C₁-C₃ alkyl;
Z is: 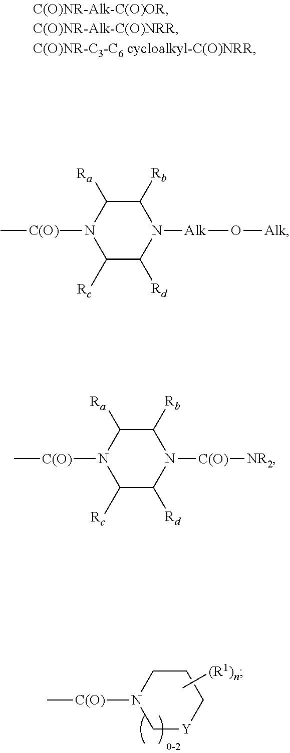
Y is O or CH₂;
R is H, C₁-C₂ alkyl optionally substituted with one or more fluoro groups;
Rₐ-R_{d} are independently selected from the group consisting of H and ═O;
R¹ is Alk, C₃-C₄ cycloalkyl, NRR, or NRC(O)NRR, wherein Alk is optionally substituted with one or more deuterium, CO₂H, or C(O)NRR, and wherein C₃-C₄ cycloalkyl is substituted with CO₂H or C(O)NRR;
m is 1 or 2, and
n is 1 or 2.

5. The compound according to claim 1, a prodrug thereof, or a pharmaceutically acceptable salt thereof, wherein:
W is selected from the group consisting of trifluoromethoxy, C(O)NRR, and C₁-C₂ alkyl, wherein the C₁-C₂alkyl is optionally substituted with one or more deuterium or fluoro, hydroxy, NRR, NRC(O)R, or methoxy that is optionally substituted with one or more deuterium, wherein at least one W is located at the ortho position;
X is O;
Alk is C₁-C₂ alkyl;
Z is CN;
R is H or C₁ alkyl; and
m is 1-2.

6. The compound according to claim 1, a prodrug thereof, or a pharmaceutically acceptable salt thereof, wherein:
W is C₁-C₂ alkyl optionally substituted with hydroxy, wherein at least one W is located at the ortho position;
X is O;
Alk is C₁-C₃ alkyl;
Z is: 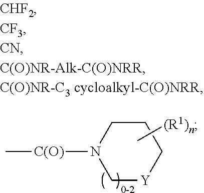
Y is CH₂;
R is H or C₁ alkyl;
R¹ is Alk, C₃ cycloalkyl, or CO₂H, wherein the C₃ cycloalkyl is substituted with CO₂H;
m is 1 or 2; and
n is 1 or 2.

7. The compound according to claim 1, a prodrug thereof or a pharmaceutically acceptable salt thereof, wherein:
W is C₁-C₂ alkyl optionally substituted with hydroxy, wherein at least one W is located at the ortho position;
X is O;
Alk is C₁-C₂ alkyl;
Z is: 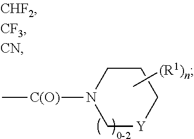
Y is CH₂;
R¹ is C₁-C₃ Alk or CO₂H;
m is 1 or 2; and
n is 2.

8. The compound according to claim 1, a prodrug thereof, or a pharmaceutically acceptable salt thereof, wherein:
W is C₁ alkyl, wherein the C₁ alkyl is located at the ortho position;
X is O;
Alk is C₁-C₂ alkyl;
Z is: 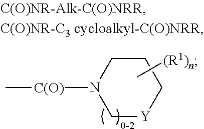
Y is CH₂,
R is H or C₁ alkyl;
R¹ is C₃ cycloalkyl substituted with CO₂H;
m is 1; and
n is 1.
